(19) United States
(12) Patent Application Publication
(10) Pub. No.: US 2024/0043829 A1
(43) Pub. Date: Feb. 8, 2024

(54) ZINC FINGER FUSION PROTEINS FOR NUCLEOBASE EDITING

(71) Applicant: Sangamo Therapeutics, Inc., Brisbane, CA (US)

(72) Inventors: Friedrich A. Fauser, Fremont, CA (US); Jeffrey C. Miller, San Rafael, CA (US); Sebastian Arangundy, Albany, CA (US)

(73) Assignee: Sangamo Therapeutics, Inc., Brisbane, CA (US)

(21) Appl. No.: 18/246,574

(22) PCT Filed: Sep. 24, 2021
(86) PCT No.: PCT/US2021/052088

§ 371 Date: Mar. 24, 2023

Related U.S. Application Data
(60) Provisional application No. 63/083,662, filed on Sep. 25, 2020.
(60) Provisional application No. 63/164,893, filed on Mar. 23, 2021.
(60) Provisional application No. 63/230,580, filed on Aug. 6, 2021.

(51) Int. Cl. C12N 15/10; C12N 9/78

(52) U.S. Cl. CPC C12N 15/102; C12N 9/78; C07K 2319/81; C12Y 305/04005; C12N 2310/20

(57) ABSTRACT

Provided herein are base editor systems comprising fusion proteins that comprise zinc finger protein and cytidine deaminase domains, as well as methods of using the base editor systems. The systems can be used to specifically alter a single base pair in a target DNA sequence.

### CROSS-REFERENCE TO RELATED APPLICATIONS

This application claims priority from U.S. Provisional Patent Application 63/083,662, filed Sep. 25, 2020; U.S. Provisional Patent Application 63/164,893, filed Mar. 23, 2021; and U.S. Provisional Patent Application 63/230,580, filed Aug. 6, 2021. The disclosures of those priority applications are incorporated by reference herein in their entirety.

### SEQUENCE LISTING

The instant application contains a Sequence Listing which has been submitted electronically in ASCII format and is hereby incorporated by reference in its entirety. The electronic copy of the Sequence Listing, created on Sep. 22, 2021, is named 025297_WO034_SL.txt and is 529,443 bytes in size.

### BACKGROUND OF THE INVENTION

Precision DNA editing of single bases has various applications in treating and understanding disorders such as genetic diseases. For example, knock-out of one or more genes can be achieved by converting regular codons into stop codons, or by mutating splice acceptor sites to introduce exon skipping and/or frameshift mutations. Further, DNA point mutations are associated with a wide range of disorders. Single base editing can be used to correct deleterious mutations or to introduce beneficial genetic modifications.

Cytidine deaminases convert the nucleobase cytosine to thymine (or the nucleoside deoxycytidine to thymidine). These enzymes function in the pyrimidine salvage pathway, predominantly operating on single-stranded DNA to convert cytosine into uracil, which is subsequently replaced by a thymine base during DNA replication or repair. A cytidine deaminase identified in the bacterium Burkholderia cenocepacia, DddA, can catalyze the deamination of cytosine to uracil within double-stranded DNA. DddA thus bypasses the requirement for unwinding of the dsDNA to ssDNA (Mok et al., Nature (2020) 583:631-7). While the Mok study reports C to T base editing at the human CCR5 locus with a DddA-derived cytosine base editor fused to transcription activator-like effector (TALE) proteins, it is unclear how broadly this approach is applicable. Further, new deaminases that operate on double-stranded DNA may have improved or altered base editing activity compared to DddA.

Thus, there continues to be a need to develop precise base editing systems for the prevention and treatment of numerous diseases.

### SUMMARY OF THE INVENTION

The present disclosure provides zinc finger protein (ZFP) based nucleobase editing systems and uses thereof. In one aspect, the present disclosure provides a system for changing a cytosine to a thymine in the genome of a cell (e.g., a eukaryotic cell or a prokaryotic cell, wherein the eukaryotic cell may be a mammalian cell such as a human cell, or a plant cell), comprising a first fusion protein and a second fusion protein, or first and second expression constructs for expressing the first and second fusion proteins, respectively, wherein a) the first fusion protein comprises: i) a first zinc finger protein (ZFP) domain that binds to a first sequence in a target genomic region in the cell, and ii) a first portion of a cytidine deaminase polypeptide (e.g., wherein the cytidine deaminase is a toxin-derived deaminase (TDD) comprising an amino acid sequence at least 90% identical to SEQ ID NO: 49, 81, 92, 95, 98, 101, 104, 107, 134, 143, 152, 157, 162, 167, 172, 177, 184, 189, 194, 199, 204, 209, 214, or 219); b) the second fusion protein comprises: i) a second ZFP domain that binds to a second sequence in the target genomic region, and ii) a second portion of the cytidine deaminase polypeptide; and c) binding of the first fusion protein and the second fusion protein to the target genomic region results in dimerization of the first and second portions, wherein the dimerized portions form an active cytidine deaminase capable of changing a cytosine to a uracil in the target genomic region. In some embodiments, the first and second portions lack cytidine deaminase activity on their own. In some embodiments, the first and second portions form an active cytidine deaminase that comprises an amino acid sequence at least 90% identical to SEQ ID NO: 49, 81, 92, 95, 98, 101, 104, 107, 134, 143, 152, 157, 162, 167, 172, 177, 184, 189, 194, 199, 204, 209, 214, or 219. In some embodiments, the first and second portions form an active cytidine deaminase that comprises the amino acid sequence of SEQ ID NO: 49, 81, 92, 95, 98, 101, 104, 107, 134, 143, 152, 157, 162, 167, 172, 177, 184, 189, 194, 199, 204, 209, 214, or 219. In some embodiments, the target genomic region may be specific to a particular allele of a gene in the cell. In some embodiments, the targeted cytosine may be between the proximal ends of the first sequence and the second sequence in the target genomic region, optionally wherein the proximal ends are no more than 100 bps apart.

Also provided are multiplex versions of the present base editor systems comprising more than one pair of the first and second fusion proteins, wherein each pair of the fusion proteins binds to a different target genomic region, optionally wherein the first and second cytidine deaminase portions of one pair of fusion proteins are different from the first and second portions of another pair of fusion proteins.

In some embodiments, the base editor system further comprises a nickase that creates a single-stranded DNA break on the unedited or edited strand, wherein the DNA break is no more than about 500 bps, optionally no more than 200 bps, optionally about 10-50 bps, from the cytosine to be edited. The nickase may be, e.g., a ZFP-based nickase, a TALE-based nickase, or a CRISPR-based nickase. In some embodiments, the nickase is a ZFP-based nickase formed by dimerization of a first nickase domain and a second nickase domain fused respectively to two ZFP domains that bind to the target genomic region, wherein the first and second nickase domains are inactive, or lack significant or specific nickase activity, on their own. In certain embodiments, one of the nickase domains is fused to the first or second ZFP-cytidine deaminase fusion protein, and the other nickase domain is fused to a third ZFP domain that binds to a third sequence in the target genomic region. Alternatively, the two nickase domains may be fused respectively to a third ZFP domain that binds a third sequence in the target genomic region and a fourth ZFP domain that binds a fourth sequence in the target genomic region. In particular embodiments, the first and second nickase domains are derived from FokI.

In some embodiments, the base editor system further comprises an inhibitory component of the cytidine deaminase, e.g., a toxin-derived deaminase inhibitor (TDDI) where the cytidine deaminase is a TDD. For example, the inhibitor may be a DddI component where the cytidine deaminase is DddA. In certain embodiments, this system comprises a third fusion protein or a third expression construct for expressing the third fusion protein in the cell, wherein the third fusion protein comprises i) a ZFP domain that binds to a third sequence in the target genomic region, and ii) an inhibitory domain for the cytidine deaminase (e.g., a TDDI where the cytidine deaminase is a TDD, such as DddI where the cytidine deaminase is DddA), and binding of the third fusion protein to the target genomic region results in the interaction of the inhibitory domain with, and thereby inhibition of the cytidine deaminase activity of, the dimerized cytidine deaminase portions.

In some embodiments of the inhibitory domain-containing base editor system, the system comprises a third fusion protein or a third expression construct for expressing the third fusion protein in the cell, and a fourth fusion protein or a fourth expression construct for expressing the fourth fusion protein in the cell, wherein the third fusion protein comprises i) a ZFP domain that binds to a third sequence in the target genomic region, and ii) a first dimerization domain; and the fourth fusion protein comprises i) an inhibitory domain for the cytidine deaminase (e.g., a TDDI where the cytidine deaminase is a TDD, such as DddI where the cytidine deaminase is DddA), and ii) a second dimerization domain capable of partnering with the first dimerization domain in the presence of a dimerization-inducing agent; and binding of the third fusion protein to the target genomic region and dimerization of the third and fourth fusion proteins result in the binding of the inhibitory domain to, and thereby inhibition of the cytidine deaminase activity of, the dimerized cytidine deaminase portions.

In some embodiments of the inhibitory domain-containing base editor system, the system comprises a third fusion protein or a third expression construct for expressing the third fusion protein in the cell, and a fourth fusion protein or a fourth expression construct for expressing the fourth fusion protein in the cell, wherein the third fusion protein comprises i) a ZFP domain that binds to a third sequence in the target genomic region, and ii) a first dimerization domain; and the fourth fusion protein comprises i) an inhibitory domain for the cytidine deaminase (e.g., a TDDI where the cytidine deaminase is a TDD, such as DddI where the cytidine deaminase is DddA), and ii) a second dimerization domain capable of partnering with the first dimerization domain in the absence of a dimerization-inhibiting agent; and binding of the third fusion protein to the target genomic region, and dimerization of the third and fourth fusion proteins, result in the binding of the inhibitory domain to, and thereby inhibition of the cytidine deaminase activity of, the dimerized cytidine deaminase portions.

In particular embodiments, the base editor systems described herein comprise both a nickase component and an inhibitory domain component described herein.

Any of the ZFP domains used in the fusion proteins described herein may independently have 2, 3, 4, 5, 6, 7, or 8 zinc fingers.

In some embodiments, the protein components of the present base editor systems are provided to the cells by means of expression cassettes or constructs. Such cassettes or constructs may be provided to the cells on the same or separate expression vectors such as viral vectors. The viral vectors may be, e.g., adeno-associated viral (AAV) vectors, adenoviral vectors, or lentiviral vectors.

In some embodiments of the base editor systems described herein, the cytidine deaminase is a TDD. In certain embodiments, the TDD comprises the amino acid sequence of SEQ ID NO: 72 (DddA), or the toxic domain of a TDD comprising said sequence (e.g., the toxic domain of SEQ ID NO: 49 or 81). In some embodiments, the cytidine deaminase is a TDD that comprises an amino acid sequence at least 20%, 30%, 40%, 50%, 60%, 70%, 80%, 90%, 92%, 94%, 95%, 96%, 97%, 98%, or 99% identical to the amino acid sequence of SEQ ID NO: 49 or 81. In certain embodiments, the first DddA portion comprises amino acids 1264-1333, 1264-1397, 1264-1404, 1264-1407, or a fragment thereof, of amino acids 1264-1427 of SEQ ID NO: 72; and the second DddA portion comprises the remainder, or a fragment thereof, of said amino acids of SEQ ID NO: 72; or vice versa; wherein the two portions form a functional cytidine deaminase. In certain embodiments, the first DddA portion comprises amino acids 1290-1333, 1290-1397, 1290-1404, 1290-1407, or a fragment thereof, of amino acids 1290-1427 of SEQ ID NO: 72; and the second DddA portion comprises the remainder, or a fragment thereof, of said amino acids of SEQ ID NO: 72; or vice versa; wherein the two portions form a functional cytidine deaminase. In some embodiments, the first and second DddA portions respectively comprise SEQ ID NOs: 82 and 83, SEQ ID NOs: 84 and 85, SEQ ID NOs: 18 and 19, SEQ ID NOs: 51 and 52, or SEQ ID NOs: 53 and 54; or vice versa.

In some embodiments of the base editor systems described herein, the cytidine deaminase is DddA that has a mutation at one or more residues selected from Y1307, T1311, S1331, V1346, H1366, N1367, N1368, P1369, E1370, G1371, T1372, F1375, V1392, P1394, P1395, 11399, P1400, V1401, K1402, A1405, and T1406 in SEQ ID NO: 72.

In some embodiments of the base editor systems described herein, the cytidine deaminase is a TDD that comprises the amino acid sequence of any one of SEQ ID NOs: 86-91 and 117-129. In certain embodiments, the cytidine deaminase comprises the toxic domain of a TDD comprising the amino acid sequence of any one of SEQ ID NOs: 86-91 and 117-129. In certain embodiments, the TDD comprises an amino acid sequence at least 20%, 30%, 40%, 50%, 60%, 70%, 80%, 90%, 92%, 94%, 95%, 96%, 97%, 98%, or 99% identical to the amino acid sequence of SEQ ID NO: 92, 95, 98, 101, 104, 107, 134, 143, 152, 157, 162, 167, 172, 177, 184, 189, 194, 199, 204, 209, 214, or 219. In particular embodiments, the cytidine deaminase is a TDD that comprises the amino acid sequence of SEQ ID NO: 92, 95, 98, 101, 104, 107, 134, 143, 152, 157, 162, 167, 172, 177, 184, 189, 194, 199, 204, 209, 214, or 219. In particular embodiments, the first and second cytidine deaminase portions respectively comprise SEQ ID NOs: 93 and 94, SEQ ID NOs: 96 and 97, SEQ ID NOs: 99 and 100, SEQ ID NOs: 102 and 103, SEQ ID NOs: 105 and 106, SEQ ID NOs: 108 and 109, SEQ ID NOs: 130 and 131, SEQ ID NOs: 132 and 133, SEQ ID NOs: 135 and 136, SEQ ID NOs: 137 and 138, SEQ ID NOs: 139 and 140, SEQ ID NOs: 141 and 142, SEQ ID NOs: 144 and 145, SEQ ID NOs: 146 and 147, SEQ ID NOs: 148 and 149, SEQ ID NOs: 150 and 151, SEQ ID NOs: 153 and 154, SEQ ID NOs: 155 and 156, SEQ ID NOs: 158 and 159, SEQ ID NOs: 160 and 161, SEQ ID NOs: 163 and 164, SEQ ID NOs: 165 and 166, SEQ ID NOs: 168 and 169, SEQ ID NOs: 170 and 171, SEQ ID NOs: 173 and 174, SEQ ID NOs: 175 and 176, SEQ ID NOs: 178 and 179, SEQ ID NOs: 180 and 181, SEQ ID NOs: 182 and 183, SEQ ID NOs: 185 and 186, SEQ ID NOs: 187 and 188, SEQ ID NOs: 190 and 191, SEQ ID NOs: 192 and 193, SEQ ID NOs: 195 and 196, SEQ ID NOs: 197 and 198, SEQ ID NOs: 200 and 201, SEQ ID NOs: 202 and 203, SEQ ID NOs: 205 and 206, SEQ ID NOs: 207 and 208, SEQ ID NOs: 210 and 211, SEQ ID NOs: 212 and 213, SEQ ID NOs: 215 and 216, SEQ ID NOs: 217 and 218, SEQ ID NOs: 220 and 221, or SEQ ID NOs: 222 and 223; or vice versa.

In a related aspect, the present disclosure also provides a fusion protein comprising i) a zinc finger protein (ZFP) domain that binds to gene (which may be a eukaryotic, e.g., human, gene) and ii) a cytidine deaminase polypeptide or a fragment thereof, e.g., wherein the cytidine deaminase is a TDD comprising an amino acid sequence at least 90% identical to SEQ ID NO: 49, 81, 92, 95, 98, 101, 104, 107, 134, 143, 152, 157, 162, 167, 172, 177, 184, 189, 194, 199, 204, 209, 214, or 219, optionally wherein the ZFP domain and the cytidine deaminase or fragment thereof are linked by a peptide linker. In some embodiments, the TDD comprises the amino acid sequence of SEQ ID NO: 49, 81, 92, 95, 98, 101, 104, 107, 134, 143, 152, 157, 162, 167, 172, 177, 184, 189, 194, 199, 204, 209, 214, or 219.

In a related aspect, the present disclosure provides a fusion protein comprising i) a zinc finger protein (ZFP) domain that binds to a gene (which may be a eukaryotic, e.g., human, gene), and ii) a cytidine deaminase inhibitory domain, e.g., wherein the cytidine deaminase is a TDD comprising an amino acid sequence at least 90% identical to SEQ ID NO: 49, 81, 92, 95, 98, 101, 104, 107, 134, 143, 152, 157, 162, 167, 172, 177, 184, 189, 194, 199, 204, 209, 214, or 219, optionally wherein the ZFP domain and the inhibitory domain are linked by a peptide linker. In some embodiments, the cytidine deaminase inhibitory domain is a TDDI, such as DddI where the cytidine deaminase is DddA. In some embodiments, the TDD comprises the amino acid sequence of SEQ ID NO: 49, 81, 92, 95, 98, 101, 104, 107, 134, 143, 152, 157, 162, 167, 172, 177, 184, 189, 194, 199, 204, 209, 214, or 219.

In a related aspect, the present disclosure provides a fusion protein comprising i) a zinc finger protein (ZFP) domain that binds to a gene (which may be a eukaryotic, e.g., human, gene), and ii) a nickase or a fragment thereof, optionally wherein the ZFP domain and the nickase or fragment thereof are linked by a peptide linker.

In one aspect, the present disclosure provides a pair of fusion proteins comprising a) a first fusion protein that comprises i) a zinc finger protein (ZFP) domain that binds to a gene (which may be a eukaryotic, e.g., human, gene), and ii) a first dimerization domain, and b) a second fusion protein that comprises i) a cytidine deaminase inhibitory domain, e.g., wherein the cytidine deaminase is a TDD comprising an amino acid sequence at least 90% identical to SEQ ID NO: 49, 81, 92, 95, 98, 101, 104, 107, 134, 143, 152, 157, 162, 167, 172, 177, 184, 189, 194, 199, 204, 209, 214, or 219, and ii) a second dimerization domain, wherein the first and second dimerization domains can dimerize in the presence of a dimerization-inducing agent. In some embodiments, the cytidine deaminase inhibitory domain is a TDDI, such as DddI where the cytidine deaminase is DddA. In some embodiments, the TDD comprises the amino acid sequence of SEQ ID NO: 49, 81, 92, 95, 98, 101, 104, 107, 134, 143, 152, 157, 162, 167, 172, 177, 184, 189, 194, 199, 204, 209, 214, or 219.

In another aspect, the present disclosure provides a pair of fusion proteins comprising a) a first fusion protein that comprises i) a zinc finger protein (ZFP) domain that binds to a gene (which may be a eukaryotic, e.g., human, gene), and ii) a first dimerization domain, and b) a second fusion protein that comprises i) a cytidine deaminase inhibitory domain, e.g., wherein the cytidine deaminase is a TDD comprising an amino acid sequence at least 90% identical to SEQ ID NO: 49, 81, 92, 95, 98, 101, 104, 107, 134, 143, 152, 157, 162, 167, 172, 177, 184, 189, 194, 199, 204, 209, 214, or 219, and ii) a second dimerization domain, wherein the first and second dimerization domains can dimerize in the absence of a dimerization-inhibiting agent. In some embodiments, the cytidine deaminase inhibitory domain is a TDDI, such as DddI where the cytidine deaminase is DddA. In some embodiments, the TDD comprises the amino acid sequence of SEQ ID NO: 49, 81, 92, 95, 98, 101, 104, 107, 134, 143, 152, 157, 162, 167, 172, 177, 184, 189, 194, 199, 204, 209, 214, or 219.

In one aspect, the present disclosure provides one or more nucleic acid molecules encoding the fusion protein(s) described herein, as well as expression constructs comprising the nucleic acid molecule(s) and viral vectors comprising the expression construct(s), optionally wherein the viral vectors may be an adeno-associated viral vector, an adenoviral vector, or a lentiviral vector. Also provided is a cell (which may be a eukaryotic cell, e.g., a mammalian cell or a plant cell) comprising a base editor system as described herein, fusion protein(s) as described herein, isolated nucleic acid molecule(s) as described herein, expression construct(s) as described herein, or viral vector(s) as described herein. In some embodiments, the mammalian cell is a human cell, such as a human embryonic stem or a human induced pluripotent stem cell.

In some aspects, the present disclosure provides a method of changing a cytosine to a thymine in a target genomic region in a cell (which may be a eukaryotic cell, e.g., a mammalian or plant cell), comprising delivering a base editor system as described herein to the cell. In some embodiments, the change of the cytosine to the thymine creates a stop codon in the target genomic region. A multiplex format of the system may target more than one genomic region (e.g., 2, 3, 4, or 5 genomic regions). The editing may be performed in vivo, ex vivo, or in vitro.

Also provided are genetically engineered cells (which may be eukaryotic cells, e.g., mammalian cells such as human iPSCs or plant cells) obtained by the present editing methods.

Engineered cells described herein (e.g., engineered human cells), including pharmaceutical compositions comprising the cells and a pharmaceutically acceptable carrier, may be used for treating a patient in need thereof (e.g., a human patient in need thereof) or used in the manufacture of a medicament for treating a patient in need thereof. In some embodiments, the patient has cancer, an autoimmune disorder, an autosomal dominant disease, or a mitochondrial disorder. In some embodiments, the patient has sickle cell disease, hemophilia, cystic fibrosis, phenylketonuria, Tay-Sachs, prion disease, color blindness, a lysosomal storage disease, Friedreich's ataxia, or prostate cancer. Kits and articles of manufacture comprising the cells are also contemplated.

Other features, objects, and advantages of the invention are apparent in the detailed description that follows. It should be understood, however, that the detailed description, while indicating embodiments and aspects of the invention, is given by way of illustration only, not limitation. Various changes and modification within the scope of the invention will become apparent to those skilled in the art from the detailed description.

### BRIEF DESCRIPTION OF THE FIGURES

FIG. 1 is a schematic illustrating a pair of ZFP-TDD fusion proteins for C to T base editing. The rectangles represent DNA-binding zinc fingers in the ZFP domains of the fusion proteins. The arrow shapes above the underlined C nucleotide represent dimerized TDD domains of the fusion proteins. The black lines between the zinc finger domains and the TDD domains represent peptide linkers.

FIG. 2A is a schematic showing ZFP designs for CCR5-targeting ZFP-TDD fusion protein pairs. C9, C10, C18, and C24 are target nucleotides for base editing. Top strand (left to right): SEQ ID NO: 227. Bottom strand (right to left): SEQ ID NO: 228.

FIG. 2B is a schematic showing an example of a construct design for a dimerized ZFP-DddA pair. FLAG: FLAG tag. NLS: nuclear localization sequence. UGI: uracil DNA glycosylase inhibitor.

FIG. 3 is a table showing the heatmap results of C to T base editing at a human CCR5 locus by a series of ZFP-DddA fusion protein pairs. The degree of editing activity corresponds to the darkness of shading within a cell. L0, L7A, and L26 represent peptide linkers used to fuse the DddA domain to the C-terminus of the ZFP domain in the fusion protein.

FIG. 4 is a table showing the heatmap results of C to T base editing at a human CCR5 locus by a series of ZFP-DddA fusion protein pairs, wherein the DddA split occurs at different positions. The degree of editing activity corresponds to the darkness of shading within a cell.

FIG. 5 is a schematic showing ZFP designs for CCR5-targeting ZFP-TDD fusion proteins. C9, C10, C18, and C24 are target nucleotides for base editing. From top to bottom: SEQ ID NO: 229 (left to right), SEQ ID NO: 230 (right to left), SEQ ID NO: 231 (left to right), SEQ ID NO: 232 (right to left), SEQ ID NO: 233 (left to right), and SEQ ID NO: 234 (right to left).

FIGS. 6A-6C are tables showing the heatmap results of C to T base editing at a human CCR5 locus by a series of ZFP-DddA fusion protein pairs with the indicated DddA mutations. The mutations are numbered with respect to SEQ ID NO: 72. The degree of editing activity corresponds to the darkness of shading within a cell

FIG. 7A is a schematic illustrating the combined use of the ZFP-TDD base editing system and a nickase system for increasing base editing efficiency. The nickase system shown here is a CRISPR/Cas-based nickase system. The illustrative gene locus is a human CCR5 locus. Top strand (left to right): SEQ ID NO: 235. Bottom strand (right to left): SEQ ID NO: 236.

FIG. 7B is a table showing the heatmap results of DddA C to T base editing at a human CCR5 locus using the approach of FIG. 7A. The degree of editing activity corresponds to the darkness of shading within a cell.

FIG. 8 is a schematic illustrating the combined use of the ZFP-TDD base editing system and a CRISPR/Cas-based nickase system.

FIG. 9 is a schematic illustrating an example of a trimeric ZFP-TDD+FokI nickase base editing system.

FIG. 10 is a schematic showing ZFP designs for combined use of CCR5-targeting ZFP-TDD fusion protein pairs with a ZFP-nickase. C9, C10, C18, and C24 are target nucleotides for base editing. Top strand (left to right): SEQ ID NO: 237. Bottom strand (right to left): SEQ ID NO: 238.

FIG. 11 is a table showing the heatmap results of DddA C to T base editing at a human CCR5 locus using the approach of FIG. 10. The degree of editing activity corresponds to the darkness of shading within a cell.

FIG. 12 is a table showing the heatmap results of C to T base editing at a human CCR5 locus by a series of ZFP-TDD fusion protein pairs. The degree of editing activity corresponds to the darkness of shading within a cell. O1: TDD1; O2: TDD2; O3: TDD3; O4: TDD4; O5: TDD5; O6: TDD6.

FIG. 13 is a table showing the heatmap results of the highest frequency of C to T base editing for any C in the CCR5 base editing window by ZFP fusion protein pairs with TDD1-TDD6. O1: TDD1; O2: TDD2; O3: TDD3; O4: TDD4; O5: TDD5; O6: TDD6.

FIG. 14 is a table showing the heatmap results of the highest frequency of C to T base editing for any C in the CCR5 base editing window by ZFP fusion protein pairs with TDD1-TDD6. O1: TDD1; O2: TDD2; O3: TDD3; O4: TDD4; O5: TDD5; O6: TDD6.

FIG. 15 is a schematic showing ZFP designs for CITTA-targeting ZFP-TDD fusion protein pairs. G2, G5, C6, C8, G10, G11, G14, C15, and C16 are target nucleotides for base editing. Top strand (left to right): SEQ ID NO: 239. Bottom strand (right to left): SEQ ID NO: 240.

FIG. 16 is a table showing the heatmap results of the highest frequency of C to T base editing at a human CIITA locus (“site 2”) by a series of ZFP-TDD fusion protein pairs. The degree of editing activity corresponds to the darkness of shading within a cell. O1: TDD1; O14: TDD14; etc.

FIG. 17 is a table showing the heatmap results of the highest frequency of C to T base editing for any C (underlined) in the CIITA base editing window and its sequence motif for DddA, TDD4, TDD6, TDDS, TDD10, TDD14, TDD15 and TDD18. Amplicon: SEQ ID NO: 244. O4: TDD4; O6: TDD6; etc.

FIG. 18 is a table showing the heatmap results of C to T base editing at a human CIITA locus (“site 2”) by a ZFP fusion protein pair with TDD6 or TDD14. L26, L21, L18, L13, L11, L9, L6, and L4 represent peptide linkers used to fuse the TDD6 or TDD14 domain to the C-terminus of the ZFP domain in the fusion protein. The degree of editing activity corresponds to the darkness of shading within a cell. O6: TDD6; O14: TDD14.

FIG. 19 is a schematic illustrating a design for inhibition of a TDD with a targeted ZFP-TDDI.

### DETAILED DESCRIPTION OF THE INVENTION

The present disclosure provides systems and methods for base editing, e.g., from cytosine (C) to thymine (T), in cellular DNA such as genomic DNA. The systems entail the use of ZFP-toxin-derived deaminase (TDD) fusion proteins (ZFP-TDDs). By providing precise gene editing in a cellular context, the present systems and methods can be used for the prevention and/or treatment of numerous diseases. It is contemplated that these systems and methods will be particularly useful for cell-based therapies that require the simultaneous knock-out of multiple human genes.

The present systems and methods can convert targeted C:G base pairs to T:A base pairs. In some embodiments, the base editing systems may also include proteins (e.g., UGI) that increase the stability of the conversion, and/or endonucleases that nick the DNA near the targeted base so as to stimulate DNA repair in the edited region and to promote the correction of the G nucleotide on the opposite strand to A, forming the edited T:A base pair.

The present systems and methods are advantageous in part due to the compact size of the ZFP domains in the fusion proteins. In comparison, the large physical size of a TALE and the long C-terminal TALE linker may limit how small the base editing window can be, as well as design density. The size and highly repetitive nature of engineered TALEs also make it challenging to deliver TALE-based base editors to human cells using common viral vectors. The present ZFP-derived base editing systems circumvent these problems. For instance, the compactness of these ZFP-derived systems may allow for packaging within a single AAV vector, in contrast to TALE base editor systems (e.g., TALE-TDDs) or CRISPR/Cas base editor systems. In addition, due to the small size of the fusion proteins herein, it is possible to include a nickase in the editing system so as to allow the generation of a DNA nick near the edited base and thereby facilitate the DNA repair machinery to change the base opposite the edited C from G to a corresponding A, forming the correct T:A base pair. The inclusion of a nickase may greatly increase the base editing efficiency.

### I. Zinc-Finger Fusion Proteins

Provided are fusion proteins that contain a DNA-binding zinc finger protein (ZFP) domain fused to a base editor domain (e.g., a cytidine deaminase domain, which may be a TDD such as one described herein), a cytidine deaminase inhibitor (e.g., a TDDI, such as DddI where the cytidine deaminase is DddA) domain, and/or a nickase domain (e.g., a FokI domain). As used herein, a “fusion protein” refers to a polypeptide where heterologous functional domains (i.e., functional domains that are not naturally present in the same protein in nature) are covalently linked (e.g., through peptidyl bonds). These fusion proteins, which can be recombinantly made, are components of the present base editor systems. In some embodiments, a ZFP fusion protein herein comprises a cytidine deaminase domain (e.g., derived from a TDD as described herein) and additionally a nickase domain and/or a UGI domain.

Other formats of the present systems also are contemplated herein. For example, instead of peptidyl links, two functional domains may be brought together by noncovalent bonds. In some embodiments, two functional domains (e.g., a ZFP domain and a cytidine deaminase inhibitor domain; or a ZFP domain and a nickase domain) each are fused to a dimerization partner (e.g., leucine zipper and those described further herein), such that the two functional domains are brought together through interaction of the dimerization partners. In certain embodiments, the dimerization of these domains may be controlled by the presence or absence of a specific agent (e.g., a small molecule or peptide). It is contemplated that such formats may substitute for fusion proteins in any aspect of the present invention.

Each component of the present base editor systems is further described in detail below.

### A. Base Editors

The ZFP-cytidine deaminase fusion proteins of the present disclosure comprise a cytidine deaminase domain in addition to a ZFP domain. The term “deaminase” or “deaminase domain,” as used herein, refers to a protein that catalyzes a deamination reaction. A cytidine deaminase domain, for example, may catalyze the deamination of cytosine to uracil, wherein the uracil is replaced by a thymine base during DNA replication or repair. The deaminase domain may be naturally-occurring or may be engineered. In some embodiments, a cytidine deaminase of the present disclosure operates on double-stranded DNA.

In some embodiments, the cytidine deaminase is derived from a toxin that may be, e.g., from a prokaryotic or eukaryotic organism. In certain embodiments, the organism may be bacteria or fungus. Such a cytidine deaminase is referred to herein as a toxin-derived deaminase (TDD). DddA and DddA orthologs are TDDs. As used herein, a cytidine deaminase “derived from” a toxin may refer to a cytidine deaminase that is the same as the naturally occurring toxin or is a modified version of the toxin that retains deaminase activity.

In some embodiments, the cytidine deaminase is DddA (SEQ ID NO: 72). In certain embodiments, the cytidine deaminase comprises the toxic domain (e.g., amino acids 1290-1427 (SEQ ID NO: 49) or 1264-1427 (SEQ ID NO: 81)) of DddA, and the fusion protein is termed ZFP-DddA. An exemplary full sequence of the DddA protein derived from Burkholderia cenocepacia is shown below:

```
(SEQ ID NO: 72)
                10 20 30 40 50
MYEAARVTDP IDHTSALAGF LVGAVLGIAL IAAVAFATFT CGFGVALLAG
                60 70 80 90 100
MMAGIGAQAL LSIGESIGKM FSSQSGNIIT GSPDVYVNSL SAAYATLSGV
              110 120 130 140 150
ACSKHNPIPL VAQGSTNIFI NGRPAARKDD KITCGATIGD GSHDTFFHGG
              160 170 180 190 200
TQTYLPVDDE VPPWLRTATD WAFTLAGLVG GLGGLLKASG GLSRAVLPCA
              210 220 230 240 250
AKFIGGYVLG EAFGRYVAGP AINKAIGGLF GNPIDVTTGR KILLAESETD
              260 270 280 290 300
YVIPSPLPVA IKRFYSSGID YAGTLGRGWV LPWEIRLHAR DGRLWYTDAQ
              310 320 330 340 350
GRESGFPMLR AGQAAFSEAD QRYLTRTPDG RYILHDLGER YYDEGQYDPE
              360 370 380 390 400
SGRIAWVRRV EDQAGQWYQF ERDSRGRVTE ILTCGGLRAV LDYETVEGRL
              410 420 430 440 450
GTVTLVHEDE RRLAVTYGYD ENGQLASVTD ANGAGVRQFA YINGLMTNHM
              460 470 480 490 500
NALGETSSYV WSKIEGEPRV VETHTSEGEN WIFEYDVAGR QTRVRHADGR
              510 520 530 540 550
TAHWRFDAQS QIVEYTDLDG AFYRIKYDAV GMPVMLMLPG DRTVMFEYDD
              560 570 580 590 600
AGRIIAETDP LGRTTRTRYD GNSLRPVEVV GPDGGAWRVE YDQQGRVVSN
              610 620 630 640 650
QDSLGRENRY EYPKALTALP SAHIDALGGR KTLEWNSLGK LVGYTDCSGK
              660 670 680 690 700
TTRTSFDAFG RICSRENALG QRITYDVRPT GEPRRVTYPD GSSETFEYDA
              710 720 730 740 750
AGTLVRYIGL GGRVQELLRN ARGQLIEAVD PAGRRVQYRY DVEGRLRELQ
              760 770 780 790 800
QDHARYTFTY SAGGRLLTET RPDGILRRFE YGEAGELLGL DIVGAPDPHA
              810 820 830 840 850
TGNRSVRTIR FERDRMGVLK VQRTPTEVTR YQHDKGDRLV KVERVPTPSG
              860 870 880 890 900
IALGIVPDAV EFEYDKGGRL VAEHGSNGSV IYTLDELDNV VSLGLPHDQT
              910 920 930 940 950
LQMLRYGSGH VHQIREGDQV VADFERDDLH REVSRTQGRL TQRSGYDPLG
              960 970 980 990 1000
RKVWQSAGID PEMLGRGSGQ LWRNYGYDAA GDLIETSDSL RGSTRESYDP
            1010 1020 1030 1040 1050
AGRLISRANP LDRKFEEFAW DAAGNLLDDA QRKSRGYVEG NRLLMWQDLR
            1060 1070 1080 1090 1100
FEYDPFGNLA TKRRGANQTQ RFTYDGQDRL ITVHTQDVRG VVETRFAYDP
            1110 1120 1130 1140 1150
LGRRIAKTDT AFDLRGMKLR AETKREVWEG LRLVQEVRET GVSSYVYSPD
            1160 1170 1180 1190 1200
APYSPVARAD TVMAEALAAT VIDSAKRAAR IFHFHTDPVG ALQEVTDEAG
            1210 1220 1230 1240 1250
EVAWAGQYAA WGKVEATNRG VTAARTDQPL RFAGQYADDS TGLHYNTERF
            1260 1270 1280 1290 1300
YDPDVGRFIN QDPIGLNGGA NVYHYAPNPV GWVDPWGLAG SYALGPYQIS
            1310 1320 1330 1340 1350
APQLPAYNGQ TVGTFYYVND AGGLESKVFS SGGPTPYPNY ANAGHVEGQS
            1360 1370 1380 1390 1400
ALFMRDNGIS EGLVFHNNPE GTCGFCVNMT ETLLPENAKM TVVPPEGAIP
            1410 1420
VKRGATGETK VFTGNSNSPK SPTKGGC
```

As used herein, unless specified otherwise, the term “DddA” refers to the DddA toxic domain.

In certain embodiments, the cytidine deaminase is a “re-wired” version of DddA (e.g., SEQ ID NO: 50).

The present disclosure also provides variants of DddA mutated at residues that form the nucleotide pocket (e.g., Y1307, T1311, 51331, V1346, H1366, N1367, N1368, P1369, E1370, G1371, T1372, F1375, V1392, P1394, P1395, 11399, P1400, V1401, K1402, A1405, T1406, or any combination thereof, wherein the numbering of the residues is with respect to SEQ ID NO: 72). The DddA may be mutated, for example, at 1, 2, 3, 4, 5, 6, 7, 8, 9, 10, 11, 12, 13, 14, 15, 16, 17, 18, 19, 20, 21, or 22 of said residues. In some embodiments, DddA is mutated at residue E1370, N1368, Y1307, T1311, 51331, K1402, or any combination thereof. In certain embodiments, DddA is mutated at residue E1370, N1368, Y1307, or any combination thereof. In certain embodiments, the mutation(s) may increase DddA efficiency, increase DddA activity, change the DddA activity window, or any combination thereof. It is contemplated that such variants may substitute for wild-type DddA in any aspect of the present invention.

In particular embodiments, the cytidine deaminase domain (e.g., derived from a TDD described herein) is a “split enzyme” comprised of first and second “half domains” or “splits” that lack cytidine deaminase activity alone but dimerize to form an active cytidine deaminase. As used herein, half domains that are “inactive” or “lack cytidine deaminase activity” may be half domains that i) lack any cytidine deaminase activity (e.g., any detectable cytidine deaminase activity), ii) lack specific cytidine deaminase activity, or iii) lack significant cytidine deaminase activity (i.e., on-target base editing activity of 1%, 2%, 3%, 4%, 5%, 6%, 7%, 8%, 9%, or 10% or more, which in particular embodiments may be 10% or more). For example, assembly of the active cytidine deaminase may be driven by the binding of half domain-linked zinc finger proteins to DNA targets in proximity to each other such that the half domains are positioned to allow assembly of a functional cytidine deaminase.

It is understood that the “half domain” pairs described herein may refer to any pair of cytidine deaminase polypeptide sequences that separately lack cytidine deaminase activity, but together form a functional cytidine deaminase domain (either wild-type or a variant discussed herein). Where the cytidine deaminase is DddA, the “split” in the DddA sequence may occur at any of a number of positions, such as, for example, at G1322, G1333, A1343, N1357, G1371, N1387, E1396, G1397, A1398, 11399, P1400, V1401, K1402, R1403, G1404, A1405, T1406, G1407, or E1408, and need not be in the middle of the protein. In some embodiments, the “split” occurs at G1322, G1333, A1343, N1357, G1371, N1387, G1397, G1404, or G1407. In certain embodiments, the “split” occurs at G1404, G1407, G1333, or G1397. In particular embodiments, the “split” occurs at G1404 or G1407. In some embodiments, the DddA half domain pairs may comprise the amino acid sequences of:
a) SEQ ID NOs: 82 and 83;
b) SEQ ID NOs: 84 and 85;
c) SEQ ID NOs: 18 and 19;
d) SEQ ID NOs: 51 and 52; or
e) SEQ ID NOs: 53 and 54.

In certain embodiments, the TDD may comprise, for example, an amino acid sequence under NCBI Accession No. WP_069977532.1 (“TDD1,” SEQ ID NO: 86), WP_021798742.1 (“TDD2,” SEQ ID NO: 87), QNM04114 (“TDD3,” SEQ ID NO: 88), WP_181981612 (“TDD4,” SEQ ID NO: 89), AXI73669.1 (“TDDS,” SEQ ID NO: 90), WP_195441564 (“TDD6,” SEQ ID NO: 91), AVT32940.1 (“TDD7,” SEQ ID NO: 117), WP_189594293.1 (“TDD8,” SEQ ID NO: 118), TCP42004.1 (“TDD9,” SEQ ID NO: 119), WP_171906854.1 (“TDD10,” SEQ ID NO: 120), WP_174422267.1 (“TDD11,” SEQ ID NO: 121), WP_059728184.1 (“TDD12,” SEQ ID NO: 122), WP_133186147.1 (“TDD13,” SEQ ID NO: 123), WP_083941146.1 (“TDD14,” SEQ ID NO: 124), WP_082507154.1 (“TDD15,” SEQ ID NO: 125), WP_044236021.1 (“TDD16,” SEQ ID NO: 126), WP_165374601.1 (“TDD17,” SEQ ID NO: 127), NLI59004.1 (“TDD18,” SEQ ID NO: 128), or KAB8140648.1 (“TDD19,” SEQ ID NO: 129), or a part of said amino acid sequence that is capable of cytidine deaminase activity (e.g., a “toxic domain”). These amino acid sequences are shown below:

NCBI Accession No. WP_069977532.1 (TDD1)
(SEQ ID NO: 86)
MSSSDAGRAFGVPENVLARFTRYPGGARRRAGRTARARRL
GIVLSAVLSATLLPAEAWAIAPPAPRTGPTLDALQQEEEV
DPDPAAMEELDDWDGGPVEPPADYTPTEVTPPTGGTAPVP
LDSAGEELVPAGTLPVRIGQASPTEEDPAPPAPSGTWDVT
VEPRATTEAAAVDGAIIKLTPPASGSTPVDVELDYGRFED
LEGTEWSSRLKLTQLPECFLTTPELEECGTPITIPTSNDP
ATGTVRATVDPADGQPQGLAAQSGGGPAVLAATDSASGAG
GTYKATSLSATGSWTAGGSGGGFSWSYPLTIPDTPAGPAP
KISLSYSSQSVDGRTSVANGQASWIGDGWDYHPGFVERRY
RSCNDDRSGTPNNDNSADKEKSDLCWASDNVVMSLGGSTT
ELVRDDTTGTWVAQNDTGARIEYKDKDGGALAAQTAGYDG
EHWVVTTRDGTRYWFGRNTLPGRGAPTNSALTVPVFGNHT
GEPCHAATYAASSCTQAWRWNLDYVEDVHGNAMVVDWKKE
QNRYAKNEKFKAAVSYDRDAYPTQILYGLRADDLAGPPAG
KVVFHAAPRCLESAATCSEAKFESKNYADKQPWWDTPATL
HCKAGDENCYVTSPTFWSRVRLSAIETQGQRTPGSTALST
VDRWTLHQSFPKQRTDTHPPLWLESITRVGFGRPDASGNQ
SSKALPAVTFLPNKVDMPNRVLKSTTDQTPDFDRLRVEVI
RTETGGETHVTYSAPCPVGGTRPTPASNGTRCFPVHWSPD
PAAFSDENLDKSGYEPPLEWFNKYVVTKVTEMDLVAEQPS
VETVYTYEGDAAWAKNTDEYGKPALRTYDQWRGYASVVTR
TGTTANTGAADATEQSQTRTRYFRGMSGDAGRAKVHVTLT
DVTGTATTVEDLLPYQGMAAETLTYTKAGGDVAARELAFP
YSRKTASRARPGLPALEAYRTGTTRTDSIQHISGDRTRAA
QNHTTYDDAYGLPTQTYSLTLSPNDSGTLVAGDERCTVTT
YVHNTAAHIIGLPDRVRATTGDCAAAPNATTGQIVSDSRT
AYDALGAFGTAPVKGLPVQVDTISGGGTSWITSARTEYDA
LGRATKVTDAAGNSTTTTYSPATGPAFEVTVTNAAGHATT
TTLDPGRGSALTVTDQNGRKTTSTYDELGRATGVWTPSRP
VNQDASVRFVYQIEDSKVPAVHTRVLRDAGTYEESIELYD
GELRPRQTQREALGGGRIVTETLYNANGSAKEVRDGYLAE
GEPARELFVPLSLDQVPSATRTAYDGLGRPVRTTTLHRGV
PRHSATTAYGGDWELSRTGMSPDGTTPLSGSRAVKATTDA
LGRPARIQHFTTQNVSAESVDTTYTYDPRGPLAQVTDAQQ
NTWTYTYDARGRKTSSTDPDAGAAYFGYNALDQQVWSKDN
QGRLQYTTYDVLGRQTELRDDSASGPLVAKWTFDTLPGAK
GHPVASTRYNDGAAFTSEVTGYDTEYRPTGNKVTIPSTPM
TTGLAGTYTYASTYTPTGKVQSVDLPATPGGLAAEKVITR
YDGEDSPTTMSGLAWYTADTFLGPYGEVLRTASGEAPRRV
WTTNVYDEDTRRLTRTTAHRETAPHPVSTTTYGYDTVGNI
TSIADQQPAGTEEQCFSYDPMGRLVHAWTDGNSAVCPRTS
TAPGAGPARADVSAGVDGGGYWHSYAFDAIGNRTKLTVHD
RTDAALDDTYTYTYGKTLPGNPQPVQPHTLTQVDAVLNEP
GSRVEPRSTYAYDTSGNTTQRVIGGDTQTLAWDRRNKLTS
VDTNNDGTPDVKYLYDASGNRLVEDDGTTRTLFLGEAEIV
VNTAGQAVDARRYYSSPGAPTTIRTTGGKTTGHKLTVMLS
DHHSTATTAVELTDTQPVTRRRFDPYGNPRGTEPTTWPDR
RTYLGVGIDDPATGLTHIGAREYDASTGRFISVDPVMDLT
DPLQMNGYTYANADPINNSDPTGLLLDARGGGTQKCVGTC
VKDVTNRKGIPLPPGEEWKHEGEAQTDFNGDGFITVFPTV
NVPAKWKKAKKYTEAFYKAVDTACFYGRESCADPEYPSRA
HSINNWKGKACKAVGGKCPERLSWGEGPAFAGGFAIAAEE
YAGRGGYRGGGARRGSPCKCFLAGTEVLMADGSTKSIEDI
KLGDEVVATDPVTGEAGAHPVSALIATENDKRFNELVIIT
SEGVERLTATHEHPFWSPSEGEWLEAGELRTGMTLRSDSG
ETLVVAGNRAFTQRARTYNLTVADLHTYYVLAGQTPVLVH
NANCGPHLKDLQKDYPRRTVGILDVGTDQLPMISGPGGQS
GLLKNLPGRTKANGEHVETHAAAFLRMNPGVRKAVLYIDY
PTGTCGTCRSTLPDMLPEGVQLWVISPRRTEKFTGLPD
NCBI Accession No. WP_021798742.1 (TDD2)
(SEQ ID NO: 87)
MVDLGAYEEPVAFDDGVADALRSAASALSGTLSGQAASRS
SWAATASTDFEGHYADVEDANARAACDDCSNIASALDALA
ADVQTMKDAAASERDRRRQAKEWADRQKDEWAPKSWIDDH
LGLDKPPAGPPETPVVDAQAPTVATWSEPAQGQAGGVSSA
RPDDLRTYSSNVTGANDTVTTQKGTLDGALSDFADRCSWC
SIDTSGITTALAAFGANNTNETRWVDTVAAAFEAAGGSGA
ISAVSDAALDASLQAAGVTQSRQPVDVTAPTIQGDPQTSG
YADDPVNTTTGNFIEPETDLAFSGGCASLGFDRVYNSLSA
GVGAFGPGWASTADQRLLVTEDGAVWVQPSGRHVVFPRLG
NGWDRAHNDTYWLHTTTDTTGPTPGDAPTTGAAGGAGVFV
VSDNAGGRWVEDRAGRPVSVSRGPGTRVDHRWDGDRLVGL
THERGRAVTIEWNDHHTRITALTANDGRRVDYGYDPAGRL
TEAASAGGTRTYGWNEAGLIATVTDPDGVVEAANTYNEHG
QVTSQRSRFGRLSHYTYLPGGVTQVADEDGGRANTWIHDQ
TGRLVGMVDADGNRQSIGWDQWGNRVQITGRDGRTTVCRY
DARGRLITRQEASGARTDYEWDEADRVVQVTVTDTTSSSH
GNTSSAGGSGPSVTSYEYEGAGRNPSTVTDPEGGVTRLTW
DQNLLTEATDPAGVRVRLGYDGHGDLVSTTNAAGDTARLV
RDGAGRVVAAITPLGHRTEYRYDEAGRLASRQDPDGALWR
YEHTTGGRLSAVVDPDGGRTVTEYGPGGVEEATTDPLGRR
LEQEWDDLGNLAGVRLPDGREWSYVHDGLSRLTETVDPAG
GLWRREYDVNGMVAATVDPTGVRRGLAWAADGSVTVSDAS
GTARVGVDGLGRPVSVSVSSAPAPGEAVPMGMSLEETVGT
GAPAPGGAGPDGPDARVVVRDLCGRPVEALDADGGLTRLM
RDAAGRLVEEISPAGRSTRYEWDRCGRLSAVIGPDGARTT
MAYDAASRLIAQDGPGGRVRVAYDRCGRLSTVTAPGRGKT
TWGYDRAGRVRSVRSPAWGLVRFGYDPAGQLTAVTNALGG
VTRYDYDECGRLVQVTDPLGHVTRRTYTAADRVETLVDPL
GRTTQAGYDAAGRQLWQTDDTGERLAFGWDEAGRLERVAT
GGEGLPGQTCCALTRPGRRVLRVTGPGGARDELVEDRLGR
LARHARGGRTVGEWSWDPDGACTAFTGPDGQRVRYAYDDA
GALVRVEGTAFGPVTVRRDTAGRLTGMDGPGLTQRWDRDE
TGHVIAYRRTKNGVTTSSRVSRDESGRVTAVDGPDGTVRY
GYDPAGQLARIEGPDGRRESFTWDKAGHLTRRSVERPGAR
PETTLYSYDPAGQLASTDGPDGRTLYTWDAAGRRTGQDGP
DGHWSYSWAPSGHLTAVTRRTPHDARTWRISRDGLGLPRR
IDDTDLAWDLSGPVPALTRFGTHTVTGLPRALAIDGTLTS
TGWRPARPTSADDPWAPPPPVVETDGARLGVGGAVGLGGL
EILGARVHDPTTFSFLSPDPLDQPPLAPWATNPYSYAANN
PLAFTDPTGLRPLTDTDFEAYKHDHGGLGGWIADHKDYLI
GGAMVIAGGVLMATGVGGPLGGMLIGAGADTIIQRATTGQ
VDYGQVAVSGLLGAAGGGAASALLKGGGRLATELGATGLR
TAITTGAASGTASGAGGSGYGYLTGPGPHTVSGFLTSTAT
GAVEGGLLGGASGAAGHGLSTTGKNVLGHFEPTPTTPQGT
SSDTIAEMLNSASQPGRTAGVLDIDGELTPLTSGRPSLPN
YIASGHVEGQAAMIMRQQQVQSATVYHDNPNGTCGYCYSQ
LPTLLPEGAALDVVPPAGTVPPSNRWHNGGPSFIGNSSEP
KPWPR
NCBI Accession No. QNM04114.1 (TDD3)
(SEQ ID NO: 88)
MSLPEYDGTTTHGVLVLDDGTQIGFTSGNGDPRYTNYRNN
GHVEQKSALYMRENNISNATVYHNNTNGTCGYCNTMTATF
LPEGATLTVVPPENAVANNSRAIDYVKTYTGTSNDPKISP
RYKGN
NCBI Accession No. WP_181981612 (TDD4)
(SEQ ID NO: 89)
MLAIEKIKSGDKVISTDPETMETSPKTVLETYIREVTTLV
HLTVNGEEIVTTVDHPFYVKNQGFIKAGELIVGDELLDSN
CNVLLVENHSVELTDEPVTVYNFQVEDFHTYHVGKCRLLV
HNANCNQEKPVLPKYDGKTTEGVMVTPDGKQISFKSGNSS
TPSYPQYKAQSASHVEGKAALYMRENGINEATVFHNNPNG
TCGFCDRQVPALLPKGAKLTVVPPSNSVANNVRAIPVPKT
YIGNSTVPKIK
NCBI Accession No. AXI73669.1 (TDD5)
(SEQ ID NO: 90)
MSSSVSGRAFRVSGVLTRITKSWTPGSARRSSASVRHRGR
AVRARSLGVTLSAVLAATLLPAEAWAIAPPAPRIGPSLVD
LQQEEPADPDQAKIDELSTWSGAPVEPPADYTPTATTPPA
GGTAPVALDGAGDDLVPVGNLPVRLGKASPTDEEPDPPAP
GGTWDVAVEPRTSTEASDVDGALITVTPPSGGATPVDIEL
DYGKFEDLFGTAWSSRLRLTQLPECFLTTPELDECTTVVD
VPSVNDPSNDTVRATIDPAASPQQGLSTQSGGGPVVLAAT
DSASGAGGTYKATPFTATGTWTAGGSGGGFSWSYPLTAPA
PPAGPAPTISLSYSSQSVDGRTSVANGQASWIGDGWDYNP
GFIERRYRSCNDDRSGTPNNAGGKDKKKSDLCWASDNLVM
SLGGSATALVHDGTTGAWVAQSDTGARIEYRTRTGSPKTA
QTGAYDGEYWVVTTRDGTRYWFGRNTIPGRTAATESALTV
PVFGNHSGEPCHATAYADSSCAQAWRWNLDYVEDVHGNAM
IVDWKKETNRYARNEKFKEAVAYHRGGYPAQILYGLRADD
LNGAPAGKVVEKTAPRCVEDAGTTCSPTGYESDNYADKQP
WWDTPATLHCKSGAKNCFVTSPTFWSSVRLTEIETHGRRT
PGSTALSLVDSWTLKQSFPKQRTDTHPPLWLESITRTGHG
APNASGEQTSRALPPVSFLPNVVDMPNRVSKGATDETPDF
DRLRVETVRTETGGEIHVDYSAPCAVGTAHPSPETNTTRC
FPVHYSPDPEALSDEVLAKKPAPVEWFNKYVVQKVTEKDR
VARQPDVVTTYAYEGGGAWGRSTDEFTKPKLRTYDQWRGY
ASVLVRKGVTGADPAAADATEQSQTRMRYFRGMSGDAGRP
TVTVKDSTGAETLGEDLAPYQGMPAETVAYTRAGGDVASR
ILAWPTSRETASQARPGLPALKAHRVATARTETVETISGG
RTRTARTVTTYDDTYGLPLTAETLTLTPDGSGGTTTGDRS
CSTNTYVHNTAKHLIGLVQRARTTVGTCAQAATASGSDVV
SDTRVSYDALDAFGAAPVRGLPFRTDTVGADGTGWVTSAR
TEYDPLGRATEVRDAKGHVSKVGFVPPTGPAFTTTSTDAK
GHTTTTALDPARGTALSVTDANGRRTTSAYDELGRTTAVW
SPSRTQGTDKASVLFDYQIEDNKVPATRTRVLRDNGTYED
SVTVYDGLLRPRQAQTEALGGGRIVTETLYNANGAPAETR
NGYLAEGEPQTELFVPLSLTQVPSASKTAYDGLGRAVRTT
VLHAGDPQHSATVRHEGDRTLTRTGMSADGTTPMPGSRST
ATWTDALGRTSKIEHFTATDLSAAIDTRYTYDARGNLAKV
TDARDNIWTYTYDARGRLTFSTDPDAGSSSFGYDVLDRQI
WSKDSRQRSQHTVYDELGRRTELRDDSAEGPLVAKWTYDT
LPGAKGLPVASTRYHEGAEFTSEVTGYDQEYRPTGSRTTI
PSTPLTTGLAGTYTYKNTYTPTGLPQSVELPATPGGLAAE
KVITRYDGEGSPRTTSGLAWYTVDTVLSPLGQVLRTASGE
APNRVWATHFYDESTGRLDRRITDRETLDPSRISETSYAH
DTVGNITSITDTQSPARVDRQCFAYDPMGRLAHAWTAKSP
GCPRSSTAQGAGPNRTDVSPSIDGAGYWHSYEFDTIGNRT
GMVVHDPADPALDDTYVYTHGVPSEGPLQPATLQPHTLTK
VDATVRGPGSTVTSSSTYAYDPSGNTTQRVIGGDTQALTW
DRRNKLMSADTDDDGTADVTYLYDASGNRLLEADATTRTL
YLGESEIVVDTAGRPVEARRYYSHPGAPTTLRTTGGRTSG
HTLTVQLTDHHNTPTASVALTGGQPVTRRMFDPYGNPRGT
EPTTWPDRRTYLGVGIDDETTGLTHIGAREYDSVTGRFIS
ADPIIDIADPLQMNGYAYANNNPVTNWDPTGLKSDECGSL
YRCGGNQVITTKTTKYQDVNTVARHFEKTASWATLAQWKA
EGLGKSPAFGKAKKLTKWKNEHYEKNWTINLVPGMARSWV
SGVDAAASAIMPFPTVQAAPLYDSLVSSLGVNTKGRAYAN
GEGLMDGLSMVGGVGAIAPGIKSGLKAAAKGCGPGNSFTP
GTEVALADGTTKPIEDIKIGDEVLATDPETGETRAEKVTA
EIRGDGTKNLVKVTIDTDGDRGTDTAEITATDGHPFWVPE
LGRWIDATDLAPGQWLRTSAGTHVQITAIKRWTETATVHN
LTVADLHTYYVLAGKTPVLVHNENCGPNLKDLPKDYDRRT
VGILDVGTDQLPMISGPGGQSGLLKNLPGRTKANTDHVEA
HTAAFLRMNPGIRKAVLYIDYPTGTCGTCGSTLPDMLPEG
VQLWVISPRKTEKFAGLPD
NCBI Accession No. WP_195441564 (TDD6)
(SEQ ID NO: 91)
MKLTYKELEIELELAGLLAVEELVLTQGLNCHAGLTLKIL
IEEEQRDELVTMSSDAGVTVRELEKTNGQVVFRGKLETVS
ARRENGLFYLYLEAWSYTMDWDRVKKSRSFQNGALTYMEV
VQRVLSGYGQSGVTDHATGGACIPEFLLQYEESDWVFLRR
LASHFGTYLLADATDACGKVYFGVPEISYGTVLDRQGYTM
EKDMLHYARVLEKEGVLSQEASCWNVTVRFFLRMWETLTE
NGIEAVVTAMRLHTEKGELVYSYVLARRAGIRREKEKNPG
IFGMSIPATVMERSGNRIRVHFEIDPEYEASEKTKYFTYA
IESSSFYCMPEEGSQVHIYFPDHDEQGAVAVHAIRSGEGA
SGSCSTPENKRFSDPSGSAMDMTPASLQFAPDAGGATVLH
LEGGGFLSLTGMDIKLKTQMGMASDKEKPMQDLMICGEQK
LTMQIGESSDDCIVMEAGTEVRSALVVQEADSSPAAVPSG
DELLSEQEAADAQAREAENNAVKEDMITKKQESKRKIVDG
VISLVTVVGLTALTVATGGLAAPFAIAAGVKATFAVADIA
EGLDGYSKMNAMDASRPANFLRDTVEGGNQTAYDITSMIT
DVAFDVVSGKALVGAFSGADKVSKVQKFAGKAMSFWNGIC
PKTKVANFLFQMGGTMLFGAVNDYLTTGKVDLKNLGLDAF
AGLAKGTLGTAGTEKIKRLLNTDNKWVEKAVGILAGTTFG
TTVDLGINKLAGRDVDLLQVIKQNLIESGLGQFFGEPIDV
VTGAFLITATDFTLPDIREDLRVQRKYNSTSREAGLLGPG
WSFSYECRLYCSGNRLHAKLDSGITAVFAWDGSHAVNVTR
GCEWLELTGEDDGWRIYDGRNYKCYHYDGQGLLTAAEDRN
GQCVRLYYEGERLTRITTPLGYSLDVEIRDGRLVQIRDHM
GRTMQYRYENGFLSDVIHMDEGVTHYEYDSNGYLERAVDQ
AKVTYLENRYDDAGRVVLQTLANGDTYRADYHPEKNRVTI
VSSVHDKAVEHWYNEFGEILETSYQDGTKERYEYGENGHR
TSRTDRLGRKTTWTYDEAGRLTEEVQPDGLRTVHRYDAAG
NEILRTDSAGRETAFEYDGHHNRTAERRTDGLQVRENRSV
YDWMGRLTETADAEGNRTQYQYGEAGGKPSVIRFADGETC
SFEYDKAGRMMAQEDACGRTEYGYNARNKRALVRDGEGNE
TRWMYDGMGRLLALYLPKAWKEQHGEYSYSYDELDRLIHT
KNPDGGHERLMRDGEGNVLKRVHPNAYDSCRDDGEGTTYD
YDSDGNNIRIHYPDGGCERIFYDSEGNRIRHVMPESYDPQ
TDDGEGFTYTYDACSRLTGVTGPDGVRQASYTYDPAGNLT
EETDAEGRCTYRSYTAFGELKEQLKPALEKDGVMLYERIT
WQYDRCGNVLLEQRHGGYWDSNGVLVKEDGAGLALRFTYD
SRNRRIRVEDGLGAVISCHYDVQGKLVYEEKAVSGEVRQV
IHYGYDRAGRLTERKEELDSGLAPLEGEPRYAVTRYRYDG
NGNRTGIVTPEGYRILRSYDACDRLVSERVVDDKNGIDRT
TSVTYDYAGNITRIVRSGKGLGEWEQGYGYDLKDRIVHVK
DCLGPVFSYEYDKNDRRIAETLPQTGMTENGKSGYPKNQN
RYRYDVYGRLLTRTDGSGTVQEENRYLPDGRLALSREADG
QEIRYAYGAHGREEETGTARSRKAGRAAQKYRYDSRGRIT
GVVNGNGNETGYDLDAWGRIQNIRQADGGEEGYTYDFAGN
VTGTRDANGGVITYRYNSQGKVCEITDQEGNSETFRYDRE
GRMVLHVDRNGSEVRTTYNVDGNPVLETGTDRNGENRVTR
SFEYDASGNVRKAVAGGFCYTYEYRPDGKLLKKSASGRTI
LSCSYHADGSLESLTDASGKPVFYEYDWRGNLSGVRDENG
DMLAAYAHTPGGKLKEICHGNGLCTRYEYDTDGNMIHLHF
QRENGETISDLWYEYDLNGNRTLKTGKCILSGDSLTDLAV
SYRYDSMDRLTSESRDGEETAYSYDFCGNRLKKLDKSGTE
EYHYNRKNQLICRFSEKEKTAYRYDLQGNLLEAAGAEGTE
VFSYNAFQQQTAVTMPDGKHLENRYDAEYLRAGTVENGTV
TSFSYHNGELLAESSPEGDTISRYIPGYGVAAGWNREKSG
YHYYHLDEQNSTAYITGGSCEIENRYEYDAFGVLKNSMEE
FHNRILYTGQQYDQTSGQYYLRARFYNPVIGRFVQEDEYR
GDGLNLYAYCKNNPVVYYDPSGYDSQYPCKEEMSAGAGES
GRKTISLPEYDGTTTHGVLVLDDGTQIGFTSGNGDPRYTN
YRNNGHVEQKSALYMRENNISNATVYHNNTNGTCGYCNTM
TATFLPEGATLTVVPPENAVANNSRAIDYVKTYTGTSNDP
KISPRYKGN
NCBI Accession No. AVT32940.1 (TDD7)
(SEQ ID NO: 117)
MGDRLPAFVDGGDTLGIFSRGGIERDLASGVAGPASSLPK
GTPGFNGLVKSHVEGHAAALMRQNGIPNAELYINRVPCGS
GNGCAAMLPHMLPEGATLRVYGPNGYDRTFTGLPD
NCBI Accession No. WP_189594293.1 (TDD8)
(SEQ ID NO: 118)
MSSRPFRKRLPGAVVRRWLGRGAVVASLSLLPQVVVPSGY
DFAAQAQSVAARKKLEDRPEAKINKVGVLRPGTSKAPKDK
SAPASRKTRERLQEASWPKSGKATAAVTATSEATVNVGGL
GMELTQEPAAPAAKSAKSTTKRKATGPAEKVTLRVHSRAT
AKKAGVNGVLLTVDPARGESNEKAEDTDKLRISLDYSSFS
DVYGGNFGPRLSLVKLPACALTTPEKKSCRTQTPVAGADN
EAESQTLTGTVPARNLKAGTPMLLAAAADSSGGGGDFSAT
PLSPTATWEAGGSTGDFTWDYPLRVPPATAGPSPNLSISY
NSASVDGRTAGENNQTSLIGEGFSITESYIERKYASCKDD
GQSGKGDLCWKYANATLVLNGKAVELVNACADKSACDTAA
LSEASGGTWKVKNEDGTRVEHLTGASGNGDNNGEYWKVTD
ASGIQYYFGKHRMPGWSDKGTTDTADDDPSTYSTWAVPVF
GDDSGEPCYKSSGFADSSCNQAWRWNLDYVVDTHDNASTY
WYSKETNYYSKNADTTVNGTAYTRGGYLNRIDYGLRSDLI
YSKPAAQQVRFTYGQRCIVTNGCSSLTKDTKANWPDVPYD
MICAANTKCTTQIGPSFFTRQRLIDITTSVWTGTGTTRRD
VDTWHLSHDFPDTGDASSPSLWLKSIQNTGKANTTTAAMP
PIVFGGIQMPNHVEGSGQDNLRYIKWRVRTIKSETGSTLT
VNYSDPDCIWGSSMPSAVDKNTRRCFPVKWSQSGTTPVTD
WFHKYVVTSVLQDDPYGHSDTGETYYDYQGGAGWAYSDDE
GLTKPSNRTWSQWRGYGKVVTTSGNSEGPRSKKSTLYMRG
LNGEKELDGTARVAKVTDSTGTAIDDSRQYAGFVRETIAY
NGSDELSGTINTPWSHKTGSHTYSWGTTEAWIVQAGETES
RTKISTGTRTVKQKTTYDTTYGMPITVEDSGDATKFGDES
CVRTSYARNTSAWLVNRVSRTETYSVPCATIPAIPADVVS
DITTAYDAKAVGAAPTQGDITATYRVASYNAADKTPVYQQ
VSSSTEDKLGRPLTETNALDRTVKTSYVPDDTGYGPLTSK
TTTDPKLYTSTTEVDPAWGAASKTTDANGNVTEWSFDALG
RLRSVWKPDRSRTLDDAASIVYAYSVNNDKETWVRTDALK
ADGKTYNSSYEIFDSLLRPRQKQVPAPNGGRVISEMLYDD
RGLAYIANSQVHDNSAPSGTLANTYTGSVPASTETVFDAA
GRATDSIFRVYGQEKWRTKTDQQGDRTAVTAAAGGTGTLT
IVDARGRVTERREFGGPAPTGTDYTRTLYEYTPGGQIKKM
TGPDGAVWTYEYDLRGRKTTSTDPDKGSITTTYNDADQPL
TATTTLDNVSRTLINDYDELGRPTGTWDGTKDNAHQLTKF
TYDSLAKGQPTASIRYVGGTTGKIYSQSVTGYDALNRPKG
TKTVIAATDPLVTAGAPQTFTTSTAYNIDGTVQSTSLPAA
AGLPAETVKNTYNSLGMLTGTDGMTDYVQHIGYSPYGEIE
ETRLGTSTEAKQLQVLNRYEDGTRRLANTHTLDQTNAGYT
SDVDYVYDATGNVKSVTDKANGKDTQCFAYDGYRRLTEAW
TPSSNDCATARSASALGGPAPYWTSWTYKPGGLRDSQTEH
KTSGDTKTVYGYPAVNTSGTGQPHTLTSVTVGSGSAKTYT
YDEHGNTTKRYSPTGTAQSLTWNIEGELTRLTEGTKTTDY
LYDANGELLIRRSPDKTVLYLGGQELHYDTATEKFTAQRY
YPAGDATAVRTETGLSWMVDDHHGTASMTVDATTQAVTRR
YTKPFGEARGTAPSVWPDDKGFLGKPADTGTGLTHIGARE
YDPTLGRFLSVDPVLAPDDHESLNGYAYANNTPVTLSDPT
GLRPDGMCGGSSSSCGGGTETWTLNSKGGWDWSYTKTYTK
KFTYRTGNGGTRTGTMTTTVRTEVGHKAVRIVFKKGPEPK
PAKKDGQCSSCWAMGTNPGYSPGATDDWIDRPKLETWQKV
VLGAISVVAAGVILAPAAIVVGEGCLAAAPVCAAEIAEAA
TGGASGGSAVVGAGVVATGAKAVTTGKSLSESQATLSVAQ
RLLATIGEEGKTAGVLELDGELIPLVSGKSSLPNYAASGH
VEGQAALIMRDRGATSGRLLIDNPSGICGYCKSQVATLLP
ENATLQVGTPLGTVTPSSRWSASRTFTGNDRDPKPWPR
NCBI Accession No. TCP42004.1 (TDD9)
(SEQ ID NO: 119)
MAFGIGTSRRGSGGGRGWGRRLVTPVAALALLAPLGEAQD
AVAQDAGAVRSGPVQPDVPKPRVSKVKEVKGLGAKKARDR
VAAGKKAGAAQAARARREQTAVWPGPDTASIELADDRRAK
AELGGASVSVVPENGRKTAASGTAQVTILDQKAADKAGVT
GVLLSATADTAGTAEVSVDYSGFASAFGGDWAQRLHLVQL
PACVLTTPEKAVCRRQTPLKTDNNASEQSVAAQVALAKAE
PGAPSAQSVASAEGPSATVLAVTAAAAGSGASPKGTGDYA
ATELSPSSAWEAGGSSGAFTWNYGFTVPPAAAGPTPPLAL
SYDSGSIDGRTATTNNQGSAVGEGFSLTESYIERSYGSCD
KDGHADVWDHCWKYDNASIVLNGKSNRLIKDDTSGKWRLE
TDDSTVTRSTGADNGDDNGEYWTVTTGDGTKYVEGENKLD
GAADQRTNSTWTVPVFGDDSGEPGYDKGDTFAERAVTQAW
RWNLDYVEDTSGNASTYWYAKDSNYYPKNKATTANASYTR
GGYLKEIRYGLRKDALFTDDADAKVVFAHAERCTVGSCTT
LTKDTAKNWPDVPFDAICSSGDSECNAAGPSFFSRKRLTG
ISTFSWNAASKAFDPVDTWELTQDYYDAGDIGDTTDHVLV
LESIKRTAKAGATAIDVNPVTFTYQLRPNRVDGTDDILPL
KRHRIETITSETGSITTVTLSQPECKRSTVLDAPQDSNTR
PCYPQFWNINGATKASVDWFHKYRVLAVAVDDPTGHNESI
EHAYDYAGAAWHYSDDPFTTKNERTWSEWRGYRDVTTYTG
ALDTTRSKSVSRYMLGMDGDKNTDGTTKSVSTAPLMDTDV
DFAALTDSDPYSGQLLQQVTYSGSQPISTSYTNFTHKNTA
SQTVPDATDHTARWVRPNSSYASTYLTASKTWRTQVTTSR
YDDLGMVTSHDDYGQKGLSGDEICTRTWYARNTEAGINSL
VSRTRTVGKECSVDDTALDLPADNKRSGDVLSDTATAYDG
ATWSDSMKPTKGLVTWTGRAKGYASGTPSWQTLTSAAPAD
FDVLGRPLKVTNAEGQPTTTAYTPVTAGPVTKIISGNPKG
FKTTSFLDPRTGQELRTYDANLKKTERVYDALGRLTQVWL
PNRDRGSESATFGPSVKFEYTIDNNDPSWVSTAALKKDGK
TYATSYAIYDAMLRPLQSQTETSNGGRLLTDTRYDTRGLP
YETYANIFDTTSTPNGTYTRAEYGEAPNQNATVEDGAGRP
TKSTLLVFGVEKWSTTTSYTGDSTATTALDGGTASRAITN
IRGHTVESREYAGKSPADAQYGDGLGVGFASTRTLYTRGG
LQKQITGPDDATWSYTYDLFGRQVEAEDPDKGTSSTEYDV
LDRATKSTDSRSKSILTAYDELGRMVGTWAGSKTDTNQRT
EYTYDKLLKGQPDKSIRYVGGKAGQAYTDTITEYDSLSRP
VAASLELPADDPFAKVGALGSASRTLSFRHAYNLDDTVKT
AEEPALGGLPSEIIDYGYNNVGQVTSVGGSTGYLLGATYS
PLGQPWEQLLGTANTADHKKVSIRNTFEDGTGRLTRSNVK
ADSQPYMLQDLNYSFDQVGNVTSITDPTTLGGTSSAETQC
FTYDSHRRLTEAWTPSQQKCSDPRSTSSLSGPAPYWTSYT
YNTAGQRTTETTRKAAGDTTTTYCYTKTDQPHELTGTTTK
GDCATRERTYTPDTTGNTTKRPGASTTQDLAWSEEGKLTK
LTENGKATDYLYDATGELLIRNTTSGERVLYTGTTELHLR
TDGTTWAQRYYAAGDQTVAMRSNESGTNKLTYLAGDHHGT
SSLAISADSTQTVSKRYMTPFGAERGKPTGTAWPDDKGFL
SKTTDKTTGLTHIGAREYDPAIGQFISTDPILDPAQPQSL
NGYSYANNTPVTAADPSGLWCDSCNDGKGWTRPDGGTRGD
ENGGKNPDGSVRGTPGFPSTRPTTVGYGNSPGAGKVITDL
GSGTPALPPPDVYQDYQPKLPGVGQMGRNGTYMPELSYEL
NVELYFRERCSFSWTEECESIRAFYTHGEDSHGLPRYWTD
VQDIPTVNTCPICENIGEDIILATLPIGKVGKLRFAPKVE
SAESMLRSLSQEGKTAGVLDINGELIPLVSGTSSLKNYAA
SGHVEGQAALIMRERGVASARLIIDNPSGICGYCRSQVPT
LLPAGATLEVTTPRGTVPPTARWSNGKTFVGNENDPKPWP
R
NCBI Accession No. WP_171906854.1 (TDD10)
(SEQ ID NO: 120)
MRGWVRAVSIPVIVGVLSTALSMPPSFADQEPVARTEATT
DGLPTNADEGQRAEPPALIPSENRIPGVGLKSEIESQPTA
ASVADGPLPSERSDSFFPALAPTPPTIVGYVPTSLAPGCA
EWGALRWTHPDSRPNGLVHLYTFELYRDSDDAMVWDQLFD
YTLTGAGVVSDVAGDCESILPDPQATPIVELGESYYAKVY
AWDGTGWSAPATSSAYPAVALPGLTDEAARGVCVCDTSTG
RLYPLNILRADPVNTATGTLTESATDLTIPGVGPAISASR
TYNSTDPTVGPLGKGWSFPYFSELESAASSVTYKAEDGQE
VEYALQGGAYRLPPGASTRLRSVSGGYQLETKSHQVIGFD
QNGRLEYARDSSGQGVSLAYATNGTLDKITDASGREVDVT
MDASGKVTAIALSDGRSVSYGYTGDLLTSVTDVRGGVTEY
EYDAAGRLAAITDPLGNEVMRSTYDAQGRVISQVDAGGGT
WGFEYVDDGAYQTTRTTDPRGGVSRDVYYNNVLVESETAG
GAITTYQYDERLRLAATVDPHGRTTRHTYDANDNLLSTTH
PNGDREAFTYSSGGDLLTETSPEGRKTTYTYDANHRVATT
TDPNGGVTSYTYNTDGQVLTETSPEGNVTEFEYDAQGNRV
ATISPEGRRTTATFDAYGRLESQTTARGHVAGADPADFTT
TFAYDVASNLTSSTDPLGHVTEYEYDLNNRRTTVIDPLDR
RTETEFDAAGRVVKIIEPGGAETVHEYDLAGNQVATTDAE
GGRTTRTFDLDAHMITMTAARGNEPGAEPADFTWGYEYDG
LGNVVEETDSAGGIVSYGYDERYRQTSVTNQANETTTTAY
DGDGNTVSVTDPLDRTVSTTYNGLNLPATVTDPAGKVSTV
IYDRDGNRTSTTTPLGHKATFTYDGDGMLVQDQTPNGNGR
ISTYTYDADGNQIRTVDPQGRFTTATFDNAGRVSSRSLWN
VTTTYGYDDAGRLTTVTGGDGAVTEYGYNTAGDLVTVTDP
NDHVTTHTYDDAHRRTATTDALNRTRTFGYDADGNQTSTV
LARGPASGDLARWTVTQSYDELGRRTGVTTGSTASTASYA
YDPVGRLTGVTDAGGTTTTVYDDAGQIASVTRGSQAYGYT
YDPRGMVKTITQPGGVTVTNTFDDDGRLATTASTNAGTTA
FSYDKNNNLTRIDNLAATGLVNRWQQRNYDRADALVSTTT
GTGTTTDPTQTVTYSRDGAGRPFVIRRGAGGTQAPGEAHF
FDAAGRLAQVCYDASSMFGQNCATADETLAYTYDGAGNRL
TETRTGGTTPGTTTYTYDAANQLTQRGNTTYSYDADGNQI
SDGATSWTYDELNRLVGIDTPTADSQLTYDGLGNRTSVTT
GATTRTFSWDINNPLPLLTSVTQGTSTTRYRYGPDAIPVN
ANINGTNHALLTEDLNSLTTTYNRTTGAKSWTTTYEPFGT
PRNTTSTGLTTAQVGLGYTGEYLDPTTGLLNLRARNYNPT
LGQFTSTDPVETPQGTPSISPYAYVDNRPTVLTDPSGACF
FIDMPWIPGCSEPSWADEVTPATNGVLAGLISAAEDTFYL
TGMALGVDWVGYDGDLAQQLFDEAAVEGNYHGETYQQAQL
VGGLVALVGGAASTAASLARICTSLVRKIRPPVASGGLAT
EVPAYAGSRTAGTLVTPDGAEFPLISGWHPPAASMPQGTP
GMNIVTKSHVEAHAAAIMRNQGLSEATLWINRAPCGGKPG
CAAMLPRMVPSGSTLTINVVPNGSAGSIADTLIIRGIG
NCBI Accession No. WP_174422267.1 (TDD11)
(SEQ ID NO: 121)
MSDSENRLTRASDSPASGKTQSESKVNTACDSLLDTAGST
YDSLKQPFSSKGGALHHVSEAVNALASLQGAPSQLLNTGI
AQIPLLDKMPGMPASVISAAHLGTPHAHSHPPSDGFPLPS
MGATIGSGCLSVLIGGLPAARVQDIGIAPTCGGLTPYFNI
ETGSSNTFIGGMRAARMGIDMTRHCNPMGHAGKSGEEAEG
AAEKGEQAASEAAEVSSRARWMGRAGKAWKVGNAAVGPAS
GVAGAASDAKHHEALAAAMMAAQTAADAAMMLLSNLMGKD
PGIEPSMGMLMDGNPTVLIGGFPMPDSQMMWHGAKHGLGK
KVKARRADRQKEAAPCRDGHPVDVVRGTAENEFVDYETRI
APGFKWERYYCSGWSEQDGELGFGFRHCFQHELRLLRTRA
IYVDALNREYPILRNAAGRYEGVFAGYELEQRDGRRFVLR
HGRLGDMTFERASEADRTARLVNHVRDGVESTLEYARNGA
LMRIDQEKGPGRRRQLIDFRYDDCGHIVELYLTDPQGETK
RIVHYRYDTAGCLAASTNPLGAVMSHGYDGRRRMVRETDA
NGYSFSYRYDSQDRCIESMGQDGLWHVSLDYQPGRTVVTR
ADGGKWTFLYDEARTVTRIVDPYGGTTERVSGDNGRILRE
IDSGGRVMRWLYDERGGNTGRMDRWGNRWPTKDEAPVLPN
PLAHTVPNTPLALQWGDARHEDLADTLLLPPEIAKIAASF
FPPQPFSASTEQCDETGRVIARTDGYGQAERLRLDATGNL
LQLCDRDGRDYCYSIASWNLRESETDPLGNTVRYRYSPKQ
EITAIVDANGNESTYTYDYKSRLTSVTRHGTVRETYAYDV
GDRLIEKRDGTGNALLRFEVGEDGLQKTRILASGETHTYK
YDHRGNFTRASTDKLDVTLTYDAYGRRTGDKRDGRGIDHS
FVGGRLESTTYFGRFVVRYEAGQAGDVMIHTPGGGIHRLR
RAADGTVLLRLGNRTNVLYGFDADGRCTGRLSWPEGRTAE
IHCVQYRYSAVGELRCVIDSTGGTIEYQYDAAHRLVGESR
DGWAVRRFEYDQGGNLLSTPTCQWMRYTEGNRLSSASCGA
FRYNSRNHLAEQIEENNRRTTYHYNSMDLLVQVKWSDRQE
SWRSEYDGLCRRIAKAMGQARTQYFWDGDRLAAEAAPDGR
LRIYVYVNEASYLPFMFIDYPSCDAEPESGSAYYVFCNQV
GLPERIESAMGLDAWRAEEIEPYGSIRVATGNAIDYDLRW
PGHWFDVETGLHYNRFRYFQPTLGRYLQSDPAGQSGGVNL
YAYSANPLVFVDVLGLECPHNDKSTTECARCEAKEEVDQR
EKRDKELAREIYHIEDKYSDSHAGIGLDPDEKKRALEDKI
DYDDLVRKREKAREDLLEAEKRLREEEIRAKYPTPEEAQL
PPYDGDTTYALMYYTDEHGKSHVVELSSGGADDEHSNYAA
AGHTEGQAAVIMRQRKITSAVVVHNNTDGTCPFCVAHLPT
LLPSGAELRVVPPRSAKAKKPGWIDVSKTFEGNARKPLDN
KNKKST
NCBI Accession No. WP_059728184.1 (TDD12)
(SEQ ID NO: 122)
MSEPANRLTRASEPSERHAAQSESKADTACESLLGTVKST
FDPFKQTFSSDGSALHHVSEAVNALASLQSAPSQLLNTGI
AQIPLLDKMPGMPAATIGVPHLGTPHAHSHPPSSGFPLPS
IGATIGSGCLSVLIGGIPAARVLDIGIAPTCGGLTPYFDI
QTGSSNTFFGGMRAARMGIDMTRHCNPMGHVGKSGGKAAG
AAEKTEEAASEAAQVTSRAKWMGRAGKAWKVGNAAVGPAS
GAAGAAADAAHGEELAAAMMAAQTAADAAMMLLGNLMGKD
PGIEPSMGTLLAGNPTVLVGGFPLPDSQMMWHGVKHGIGK
KVRARIANRRKEVSPCTDGHPVDVVRGTAENEFVDYETKI
APAFKWERYYCSGWSEQDGALGFGFRHCFQHELRLLRTRA
IYVDALNREYPILRNAAGRYEGVFAGYELEQRDGRRFLLR
HGRHGDMTFERENEADRTARFVSHVRDDVECTLEYARNGA
LARIAQEDARGLRRQLIDFRYDDRGHIVELCLTDPRGQTR
RLAHYRYDAAGCLTVVTDPLGAVTSHGYDDRRRMVRETDA
NGYSFSYRYDSQGRCIETVGQDGLLHVVLDYQPGRTVVTR
ADGGKWTFLYDNARTVTRIVDPYGGMTERVIGGDGRILRE
IDSGGRVMRWLYDERGRNTGRMDRWGNCWPTRDEAPVLPN
PLAHTVPVTPLDLQWGEVSPAELTDSVLLSPEIQKVAESL
FQQPAFSPSEQHDARGQVVARTDEHGGVERFRRDAAGNII
QVCDKDGRAHHYGIASWSLRESETDSLGNTVRYRYSNKQE
ITSIVDANGNESAYTYDYKGRITSVMRHGVVRETYTYDAG
DRLIEKRDGAGNLLLRFEVGENGLHSKRILASGETHTYEY
DRRGNFTKASTDKFDVTRTYDAHGRRTGDKRDGRGIEHVY
GDGRLCSTTYFERFTVRYEAEADGEVLIHAPVGGTHRLQR
SSDGQILLRLGNGANVLCRFDAHGRCVGRLVWPEGRPKEC
HRVAYQYSAMGELRRVIANTTGTTEYLYDDAHRLIGESHD
GWPVRRFEYDCGGNLLSAPTCQWMRYTEGNRLATASRGAF
YYNDRNHLAEQIGENNHRTSYHYNSMDLLVKVTWSDWPEV
WTAEYDGLCRRIAKAMGPARTEYYWDGDRLAAEIAPNGQL
RIYVYVNETSYLPFMFIDYDGCDAAPESGRGYYVFSNQVG
LPEWIEDIAGACVWRAMEIDPYGAIRVAPGNELGYNLRWP
GHWLDPETGLHYNRFRSYHSALGRYLQSDPAGQSGGINLY
AYTANPLVFVDVLGRECPHLNESSSECSQCENREEAERIR
KEMLQSISRRMDIEGDVTGHPGILLTQAELTGKYSHYAEE
YKQLLKDIDTKREAEEAALLREAYPSMEGATLPPFDGKTT
IGLMFYTDASGQYQVKKLFSGEKVLSNYDATGHVEGKAAL
IMRNEKITEAVVMHNHPSGTCNYCDKQVETLLPKNATLRV
IPPENAKAPTSYWNDQPTTYRGDGKDPKAPSKK
NCBI Accession No. WP_133186147.1 (TDD13)
(SEQ ID NO: 123)
MSTPPGNPASPANEPPPPPAPLISPTGNTSVDALASAVNA
GAQPFQQLGNPKANTLDRVTNVVSGAVGSLGALDQLLNTG
MAMIPGANLVPGMPAAFIGVPHLGVPHAHAHPPSDGVPMP
SCGVTIGSGCLSVLYGGMPAARVLDIGLAPTCGGLAPIFE
ICTGSSNTFIGGARAARMALDLTRHCNPLGMSGAGHAEQD
AEKASALKRAMHIAGMAAPVASGGLTAADQAVDGAGAAAV
EMTAAQTAADAIAMAMSNLMGKDPGVEPGVGTLIDGDASV
LIGGFPMPDALAMLMLGWGLRKKAHAPEGAGEPKRTEQGE
CKGGHPVDVVRGTAENQFTDYATLDAPEFKWERYYRSDWS
ERDGALGFGFRHSFQHELRLLRTRAIYVDGHGRAYAFGRS
ASGRYEDVFAGYELEQQGENRFVLLQATRGEFTFERASAA
QASARLVRHVHEGVESALRYAGDGTLRHIEQTAQREQRHR
MIDLLYDARGHVVEMRVTDPRGAVLCAARYRYDATGCLVA
STDALGASMTYGYDAWRRMIRETDANGYAFSYRYDSDGRC
VESAGQDGLWRVLLDYQPGRTVVTQADGGRWTYLYDAART
VTRIVDPYGGATERVIGDDGRIVEEVDSGGRVMRWLYDER
GENTGRQDRWGNRWPTRDEAPVLPNPLAHVVPARPLELLW
GDARPEDFTDRLLLPPEIEAVAAAAFAPSAAVPKPAEQRD
GAGRVIRRTDESGHAECLHRDAAGNVVQLRDKDGRYYGYA
IASWNLRESETDPLGNTVRYRYSSKQNITAVVDANGNESR
YTYDYKSRLTRVARHDTIRESYVYDTGDRLIEKRDGAGNT
LLRFEVGENGLHSKRILASGETHTYEYDRRGNFTRASTDK
FEVTLTYDAFGRRTGDKRDGRGVEHSFVGQRLESTTWFGR
FVVRYETGPSGDVMIHTPGGDVHRLQRAADGTVLLRLSNS
TNVLYKFDENGRCAGRLTWPDGHTSANRCVQYRYSAVGEL
RQVIDSKGGTTEYQYDDAHRLVGESREGWAFRRFEYDRGG
NLLSTPTCQWMRYTEGNRLSGAACGAFCYNSRNHLAEQIG
ENNRRTTWHYNSMDLLVRVQWSDRQENWSAEYDGLCRRIA
KAMGQARTQYFWDGDRLAAEVAPNGQLRIYAYVNETSYLP
FMFIDYDGCDAAPESGRTYYVFCNQVGLPEWIEDISRGCV
WGVNEIDPYGAICVAPDNELEYNLRWPGHWEDPETDLHYN
RFRSYSPVLGRYLQSDPAGQAGGINLYAHTANPLVFIDVL
GRECPHGNESSSECSQCADREEAERINAKILQLISKKMSI
EDAVTGHPGELIPLPHFEIDKEYSHYAKEYKQLLADIDAL
AEAREDALLREQFPSMDAVTLPPFDGKTTIGYMFYTDANG
QYHVRKLYSGGKVLSNYDSSGHVEGMAALIMRKGRITEAV
VMHNHPSGTCHYCNGQVETLLPKNAKLKVIPPANAKAPTK
YWYDQPVDYLGNSNDPKPPS
NCBI Accession No. WP_083941146.1 (TDD14)
(SEQ ID NO: 124)
GSSGKNVRMPRDYASELPEYDGKTTHGVLVTNEGKVIQLR
SGGKEEPYTGYKAVSASHVEGKAAIWIRENGSSGGTVYHN
NTTGTCGYCNSQVKALLPEGVELKIVPPTNAVAKNAQARA
VPTINVGNGTQPGRKQK
NCBI Accession No. WP_082507154.1 (TDD15)
(SEQ ID NO: 125)
MDAETGLVYFQARYYDPQLGRFITQDPYEGDWKTPLSLHH
YLYAYANPTTYVDLNGYYARDANEVQRYIIAESNCAKTGS
CDAVTALREPSEARQRSAANCKSLDRCREIADDAARSEGD
ISARIKALQKDLRNGIEANPTTGIKTIWELDKQLEARNIS
AGAVREAGRHVRWRAFVENRELTDHEKVAPAAEMYGVLSG
GRIVIARAVARSSVTRASITQESKTIGVTAEVAPNESLRN
TSGDLRASANSARNQPYGNGQSASASPSTNSAGSSGKNVR
LPRDYASELPEYDGKTTYGVLVTNEGKVIQLRSGGKEVPY
SGYKAVSASHVEGKAAIWIRENASSGGTVYHNNTTGTCGY
CNSQVKALLPEGVELKIVPPANAVARNSQAKAIPTINVGN
ATQPGRKP
NCBI Accession No. WP_044236021.1 (TDD16)
(SEQ ID NO: 126)
MLASTWLDLVIGVDLHFELVPPVMAPVPFPHPFVGLVFDP
WGLLGGLVISNVMSVATGGSLQGPVLINLMPATTTGTDAK
NWMLLPHFIIPPGVMWAPMVRVPKPSIIPGKPIGLELPIP
PPGDAVVITGSKTVHAMGANLCRLGDIALSCSDPIRLPTA
AILTIPKGMPVLVGGPPALDLMAAAFALIKCKWVANRLHK
LVNRIKNARLRNLLNRVVCFFTGHPVDVATGRVMTQATDF
ELPGPLPLQFERVYASSWADRASPVGRGWSHSLDQAVWLE
PGKVVYRAEDGREIELDTFELPGRMLQPGQESFEPLNRLL
FRCLDGHRWEVESAEGLVHEFAPVAGDADPAMARLTRKRS
RQGHAITLHYDGKGCLTWVQDSGGRIVRFEHDEAGHLTQV
SLPHPTQPGWLPHTRYIYSPEGDLVEVVDPLGHRTRYEYV
GHLLVRETDRTGLSFYFGYDGTGPGAYCIRTWGDGGIYDH
EIDYDKVNRVTFVTDSLGATTTYEMNVANAVVKVIDPRGG
ETRYEYNDVLWKTEEVEPAGGATRYEYDARGNCTKSTGPD
GATVQVEYDARNVPIRAVNPCGEEWQWVYDAQGQLVERID
PLGETTRYEYDKGMVVTITEASGVTTAEYDDSRNLRRVQG
PSEAETSYVYDALGRMVVKRSPARVAERLHYDACGRLVTV
EQPDGNVWRLAYDGEGNLTEIQDHHQRVRMRYGGYHQMVS
RQEAEDTTLFRYDSEGRLVAIENEAGEIYQYELDSCGRAG
LERGEDGGCWKYERDAAGRVIKLRKPSGAEARLIYDAMGR
LVEVRRSDSAVERFRYRKDGALIEAENSTIQVKFERDALG
RVVREMQGGHWVESSYERGARTWVASSLGVHSAIMRDERR
SVVAMTAGRGVDEWRVELSRDAFGLETERKLSSGIVSTWA
RDALGRPRHRGVAHSNNVLFGVEYQWAPGSRLVALIDTER
GTTAFHFDERSRLVGAKLPGGRIDRREPDRIGNIYRAQDQ
RDRTYSDGGILRGAGETRYTHDLDGNLTQKVLPDGATWSY
SYNAAGCLKEVERPDGTRVTFAYDALGRRVSKRWGENEVW
WLWDRHVPLHEISTRAEPITWLFEPESFAPIAKIEGDRHY
DILCDHLGAPTVVLDEAGVVTWRARLDIHAAVQPEIAETE
CPWRWPGQYEDQETGLYYNRFRYYDPEADRYISQDPLGPV
GGLNLYSYAADPLTWSDPLGLQPDPPPPPTPMGNTLPGWD
GGKTQGWFVYPDGTERHLISGYDGPSKFTQGIPGMNGNIK
SHVEAHAAALMRQYELSKATLYINRVPCPGVRGCDALLAR
MLPEGVQLEIIGPNGFKKTYTGLPDPKLKPKGCS
NCBI Accession No. WP_165374601.1 (TDD17)
(SEQ ID NO: 127)
MTACSDSPRLPPSLLELPDTPCPEPDEAASPFPAELPHSA
TVEAGAIAGSFGVTSTGEATYTIPLVVPPGRAGMQPELAV
QYDSASGEGVLGMGFSVTGLSAVTRCPRNLAQDGEIRAVR
YDEGDALCLDGKRLVEVGGGGEVVEYRTVPDTFARVVASY
EGGWDRARGPKRLRVFTRAGRVLEYGGEPSGQVLAKGGVI
RAWWATRVSDRSGNTIDFHYQNETSASEGYTVEHAPRRIE
YTGHPRAAATRAIEFVYAPRRPGTGRVLYSRGMALRSSQQ
LDRIRMLGPGGALVREYRFSYTSGPATGRRLLNAVRECAA
DGRCKPATRFRWHHGTGPGFAEVGTRLRVPESERGSLMTM
DATGDGRDDLVTTDLDLPVDDDNPITNFFVAPNRMAEGGS
SSFGALALAHQEMHHAPPSPVQPELGTPIDYNDDGRMDIF
LHDVHGRYPDWHVLLATPEGTERRKSTGIRRKFGIDAPPP
LDLNSRNASAHLADVDGDGIADLLQCEDTGSVFTDWTLHL
WRPAASGFEPEPSRIPALRGHPCNAETHLADVDSDGKVDL
LVYEATITGNGTLFGTTFEALSFVRPGEWTKRATGLPVLK
AGSGGRVIVLDVNGDGLPDAVETGFDDGQLRTFINTGDGF
AAGVSSLPSFVEDADAFAKLAAPIDHNSDGRQDLLMPIRE
PGGPVLWKILQATGSTGDGTFAVIDARLPVSEVLVDREIT
LAHPWAPRVTDVDGDGNQDVVLAVGKELRVFRSRLREEDL
LWTVSDGMSAYDPEEAGHVPKVQIEYSHLSAAEPGVRGEQ
RTYLPRYDTGEPGDGACDYPVRCALGPRRVVSRYAVNNGA
DRLRTFQVAYRNGKYHRLGRGFLGFGVRIVRDAASGAGSA
EFFDNVTFDPSDRSFPLAGHVVREWRWTPEPQQKGVSRVE
LSYTERLIHAILTNRGKSYFTLPVYQKQRREQGEHRRDSG
KTLEEYVRDTWYAPTQVVSRTERLVSAWDAFGNIREESTS
TAGVDLTLKVKRTERNDEDAWLIGLLETQQECSRALSIEQ
CRTSSRAYDRHGRVRTESAGSDDDDPETVVRVRYTRDAFG
NVIHTRAEDAFGGRRKACVSYDAEGVFPYAQRNPEGHVTY
TRYDAGHGALEAVVDPNGLATQWAHDGLGRITEERRPDGT
TTRATLSRTRDGGPRGDAWRVLRRTATDGGADETVELDGF
GRPIRGWAYKARTDDGPAERVVQEIAFDQSGERVARRSLP
AAEGTPRERMQVETYGHDATGRIAWHRAAWGAETRYRYLG
RTVEVEGPGGRVTTIENDALGRPVRIVDPEGGVTSYAYGP
FGGLWTVTDPGDAKTTTERDAYGRVRRHIDPDRGTAVAHY
DGFGQQTSTVDALGREVSWKHDRLGRAVERSDEDGTTTWT
WDEAEHGVGKLAEVASPEGHRTTYRYDALGRLREEELAIE
GERFATTVDYDGHSRPFRLWYPQAEGERRFGVRRIFDAHG
HLVGLRNERSREMFWRLEDTDEAGRIRIEEFGNGVTTERS
YHETKGRLRRVATMKDHVVLQDLWYGYDDRLNLSSRRDDR
LERTEHFRYDKLDRLTCAARHERFCLFETTYAPNGNIREK
PDVGEYTYDPEHPHAVRTAGADVFAYDAVGNQVRRPGVEE
IRYTAFDLPASITLAGGTGTVDLDYDGDQRRIRKTTPMEQ
TVYAGDLYERVTDLATGVVEHRYTVRSSERAVAVVTKRAG
GEARTLYIHVDHLGSVDLLTEGRGEDAGREVERRSYDAFG
ARRDPVTWRRAPKAEAPPALLARGFTGHGSDDELGLVHMK
GRLYDPKIGRFTTPDPVVSRPLFGQSWNAYSYVLNNPLAY
VDPSGFQEAVPEDRGGSSRAAGAEFTSDELGLPPIEELVV
ARFPEHEARSDADANAMGAEVGGAVPPVDVGVYGTSAGFV
PQPGPSSPEHASAASVVGEGLLGAGEGTGELALRVARSLV
LSALTFGGYGTYELGRAMWDGYKENGVVGALNAVNPLYQI
GRGAADTALAIDRDDYRAAGAAGVKTVIIGAATVFGAGRG
LGALEEATTAAGIARGAPSLPVYTGGKTTGVLRTATGDMP
LVSGYKGPSASMPRGTPGMNGRIKSHVEAHAAAVMRERGI
KDATLHINQVPCSSATGCGAMLPRMLPEGAQLRVLGPDGY
DQVFIGLPD
NCBI Accession No. NLI59004.1 (TDD18)
(SEQ ID NO: 128)
MVIIGRIDTNESTVSLYQWSLLPATDTNCYKEITVEQYKN
NQLVRKVSFSKAFVVNYTESYSNHVGVGTFTLYVRQFCGK
DIEVTSQELNSVSNLTPNLPNSVEKDVEVVEIAEKQAVVK
SDTSNLKQSNMSITDRLAKQKEKQDNTNIIDNRPKLPDYD
GKTTHGILVTPNSEHIPFSSGNPNPNYKNYIPASHVEGKS
AIYMRENGITSGTIYYNNTDGTCPYCDKMLSTLLEEGSVL
EVIPPINAKAPKPSWVDKPKTYIGNNKVPKPNK
NCBI Accession No. KAB8140648.1 (TDD19)
(SEQ ID NO: 129)
MLYAYGPESVVAERTIVGTTVADAGKAAFRVLDDTLAEGV
EHSANKADEAGELIEAVVEQCLRNSFSADTLVTTASGLRP
ISTIAVGELVLAWDATTRSTGYYPVTAVMLHTDAAQVHLS
VGGEHVETTPEHPFYTLERGWVAAGDLWDGAHVRRADGSY
ALTLVLWLDAEPQVMYNLTVATAHTFFVGVERALVHNAGC
PGDALPPYGTKGSKTTGILDTGNESILLESGENGPGMMVP
RDTPGMSGAMPNRAHVEGHTAAIMRNENIRLADLYINRMP
CSGAYGCMVNLPHMLPEGSILRIHVRAKLSDPWTTLPPFV
GISDTLWPPSGLNPKIVLP

In some embodiments, said sequences do not include a signal sequence, if present.

In some embodiments, the cytidine deaminase may comprise the toxic domain of a TDD. Examples of toxic domains for TDD1-TDD19 are as follows: TDD1 (SEQ ID NO: 92), TDD2 (SEQ ID NO: 95 or 134), TDD3 (SEQ ID NO: 98), TDD4 (SEQ ID NO: 101 or 143), TDDS (SEQ ID NO: 104), TDD6 (SEQ ID NO: 107 or 152), TDD7 (SEQ ID NO: 157), TDD8 (SEQ ID NO: 162), TDD9 (SEQ ID NO: 167), TDD10 (SEQ ID NO: 172), TDD11 (SEQ ID NO: 177), TDD12 (SEQ ID NO: 184), TDD13 (SEQ ID NO: 189), TDD14 (SEQ ID NO: 194), TDD15 (SEQ ID NO: 199), TDD16 (SEQ ID NO: 204), TDD17 (SEQ ID NO: 209), TDD18 (SEQ ID NO: 214), and TDD19 (SEQ ID NO: 219), e.g., as shown in Table 9. The toxic domains of TDD1-TDD19 may be split into half domains, e.g., as shown in Table 9. In some embodiments, the toxic domains of TDD1-TDD19 are split into half domains at the residues indicated in Table 9. In certain embodiments, TDD half domain pairs may comprise the amino acid sequences of SEQ ID NOs: 93 and 94, SEQ ID NOs: 96 and 97, SEQ ID NOs: 99 and 100, SEQ ID NOs: 102 and 103, SEQ ID NOs: 105 and 106, SEQ ID NOs: 108 and 109, SEQ ID NOs: 130 and 131, SEQ ID NOs: 132 and 133, SEQ ID NOs: 135 and 136, SEQ ID NOs: 137 and 138, SEQ ID NOs: 139 and 140, SEQ ID NOs: 141 and 142, SEQ ID NOs: 144 and 145, SEQ ID NOs: 146 and 147, SEQ ID NOs: 148 and 149, SEQ ID NOs: 150 and 151, SEQ ID NOs: 153 and 154, SEQ ID NOs: 155 and 156, SEQ ID NOs: 158 and 159, SEQ ID NOs: 160 and 161, SEQ ID NOs: 163 and 164, SEQ ID NOs: 165 and 166, SEQ ID NOs: 168 and 169, SEQ ID NOs: 170 and 171, SEQ ID NOs: 173 and 174, SEQ ID NOs: 175 and 176, SEQ ID NOs: 178 and 179, SEQ ID NOs: 180 and 181, SEQ ID NOs: 182 and 183, SEQ ID NOs: 185 and 186, SEQ ID NOs: 187 and 188, SEQ ID NOs: 190 and 191, SEQ ID NOs: 192 and 193, SEQ ID NOs: 195 and 196, SEQ ID NOs: 197 and 198, SEQ ID NOs: 200 and 201, SEQ ID NOs: 202 and 203, SEQ ID NOs: 205 and 206, SEQ ID NOs: 207 and 208, SEQ ID NOs: 210 and 211, SEQ ID NOs: 212 and 213, SEQ ID NOs: 215 and 216, SEQ ID NOs: 217 and 218, SEQ ID NOs: 220 and 221, or SEQ ID NOs: 222 and 223.

As used herein, unless specified otherwise, the term “TDD” refers to the TDD toxic domain.

Where the present disclosure refers to a cytidine deaminase (e.g., a TDD described herein), it is contemplated that other cytidine deaminases can be used in the fusion proteins and cell editing systems described herein. The cytidine deaminase can comprise wild-type or evolved domains. In certain embodiments, the cytidine deaminase may be, e.g., apolipoprotein B mRNA-editing complex 1 (APOBEC1) domain or an Activation Induced Deaminase (AID).

The present disclosure also provides other potential cytidine deaminases. Such cytidine deaminases may be used, e.g., in the fusion proteins and cell editing systems described herein. In some embodiments, the cytidine deaminases are functional analogs of a TDD described herein. A functional analog of a TDD is a molecule having the same or substantially the same biological function as said TDD (i.e., cytidine deaminase function). For example, the functional analog may be an isoform or a variant of the TDD, e.g., containing a portion of the TDD with or without additional amino acid residues and/or containing mutations relative to the TDD (e.g., a variant with at least 70%, 75%, 80%, 85%, 90%, 95%, 96%, 97%, 98%, or 99% sequence identity to the TDD (e.g., a TDD comprising the amino acid sequence of any one of SEQ ID NOs: 72, 86-91, and 117-129) or its toxic domain (e.g., a toxic domain comprising the amino acid sequence of SEQ ID NO: 49, 81, 92, 98, 101, 104, 107, 134, 143, 152, 157, 162, 167, 172, 177, 184, 189, 194, 199, 204, 209, 214, or 219)). In certain embodiments, the functional analogs are orthologs of a TDD described herein. In certain embodiments, a TDD ortholog may comprise an amino acid sequence at least 20%, 25%, 30%, 35%, 40%, 45%, 50%, 55%, 60%, 65%, 70%, 75%, 80%, 85%, 90%, 95%, 96%, 97%, 98%, or 99% identical to the amino acid sequence of said TDD (e.g., a TDD comprising the amino acid sequence of any one of SEQ ID NOs: 72, 86-91, and 117-129). In certain embodiments, a TDD ortholog may comprise a toxic domain with an amino acid sequence that is at least 20%, 25%, 30%, 35%, 40%, 45%, 50%, 55%, 60%, 65%, 70%, 75%, 80%, 85%, 90%, 95%, 96%, 97%, 98%, or 99% identical to the amino acid sequence of the toxic domain of a TDD described herein (e.g., a toxic domain comprising the amino acid sequence of SEQ ID NO: 49, 81, 92, 95, 98, 101, 104, 107, 134, 143, 152, 157, 162, 167, 172, 177, 184, 189, 194, 199, 204, 209, 214, or 219).

The term “percent identical” in the context of amino acid or nucleotide sequences refers to the percent of residues in two sequences that are the same when aligned for maximum correspondence. The percent identity of two sequences may be obtained by, e.g., BLAST® using default parameters (available at the U.S. National Library of Medicine's National Center for Biotechnology Information website). In some embodiments, the length of a reference sequence aligned for comparison purposes is at least 30%, (e.g., at least 40, 50, 70, 80, or 90%, or 100%) of the reference sequence.

In certain embodiments, a cytidine deaminase described herein may target a cytidine in an AC sequence, a TC sequence, a GC sequence, a CC sequence, an AAC sequence, a TAC sequence, a GAC sequence, a CAC sequence, an ATC sequence, a TTC sequence, a GTC sequence, a CTC sequence, an AGC sequence, a TGC sequence, a GGC sequence, a CGC sequence, an ACC sequence, a TCC sequence, a GCC sequence, a CCC sequence, or any combination thereof. In certain embodiments, a cytidine deaminase described herein has increased efficiency and/or activity compared to DddA. In some embodiments, the increased efficiency or activity may be, e.g., at any one or combination of the above target sequences.

It is also contemplated that adenine deaminases (e.g., TadA) may be used in the fusion proteins and cell editing systems described herein for conversion of A:T base pairs to G:C base pairs. In certain embodiments, a TDD may be mutated at residues that form the nucleotide pocket (e.g., a residue or combination of residues as described above for DddA) to allow the enzyme to act as an adenine deaminase, and/or to reduce TC sequence bias within the base editing window.

### B. Zinc Finger Protein Domains

The fusion proteins described herein (such as ZFP-cytidine deaminase (e.g., ZFP-TDD), ZFP-cytidine deaminase inhibitor (e.g., ZFP-TDDI), or ZFP-nickase fusion proteins) comprise zinc finger protein (ZFP) domains. A “zinc finger protein” or “ZFP” refers to a protein having DNA-binding domains that are stabilized by zinc. ZFPs bind to DNA in a sequence-specific manner. The individual DNA-binding domains are referred to as “fingers.” A ZFP has at least one finger, and each finger binds from two to four base pairs of nucleotides, typically three or four base pairs of DNA (contiguous or noncontiguous). Each zinc finger typically comprises approximately 30 amino acids and chelates zinc. An engineered ZFP can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers that bind the particular triplet or quadruplet sequence. See, e.g., ZFP design methods described in detail in U.S. Pat. Nos. 5,789,538; 5,925,523; 6,007,988; 6,013,453; 6,140,081; 6,200,759; 6,453,242; 6,534,261; 6,979,539; and 8,586,526; and International Pat. Pubs. WO 95/19431; WO 96/06166; WO 98/53057; WO 98/53058; WO 98/53059; WO 98/53060; WO 98/54311; WO 00/27878; WO 01/60970; WO 01/88197; WO 02/016536; WO 02/099084; and WO 03/016496.

The ZFP domain of the present ZFP fusion proteins may include at least three (e.g., four, five, six, seven, eight, nine, ten, eleven, twelve, thirteen, or more) zinc fingers. Individual zinc fingers are typically spaced at three base pair intervals when bound to DNA. unless they are connected by engineered linkers capable of skipping one or more bases (see, e.g., Paschon et al., Nat Commun. (2019) 10:1133 and U.S. Pat. Nos. 8,772,453; 9,163,245; 9,394,531; and 9,982,245). A ZFP domain having three fingers typically recognizes a target site that includes 9 or 12 nucleotides. A ZFP domain having four fingers typically recognizes a target site that includes 12 to 15 nucleotides. A ZFP domain having five fingers typically recognizes a target site that includes 15 to 18 nucleotides. A ZFP domain having six fingers can recognize target sites that include 18 to 21 nucleotides.

The target specificity of the ZFP domain may be improved by mutations to the ZFP backbone as described in, e.g., U.S. Pat. Pub. 2018/0087072. The mutations include those made to residues in the ZFP backbone that can interact non-specifically with phosphates on the DNA backbone but are not involved in nucleotide target specificity. In some embodiments, these mutations comprise mutating a cationic amino acid residue to a neutral or anionic amino acid residue. In some embodiments, these mutations comprise mutating a polar amino acid residue to a neutral or non-polar amino acid residue. In further embodiments, mutations are made at positions (−4), (−5), (−9) and/or (−14) relative to the DNA-binding helix. In some embodiments, a zinc finger may comprise one or more mutations at positions (−4), (−5), (−9) and/or (−14). In further embodiments, one or more zinc fingers in a multi-finger ZFP domain may comprise mutations at positions (−4), (−5), (−9) and/or (−14). In some embodiments, the amino acids at positions (−4), (−5), (−9) and/or (−14) (e.g., an arginine (R) or lysine (K)) are mutated to an alanine (A), leucine (L), Ser (S), Asp (N), Glu (E), Tyr (Y), and/or glutamine (Q). In some embodiments, the R residue at position (−4) is mutated to Q.

Alternatively, the DNA-binding domain may be derived from a nuclease. For example, the recognition sequences of homing endonucleases and meganucleases such as I-Scel, I-SceI, I-CeuI, PI-PspI, PI-Sce, I-SceIV, I-csmI, I-PanI, i-SceII, I-PpoI, I-SceIII, I-CreI, I-TevI, I-TevII and I-TevIII are known. See also U.S. Pat. Nos. 5,420,032 and 6,833,252; Belfort et al., Nucleic Acids Res. (1997) 25:3379-88; Dujon et al., Gene (1989) 82:115-8; Perler et al., Nucleic Acids Res. (1994) 22:1125-7; Jasin, Trends Genet. (1996) 12:224-8; Gimble et al., J Mol Biol. (1996) 263:163-80; Argast et al., J Mol Biol. (1998) 280:345-53; and the New England Biolabs catalogue. In addition, the DNA-binding specificity of homing endonucleases and meganucleases can be engineered to bind non-natural target sites. See, for example, Chevalier et al., Mol Cell (2002) 10:895-905; Epinat et al., Nucleic Acids Res. (2003) 31:2952-62; Ashworth et al., Nature (2006) 441:656-59; Paques et al., Current Gene Therapy (2007) 7:49-66; and U.S. Pat. Pub. 2007/0117128.

In some embodiments, the present ZFP fusion proteins comprise one or more zinc finger domains. The domains may be linked together via an extendable flexible linker such that, for example, one domain comprises one or more (e.g., 3, 4, 5, or 6) zinc fingers and another domain comprises additional one or more (e.g., 3, 4, 5, or 6) zinc fingers. In some embodiments, the linker is a standard inter-finger linker such that the finger array comprises one DNA-binding domain comprising 8, 9, 10, 11 or 12 or more fingers. In other embodiments, the linker is an atypical linker such as a flexible linker. For example, two ZFP domains may be linked to a cytidine deaminase, inhibitor, or nickase domain (“domain”) such as those described herein in the configuration (from N terminus to C terminus) ZFP-ZFP-domain, domain-ZFP-ZFP, ZFP-domain-ZFP, or ZFP-domain-ZFP-domain (two ZFP-domain fusion proteins are fused together via a linker).

In some embodiments, the ZFP fusion proteins are “two-handed,” i.e., they contain two zinc finger clusters (two ZFP domains) separated by intervening amino acids so that the two ZFP domains bind to two discontinuous target sites. An example of a two-handed type of zinc finger binding protein is SIP1, where a cluster of four zinc fingers is located at the amino terminus of the protein and a cluster of three fingers is located at the carboxyl terminus (see Remade et al., EMBO J. (1999) 18(18):5073-84). Each cluster of zinc fingers in these proteins is able to bind to a unique target sequence and the spacing between the two target sequences can comprise many nucleotides.

The DNA-binding ZFP domains of the ZFP fusion proteins described herein direct the proteins to DNA target regions. In some embodiments, the DNA target region is at least 8 bps in length. For example, the target region may be 8 bps to 40 bps in length, such as 12, 13, 14, 15, 16, 17, 18, 19, 20, 21, 22, 23, 24, 25, 26, 27, 28, 29, 30, 31, 32, 33, 34, 35, or 36 bps in length.

In certain embodiments, the ZFP binds to a target site that is 1 to 100 (or any number therebetween) nucleotides on either side of the targeted base. In other embodiments, the ZFP binds to a target site that is 1 to 50 (or any number therebetween) nucleotides on either side of the targeted base.

### C. Base Editor Inhibitors

In some embodiments, the base editor systems described herein may include an inhibitor of the editor to better regulate temporally and spatially the base editing activity of the systems. For example, where the cytidine deaminase is a TDD as described herein, the inhibitor may be a TDDI that inhibits said TDD. Where the editor is the cytidine deaminase DddA, the inhibitor may be, e.g., DddI. In some embodiments, DddI has the amino acid sequence shown below.

(SEQ ID NO: 73)
MYADDFDGEI EIDEVDSLVE FLSRRPAFDA NNFVLTFEES
GFPQLNIFAK NDIAVVYYMD IGENFVSKGN SASGGTEKFY
ENKLGGEVDL SKDCVVSKEQ MIEAAKQFFA TKQRPEQLTW SEL

Thus, in some embodiments, the base editor systems include a TDDI component in addition to ZFP-TDD fusion proteins. The TDDI component may be brought in close proximity to the TDD complex through a DNA-binding domain covalently fused to it, or through dimerization with a DNA-binding domain not covalently bound to it.

In some embodiments, the present base editing system comprises a ZFP-inhibitor fusion protein comprising a ZFP domain and an inhibitor domain, wherein the ZFP domain binds to a sequence in the DNA target region close (e.g., within 50-100 nt) to the ZFP-cytidine deaminase fusion proteins' binding sites. When this ZFP-inhibitor fusion protein is introduced to the cell, the inhibitor domain will be brought within close proximity to the cytidine deaminase complex and bind to the complex, thereby inhibiting the base editing activity of the cytidine deaminase at that locus. The presence of the sequence bound by the ZFP domain of ZFP-inhibitor determines the inhibitory activity of the inhibitor.

In some embodiments, the binding of the inhibitor domain to the cytidine deaminase complex may be regulated by an agent (e.g., a small molecule or a peptide). For example, the inhibitor domain may be fused to a dimerization domain, and its dimerization partner may be fused to a ZFP domain that binds to a sequence in the DNA target region close (e.g., within 50-100 nt) to the ZFP-cytidine deaminase fusion proteins' binding sites. The dimerization domains of the inhibitor and the ZFP may dimerize in the presence of a dimerization-inducing agent (e.g., a small molecule or peptide). In the presence of the agent, the inhibitor domain will be brought within close proximity to the DNA target region through dimerization, leading to binding and inactivation of the cytidine deaminase complex. Once the agent is withdrawn, the inhibitor domain will no longer be sequestered near the DNA target region and will detach from the cytidine deaminase complex, allowing the base editing process to proceed. Examples of such agents and dimerizing domains are shown in Table 1 below:

TABLE 1
Dimerization Domains and Dimerization-Inducing Agents

| | Dimerization Partners | Dimerizing Agent |
|---|---|---|
| FKBP | FKBP | FK1012 |
| FKBP | Calcineurin A (can) | FK506 |
| FKBP | CyP-Fas | FKCsA |
| FKBP | FRB (FKBP-rapamycin-binding) | Rapamycin |
| | domain of mTOR | |
| GyrB | GyrB | Coumermycin |
| GAI | GID1 (gibberellin insensitive dwarf 1) | Gibberellin |
| ABI | PYL | Abscisic acid |
| ABI | PYR^{Mandi} | Mandipropamid |
| SNAP-tag | HaloTag | HaXS |
| eDHFR | HaloTag | TMp-HTag |
| Bcl-xL | Fab (AZ1) | ABT-737 |

Conversely, the dimerization of the domains fused to the ZFP and the inhibitor domains may be inhibited, rather than promoted, by a dimerization-inhibiting agent (e.g., a small molecule or peptide) such that the presence of the agent will permit activity of the cytidine deaminase complex. If the agent is withdrawn, the inhibitor domain will be able to bind to the cytidine deaminase complex, inhibiting the base editing process.

### D. Uracil DNA Glycosylase Inhibitors

The term “uracil glycosylase inhibitor” or “UGI” as used herein, refers to a protein that can inhibit a uracil-DNA glycosylase base-excision repair enzyme. Upon detecting a G:U mismatch, the cell responds through base excision repair, initiated by excision of the mismatched uracil by uracil N-glycosylase (UNG). In some embodiments, a base editor system described herein further comprises one or more UGIs to protect the edited G:U intermediate from excision by UNG. In certain embodiments, a ZFP-cytidine deaminase (e.g., ZFP-TDD) fusion protein described herein may comprise one or more UGI domains, e.g., attached by a linker described herein. In some embodiments, the linker is an SGGS linker (SEQ ID NO: 245). The UGI domain(s) may be located at the N-terminus, the C-terminus, or any combination thereof, of the fusion protein (e.g., one UGI domain at the C-terminus, one UGI domain at the N-terminus, two UGI domains at the C-terminus, two UGI domains at the N-terminus, or any combination thereof). Additionally or alternatively, one or more UGI domains may be on a separate ZFP fusion protein (“ZFP-UGI”). In particular embodiments, the UGI domain comprises the amino acid sequence of SEQ ID NO: 20.

### E. Nickases

In some embodiments, a base editor system described herein further comprises a nickase to create a single-stranded DNA break in the vicinity of the edited DNA target region (e.g., within 10, 20, 30, 40, 50, 60, 70, 80, 90, or 100 nt from the edited base). The creation of the nick attracts DNA repair machinery such that the region downstream of the nick is excised and replaced, resulting in a fully edited double-stranded DNA target region. The nick may be, for example, 5′ or 3′ of the edited base on the same strand or the opposite strand.

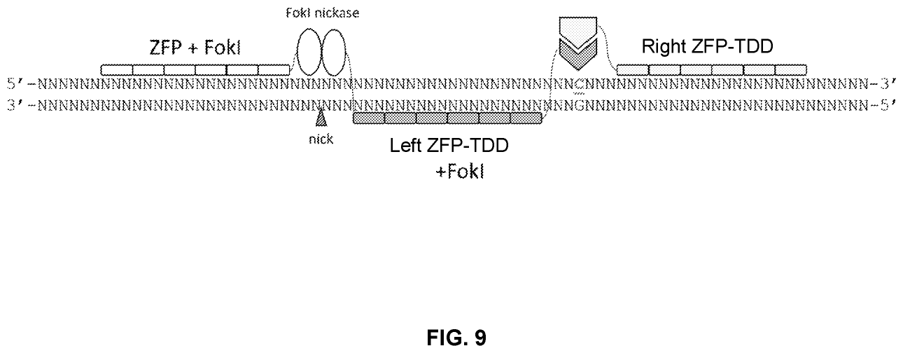

In some embodiments, the base editor system described herein has a trimeric architecture to include nickase function. For example, one domain of a dimeric nickase may be fused to a ZFP-cytidine deaminase (e.g., a ZFP-TDD as described herein) and the other domain may be fused to an independent ZFP, such that binding of both ZFP domains to their DNA target regions results in an active nickase capable of producing a single-strand break. See, e.g., FIG. 9.

In some embodiments, the base editor system described herein has a tetrameric architecture to include nickase function. In addition to the two ZFP-cytidine deaminase (e.g., ZFP-TDD as described herein) fusion proteins, such a system also comprises two ZFP-nickase proteins, wherein one domain of a dimeric nickase is fused to a first ZFP domain and the other domain fused to a second ZFP domain, such that binding of both ZFP domains to their DNA target regions results in an active nickase capable of producing a single-strand break.

In some embodiments, the nickase may be, for example, a ZFN nickase, a TALEN nickase, or a CRISPR/Cas nickase. In certain embodiments, the nickase is derived from a FokI DNA cleavage domain. In some embodiments, the Fokl nickase comprises one or more mutations as compared to a parental Fokl nickase, e.g., mutations to change the charge of the cleavage domain; mutations to residues that are predicted to be close to the DNA backbone based on molecular modeling and that show variation in Fokl homologs; and/or mutations at other residues (see, e.g., U.S. Pat. No. 8,623,618 and Guo et al., J Mol Biol. (2010) 400(1):96-107).

In the ZFP fusion proteins described herein, the nickase domain(s) may be positioned on either side of the DNA-binding ZFP domain, including at the N- or C-terminal side of the fusion molecule (N- and/or C-terminal to the ZFP domain). In some embodiments, a ZFP-cytidine deaminase (e.g., ZFP-TDD as described herein) fusion protein described herein comprises a cytidine deaminase domain at the N- or C- terminus and a nickase domain at the opposite terminus.

### F. Peptide Linkers

In the fusion proteins described herein, the ZFP, cytidine deaminase (e.g., a TDD as described herein), inhibitor (e.g., a TDDI, such as DddI where the cytidine deaminase is DddA), nickase, and/or UGI domains may be positioned in any order relative to each other. In some embodiments, the domains may be associated with each other by direct peptidyl linkages, peptide linkers, or any combination thereof. In some embodiments, two or more of the domains may be associated with each other by dimerization (e.g., through a leucine zipper, a STAT protein N-terminal domain, or an FK506 binding protein).

In some embodiments, the ZFP, cytidine deaminase (e.g., a TDD as described herein), inhibitor (e.g., a TDDI, such as DddI where the cytidine deaminase is DddA), UGI, and/or nickase domains, and/or the zinc fingers within the ZFP domain, may be linked through a peptide linker, e.g., a noncleavable peptide linker of about 5 to 200 amino acids (e.g., 5, 6, 7, 8, 9, 10, 11, 12, 13, 14, 15, 16, 17, 18, 19, 20, 21, 22, 23, 24, 25, or 26 or more amino acids). Preferred linkers are typically flexible amino acid subsequences that are synthesized as a recombinant fusion protein. See, e.g., U.S. Pat. Nos. 6,479,626; 6,903,185; 7,153,949; 8,772,453; and 9,163,245; and PCT Patent Pub. WO 2011/139349. The proteins described herein may include any combination of suitable linkers.

In some embodiments, the peptide linker is three to 30 amino acid residues in length and is rich in G and/or S. Non-limiting examples of such linkers are SGGS linkers (SEQ ID NO: 245) as well as G4S-type linkers, i.e., linkers containing one or more (e.g., 2, 3, or 4) GGGGS (SEQ ID NO: 71) motifs, or variations of the motif (such as ones that have one, two, or three amino acid insertions, deletions, and substitutions from the motif).

In particular embodiments, a peptide linker used in a fusion protein described herein may be L0 (LRGSQLVKS; SEQ ID NO: 15), L7A (LRGSQLVKSKSEAAAR; SEQ ID NO: 16), L26 (LRGSQLVKSKSEAAARGGGGSGGGGS; SEQ ID NO: 17), L21 (LRGSQLVKSKSEAAARGGGGS; SEQ ID NO: 110), L18 (LRGSQLVKSKSEAAARGS; SEQ ID NO: 111), L13 (LRGSQLVKSKSGS; SEQ ID NO: 112), L11 (LRGSQLVKSGS; SEQ ID NO: 113), L9 (LRGSQLVGS; SEQ ID NO: 114), L6 (LRGSGS; SEQ ID NO: 115), or L4 (LRGS; SEQ ID NO: 116).

### II. Base Editor Systems

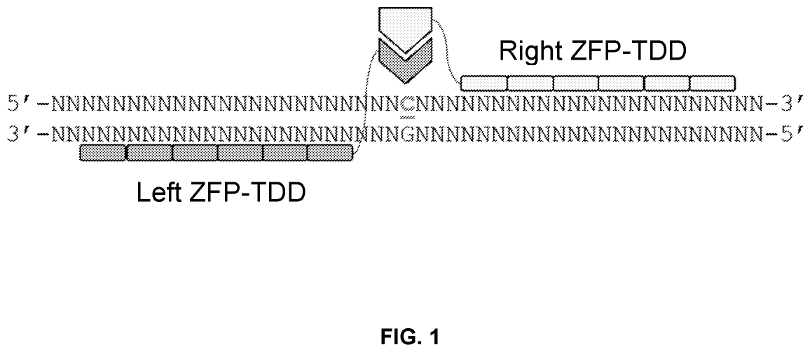

The present disclosure provides base editor systems comprising the ZFP fusion proteins described herein. The base editor systems can be used to edit a cytosine base to a uracil base in a DNA target region, wherein the uracil is replaced by a thymine base during DNA replication or repair. In certain embodiments, the editing results in the change of a targeted C:G base pair to a T:A base pair. FIG. 1 illustrates a base editing system of the present disclosure.

Base editor systems as described herein can be used to knock out a gene (e.g., by changing a regular codon into a stop codon and/or by mutating a splice acceptor site to introduce exon skipping and/or frameshift mutations); introduce mutations into a control element of a gene (e.g., a promoter or enhancer region) to increase or reduce expression; correct disease-causing mutations (e.g., point mutations); and/or induce mutations that result in therapeutic benefits. The target DNA may be in a chromosome or in an extrachromosomal sequence (e.g., mitochondrial DNA) in a cell. The base editing may be performed in vitro, ex vivo, or in vivo.

In some embodiments, a base editor system described herein performs one or more codon conversions, e.g., CAA to TAA; CAG to TAG; CGA to TGA; or TGG to TAG, TGA, or TAA; or any combination thereof; thereby introducing stop codon(s).

The base editor systems of the present disclosure may comprise, in addition to ZFP-cytidine deaminase (e.g., ZFP-TDD as described herein) fusion proteins, components such as inhibitor domains (e.g., a TDDI, such as DddI where the cytidine deaminase is DddA), UGIs, and nickases, or any combination thereof, as described herein that may help regulate or improve the editing activity of the system. In certain embodiments, the system may be packaged within a single viral vector (e.g., an AAV vector).

In some embodiments, a base editor system of the present disclosure comprises a pair of ZFP-cytidine deaminase (e.g., ZFP-TDD as described herein) fusion proteins each comprising a cytidine deaminase half domain that lacks cytidine deaminase activity on its own, wherein binding of the ZFPs to their respective nucleotide targets results in an active cytidine deaminase molecule capable of editing a targeted C base to T (e.g., by replacing C with U, which is replaced by T during DNA replication or repair).

For example, in some embodiments, the base editor system may comprise: a) a first fusion protein (ZFP-TDD left) comprising: i) a first ZFP domain that binds to nucleotides of a double-stranded DNA target region on one side of the base targeted for editing; and ii) a TDD N-half domain; and b) a second fusion protein (ZFP-TDD right) comprising: i) a second ZFP domain that binds to nucleotides of the double-stranded DNA target region on the other side of the base targeted for editing; and ii) a TDD C-half domain; wherein binding of the ZFP-TDD left and the ZFP-TDD right to their respective nucleotides results in an active TDD molecule capable of editing the DNA target region by changing the C base to T. The ZFP-TDDs and/or DNA target regions may be, e.g., as described herein.

In some embodiments, the base editor system may comprise: a) a first fusion protein (ZFP-TDDI) that binds to nucleotides within a first DNA target region, comprising: i) a zinc finger protein (ZFP) domain that binds to nucleotides within a first DNA target region; and

ii) a TDDI domain; b) a second fusion protein (ZFP-TDD left) comprising: i) a ZFP domain that binds to nucleotides of a second DNA target region on one side of the base targeted for editing; and ii) a TDD N-half domain; and c) a third fusion protein (ZFP-TDD right) comprising: i) a ZFP domain that binds to nucleotides of the second DNA target region on the other side of the base targeted for editing; and ii) a TDD C-half domain; wherein binding of ZFP-TDD left and ZFP-TDD right to their respective nucleotides results in an active TDD molecule capable of editing the second DNA target region by changing the C base to T; and wherein binding of ZFP-TDDI to the first DNA target region prevents editing of the second DNA target region by the TDD. The ZFP-TDDs, ZFP-TDDI, and DNA target regions may be, e.g., as described herein.

In some embodiments, the base editor system may comprise: a) a first fusion protein comprising: i) a zinc finger protein (ZFP) domain that binds to nucleotides within a first DNA target region, and ii) a dimerization domain; b) a second fusion protein comprising: i) a TDDI domain; and ii) a dimerization domain that partners with the dimerization domain of a); c) a third fusion protein (ZFP-TDD left) comprising: i) a ZFP domain that binds to nucleotides of a second DNA target region on one side of the base targeted for editing, and ii) a TDD N-half domain; and d) a fourth fusion protein (ZFP-TDD right) comprising: i) a ZFP domain that binds to nucleotides of the second DNA target region on the other side of the base targeted for editing, and ii) a TDD C-half domain; wherein binding of ZFP-TDD left and ZFP-TDD right to their respective nucleotides results in an active TDD molecule capable of editing the second DNA target region by changing the C base to T; and wherein dimerization of the fusion proteins of a) and b) to form ZFP-TDDI and binding of the ZFP of a) to the first DNA target region prevents editing of the second DNA target region by the TDD. The ZFP-TDDs, ZFP-TDDI, and/or DNA target regions may be, e.g., as described herein.

In some embodiments, the dimerization domains of the fusion proteins of a) and b) partner to form ZFP-TDDI in the presence of a dimerization-inducing agent, resulting in inhibition of TDD activity.

In some embodiments, the dimerization domains of the fusion proteins of a) and b) are inhibited from partnering to form ZFP-TDDI in the presence of a dimerizing-inhibiting agent, permitting TDD activity.

In some embodiments, the ZFP-TDDI is specific for a sequence to be protected from TDD base editing activity. For example, the ZFP domain may bind to an allele to be preserved in its unedited form (e.g., where another allele, such as a mutated allele, is targeted for editing), or a known site of off-target editing. In some embodiments, the TDD base editing may convert a regular codon into a stop codon in the unprotected allele.

In some embodiments, expression of ZFP-TDDI (or components thereof) may be under the control of an inducible promoter. In certain embodiments, such a system may be used as a “kill switch,” wherein ZFP-TDDI protects an essential gene in a cell from being edited, and reducing or eliminating expression of ZFP-TDDI results in the death of the cell.

Where assembly of ZFP-TDDI is under the control of a dimerization-inducing or dimerization-inhibiting agent, base editing may be conditional upon the presence or absence of the agent. Such a conditional system may also be used for a “kill switch,” e.g., wherein ZFP-TDDI protects an essential gene in a cell from being edited in the presence of a dimerization-inducing agent or in the absence of a dimerization-inhibiting agent, and removing or administering the agent, respectively, results in the death of the cell.

In certain embodiments, a base editor system of the present disclosure may be a multiplex system comprising more than one ZFP-TDD left and ZFP-TDD right pair; such a system may be capable of editing more than one DNA target region at a time. In particular embodiments, to increase editing specificity, the multiplex system comprises ZFP-TDD pairs wherein the TDD N-half and C-half domains are split at a different position in the TDD sequence (e.g., a position described herein) for each pair. In certain embodiments, the DNA target regions edited by the ZFP-TDD pairs of the multiplex system may be in different genes. In certain embodiments, the DNA target regions may be in the same gene.

In any of the above embodiments, the TDD and TDDI may be any described herein. In certain embodiments, the TDD may be DddA and the TDDI may be Dddl. It is also contemplated that other cytidine deaminases and inhibitors may be used in place of the TDD and TDDI. In particular embodiments, a multiplex system described herein may comprise a first ZFP-cytidine deaminase pair and a second ZFP-cytidine deaminase pair, wherein the first and second pairs utilize different cytidine deaminases (e.g., selected from those described herein).

In some embodiments, the systems and methods described herein produce targeted editing of the DNA target region in at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95% or 100% of the cells. In some embodiments, the edited cells exhibit little to no off-target indels (e.g., less than 5%, 4%, 3%, 2%, 1%, 0.5%, 0.2%, or 0.1% off-target indels). In some embodiments, the edited cells exhibit little to no off-target base editing (e.g., less than 5%, 4%, 3%, 2%, 1%, 0.5%, 0.2%, or 0.1% off-target base editing); however, as base editing of off-target sites may not be prone to translocations or other genomic arrangements, higher percentages may also be contemplated.

The present disclosure also provides nucleic acid molecules encoding the ZFP fusion proteins described herein, which may be part of a viral or non-viral vector. Further, the present disclosure provides a cell or population of cells comprising a base editor system as described herein, as well as descendants of such cells, wherein the cells comprise one or more edited bases.

### III. Delivery of ZFP Fusion Proteins

A ZFP fusion protein of the present disclosure may be introduced to target cells as a protein, through a variety of methods (e.g., electroporation, fusion of the protein to a receptor ligand, lipid nanoparticles, cationic or anionic liposomes, or a nuclear localization signal (e.g., in combination with liposomes)). In other embodiments, the fusion protein is introduced to target cells through a nucleic acid molecule encoding it, for example, a DNA plasmid or mRNA. The nucleic acid molecule may be in a nucleic acid expression vector, which may include expression control sequences such as promoters, enhancers, transcription signal sequences, and transcription termination sequences that allow expression of the coding sequence for the ZFP fusion proteins.

In some embodiments, the promoter on the vector for directing ZFP fusion protein expression is a constitutively active promoter or an inducible promoter. Suitable promoters include, without limitation, a Rous sarcoma virus (RSV) long terminal repeat (LTR) promoter (optionally with an RSV enhancer), a cytomegalovirus (CMV) promoter (optionally with a CMV enhancer), a CMV immediate early promoter, a simian virus 40 (SV40) promoter, a dihydrofolate reductase (DHFR) promoter, a β-actin promoter, a phosphoglycerate kinase (PGK) promoter, an EFlα promoter, a Moloney murine leukemia virus (MoMLV) LTR, a creatine kinase-based (CK6) promoter, a transthyretin promoter (TTR), a thymidine kinase (TK) promoter, a tetracycline responsive promoter (TRE), a hepatitis B Virus (HBV) promoter, a human α1-antitrypsin (hAAT) promoter, chimeric liver-specific promoters (LSPs), an E2 factor (E2F) promoter, the human telomerase reverse transcriptase (hTERT) promoter, a CMV enhancer/chicken β-actin/rabbit β-globin promoter (CAG promoter; Niwa et al., Gene (1991) 108(2):193-9), and an RU-486-responsive promoter. In addition, the promoter may include one or more self-regulating elements whereby the ZFP fusion protein can bind to and repress its own expression level to a preset threshold. See U.S. Pat. No. 9,624,498.

Any method of introducing the nucleotide sequence into a cell may be employed, including but not limited to, electroporation, calcium phosphate precipitation, microinjection, cationic or anionic liposomes, liposomes in combination with a nuclear localization signal, naturally occurring liposomes (e.g., exosomes), or viral transduction. In certain embodiments, the nucleotide sequence is in the form of mRNA and is delivered to a cell via electroporation.

For in vivo delivery of an expression vector, viral transduction may be used. A variety of viral vectors known in the art may be adapted by one of skill in the art for use in the present disclosure, for example, vaccinia vectors, adenoviral vectors, lentiviral vectors, poxyviral vectors, adeno-associated viral (AAV) vectors, retroviral vectors, and hybrid viral vectors. In some embodiments, the viral vector used herein is a recombinant AAV (rAAV) vector. Any suitable AAV serotype may be used. For example, the AAV may be AAV1, AAV2, AAV3, AAV3b, AAV4, AAV5, AAV6, AAV7, AAV8, AAV8.2, AAV9, AAV.PHP.B, AAV.PHP.eB, or AAVrh10, or of a novel serotype or a pseudotype such as AAV2/8, AAV2/5, AAV2/6, AAV2/9, or AAV2/6/9. In some embodiments, the expression vector is an AAV viral vector and is introduced to the target human cell by a recombinant AAV virion whose genome comprises the construct, including having the AAV Inverted Terminal Repeat (ITR) sequences on both ends to allow the production of the AAV virion in a production system such as an insect cell/baculovirus production system or a mammalian cell production system. The AAV may be engineered such that its capsid proteins have reduced immunogenicity or enhanced transduction ability in humans. Viral vectors described herein may be produced using methods known in the art. Any suitable permissive or packaging cell type may be employed to produce the viral particles. For example, mammalian (e.g., 293) or insect (e.g., sf9) cells may be used as the packaging cell line.

Any type of cell may be targeted for the base editing methods described herein. For example, the cells may be eukaryotic or prokaryotic. In some embodiments, the cells are mammalian (e.g., human) cells or plant cells. Human cells may can include, for example, T cells, Natural Killer (NK) cells, NK T cells, alpha-beta T cells, gamma-delta T-cells, cytotoxic T lymphocytes (CTL), regulatory T cells, B cells, human embryonic stem cells, tumor-infiltrating lymphocytes (TIL) or a pluripotent stem cell from which lymphoid cells may be differentiated (e.g., an induced pluripotent stem cell (iPSC)). In some embodiments, the systems can be used to modify pluripotent stem cells prior to their differentiation into multiple cell types. For example, a lymphoid cell precursor may be modified prior to differentiation into lymphoid cell types such as regulatory T cells, effector T cells, natural killer cells, etc. The multiplex base editor systems of the present disclosure (comprising more than one ZFP-cytidine deaminase (e.g., ZFP-TDD) pair), in particular, can be used to prepare cells with multiple base edits at once, including pluripotent cells. In some embodiments, the multiplex systems may be used to prepare, e.g., allogeneic T cells. Where the systems comprise a ZFP-cytidine deaminase inhibitor (e.g., ZFP-TDDI) that can be induced to assemble in the presence or absence of a dimerization-regulating agent, as described herein, it is contemplated that the edited cells may be placed under the control of a “kill switch” activated upon administration of the agent.

For agricultural applications, any method for introduction of proteins or nucleic acid molecules to a plant cell is also contemplated, such as Agrobacterium tumefaciens-mediated T-DNA delivery.

### IV. Pharmaceutical Applications

The present disclosure provides methods of editing a cytosine to a thymine base in cellular DNA, comprising delivering a base editor system described herein to a cell (e.g., from a patient), resulting in the replacement of a targeted C base with a T base. The cell may be within a patient (in vivo treatment), or a method as described herein may be performed on a cell removed from a patient and then the edited cell delivered to the patient (ex vivo treatment). In some embodiments, the cells are further manipulated ex vivo prior to use as a treatment. The term “treating” encompasses alleviation of symptoms, prevention of onset of symptoms, slowing of disease progression, improvement of quality of life, and increased survival. In some embodiments, a patient treated by the methods described herein is a mammal, e.g., a human.

In some embodiments, the methods of the present disclosure are used to edit a gene or regulatory sequence associated with a disease. For example, in certain embodiments, the base editing may correct a point mutation in a DNA sequence to restore normal gene expression or activity. In certain embodiments, the base editing may introduce a stop codon into a deleterious gene (e.g., an oncogene). In certain embodiments, the base editing may introduce a mutation that results in a therapeutic benefit.

In some embodiments, the patient has cancer. In certain embodiments, the cell from the patient is further modified before or after base editing to provide resistance to a chemotherapeutic agent. The patient may then be treated with the chemotherapeutic agent, which in some embodiments may result in greater survival of edited over unedited cells.

In some embodiments, the patient has an autoimmune disorder.

In some embodiments, the patient has an autosomal dominant disease, such as autosomal dominant polycystic kidney disease.

In some embodiments, the patient has a mitochondrial disorder.

In some embodiments, the patient has sickle cell disease, hemophilia (e.g., hemophilia A, B, or C), cystic fibrosis, phenylketonuria, Tay-Sachs, prion disease, color blindness, a lysosomal storage disease (e.g., Fabry disease), Friedreich's ataxia, or prostate cancer.

In some embodiments, the methods of the present disclosure may target base editing to a particular allele of a gene, e.g., a wild-type or mutated allele. In certain embodiments, the allele may be associated with cancer. For example, the methods may target the V617F mutated allele of JAK2, which leads to constitutive tyrosine phosphorylation activity and plays a critical role in the expansion of myeloproliferative neoplasms. Knocking out expression of the allele with the V617F mutation, e.g., by introducing a stop codon, may facilitate successful treatment of JAK2 V617F disorders.

The present disclosure further provides a pharmaceutical composition comprising elements of a base editor system described herein, such as a ZFP-cytidine deaminase (e.g., ZFP-TDD as described herein) pair and optionally a cytidine deaminase inhibitor (e.g., TDDI, such as Dddl where the cytidine deaminase is DddA) component (e.g., a ZFP-cytidine deaminase inhibitor component), or nucleotide sequences encoding said elements (e.g., in viral or non-viral vectors as described herein). The pharmaceutical composition may further comprise a pharmaceutically acceptable carrier such as water, saline (e.g., phosphate-buffered saline), dextrose, glycerol, sucrose, lactose, gelatin, dextran, albumin, or pectin. In addition, the composition may contain auxiliary substances, such as, wetting or emulsifying agents, pH-buffering agents, stabilizing agents, or other reagents that enhance the effectiveness of the pharmaceutical composition. The pharmaceutical composition may contain delivery vehicles such as liposomes, nanocapsules, microparticles, microspheres, lipid particles, and vesicles.

In some embodiments, the base editor systems described herein can be engineered to target to a genomic locus chosen from 2B4 (CD244), 4-1BB (CD137), A2aR, AAVS1, ACTB, AID, ALB, B2M, B7.1, B7.2, B7-H2, B7-H3, B7-H4, B7-H6, BAFFR, BCL11A, BLAME (SLAMF8), BTLA, butyrophilins, CIITA, CCR5, CD100 (SEMA4D), CD103, CD3zeta, CD4, CD5, CD7, CD11a, CD11b, CD11c, CD11d, CD150, IPO-3), CD160, CD160 (BY55), CD18, CD19, CD2, CD27, CD28, CD29, CD30, CD4, CD40, CD47, CD48, CD49a, CD49D, CD49f, CD52, CD69, CD7, CD83, CD84, CD8alpha, CD8beta, CD96 (Tactile), CDS, CEACAM1, CISH, CRTAM, CTLA4, CXCR4, DCK, DGK, DGKA, DGKB, DGKD, DGKE, DGKG, DGKI, DGKK, DGKQ, DGKZ, DHFR, DNAM1 (CD226), EP2/4 receptors, adenosine receptors including A2AR, FAS, FASLG, GADS, GITR, GM-CSF, gp49B, HHLA2, HLA-A, HLA-B, HLA-C, HLA-DPA1, HLA-DPB1, HIV-LTR (long terminal repeat), HLA-DQA1, HLA-DQB1, HLA-DRA, HLA-DRB1, HLA-I, HVEM, HVEM, IA4, ICAM-1, ICOS, ICOS, ICOS (CD278), IFN-alpha/beta/gamma, IL-1 beta, IL-12, IL-15, IL-18, IL-23, IL2R beta, IL2R gamma, IL2RA, IL-6, IL7R alpha, ILT-2, ILT-4, immunoglobulin heavy chain loci, immunoglobulin light chain loci, ITGA4, ITGA4, ITGA6, ITGAD, ITGAE, ITGAL, ITGAM, ITGAX, ITGB1, ITGB2, ITGB7, MR family receptors, KLRG1, Lag-3, LAIR-1, LAT, LIGHT, LTBR, Ly9 (CD229), MNK1/2, NKG2C, NKG2D, NKp30, NKp44, NKp46, NKp80 (KLRF1), OX2R, OX40, PAG/Cbp, PD-1, PD-L1, PD-L2, PGE2 receptors, PIR-B, PPP1R12C, PRNP1, PSGL1, PTPN2, RANCE/RANKL, RFX5, ROSA26, SELPLG (CD162), SIRPalpha (CD47), SLAM (SLAMF1, SLAMF4 (CD244, 2B4), SLAMF5, SLAMF6 (NTB-A, Ly108), SLAMF7, SLP-76, SOCS1, SOCS3, Tetherin, TGFBR2, TIGIT, TIM-1, TIM-3, TIM-4, TMIGD2, TRA, TRAC, TRB, TRD, TRG, TNF, TNF-alpha, TNFR2, TRIMS, TUBA1, VISTA, VLA1, or VLA-6.

It is understood that the ZFP fusion proteins and base editor systems described herein may be used in a method of treatment described herein, may be for use in a treatment described herein, or may be used in the manufacture of a medicament for a treatment described herein.

### V. Agricultural Applications

The described systems and methods of editing a cytosine to a thymine base in cellular DNA may also be used in agricultural applications. For example, in certain embodiments, the base editing may correct one or more point mutations in a DNA sequence to restore normal gene expression or activity. In certain embodiments, the base editing may introduce a stop codon into one or more deleterious genes. In certain embodiments, the base editing may introduce one or more beneficial mutations. In particular embodiments, the systems and methods described herein are used to edit a crop plant.

Unless otherwise defined herein, scientific and technical terms used in connection with the present disclosure shall have the meanings that are commonly understood by those of ordinary skill in the art. Exemplary methods and materials are described below, although methods and materials similar or equivalent to those described herein can also be used in the practice or testing of the present disclosure. In case of conflict, the present specification, including definitions, will control. Generally, nomenclature used in connection with, and techniques of, cardiology, medicine, medicinal and pharmaceutical chemistry, and cell biology described herein are those well-known and commonly used in the art. Enzymatic reactions and purification techniques are performed according to manufacturer's specifications, as commonly accomplished in the art or as described herein. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. It should also be noted that the term “or” is generally employed in its sense including “and/or” unless the content clearly dictates otherwise. As used herein the term “about” refers to a numerical range that is 10%, 5%, or 1% plus or minus from a stated numerical value within the context of the particular usage. Further, headings provided herein are for convenience only and do not interpret the scope or meaning of the claimed embodiments.

All publications and other references mentioned herein are incorporated by reference in their entirety. Although a number of documents are cited herein, this citation does not constitute an admission that any of these documents forms part of the common general knowledge in the art.

In order that this invention may be better understood, the following examples are set forth. These examples are for purposes of illustration only and are not to be construed as limiting the scope of the invention in any manner.

### EXAMPLES

### Example 1: ZFP-TDD Design

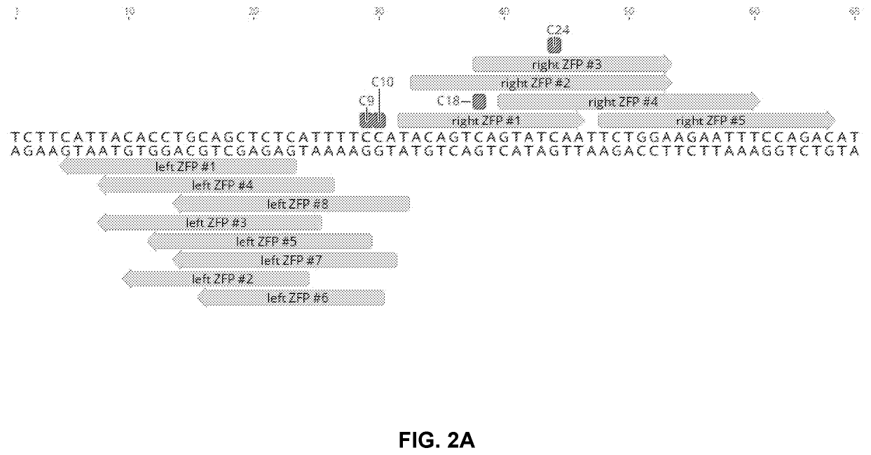

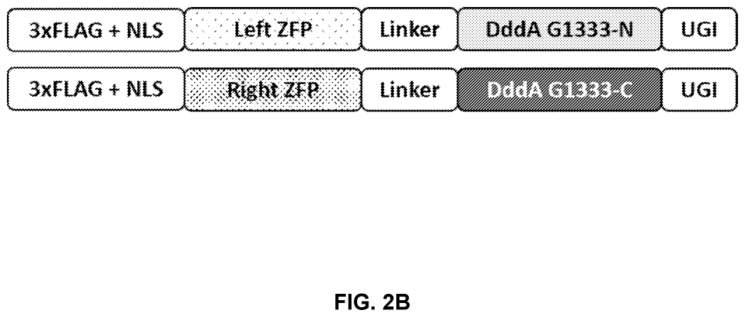

To prepare ZFP-DddA fusion protein pairs, the DddA peptide was split into two halves (each lacking cytidine deaminase activity) at residue G1333, as described in Mok et at, supra (“DddA-G1333′), as well as at residues G1404 (”DddA-G1404″) and G1407 (“DddA-G1407”). Eight left ZFPs and five right ZFPs were designed to target the DddA halves to a site at the human CCR5 locus, such that the halves could dimerize at the target site and restore the catalytic activity of DddA. The left and right ZFP pairs cover a broad variety of different base editing windows from 2-bp to 24-bp (FIG. 2A).

The N-terminal half of each split DddA pair was fused to the C-terminus of a left ZFP and the C-terminal half was fused to the C-terminus of a right ZFP, and vice-versa. For DddA-G1333, one of three different linkers (LO, L7A and L26) was used, whereas for DddA-G1404 and DddA-G1407, the L26 linker was used. For all other experiments, unless otherwise indicated, the L26 linker was used. A UGI (uracil DNA glycosylase inhibitor) domain was also fused to the C-terminus of each N-terminal and C-terminal half. All ZFP-DddA fusion constructs further contained a 3×FLAG tag as well as an SV40 nuclear localization signal fused to the N-terminus of the ZFP. An example of a left and right ZFP pair is shown in FIG. 2B.

The above-described sequences and the sequences of several prepared constructs are shown in Table 2 below. Finger sequences are underlined and bolded in Left ZFPs #1-8 and Right ZFPs #1-5. The ZFPs in Table 2 target the CCR5 locus.

TABLE 2
Sequences of ZFP-DddA Components and Constructs (CCR5 Locus ZFPs)

| SEQ | Description | Sequence |
|---|---|---|
| 1 | 3xFlag + NLS | MDYKDHDGDYKDHDIDYKDDDDKMAPKKKRKVGIHGVPAAMA |
| 2 | Left ZFP #1 | ERPFQCRICMRNFSRSDSLSVHIRTHTGEKPFACDICGRKFAQSGS |
| | | LTRHTKIHTGSQKPFQCRICMRNFSTSGHLSRHIRTHTGEKPFACD |
| | | ICGRKFAQSGDLTRHTKIHTHPRAPIPKPFQCRICMRNFSMVCCRT |
| | | LHIRTHTGEKPFACDICGRKFARSANLTRHTKIH |
| 3 | Left ZFP #2 | ERPFQCRICMRNFSRPYTLRLHIRTHTGEKPFACDICGRKFARKYY |
| | | LAKHTKIHTGSQKPFQCRICMRNFSDDWNLSQHIRTHTGEKPFACD |
| | | ICGRKFARSANLTRHTKIHTGEKPFQCRICMRKFAQSAHRITHTKI |
| | | H |
| 4 | Left ZFP #3 | ERPFQCRICMRNFSQSGALARHIRTHTGEKPFACDICGRKFALKQH |
| | | LTRHTKIHTGSQKPFQCRICMRNFSQSGDLTRHIRTHTGEKPFACD |
| | | ICGRKFAQSSDLRRHTKIHTGSQKPFQCRICMRNFSQSAHRKNHIR |
| | | THTGEKPFACDICGRKFARSAVRKNHTKIH |
| 5 | Left ZFP #4 | ERPFQCRICMRNFSQSGALARHIRTHTGEKPFACDICGRKFALKQH |
| | | LTRHTKIHTGSQKPFQCRICMRNFSQSGDLTRHIRTHTGEKPFACD |
| | | ICGRKFAQSSDLRRHTKIHTHPRAPIPKPFQCRICMRNFSRSANLA |
| | | RHIRTHTGEKPFACDICGRKFATNQNRITHTKIH |
| 6 | Left ZFP #5 | ERPFQCRICMRNFSRSDHLSAHIRTHTGEKPFACDICGRKFACRRN |
| | | LRNHTKIHTGSQKPFQCRICMRNFSMVCCRTLHIRTHTGEKPFACD |
| | | ICGRKFARSANLTRHTKIHTGSQKPFQCRICMRNFSTSSNRKTHIR |
| | | THTGEKPFACDICGRKFAQSGHLSRHTKIH |
| 7 | Left ZFP #6 | ERPFQCRICMRNFSDDWNLSQHIRTHTGEKPFACDICGRKFARSAN |
| | | LTRHTKIHTGSQKPFQCRICMRKFAQSAHRITHTKIHTGEKPFQCR |
| | | ICMRNFSQSANRTTHIRTHTGEKPFACDICGRKFAQNAHRKTHTKI |
| | | H |
| 8 | Left ZFP #7 | ERPFQCRICMRNFSQSGDLTRHIRTHTGEKPFACDICGRKFAQSSD |
| | | LRRHTKIHTGSQKPFQCRICMRNFSQSAHRKNHIRTHTGEKPFACD |
| | | ICGRKFARSAVRKNHTKIHTGSQKPFQCRICMRNFSQSANRTTHIR |
| | | THTGEKPFACDICGRKFARKYYLAKHTKIH |
| 9 | Left ZFP #8 | ERPFQCRICMRNFSQSGDLTRHIRTHTGEKPFACDICGRKFAQSSD |
| | | LRRHTKIHTHPRAPIPKPFQCRICMRNFSRSANLARHIRTHTGEKP |
| | | FACDICGRKFATNQNRITHTKIHTGSQKPFQCRICMRNFSQSGDLT |
| | | RHIRTHTGEKPFACDICGRKFARKDPLKEHTKIH |
| 10 | Right ZFP #1 | ERPFQCRICMRKFAQSGNRTTHTKIHTGEKPFQCRICMRNFSTSSN |
| | | RKTHIRTHTGEKPFACDICGRKFAAQWTRACHTKIHTGSQKPFQCR |
| | | ICMRNFSLRHHLTRHIRTHTGEKPFACDICGRKFADRTGLRSHTKI |
| | | H |
| 11 | Right ZFP #2 | ERPFQCRICMRNFSQSGHLARHIRTHTGEKPFACDICGRKFANRHD |
| | | RAKHTKIHTPNPHRRTDPSHKPFQCRICMRNFSQSADRTKHIRTHT |
| | | GEKPFACDICGRKFAQSGSLTRHTKIHTHPRAPIPKPFQCRICMRN |
| | | FSDRSTRITHIRTHTGEKPFACDICGRKFAQNATRINHTKIH |
| 12 | Right ZFP #3 | ERPFQCRICMRNFSQSGHLARHIRTHTGEKPFACDICGRKFANRHD |
| | | RAKHTKIHTHPRAPIPKPFQCRICMRKFAQSGNRTTHTKIHTGEKP |
| | | FQCRICMRNFSTSSNRKTHIRTHTGEKPFACDICGRKFAAQWTRAC |
| | | HTKIH |
| 13 | Right ZFP #4 | ERPFQCRICMRNFSDIGYRAAHIRTHTGEKPFACDICGRKFAQSGN |
| | | LARHTKIHTHPRAPIPKPFQCRICMRNFSQSGHLARHIRTHTGEKP |
| | | FACDICGRKFANRHDRAKHTKIHTPNPHRRTDPSHKPFQCRICMRN |
| | | FSQSADRTKHIRTHTGEKPFACDICGRKFAQSGSLTRHTKIH |
| 14 | Right ZFP #5 | ERPFQCRICMRNFSDRSNLSRHIRTHTGEKPFACDICGRKFAQSGD |
| | | LTRHTKIHTGSQKPFQCRICMRNFSDIGYRAAHIRTHTGEKPFACD |
| | | ICGRKFAQSGNLARHTKIHTHPRAPIPKPFQCRICMRNFSQSGHLA |
| | | RHIRTHTGEKPFACDICGRKFANRHDRAKHTKIH |
| 15 | L0 | LRGSQLVKS |
| 16 | L7A | LRGSQLVKSKSEAAAR |
| 17 | L26 | LRGSQLVKSKSEAAARGGGGSGGGGS |
| 18 | G1333-N | GSYALGPYQISAPQLPAYNGQTVGTFYYVNDAGGLESKVFSSGG |
| 19 | G1333-C | PTPYPNYANAGHVEGQSALFMRDNGISEGLVFHNNPEGTCGFCVNM |
| | | TETLLPENAKMTVVPPEGAIPVKRGATGETKVFTGNSNSPKSPTKG |
| | | GC |
| 82 | G1404-N | GSYALGPYQISAPQLPAYNGQTVGTFYYVNDAGGLESKVFSSGGPT |
| | | PYPNYANAGHVEGQSALFMRDNGISEGLVFHNNPEGTCGFCVNMTE |
| | | TLLPENAKMTVVPPEGAIPVKRG |
| 83 | G1404-C | ATGETKVFTGNSNSPKSPTKGGC |
| 84 | G1407-N | GSYALGPYQISAPQLPAYNGQTVGTFYYVNDAGGLESKVFSSGGPT |
| | | PYPNYANAGHVEGQSALFMRDNGISEGLVFHNNPEGTCGFCVNMTE |
| | | TLLPENAKMTVVPPEGAIPVKRGATG |
| 85 | G1407-C | ETKVFTGNSNSPKSPTKGGC |
| 20 | UGI | TNLSDIIEKETGKQLVIQESILMLPEEVEEVIGNKPESDILVHTAY |
| | | DESTDENVMLLTSDAPEYKPWALVIQDSNGENKIKML |
| 21 | G1333-N + UGI | GSYALGPYQISAPQLPAYNGQTVGTFYYVNDAGGLESKVFSSGGSG |
| | | GSTNLSDIIEKETGKQLVIQESILMLPEEVEEVIGNKPESDILVHT |
| | | AYDESTDENVMLLTSDAPEYKPWALVIQDSNGENKIKML |
| 22 | G1333-C + UGI | PTPYPNYANAGHVEGQSALFMRDNGISEGLVFHNNPEGTCGFCVNM |
| | | TETLLPENAKMTVVPPEGAIPVKRGATGETKVFTGNSNSPKSPTKG |
| | | GCSGGSTNLSDIIEKETGKQLVIQESILMLPEEVEEVIGNKPESDI |
| | | LVHTAYDESTDENVMLLTSDAPEYKPWALVIQDSNGENKIKML |
| 23 | Left ZFP | MDYKDHDGDYKDHDIDYKDDDDKMAPKKKRKVGIHGVPAAMAERPF |
| | #4_L0_G1333-N | QCRICMRNFSQSGALARHIRTHTGEKPFACDICGRKFALKQHLTRH |
| | (incl UGI) | TKIHTGSQKPFQCRICMRNFSQSGDLTRHIRTHTGEKPFACDICGR |
| | | KFAQSSDLRRHTKIHTHPRAPIPKPFQCRICMRNFSRSANLARHIR |
| | | THTGEKPFACDICGRKFATNQNRITHTKIHLRGSQLVKSGSYALGP |
| | | YQISAPQLPAYNGQTVGTFYYVNDAGGLESKVFSSGGSGGSTNLSD |
| | | IIEKETGKQLVIQESILMLPEEVEEVIGNKPESDILVHTAYDESTD |
| | | ENVMLLTSDAPEYKPWALVIQDSNGENKIKML* |
| 24 | Left ZFP | MDYKDHDGDYKDHDIDYKDDDDKMAPKKKRKVGIHGVPAAMAERPF |
| | #1_L7A_G1333-N | QCRICMRNFSRSDSLSVHIRTHTGEKPFACDICGRKFAQSGSLTRH |
| | (incl UGI) | TKIHTGSQKPFQCRICMRNFSTSGHLSRHIRTHTGEKPFACDICGR |
| | | KFAQSGDLTRHTKIHTHPRAPIPKPFQCRICMRNFSMVCCRTLHIR |
| | | THTGEKPFACDICGRKFARSANLTRHTKIHLRGSQLVKSKSEAAAR |
| | | GSYALGPYQISAPQLPAYNGQTVGTFYYVNDAGGLESKVFSSGGSG |
| | | GSTNLSDIIEKETGKQLVIQESILMLPEEVEEVIGNKPESDILVHT |
| | | AYDESTDENVMLLTSDAPEYKPWALVIQDSNGENKIKML* |
| 25 | Left ZFP | MDYKDHDGDYKDHDIDYKDDDDKMAPKKKRKVGIHGVPAAMAERPF |
| | #5_L7A_G1333-N | QCRICMRNFSRSDHLSAHIRTHTGEKPFACDICGRKFACRRNLRNH |
| | (incl UGI) | TKIHTGSQKPFQCRICMRNFSMVCCRTLHIRTHTGEKPFACDICGR |
| | | KFARSANLTRHTKIHTGSQKPFQCRICMRNFSTSSNRKTHIRTHTG |
| | | EKPFACDICGRKFAQSGHLSRHTKIHLRGSQLVKSKSEAAARGSYA |
| | | LGPYQISAPQLPAYNGQTVGTFYYVNDAGGLESKVESSGGSGGSTN |
| | | LSDIIEKETGKQLVIQESILMLPEEVEEVIGNKPESDILVHTAYDE |
| | | STDENVMLLTSDAPEYKPWALVIQDSNGENKIKML* |
| 26 | Left ZFP | MDYKDHDGDYKDHDIDYKDDDDKMAPKKKRKVGIHGVPAAMAERPF |
| | #8_L7A_G1333-N | QCRICMRNFSQSGDLTRHIRTHTGEKPFACDICGRKFAQSSDLRRH |
| | (incl UGI) | TKIHTHPRAPIPKPFQCRICMRNFSRSANLARHIRTHTGEKPFACD |
| | | ICGRKFATNQNRITHTKIHTGSQKPFQCRICMRNFSQSGDLTRHIR |
| | | THTGEKPFACDICGRKFARKDPLKEHTKIHLRGSQLVKSKSEAAAR |
| | | GSYALGPYQISAPQLPAYNGQTVGTFYYVNDAGGLESKVFSSGGSG |
| | | GSTNLSDIIEKETGKQLVIQESILMLPEEVEEVIGNKPESDILVHT |
| | | AYDESTDENVMLLTSDAPEYKPWALVIQDSNGENKIKML* |
| 27 | Left ZFP | MDYKDHDGDYKDHDIDYKDDDDKMAPKKKRKVGIHGVPAAMAERPF |
| | #3_L7A_G1333-C | QCRICMRNFSQSGALARHIRTHTGEKPFACDICGRKFALKQHLTRH |
| | (incl UGI) | TKIHTGSQKPFQCRICMRNFSQSGDLTRHIRTHTGEKPFACDICGR |
| | | KFAQSSDLRRHTKIHTGSQKPFQCRICMRNFSQSAHRKNHIRTHTG |
| | | EKPFACDICGRKFARSAVRKNHTKIHLRGSQLVKSKSEAAARPTPY |
| | | PNYANAGHVEGQSALFMRDNGISEGLVFHNNPEGTCGFCVNMTETL |
| | | LPENAKMTVVPPEGAIPVKRGATGETKVFTGNSNSPKSPTKGGCSG |
| | | GSTNLSDIIEKETGKQLVIQESILMLPEEVEEVIGNKPESDILVHT |
| | | AYDESTDENVMLLTSDAPEYKPWALVIQDSNGENKIKML* |
| 28 | Left ZFP | MDYKDHDGDYKDHDIDYKDDDDKMAPKKKRKVGIHGVPAAMAERPF |
| | #1_L26_G1333-N | QCRICMRNFSRSDSLSVHIRTHTGEKPFACDICGRKFAQSGSLTRH |
| | (incl UGI) | TKIHTGSQKPFQCRICMRNESTSGHLSRHIRTHTGEKPFACDICGR |
| | | KFAQSGDLTRHTKIHTHPRAPIPKPFQCRICMRNFSMVCCRTLHIR |
| | | THTGEKPFACDICGRKFARSANLTRHTKIHLRGSQLVKSKSEAAAR |
| | | GGGGSGGGGSGSYALGPYQISAPQLPAYNGQTVGTFYYVNDAGGLE |
| | | SKVFSSGGSGGSTNLSDIIEKETGKQLVIQESILMLPEEVEEVIGN |
| | | KPESDILVHTAYDESTDENVMLLTSDAPEYKPWALVIQDSNGENKI |
| | | KML* |
| 29 | Left ZFP | MDYKDHDGDYKDHDIDYKDDDDKMAPKKKRKVGIHGVPAAMAERPF |
| | #3_L26_G1333-N | QCRICMRNFSQSGALARHIRTHTGEKPFACDICGRKFALKQHLTRH |
| | (incl UGI) | TKIHTGSQKPFQCRICMRNFSQSGDLTRHIRTHTGEKPFACDICGR |
| | | KFAQSSDLRRHTKIHTGSQKPFQCRICMRNFSQSAHRKNHIRTHTG |
| | | EKPFACDICGRKFARSAVRKNHTKIHLRGSQLVKSKSEAAARGGGG |
| | | SGGGGSGSYALGPYQISAPQLPAYNGQTVGTFYYVNDAGGLESKVF |
| | | SSGGSGGSTNLSDIIEKETGKQLVIQESILMLPEEVEEVIGNKPES |
| | | DILVHTAYDESTDENVMLLTSDAPEYKPWALVIQDSNGENKIKML* |
| 30 | Left ZFP | MDYKDHDGDYKDHDIDYKDDDDKMAPKKKRKVGIHGVPAAMAERPF |
| | #4_L26_G1333-N | QCRICMRNFSQSGALARHIRTHTGEKPFACDICGRKFALKQHLTRH |
| | (incl UGI) | TKIHTGSQKPFQCRICMRNFSQSGDLTRHIRTHTGEKPFACDICGR |
| | | KFAQSSDLRRHTKIHTHPRAPIPKPFQCRICMRNFSRSANLARHIR |
| | | THTGEKPFACDICGRKFATNQNRITHTKIHLRGSQLVKSKSEAAAR |
| | | GGGGSGGGGSGSYALGPYQISAPQLPAYNGQTVGTFYYVNDAGGLE |
| | | SKVFSSGGSGGSTNLSDIIEKETGKQLVIQESILMLPEEVEEVIGN |
| | | KPESDILVHTAYDESTDENVMLLTSDAPEYKPWALVIQDSNGENKI |
| | | KML* |
| 31 | Left ZFP | MDYKDHDGDYKDHDIDYKDDDDKMAPKKKRKVGIHGVPAAMAERPF |
| | #5_L26_G1333-N | QCRICMRNFSRSDHLSAHIRTHTGEKPFACDICGRKFACRRNLRNH |
| | (incl UGI) | TKIHTGSQKPFQCRICMRNFSMVCCRTLHIRTHTGEKPFACDICGR |
| | | KFARSANLTRHTKIHTGSQKPFQCRICMRNFSTSSNRKTHIRTHTG |
| | | EKPFACDICGRKFAQSGHLSRHTKIHLRGSQLVKSKSEAAARGGGG |
| | | SGGGGSGSYALGPYQISAPQLPAYNGQTVGTFYYVNDAGGLESKVF |
| | | SSGGSGGSTNLSDIIEKETGKQLVIQESILMLPEEVEEVIGNKPES |
| | | DILVHTAYDESTDENVMLLTSDAPEYKPWALVIQDSNGENKIKML* |
| 32 | Left ZFP | MDYKDHDGDYKDHDIDYKDDDDKMAPKKKRKVGIHGVPAAMAERPF |
| | #8_L26_G1333-N | QCRICMRNFSQSGDLTRHIRTHTGEKPFACDICGRKFAQSSDLRRH |
| | (incl UGI) | TKIHTHPRAPIPKPFQCRICMRNFSRSANLARHIRTHTGEKPFACD |
| | | ICGRKFATNQNRITHTKIHTGSQKPFQCRICMRNFSQSGDLTRHIR |
| | | THTGEKPFACDICGRKFARKDPLKEHTKIHLRGSQLVKSKSEAAAR |
| | | GGGGSGGGGSGSYALGPYQISAPQLPAYNGQTVGTFYYVNDAGGLE |
| | | SKVFSSGGSGGSTNLSDIIEKETGKQLVIQESILMLPEEVEEVIGN |
| | | KPESDILVHTAYDESTDENVMLLTSDAPEYKPWALVIQDSNGENKI |
| | | KML* |
| 33 | Left ZFP | MDYKDHDGDYKDHDIDYKDDDDKMAPKKKRKVGIHGVPAAMAERPF |
| | #3_L26_G1333-C | QCRICMRNFSQSGALARHIRTHTGEKPFACDICGRKFALKQHLTRH |
| | (incl UGI) | TKIHTGSQKPFQCRICMRNFSQSGDLTRHIRTHTGEKPFACDICGR |
| | | KFAQSSDLRRHTKIHTGSQKPFQCRICMRNFSQSAHRKNHIRTHTG |
| | | EKPFACDICGRKFARSAVRKNHTKIHLRGSQLVKSKSEAAARGGGG |
| | | SGGGGSPTPYPNYANAGHVEGQSALFMRDNGISEGLVFHNNPEGTC |
| | | GFCVNMTETLLPENAKMTVVPPEGAIPVKRGATGETKVFTGNSNSP |
| | | KSPTKGGCSGGSTNLSDIIEKETGKQLVIQESILMLPEEVEEVIGN |
| | | KPESDILVHTAYDESTDENVMLLTSDAPEYKPWALVIQDSNGENKI |
| | | KML* |
| 34 | Left ZFP | MDYKDHDGDYKDHDIDYKDDDDKMAPKKKRKVGIHGVPAAMAERPF |
| | #4_L26_G1333-C | QCRICMRNFSQSGALARHIRTHTGEKPFACDICGRKFALKQHLTRH |
| | (incl UGI) | TKIHTGSQKPFQCRICMRNFSQSGDLTRHIRTHTGEKPFACDICGR |
| | | KFAQSSDLRRHTKIHTHPRAPIPKPFQCRICMRNFSRSANLARHIR |
| | | THTGEKPFACDICGRKFATNQNRITHTKIHLRGSQLVKSKSEAAAR |
| | | GGGGSGGGGSPTPYPNYANAGHVEGQSALFMRDNGISEGLVFHNNP |
| | | EGTCGFCVNMTETLLPENAKMTVVPPEGAIPVKRGATGETKVFTGN |
| | | SNSPKSPTKGGCSGGSTNLSDIIEKETGKQLVIQESILMLPEEVEE |
| | | VIGNKPESDILVHTAYDESTDENVMLLTSDAPEYKPWALVIQDSNG |
| | | ENKIKML* |
| 35 | Left ZFP | MDYKDHDGDYKDHDIDYKDDDDKMAPKKKRKVGIHGVPAAMAERPF |
| | #8_L26_G1333-C | QCRICMRNFSQSGDLTRHIRTHTGEKPFACDICGRKFAQSSDLRRH |
| | (incl UGI) | TKIHTHPRAPIPKPFQCRICMRNFSRSANLARHIRTHTGEKPFACD |
| | | ICGRKFATNQNRITHTKIHTGSQKPFQCRICMRNFSQSGDLTRHIR |
| | | THTGEKPFACDICGRKFARKDPLKEHTKIHLRGSQLVKSKSEAAAR |
| | | GGGGSGGGGSPTPYPNYANAGHVEGQSALFMRDNGISEGLVFHNNP |
| | | EGTCGFCVNMTETLLPENAKMTVVPPEGAIPVKRGATGETKVFTGN |
| | | SNSPKSPTKGGCSGGSTNLSDIIEKETGKQLVIQESILMLPEEVEE |
| | | VIGNKPESDILVHTAYDESTDENVMLLTSDAPEYKPWALVIQDSNG |
| | | ENKIKML* |
| 36 | Right ZFP | MDYKDHDGDYKDHDIDYKDDDDKMAPKKKRKVGIHGVPAAMAERPF |
| | #4_L0_G1333-C | QCRICMRNFSDIGYRAAHIRTHTGEKPFACDICGRKFAQSGNLARH |
| | (incl UGI) | TKIHTHPRAPIPKPFQCRICMRNFSQSGHLARHIRTHTGEKPFACD |
| | | ICGRKFANRHDRAKHTKIHTPNPHRRTDPSHKPFQCRICMRNFSQS |
| | | ADRTKHIRTHTGEKPFACDICGRKFAQSGSLTRHTKIHLRGSQLVK |
| | | SPTPYPNYANAGHVEGQSALFMRDNGISEGLVFHNNPEGTCGFCVN |
| | | MTETLLPENAKMTVVPPEGAIPVKRGATGETKVFTGNSNSPKSPTK |
| | | GGCSGGSTNLSDIIEKETGKQLVIQESILMLPEEVEEVIGNKPESD |
| | | ILVHTAYDESTDENVMLLTSDAPEYKPWALVIQDSNGENKIKML* |
| 37 | Right ZFP | MDYKDHDGDYKDHDIDYKDDDDKMAPKKKRKVGIHGVPAAMAERPF |
| | #5_L0_G1333-C | QCRICMRNFSDRSNLSRHIRTHTGEKPFACDICGRKFAQSGDLTRH |
| | (incl UGI) | TKIHTGSQKPFQCRICMRNFSDIGYRAAHIRTHTGEKPFACDICGR |
| | | KFAQSGNLARHTKIHTHPRAPIPKPFQCRICMRNFSQSGHLARHIR |
| | | THTGEKPFACDICGRKFANRHDRAKHTKIHLRGSQLVKSPTPYPNY |
| | | ANAGHVEGQSALFMRDNGISEGLVFHNNPEGTCGFCVNMTETLLPE |
| | | NAKMTVVPPEGAIPVKRGATGETKVFTGNSNSPKSPTKGGCSGGST |
| | | NLSDIIEKETGKQLVIQESILMLPEEVEEVIGNKPESDILVHTAYD |
| | | ESTDENVMLLTSDAPEYKPWALVIQDSNGENKIKML* |
| 38 | Right ZFP | MDYKDHDGDYKDHDIDYKDDDDKMAPKKKRKVGIHGVPAAMAERPF |
| | #1_L7A_G1333-C | QCRICMRKFAQSGNRTTHTKIHTGEKPFQCRICMRNFSTSSNRKTH |
| | (incl UGI) | IRTHTGEKPFACDICGRKFAAQWTRACHTKIHTGSQKPFQCRICMR |
| | | NFSLRHHLTRHIRTHTGEKPFACDICGRKFADRTGLRSHTKIHLRG |
| | | SQLVKSKSEAAARPTPYPNYANAGHVEGQSALFMRDNGISEGLVFH |
| | | NNPEGTCGFCVNMTETLLPENAKMTVVPPEGAIPVKRGATGETKVF |
| | | TGNSNSPKSPTKGGCSGGSTNLSDIIEKETGKQLVIQESILMLPEE |
| | | VEEVIGNKPESDILVHTAYDESTDENVMLLTSDAPEYKPWALVIQD |
| | | SNGENKIKML* |
| 39 | Right ZFP | MDYKDHDGDYKDHDIDYKDDDDKMAPKKKRKVGIHGVPAAMAERPF |
| | #5_L7A_G1333-C | QCRICMRNFSDRSNLSRHIRTHTGEKPFACDICGRKFAQSGDLTRH |
| | (incl UGI) | TKIHTGSQKPFQCRICMRNFSDIGYRAAHIRTHTGEKPFACDICGR |
| | | KFAQSGNLARHTKIHTHPRAPIPKPFQCRICMRNFSQSGHLARHIR |
| | | THTGEKPFACDICGRKFANRHDRAKHTKIHLRGSQLVKSKSEAAAR |
| | | PTPYPNYANAGHVEGQSALFMRDNGISEGLVFHNNPEGTCGFCVNM |
| | | TETLLPENAKMTVVPPEGAIPVKRGATGETKVFTGNSNSPKSPTKG |
| | | GCSGGSTNLSDIIEKETGKQLVIQESILMLPEEVEEVIGNKPESDI |
| | | LVHTAYDESTDENVMLLTSDAPEYKPWALVIQDSNGENKIKML* |
| 40 | Right ZFP | MDYKDHDGDYKDHDIDYKDDDDKMAPKKKRKVGIHGVPAAMAERPF |
| | #4_L7A_G1333-C | QCRICMRNFSDIGYRAAHIRTHTGEKPFACDICGRKFAQSGNLARH |
| | (incl UGI) | TKIHTHPRAPIPKPFQCRICMRNFSQSGHLARHIRTHTGEKPFACD |
| | | ICGRKFANRHDRAKHTKIHTPNPHRRTDPSHKPFQCRICMRNFSQS |
| | | ADRTKHIRTHTGEKPFACDICGRKFAQSGSLTRHTKIHLRGSQLVK |
| | | SKSEAAARPTPYPNYANAGHVEGQSALFMRDNGISEGLVFHNNPEG |
| | | TCGFCVNMTETLLPENAKMTVVPPEGAIPVKRGATGETKVFTGNSN |
| | | SPKSPTKGGCSGGSTNLSDIIEKETGKQLVIQESILMLPEEVEEVI |
| | | GNKPESDILVHTAYDESTDENVMLLTSDAPEYKPWALVIQDSNGEN |
| | | KIKML* |
| 41 | Right ZFP | MDYKDHDGDYKDHDIDYKDDDDKMAPKKKRKVGIHGVPAAMAERPF |
| | #1_L7A_G1333-N | QCRICMRKFAQSGNRTTHTKIHTGEKPFQCRICMRNFSTSSNRKTH |
| | (incl UGI) | IRTHTGEKPFACDICGRKFAAQWTRACHTKIHTGSQKPFQCRICMR |
| | | NFSLRHHLTRHIRTHTGEKPFACDICGRKFADRTGLRSHTKIHLRG |
| | | SQLVKSKSEAAARGSYALGPYQISAPQLPAYNGQTVGTFYYVNDAG |
| | | GLESKVFSSGGSGGSTNLSDIIEKETGKQLVIQESILMLPEEVEEV |
| | | IGNKPESDILVHTAYDESTDENVMLLTSDAPEYKPWALVIQDSNGE |
| | | NKIKML* |
| 42 | Right ZFP | MDYKDHDGDYKDHDIDYKDDDDKMAPKKKRKVGIHGVPAAMAERPF |
| | #1_L26_G1333-C | QCRICMRKFAQSGNRTTHTKIHTGEKPFQCRICMRNFSTSSNRKTH |
| | (incl UGI) | IRTHTGEKPFACDICGRKFAAQWTRACHTKIHTGSQKPFQCRICMR |
| | | NFSLRHHLTRHIRTHTGEKPFACDICGRKFADRTGLRSHTKIHLRG |
| | | SQLVKSKSEAAARGGGGSGGGGSPTPYPNYANAGHVEGQSALFMRD |
| | | NGISEGLVFHNNPEGTCGFCVNMTETLLPENAKMTVVPPEGAIPVK |
| | | RGATGETKVFTGNSNSPKSPTKGGCSGGSTNLSDIIEKETGKQLVI |
| | | QESILMLPEEVEEVIGNKPESDILVHTAYDESTDENVMLLTSDAPE |
| | | YKPWALVIQDSNGENKIKML* |
| 43 | Right ZFP | MDYKDHDGDYKDHDIDYKDDDDKMAPKKKRKVGIHGVPAAMAERPF |
| | #5_L26_G1333-C | QCRICMRNFSDRSNLSRHIRTHTGEKPFACDICGRKFAQSGDLTRH |
| | (incl UGI) | TKIHTGSQKPFQCRICMRNFSDIGYRAAHIRTHTGEKPFACDICGR |
| | | KFAQSGNLARHTKIHTHPRAPIPKPFQCRICMRNFSQSGHLARHIR |
| | | THTGEKPFACDICGRKFANRHDRAKHTKIHLRGSQLVKSKSEAAAR |
| | | GGGGSGGGGSPTPYPNYANAGHVEGQSALFMRDNGISEGLVFHNNP |
| | | EGTCGFCVNMTETLLPENAKMTVVPPEGAIPVKRGATGETKVFTGN |
| | | SNSPKSPTKGGCSGGSTNLSDIIEKETGKQLVIQESILMLPEEVEE |
| | | VIGNKPESDILVHTAYDESTDENVMLLTSDAPEYKPWALVIQDSNG |
| | | ENKIKML* |
| 44 | Right ZFP | MDYKDHDGDYKDHDIDYKDDDDKMAPKKKRKVGIHGVPAAMAERPF |
| | #4_L26_G1333-C | QCRICMRNFSDIGYRAAHIRTHTGEKPFACDICGRKFAQSGNLARH |
| | (incl UGI) | TKIHTHPRAPIPKPFQCRICMRNFSQSGHLARHIRTHTGEKPFACD |
| | | ICGRKFANRHDRAKHTKIHTPNPHRRTDPSHKPFQCRICMRNFSQS |
| | | ADRTKHIRTHTGEKPFACDICGRKFAQSGSLTRHTKIHLRGSQLVK |
| | | SKSEAAARGGGGSGGGGSPTPYPNYANAGHVEGQSALFMRDNGISE |
| | | GLVFHNNPEGTCGFCVNMTETLLPENAKMTVVPPEGAIPVKRGATG |
| | | ETKVFTGNSNSPKSPTKGGCSGGSTNLSDIIEKETGKQLVIQESIL |
| | | MLPEEVEEVIGNKPESDILVHTAYDESTDENVMLLTSDAPEYKPWA |
| | | LVIQDSNGENKIKML* |
| 45 | Right ZFP | MDYKDHDGDYKDHDIDYKDDDDKMAPKKKRKVGIHGVPAAMAERPF |
| | #1_L26_G1333-N | QCRICMRKFAQSGNRTTHTKIHTGEKPFQCRICMRNFSTSSNRKTH |
| | (incl UGI) | IRTHTGEKPFACDICGRKFAAQWTRACHTKIHTGSQKPFQCRICMR |
| | | NFSLRHHLTRHIRTHTGEKPFACDICGRKFADRTGLRSHTKIHLRG |
| | | SQLVKSKSEAAARGGGGSGGGGSGSYALGPYQISAPQLPAYNGQTV |
| | | GTFYYVNDAGGLESKVFSSGGSGGSTNLSDIIEKETGKQLVIQESI |
| | | LMLPEEVEEVIGNKPESDILVHTAYDESTDENVMLLTSDAPEYKPW |
| | | ALVIQDSNGENKIKML* |
| 46 | Right ZFP | MDYKDHDGDYKDHDIDYKDDDDKMAPKKKRKVGIHGVPAAMAERPF |
| | #5_L26_G1333-N | QCRICMRNFSDRSNLSRHIRTHTGEKPFACDICGRKFAQSGDLTRH |
| | (incl UGI) | TKIHTGSQKPFQCRICMRNFSDIGYRAAHIRTHTGEKPFACDICGR |
| | | KFAQSGNLARHTKIHTHPRAPIPKPFQCRICMRNFSQSGHLARHIR |
| | | THTGEKPFACDICGRKFANRHDRAKHTKIHLRGSQLVKSKSEAAAR |
| | | GGGGSGGGGSGSYALGPYQISAPQLPAYNGQTVGTFYYVNDAGGLE |
| | | SKVFSSGGSGGSTNLSDIIEKETGKQLVIQESILMLPEEVEEVIGN |
| | | KPESDILVHTAYDESTDENVMLLTSDAPEYKPWALVIQDSNGENKI |
| | | KML* |

SEQ: SEQ ID NO.

### Example 2: ZFP-DddA Base Editing in K562 Cells

To assay base editing in cells using same-linker ZFP-DddA pairs prepared according to the method described above, K562 (ATCC, CCL243) cells were obtained from the ATCC and were maintained in RPMI1640 with 10% FBS and 1×penicillin-streptomycin-glutamine (PSG) (Gibco, 10378-016) at 37 ° C. with 5% CO₂. 400 ng of pDNA encoding paired ZFP-DddA was electroporated into K562 cells using the SF cell line 96-well Nucleofector kit (Lonza, V4SC-2960) following the manufacturer's instructions. In brief, cells were washed twice with 1×PBS (divalent cation-free) and resuspended at 2×10⁵ cells per 15 μL of supplemented SF cell line 96-well Nucleofector solution. For each transfection, 15 μL of the cell suspension was mixed with 5 μL of pDNA and transferred to the Lonza Nucleocuvette plate, then electroporated using the protocol for K562 cells (Nucleofector program 96-FF-120) on an Amaxa Nucleofector 96-well Shuttle System (Lonza). Electroporated cells were incubated at room temperature for 10 min and then transferred to 150 μL of prewarmed complete medium in a 96-well tissue culture plate. Cells were incubated for 72 h and then harvested for base editing quantification.

PCR primers for the CCR5 locus were designed using Primer3 with the following optimal conditions: amplicon size of 200 nucleotides; a melting temperature of 60° C.; primer length of 20 nucleotides; and GC content of 50%. Sequences for the primers and amplicon are shown in Table 3 below.

TABLE 3
CCR5 Primer and Amplicon Sequences

| | Descrip- | |
|---|---|---|
| SEQ | tion | Sequence |
| 74 | CCR5 | ACACTCTTTCCCTACACGACGCTCTTCCGATCTNNNN |
| | forward | CAAGTGTGATCACTTGGGTGG |
| | primer | |
| 75 | CCR5 | TGGAGTTCAGACGTGTGCTCTTCCGATCTGGATTCCC |
| | reverse | GAGTAGCAGATG |
| | primer | |
| 76 | CCR5 NGS | NNNNcaagtgtgatcacttgggtggtggctgtgtttg |
| | amplicon | cgtctctcccaggaatcatctttaccagatctcaaaa |
| | | agaaggtcttcattacacctgcagctctcattttcca |
| | | ctacagtcagtatcaattctggaagaatttcagacat |
| | | taaagatagtcatcttggggctggtcctgccgctgct |
| | | tgtcatggtcatctgctactcgggaatcc |

SEQ: SEQ ID NO.

Adaptors were added for a second PCR reaction to add the Illumina library sequences (forward primer: ACACGACGCTCTTCCGATCT (SEQ ID NO: 47); reverse primer: GACGTGTGCTCTTCCGAT (SEQ ID NO: 48)). The CCR5 locus was amplified in 25 μL using 100 ng of genomic DNA with AccuPrime HiFi (Invitrogen). Primers were used at a final concentration of 0.1 μM with the following thermocycling conditions: initial melt of 95° C. for 5 min; 35 cycles of 95° C. for 30 s, 55° C. for 30 s and 68 ° C. for 40 s; and a final extension at 68° C. for 10 min. PCR products were diluted 1:20 in water. 2μL of diluted PCR product was used in a 20 μL PCR reaction to add the Illumina library sequences with Phusion High-Fidelity PCR MasterMix with HF Buffer (NEB). Primers were used at a final concentration of 0.5 μM with the following conditions: initial melt of 98° C. for 30 s; 12 cycles of 98° C. for 10 s, 60° C. for 30 s and 72° C. for 40 s; and a final extension at 72 ° C. for 10 min. A second PCR reaction was then performed to add sample specific sequence barcodes. PCR libraries were purified using the QIAquick PCR purification kit (Qiagen). Samples were quantified with the Qubit dsDNA HS Assay kit (Invitrogen) and diluted to 2 nM. The libraries were then run according to the manufacturer's instructions on either an Illumina MiSeq using a standard 300-cycle kit or an Illumina NextSeq 500 using a mid-output 300-cycle kit.

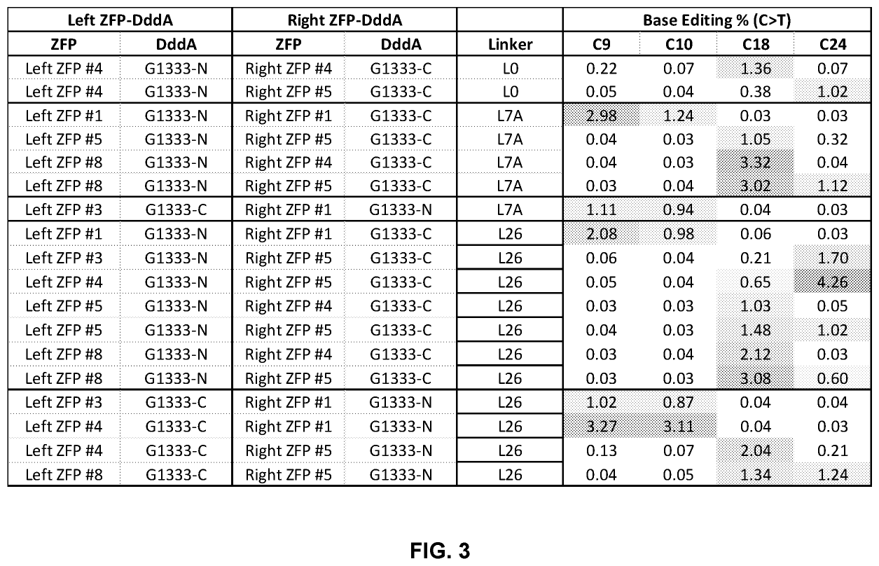

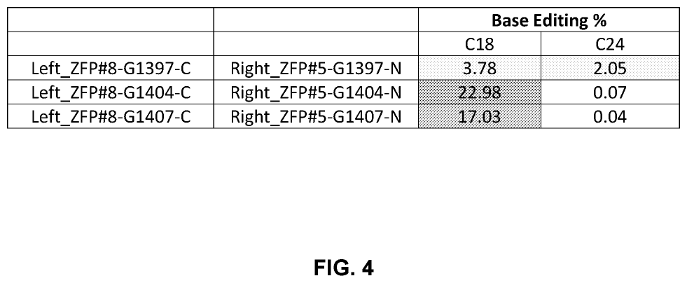

Results using DddA-G1333 are shown in FIG. 3. Base editing of >3% was achieved at all four positions in the CCR5 base editing window (C9, C10, C18, and C24) with no noticeable indels. FIG. 4 provides results for DddA-1397, DddA-G1404, and DddA-G1407 at positions C18 and C24. Notably, DddA-G1404 and DddA-G1407 showed increased efficiency and activity, particularly at C18. Base editing was not seen for any of the 17 GFP controls (data not shown).

### Example 3: “Re-Wired” DddA Design

The DddA polypeptide chain was reconnected without performing standard circular permutation by making residue 1398 the new N-terminus, linking the current C-terminus to residue 1334, linking residue 1397 to the current N-terminus, and making residue 1333 the new C-terminus, as shown below (“re-wired” DddA full):

>DddA full (residues 1290-1427 of SEQ ID NO: 72) (disordered residues italicized; 1333 and 1397 bolded):

(SEQ ID NO: 49)
GSYALGPYQISAPQLPAYNGQTVGTFYYVNDAGGLESKVESSGGPTPYPN
YANAGHVEGQSALFMRDNGISEGLVFHNNPEGTCGFCVNMTETLLPENAK
MTVVPPEGAIPVKRGATGETKVFTGNSNSPKSPTKGGC

>re-wired DddA full (1398-C_term:1334-1397:linker (double underlined):N-term-1333, wherein single underlines indicate near junctions created by re-wiring):

(SEQ ID NO: 50)
AIPVKRGATGETKVFTGNSNSPKSPTKGGCPTPYPNYANAGHVEGQSALF
MRDNGISEGLVFHNNPEGTCGFCVNMTETLLPENAKMTVVPPEGGGSGGS
GSYALGPYQISAPQLPAYNGQTVGTFYYVNDAGGLESKVFSSGG

Two different strategies were then identified to split the re-wired DddA into two halves to make a functional non-toxic base editor, re-wired_G1309 and re-wired N1357:

>re-wired_G1309-N:

(SEQ ID NO: 51)
AIPVKRGATGETKVFTGNSNSPKSPTKGGCPTPYPNYANAGHVEGQSALF
MRDNGISEGLVFHNNPEGTCGFCVNMTETLLPENAKMTVVPPEGGGSGGS
GSYALGPYQISAPQLPAYNG

>re-wired_G1309-C:

(SEQ ID NO: 52)
QTVGTFYYVNDAGGLESKVFSSGG

>re-wired_N1357-N:

(SEQ ID NO: 53)
AIPVKRGATGETKVFTGNSNSPKSPTKGGCPTPYPNYANAGHVEGQSALF
MRDN

>re-wired_N1357-C:

(SEQ ID NO: 54)
GISEGLVFHNNPEGTCGFCVNMTETLLPENAKMTVVPPEGGGSGGSGSYA
LGPYQISAPQLPAYNGQTVGTFYYVNDAGGLESKVFSSGG

Respective ZFP-DddA base editors for the CCR5 locus then were designed based on these split re-wired DddA architectures. See, e.g., Table 4. It is contemplated that when tested in K562 cells according to the protocols described above, the re-wired ZFP-DddA pairs will be able to perform C to T base editing. Such re-wired pairs may increase the specificity of multiplex base editor applications, as only the left and right arm of each split pair can form functional DddA.

TABLE 4
Sequences of Re-Wired ZFP-DddA Constructs (CCR5 Locus)

| SEQ | Description | Sequence |
|---|---|---|
| 55 | Left | MDYKDHDGDYKDHDIDYKDDDDKMAPKKKRKVGIHGVPAAMA |
| | ZFP#8_L26_rewired_ | ERPFQCRICMRNFSQSGDLTRHIRTHTGEKPFACDICGRKFA |
| | G1309-N | QSSDLRRHTKIHTHPRAPIPKPFQCRICMRNFSRSANLARHI |
| | (incl UGI) | RTHTGEKPFACDICGRKFATNQNRITHTKIHTGSQKPFQCRI |
| | | CMRNFSQSGDLTRHIRTHTGEKPFACDICGRKFARKDPLKEH |
| | | TKIHLRGSQLVKSKSEAAARGGGGSGGGGSAIPVKRGATGET |
| | | KVFTGNSNSPKSPTKGGCPTPYPNYANAGHVEGQSALFMRDN |
| | | GISEGLVFHNNPEGTCGFCVNMTETLLPENAKMTVVPPEGGG |
| | | SGGSGSYALGPYQISAPQLPAYNGSGGSTNLSDIIEKETGKQ |
| | | LVIQESILMLPEEVEEVIGNKPESDILVHTAYDESTDENVML |
| | | LTSDAPEYKPWALVIQDSNGENKIKML* |
| 56 | Left | MDYKDHDGDYKDHDIDYKDDDDKMAPKKKRKVGIHGVPAAMA |
| | ZFP#4_L26_rewired_ | ERPFQCRICMRNFSQSGALARHIRTHTGEKPFACDICGRKFA |
| | G1309-N | LKQHLTRHTKIHTGSQKPFQCRICMRNFSQSGDLTRHIRTHT |
| | (incl UGI) | GEKPFACDICGRKFAQSSDLRRHTKIHTHPRAPIPKPFQCRI |
| | | CMRNFSRSANLARHIRTHTGEKPFACDICGRKFATNQNRITH |
| | | TKIHLRGSQLVKSKSEAAARGGGGSGGGGSAIPVKRGATGET |
| | | KVFTGNSNSPKSPTKGGCPTPYPNYANAGHVEGQSALFMRDN |
| | | GISEGLVFHNNPEGTCGFCVNMTETLLPENAKMTVVPPEGGG |
| | | SGGSGSYALGPYQISAPQLPAYNGSGGSTNLSDIIEKETGKQ |
| | | LVIQESILMLPEEVEEVIGNKPESDILVHTAYDESTDENVML |
| | | LTSDAPEYKPWALVIQDSNGENKIKML* |
| 57 | Right | MDYKDHDGDYKDHDIDYKDDDDKMAPKKKRKVGIHGVPAAMA |
| | ZFP#5_L26_rewired_ | ERPFQCRICMRNFSDRSNLSRHIRTHTGEKPFACDICGRKFA |
| | G1309-C | QSGDLTRHTKIHTGSQKPFQCRICMRNFSDIGYRAAHIRTHT |
| | (incl UGI) | GEKPFACDICGRKFAQSGNLARHTKIHTHPRAPIPKPFQCRI |
| | | CMRNFSQSGHLARHIRTHTGEKPFACDICGRKFANRHDRAKH |
| | | TKIHLRGSQLVKSKSEAAARGGGGSGGGGSQTVGTFYYVNDA |
| | | GGLESKVFSSGGSGGSTNLSDIIEKETGKQLVIQESILMLPE |
| | | EVEEVIGNKPESDILVHTAYDESTDENVMLLTSDAPEYKPWA |
| | | LVIQDSNGENKIKML* |
| 58 | Right | MDYKDHDGDYKDHDIDYKDDDDKMAPKKKRKVGIHGVPAAMA |
| | ZFP#2_L26_rewired_ | ERPFQCRICMRNFSQSGHLARHIRTHTGEKPFACDICGRKFA |
| | G1309-C | NRHDRAKHTKIHTPNPHRRTDPSHKPFQCRICMRNFSQSADR |
| | (incl UGI) | TKHIRTHTGEKPFACDICGRKFAQSGSLTRHTKIHTHPRAPI |
| | | PKPFQCRICMRNFSDRSTRITHIRTHTGEKPFACDICGRKFA |
| | | QNATRINHTKIHLRGSQLVKSKSEAAARGGGGSGGGGSQTVG |
| | | TFYYVNDAGGLESKVFSSGGSGGSTNLSDIIEKETGKQLVIQ |
| | | ESILMLPEEVEEVIGNKPESDILVHTAYDESTDENVMLLTSD |
| | | APEYKPWALVIQDSNGENKIKML* |
| 59 | Left | MDYKDHDGDYKDHDIDYKDDDDKMAPKKKRKVGIHGVPAAMA |
| | ZFP#8_L26_rewired_ | ERPFQCRICMRNFSQSGDLTRHIRTHTGEKPFACDICGRKFA |
| | G1309-C | QSSDLRRHTKIHTHPRAPIPKPFQCRICMRNFSRSANLARHI |
| | (incl UGI) | RTHTGEKPFACDICGRKFATNQNRITHTKIHTGSQKPFQCRI |
| | | CMRNFSQSGDLTRHIRTHTGEKPFACDICGRKFARKDPLKEH |
| | | TKIHLRGSQLVKSKSEAAARGGGGSGGGGSQTVGTFYYVNDA |
| | | GGLESKVFSSGGSGGSTNLSDIIEKETGKQLVIQESILMLPE |
| | | EVEEVIGNKPESDILVHTAYDESTDENVMLLTSDAPEYKPWA |
| | | LVIQDSNGENKIKML* |
| 60 | Left | MDYKDHDGDYKDHDIDYKDDDDKMAPKKKRKVGIHGVPAAMA |
| | ZFP#4_L26_rewired_ | ERPFQCRICMRNFSQSGALARHIRTHTGEKPFACDICGRKFA |
| | G1309-C | LKQHLTRHTKIHTGSQKPFQCRICMRNFSQSGDLTRHIRTHT |
| | (incl UGI) | GEKPFACDICGRKFAQSSDLRRHTKIHTHPRAPIPKPFQCRI |
| | | CMRNFSRSANLARHIRTHTGEKPFACDICGRKFATNQNRITH |
| | | TKIHLRGSQLVKSKSEAAARGGGGSGGGGSQTVGTFYYVNDA |
| | | GGLESKVFSSGGSGGSTNLSDIIEKETGKQLVIQESILMLPE |
| | | EVEEVIGNKPESDILVHTAYDESTDENVMLLTSDAPEYKPWA |
| | | LVIQDSNGENKIKML* |
| 61 | Right | MDYKDHDGDYKDHDIDYKDDDDKMAPKKKRKVGIHGVPAAMA |
| | ZFP#5_L26_rewired_ | ERPFQCRICMRNFSDRSNLSRHIRTHTGEKPFACDICGRKFA |
| | G1309-N | QSGDLTRHTKIHTGSQKPFQCRICMRNFSDIGYRAAHIRTHT |
| | (incl UGI) | GEKPFACDICGRKFAQSGNLARHTKIHTHPRAPIPKPFQCRI |
| | | CMRNFSQSGHLARHIRTHTGEKPFACDICGRKFANRHDRAKH |
| | | TKIHLRGSQLVKSKSEAAARGGGGSGGGGSAIPVKRGATGET |
| | | KVFTGNSNSPKSPTKGGCPTPYPNYANAGHVEGQSALFMRDN |
| | | GISEGLVFHNNPEGTCGFCVNMTETLLPENAKMTVVPPEGGG |
| | | SGGSGSYALGPYQISAPQLPAYNGSGGSTNLSDIIEKETGKQ |
| | | LVIQESILMLPEEVEEVIGNKPESDILVHTAYDESTDENVML |
| | | LTSDAPEYKPWALVIQDSNGENKIKML* |
| 62 | Right | MDYKDHDGDYKDHDIDYKDDDDKMAPKKKRKVGIHGVPAAMA |
| | ZFP#2_L26_rewired_ | ERPFQCRICMRNFSQSGHLARHIRTHTGEKPFACDICGRKFA |
| | G1309-N | NRHDRAKHTKIHTPNPHRRTDPSHKPFQCRICMRNFSQSADR |
| | (incl UGI) | TKHIRTHTGEKPFACDICGRKFAQSGSLTRHTKIHTHPRAPI |
| | | PKPFQCRICMRNFSDRSTRITHIRTHTGEKPFACDICGRKFA |
| | | QNATRINHTKIHLRGSQLVKSKSEAAARGGGGSGGGGSAIPV |
| | | KRGATGETKVFTGNSNSPKSPTKGGCPTPYPNYANAGHVEGQ |
| | | SALFMRDNGISEGLVFHNNPEGTCGFCVNMTETLLPENAKMT |
| | | VVPPEGGGSGGSGSYALGPYQISAPQLPAYNGSGGSTNLSDI |
| | | IEKETGKQLVIQESILMLPEEVEEVIGNKPESDILVHTAYDE |
| | | STDENVMLLTSDAPEYKPWALVIQDSNGENKIKML* |
| 63 | Left | MDYKDHDGDYKDHDIDYKDDDDKMAPKKKRKVGIHGVPAAMA |
| | ZFP#8_L26_rewired_ | ERPFQCRICMRNFSQSGDLTRHIRTHTGEKPFACDICGRKFA |
| | G1357-N | QSSDLRRHTKIHTHPRAPIPKPFQCRICMRNFSRSANLARHI |
| | (incl UGI) | RTHTGEKPFACDICGRKFATNQNRITHTKIHTGSQKPFQCRI |
| | | CMRNFSQSGDLTRHIRTHTGEKPFACDICGRKFARKDPLKEH |
| | | TKIHLRGSQLVKSKSEAAARGGGGSGGGGSAIPVKRGATGET |
| | | KVFTGNSNSPKSPTKGGCPTPYPNYANAGHVEGQSALFMRDN |
| | | SGGSTNLSDIIEKETGKQLVIQESILMLPEEVEEVIGNKPES |
| | | DILVHTAYDESTDENVMLLTSDAPEYKPWALVIQDSNGENKI |
| | | KML* |
| 64 | Left | MDYKDHDGDYKDHDIDYKDDDDKMAPKKKRKVGIHGVPAAMA |
| | ZFP#4_L26_rewired_ | ERPFQCRICMRNFSQSGALARHIRTHTGEKPFACDICGRKFA |
| | G1357-N | LKQHLTRHTKIHTGSQKPFQCRICMRNFSQSGDLTRHIRTHT |
| | (incl UGI) | GEKPFACDICGRKFAQSSDLRRHTKIHTHPRAPIPKPFQCRI |
| | | CMRNFSRSANLARHIRTHTGEKPFACDICGRKFATNQNRITH |
| | | TKIHLRGSQLVKSKSEAAARGGGGSGGGGSAIPVKRGATGET |
| | | KVFTGNSNSPKSPTKGGCPTPYPNYANAGHVEGQSALFMRDN |
| | | SGGSTNLSDIIEKETGKQLVIQESILMLPEEVEEVIGNKPES |
| | | DILVHTAYDESTDENVMLLTSDAPEYKPWALVIQDSNGENKI |
| | | KML* |
| 65 | Right | MDYKDHDGDYKDHDIDYKDDDDKMAPKKKRKVGIHGVPAAMA |
| | ZFP#5_L26_rewired_ | ERPFQCRICMRNFSDRSNLSRHIRTHTGEKPFACDICGRKFA |
| | G1357-C | QSGDLTRHTKIHTGSQKPFQCRICMRNFSDIGYRAAHIRTHT |
| | (incl UGI) | GEKPFACDICGRKFAQSGNLARHTKIHTHPRAPIPKPFQCRI |
| | | CMRNFSQSGHLARHIRTHTGEKPFACDICGRKFANRHDRAKH |
| | | TKIHLRGSQLVKSKSEAAARGGGGSGGGGSGISEGLVFHNNP |
| | | EGTCGFCVNMTETLLPENAKMTVVPPEGGGSGGSGSYALGPY |
| | | QISAPQLPAYNGQTVGTFYYVNDAGGLESKVFSSGGSGGSTN |
| | | LSDIIEKETGKQLVIQESILMLPEEVEEVIGNKPESDILVHT |
| | | AYDESTDENVMLLTSDAPEYKPWALVIQDSNGENKIKML* |
| 66 | Right | MDYKDHDGDYKDHDIDYKDDDDKMAPKKKRKVGIHGVPAAMA |
| | ZFP#2_L26_rewired_ | ERPFQCRICMRNFSQSGHLARHIRTHTGEKPFACDICGRKFA |
| | G1357-C | NRHDRAKHTKIHTPNPHRRTDPSHKPFQCRICMRNFSQSADR |
| | (incl UGI) | TKHIRTHTGEKPFACDICGRKFAQSGSLTRHTKIHTHPRAPI |
| | | PKPFQCRICMRNFSDRSTRITHIRTHTGEKPFACDICGRKFA |
| | | QNATRINHTKIHLRGSQLVKSKSEAAARGGGGSGGGGSGISE |
| | | GLVFHNNPEGTCGFCVNMTETLLPENAKMTVVPPEGGGSGGS |
| | | GSYALGPYQISAPQLPAYNGQTVGTFYYVNDAGGLESKVESS |
| | | GGSGGSTNLSDIIEKETGKQLVIQESILMLPEEVEEVIGNKP |
| | | ESDILVHTAYDESTDENVMLLTSDAPEYKPWALVIQDSNGEN |
| | | KIKML* |
| 67 | Left | MDYKDHDGDYKDHDIDYKDDDDKMAPKKKRKVGIHGVPAAMA |
| | ZFP#8_L26_rewired_ | ERPFQCRICMRNFSQSGDLTRHIRTHTGEKPFACDICGRKFA |
| | G1357-C | QSSDLRRHTKIHTHPRAPIPKPFQCRICMRNFSRSANLARHI |
| | (incl UGI) | RTHTGEKPFACDICGRKFATNQNRITHTKIHTGSQKPFQCRI |
| | | CMRNFSQSGDLTRHIRTHTGEKPFACDICGRKFARKDPLKEH |
| | | TKIHLRGSQLVKSKSEAAARGGGGSGGGGSGISEGLVFHNNP |
| | | EGTCGFCVNMTETLLPENAKMTVVPPEGGGSGGSGSYALGPY |
| | | QISAPQLPAYNGQTVGTFYYVNDAGGLESKVFSSGGSGGSTN |
| | | LSDIIEKETGKQLVIQESILMLPEEVEEVIGNKPESDILVHT |
| | | AYDESTDENVMLLTSDAPEYKPWALVIQDSNGENKIKML* |
| 68 | Left | MDYKDHDGDYKDHDIDYKDDDDKMAPKKKRKVGIHGVPAAMA |
| | ZFP#4_L26_rewired_ | ERPFQCRICMRNFSQSGALARHIRTHTGEKPFACDICGRKFA |
| | G1357-C | LKQHLTRHTKIHTGSQKPFQCRICMRNFSQSGDLTRHIRTHT |
| | (incl UGI) | GEKPFACDICGRKFAQSSDLRRHTKIHTHPRAPIPKPFQCRI |
| | | CMRNFSRSANLARHIRTHTGEKPFACDICGRKFATNQNRITH |
| | | TKIHLRGSQLVKSKSEAAARGGGGSGGGGSGISEGLVFHNNP |
| | | EGTCGFCVNMTETLLPENAKMTVVPPEGGGSGGSGSYALGPY |
| | | QISAPQLPAYNGQTVGTFYYVNDAGGLESKVFSSGGSGGSTN |
| | | LSDIIEKETGKQLVIQESILMLPEEVEEVIGNKPESDILVHT |
| | | AYDESTDENVMLLTSDAPEYKPWALVIQDSNGENKIKML* |
| 69 | Right | MDYKDHDGDYKDHDIDYKDDDDKMAPKKKRKVGIHGVPAAMA |
| | ZFP#5_L26_rewired_ | ERPFQCRICMRNFSDRSNLSRHIRTHTGEKPFACDICGRKFA |
| | G1357-N | QSGDLTRHTKIHTGSQKPFQCRICMRNFSDIGYRAAHIRTHT |
| | (incl UGI) | GEKPFACDICGRKFAQSGNLARHTKIHTHPRAPIPKPFQCRI |
| | | CMRNFSQSGHLARHIRTHTGEKPFACDICGRKFANRHDRAKH |
| | | TKIHLRGSQLVKSKSEAAARGGGGSGGGGSAIPVKRGATGET |
| | | KVFTGNSNSPKSPTKGGCPTPYPNYANAGHVEGQSALFMRDN |
| | | SGGSTNLSDIIEKETGKQLVIQESILMLPEEVEEVIGNKPES |
| | | DILVHTAYDESTDENVMLLTSDAPEYKPWALVIQDSNGENKI |
| | | KML* |
| 70 | Right | MDYKDHDGDYKDHDIDYKDDDDKMAPKKKRKVGIHGVPAAMA |
| | ZFP#2_L26_rewired_ | ERPFQCRICMRNFSQSGHLARHIRTHTGEKPFACDICGRKFA |
| | G1357-N | NRHDRAKHTKIHTPNPHRRTDPSHKPFQCRICMRNFSQSADR |
| | (incl UGI) | TKHIRTHTGEKPFACDICGRKFAQSGSLTRHTKIHTHPRAPI |
| | | PKPFQCRICMRNFSDRSTRITHIRTHTGEKPFACDICGRKFA |
| | | QNATRINHTKIHLRGSQLVKSKSEAAARGGGGSGGGGSAIPV |
| | | KRGATGETKVFTGNSNSPKSPTKGGCPTPYPNYANAGHVEGQ |
| | | SALFMRDNSGGSTNLSDIIEKETGKQLVIQESILMLPEEVEE |
| | | VIGNKPESDILVHTAYDESTDENVMLLTSDAPEYKPWALVIQ |
| | | DSNGENKIKML* |

SED: SEQ ID NO.

### Example 4: Reshaping of the ZFP-DddA Binding Pocket

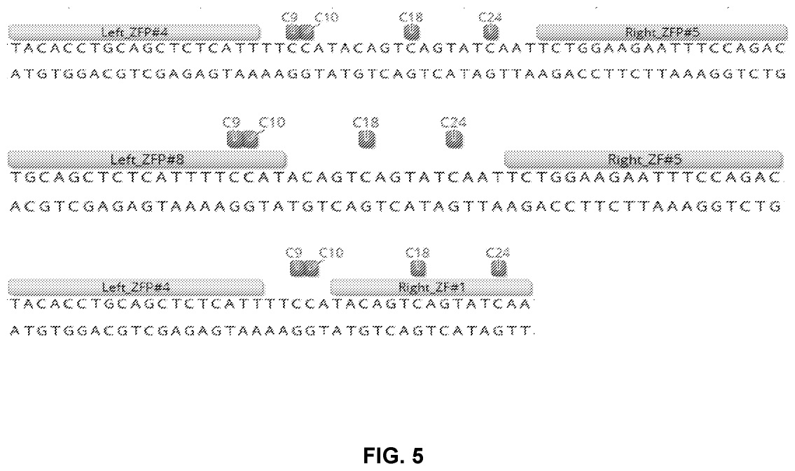

DddA-derived cytosine base editors are restricted to C to T editing and have a strong preference for TC dinucleotides within the base editing window. Various residues were identified for saturation mutagenesis to relax these restrictions and to increase the efficiency and/or activity of the enzyme, including Y1307, T1311, 51331, V1346, H1366, N1367, N1368, P1369, E1370, G1371, T1372, F1375, V1392, P1394, P1395, 11399, P1400, V1401, K1402, A1405, and T1406. The mutations are numbered with respect to SEQ ID NO: 72. Based on structural alignments between DddA and other base editors, including adenine deaminases, it was determined that these residues form the nucleotide pocket. DddA variants with mutations at positions E1370, N1368, and Y1307 were tested in K562 cells according to the protocols described above, using the left and right ZFP pairs shown in FIG. 5.

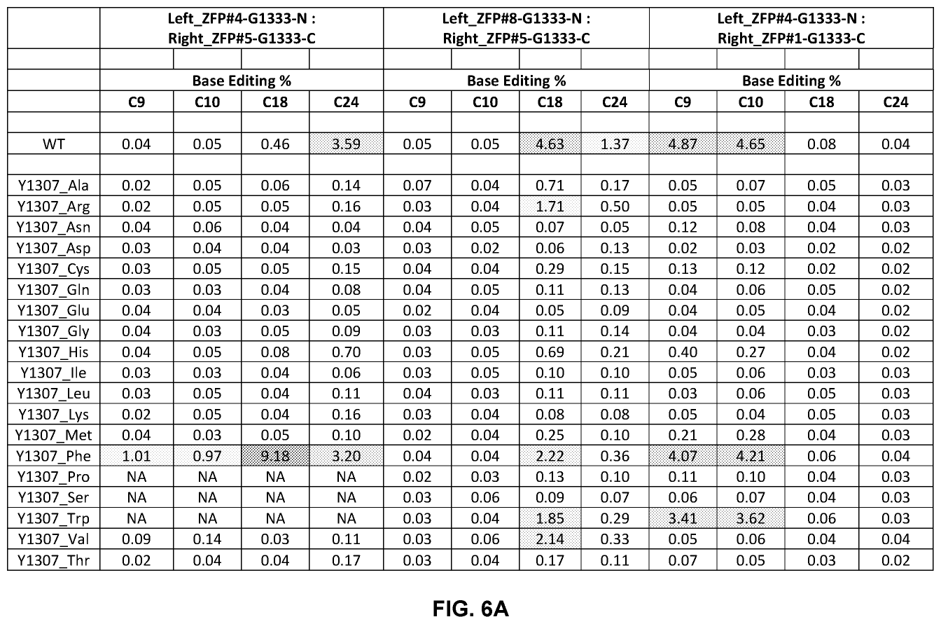

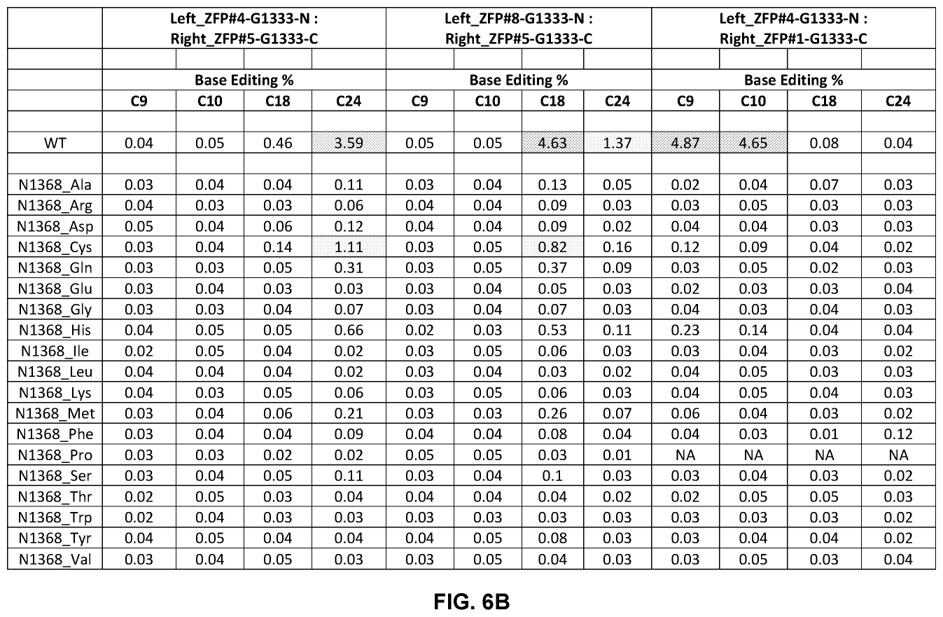

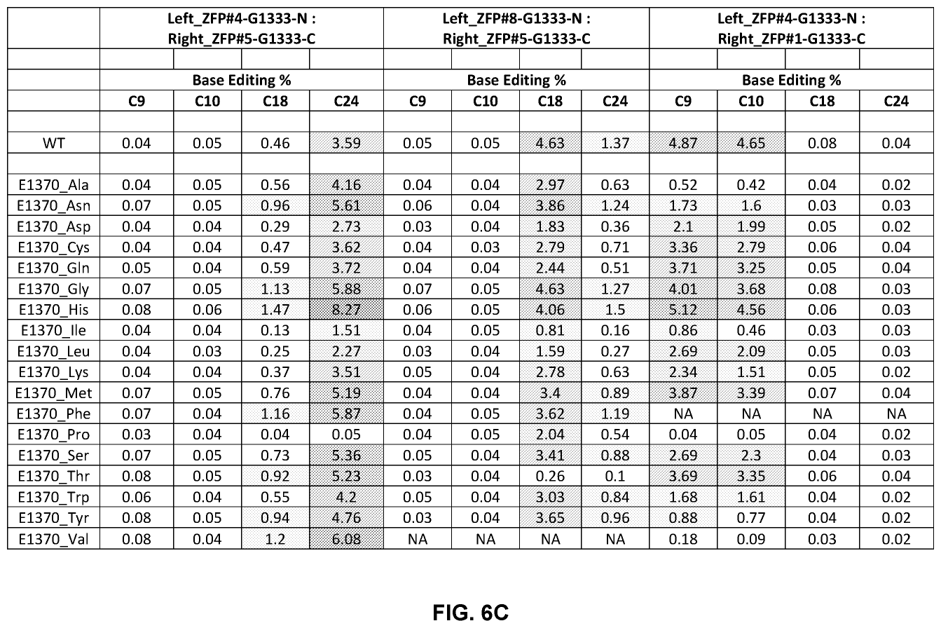

As shown in FIGS. 6A-6C, certain residue changes gave rise to an increase in efficiency/activity. Further, some residue changes altered the activity window of the DddA enzyme; such alterations may increase the precision and specificity of DddA-based reagents. Y1307 and N1368 both appeared sensitive to changes, with some mutations altering the activity profile of Y1307 (e.g., an almost 20x increase in activity at C18 in certain cases, and ability to access C9 and C10). E1370 appeared less sensitive to changes, with certain mutations showing a beneficial effect (e.g., E1370H, in the context of “Left_ZFP#4-G1333-N: Right_ZFP#5-G1333 -C”).

### Example 5: Combined ZFP-TDD+Nickase Approach to Base Editing

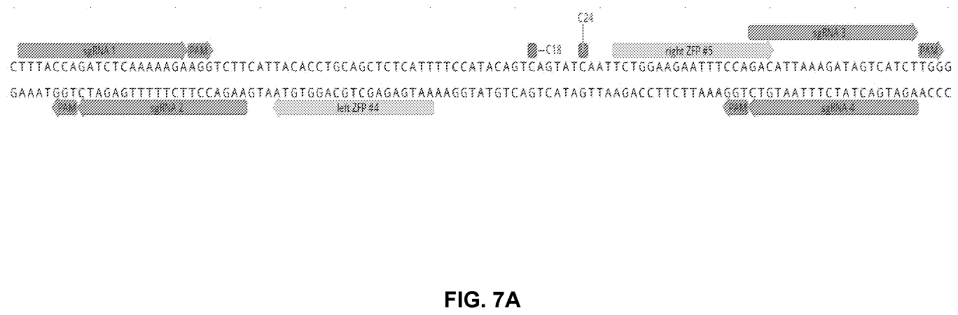

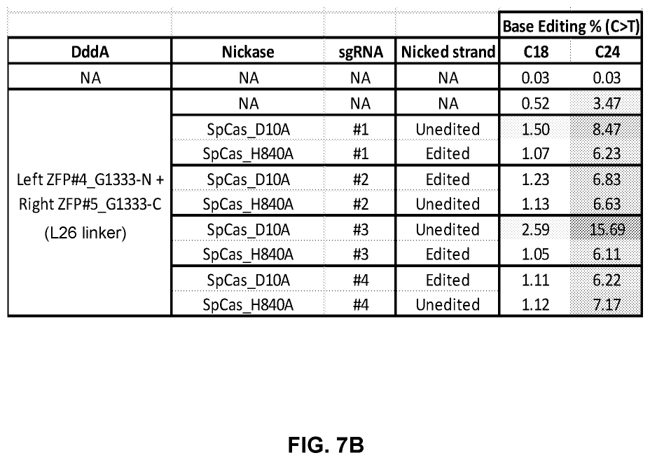

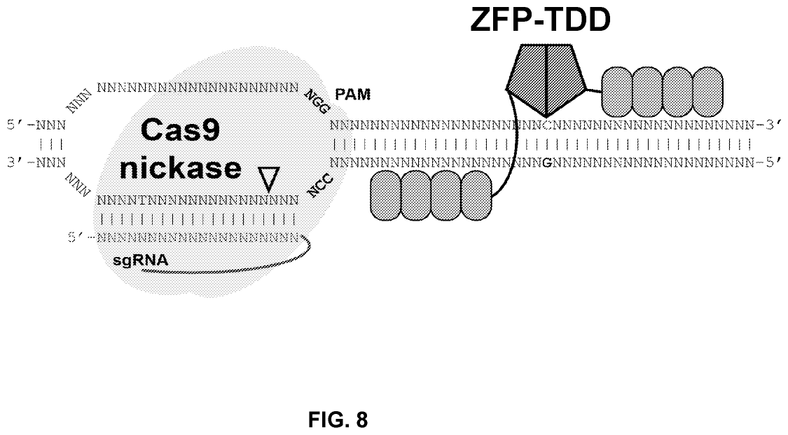

The efficiency of base editors can be increased by nicking the unmodified DNA strand with a nickase. The unmodified DNA strand then is recognized as newly synthesized by the cell, and the natural DNA repair machinery repairs the nicked DNA strand using the modified strand as a template. The unmodified strand can be nicked using a FokI-derived ZFN or TALEN or a CRISPR/Cas-derived nickase. FIGS. 7A and 7B demonstrate a ZFP-TDD base editing design and results, respectively, with a CRISPR/Cas9 nickase. However, all three approaches require the delivery of two additional constructs (two peptides for ZFN or TALEN nickases; one peptide and one sgRNA for CRISPR/Cas nickases; FIG. 8).

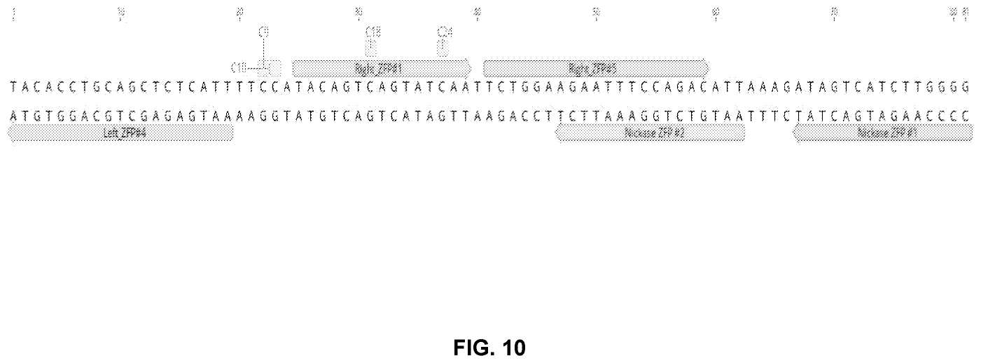

A trimeric ZFP-TDD base editor architecture was developed to overcome this limitation, facilitating delivery and also making it more likely that the base editing and DNA nicking will happen simultaneously, increasing editing efficiency. With such a trimeric architecture, one half of a dimeric Fokl nickase may be fused to the N-terminus of the left or right ZFP-TDD and the corresponding other half of the Fokl nickase may be targeted to the site of interest through an independent ZFP-Fokl peptide (FIG. 9). Sequences for nickase experiments using DddA may be found in Table 5 below, with the ZFP design shown in FIG. 10 (Left_ZFP#4+Right_ZFP#1+Nickase_ZFP #2, or Left_ZFP#4+Right_ZFP#5+Nickase ZFP #1).

TABLE 5
Sequences of ZFP-Nickase Constructs (CCR5 Locus)

| SEQ | Description | Sequence |
|---|---|---|
| 77 | FokI(ELD)- | MDYKDHDGDYKDHDIDYKDDDDKMAPKKKRKVGIHGVPAAMGQLVKSE |
| | Right_ZFP#5- | LEEKKSELRHKLKYVPHEYIELIEIARNSTQDRILEMKVMEFFMKVYG |
| | G1333-C | YRGKHLGGSRKPDGAIYTVGSPIDYGVIVDTKAYSGGYNLPIGQADEM |
| | | ERYVEENQTRDKHLNPNEWWKVYPSSVTEFKFLFVSGHFKGNYKAQLT |
| | | RLNHITNCNGAVLSVEELLIGGEMIKAGTLTLEEVRRKFNNGEINFSG |
| | | AQGSTLDFRPFQCRICMRNFSDRSNLSRHIRTHTGEKPFACDICGRKF |
| | | AQSGDLTRHTKIHTGSQKPFQCRICMRNFSDIGYRAAHIRTHTGEKPF |
| | | ACDICGRKFAQSGNLARHTKIHTHPRAPIPKPFQCRICMRNFSQSGHL |
| | | ARHIRTHTGEKPFACDICGRKFANRHDRAKHTKIHLRGSQLVKSKSEA |
| | | AARGGGGSGGGGSPTPYPNYANAGHVEGQSALFMRDNGISEGLVFHNN |
| | | PEGTCGFCVNMTETLLPENAKMTVVPPEGAIPVKRGATGETKVFTGNS |
| | | NSPKSPTKGGCSGGSTNLSDIIEKETGKQLVIQESILMLPEEVEEVIG |
| | | NKPESDILVHTAYDESTDENVMLLTSDAPEYKPWALVIQDSNGENKIK |
| | | ML* |
| 78 | Nickase #1 | MDYKDHDGDYKDHDIDYKDDDDKMAPKKKRKVGIHGVPAAMGQLVKSE |
| | (ZFP-FokI | LEEKKSELRHKLKYVPHEYIELIEIARNSTQDRILEMKVMEFFMKVYG |
| | (KKR_F450N)) | YRGKHLGGSRKPNGAIYTVGSPIDYGVIVDTKAYSGGYNLPIGQADEM |
| | | QRYVKENQTRNKHINPNEWWKVYPSSVTEFKFLFVSGHFKGNYKAQLT |
| | | RLNRKTNCNGAVLSVEELLIGGEMIKAGTLTLEEVRRKFNNGEINFSG |
| | | AQGSTLDFRPFQCRICMRNFSCSNNLPTHIRTHTGEKPFACDICGRKF |
| | | ADRSNLTRHTKIHTGSQKPFQCRICMRNFSTSGNLTRHIRTHTGEKPF |
| | | ACDICGRKFAQAENLKSHTKIHTGEKPFQCRICMRKFADRSTLRQHTK |
| | | IHLRQKD* |
| 79 | FokI(ELD)- | MDYKDHDGDYKDHDIDYKDDDDKMAPKKKRKVGIHGVPAAMGQLVKSE |
| | Right_ZFP#1- | LEEKKSELRHKLKYVPHEYIELIEIARNSTQDRILEMKVMEFFMKVYG |
| | G1333-N | YRGKHLGGSRKPDGAIYTVGSPIDYGVIVDTKAYSGGYNLPIGQADEM |
| | | ERYVEENQTRDKHLNPNEWWKVYPSSVTEFKFLFVSGHFKGNYKAQLT |
| | | RLNHITNCNGAVLSVEELLIGGEMIKAGTLTLEEVRRKFNNGEINFSG |
| | | AQGSTLDFRPFQCRICMRKFAQSGNRTTHTKIHTGEKPFQCRICMRNF |
| | | STSSNRKTHIRTHTGEKPFACDICGRKFAAQWTRACHTKIHTGSQKPF |
| | | QCRICMRNFSLRHHLTRHIRTHTGEKPFACDICGRKFADRTGLRSHTK |
| | | IHLRGSQLVKSKSEAAARGGGGSGGGGSGSYALGPYQISAPQLPAYNG |
| | | QTVGTFYYVNDAGGLESKVFSSGGSGGSTNLSDIIEKETGKQLVIQES |
| | | ILMLPEEVEEVIGNKPESDILVHTAYDESTDENVMLLTSDAPEYKPWA |
| | | LVIQDSNGENKIKML* |
| 80 | Nickase #2 | MDYKDHDGDYKDHDIDYKDDDDKMAPKKKRKVGIHGVPAAMGQLVKSE |
| | (ZFP-FokI | LEEKKSELRHKLKYVPHEYIELIEIARNSTQDRILEMKVMEFFMKVYG |
| | (KKR F450N)) | YRGKHLGGSRKPNGAIYTVGSPIDYGVIVDTKAYSGGYNLPIGQADEM |
| | | QRYVKENQTRNKHINPNEWWKVYPSSVTEFKFLFVSGHFKGNYKAQLT |
| | | RLNRKTNCNGAVLSVEELLIGGEMIKAGTLTLEEVRRKFNNGEINFSG |
| | | AQGSTLDFRPFQCRICMRKFARNADRKKHTKIHTGEKPFQCRICMRNF |
| | | STSSNRKTHIRTHTGEKPFACDICGRKFAQSGHLSRHTKIHTHPRAPI |
| | | PKPFQCRICMRNFSDRSALSRHIRTHTGEKPFACDICGRKFATSSNRK |
| | | THTKIHLRQKD* |

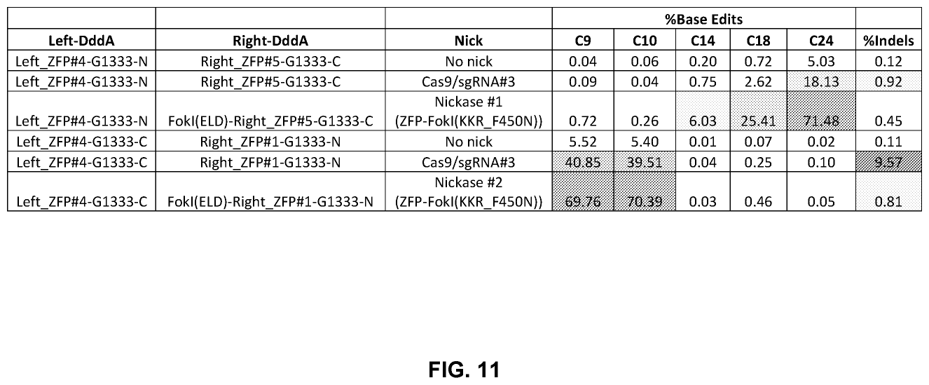

The trimeric ZFP-DddA-nickase system was tested in K562 cells according to the protocols described above. As shown in FIG. 11, the trimeric ZFP-DddA-nickase system demonstrated a higher level of base editing activity than CRISPR-based nickases, with around 70% base edits in some cases, and a lower level of indels that approached background. In addition to outperforming the CRISPR-based nickase system, the trimeric ZFP-TDD-nickase system may be highly advantageous in its compact size, which may fit into a single viral vector such as AAV, unlike other platforms such as CRISPR/Cas and TALE-TDD base editor systems.

### Example 6: Base Editing Activity of TDDs in K562 Cells

19 other potential cytidine deaminases were identified (Table 6) and were tested for base editing activity.

TABLE 6
TDD Information

| No. | NCBI No. | SEQ | Organism |
|---|---|---|---|
| TDD1 | WP_069977532.1 | 86 | Streptomyces rubrolavendulae |
| TDD2 | WP_021798742.1 | 87 | Propionibacterium acidifaciens |
| TDD3 | QNM04114 | 88 | Lachnospiraceae bacterium sunii NSJ-8 |
| TDD4 | WP_181981612 | 89 | Ruminococcus bicirculans |
| TDD5 | AXI73669.1 | 90 | Streptomyces cavourensis |
| TDD6 | WP_195441564 | 91 | Roseburia intestinalis |
| TDD7 | AVT32940.1 | 117 | Plantactinospora sp BCI |
| TDD8 | WP_189594293.1 | 118 | Streptomyces massasporeous |
| TDD9 | TCP42004.1 | 119 | Streptomyces sp. BK438 |
| TDD10 | WP_171906854.1 | 120 | Jiangella alba |
| TDD11 | WP_174422267.1 | 121 | Burkholderia diffusa |
| TDD12 | WP_059728184.1 | 122 | Burkholderia ubonensis |
| TDD13 | WP_133186147.1 | 123 | Paraburkholderia guartelaensis |
| TDD14 | WP_083941146.1 | 124 | Pseudoduganella violaceinigra |
| TDD15 | WP_082507154.1 | 125 | Duganella sp. Root336D2 |
| TDD16 | WP_044236021.1 | 126 | Chondromyces apiculatus |
| TDD17 | WP_165374601.1 | 127 | Sorangium cellulosum |
| TDD18 | NLI59004.1 | 128 | Clostridium sp. |
| TDD19 | KAB8140648.1 | 129 | Chloroflexia bacterium SDU3 |

SEQ: SEQ ID NO:

TDDs described above were substituted for DddA in the base editing systems described in the above Examples, and were tested in K562 cells according to the described protocols for base editing at a CCR5 locus, using the CCR5-targeting ZFPs described above, and/or at a CIITA locus (“site 2”), using the CITTA-targeting ZFPs described below (see Table 7). Sequences for the CIITA primers and amplicon are shown in Table 8 below.

TABLE 7
CIITA Site 2 Zinc Finger Proteins

| SEQ | Description | Sequence |
|---|---|---|
| 241 | CIITA_site_ | ERPFQCRICMRNFSRSAHLSRHIRTHTGEKPFAC |
| | 2_left_6 | DICGRKFATSGHLSRHTKIHTHPRAPIPKPFQCR |
| | | ICMRNFSDSSHRTRHIRTHTGEKPFACDICGRKF |
| | | AAKWNLDAHTKIHTGSQKPFQCRICMRNFSRPYT |
| | | LRLHIRTHTGEKPFACDICGRKFALRHHLTRHTK |
| | | IH |
| 242 | CIITA site_ | ERPFQCRICMRNFSQSGHLARHIRTHTGEKPFAC |
| | 2_right_1 | DICGRKFARKWTLQGHTKIHTGSQKPFQCRICMR |
| | | NFSIRSTLRDHIRTHTGEKPFACDICGRKFAHRS |
| | | SLRRHTKIHTGSQKPFQCRICMRNFSQSGNLARH |
| | | IRTHTGEKPFACDICGRKFARNVDLIHHTKIH |
| 243 | CIITA site_ | ERPFQCRICMRNFSIRSTLRDHIRTHTGEKPFAC |
| | 2_right_5 | DICGRKFAHRSSLRRHTKIHTGSQKPFQCRICMR |
| | | NFSQSGNLARHIRTHTGEKPFACDICGRKFARNV |
| | | DLIHHTKIHTGSQKPFQCRICMRNFSRSDVLSEH |
| | | IRTHTGEKPFACDICGRKFATSGHLSRHTKIH |

SEQ: SEQ ID NO.

One member of each TDD split was fused to the C-terminus of a left ZFP, and the other member was fused to the C-terminus of a right ZFP, using the L26 linker (SEQ ID NO: 17). A UGI (uracil DNA glycosylase inhibitor) domain (SEQ ID NO: 20) was also fused to the C-terminus of each N-terminal and C-terminal half with an SGGS linker (SEQ ID NO: 245). All ZFP fusion constructs further contained a 3×FLAG tag as well as an SV40 nuclear localization signal (SEQ ID NO: 1) fused to the N-terminus of the ZFP.

TABLE 8
CIITA Site 2 Primer and Amplicon Sequences

| SEQ | Description | Sequence |
|---|---|---|
| 224 | CIITA site | ACACGACGCTCTTCCGATCTNNNNCTGGGGCAGC |
| | 2 forward | TGATCACATGT |
| | primer | |
| 225 | CIITA site | GACGTGTGCTCTTCCGATCTCTTCCATCCCCTCC |
| | 2 reverse | CCAAG |
| | primer | |
| 226 | CIITA site | NNNNCTGGGGCAGCTGATCACATGTTTTCTCTGC |
| | 2 NGS | AGCCTTCCCAGAGGAGCTTCCGGCAGACCTGAAG |
| | amplicon | CACTGGAAGCCAGGTGTGCAGGGCAGGTGGGCTG |
| | | GGGTTGGGAAGGGTGGATGCCTTGGGGAGGGGAT |
| | | GGAAG |

SEQ: SEQ ID NO.

Sequences used for the TDDs are included in Table 9 below. For certain TDDs, a variant toxic domain was also tested (indicated by “b” after the TDD indicator, e.g., “TDD2b” for TDD2).

TABLE 9
Sequences of TDD Toxic Domains and Splits

| No. | Description | Sequence | SEQ |
|---|---|---|---|
| TDD1 | toxic domain | VAGNRAFTQRARTYNLTVADLHTYYVLAGQTPVLVH | 92 |
| | | NANCGPHLKDLQKDYPRRTVGILDVGTDQLPMISGPGG | |
| | | QSGLLKNLPGRTKANGEHVETHAAAFLRMNPGVRKAV | |
| | | LYIDYPTGTCGTCRSTLPDMLPEGVQLWVISPRRTEKFT | |
| | | GLPD | |
| | G2278-N | VAGNRAFTQRARTYNLTVADLHTYYVLAGQTPVLVH | 93 |
| | | NANCGPHLKDLQKDYPRRTVGILDVGTDQLPMISGPGG | |
| | G2278-C | QSGLLKNLPGRTKANGEHVETHAAAFLRMNPGVRKAV | 94 |
| | | LYIDYPTGTCGTCRSTLPDMLPEGVQLWVISPRRTEKFT | |
| | | GLPD | |
| | S2346-N | VAGNRAFTQRARTYNLTVADLHTYYVLAGQTPVLVH | 130 |
| | | NANCGPHLKDLQKDYPRRTVGILDVGTDQLPMISGPGG | |
| | | QSGLLKNLPGRTKANGEHVETHAAAFLRMNPGVRKAV | |
| | | LYIDYPTGTCGTCRSTLPDMLPEGVQLWVIS | |
| | S2346-C | PRRTEKFTGLPD | 131 |
| TDD2 | toxic domain | LSTTGKNVLGHFEPTPTTPQGTSSDTIAEMLNSASQPGR | 95 |
| | | TAGVLDIDGELTPLTSGRPSLPNYIASGHVEGQAAMIM | |
| | | RQQQVQSATVYHDNPNGTCGYCYSQLPTLLPEGAALD | |
| | | VVPPAGTVPPSNRWHNGGPSFIGNSSEPKPWPR | |
| | G1794-N | LSTTGKNVLGHFEPTPTTPQGTSSDTIAEMLNSASQPGR | 96 |
| | | TAGVLDIDGELTPLTSG | |
| | G1794-C | RPSLPNYIASGHVEGQAAMIMRQQQVQSATVYHDNPN | 97 |
| | | GTCGYCYSQLPTLLPEGAALDVVPPAGTVPPSNRWHN | |
| | | GGPSFIGNSSEPKPWPR | |
| | P1861-N | LSTTGKNVLGHFEPTPTTPQGTSSDTIAEMLNSASQPGR | 132 |
| | | TAGVLDIDGELTPLTSGRPSLPNYIASGHVEGQAAMIM | |
| | | RQQQVQSATVYHDNPNGTCGYCYSQLPTLLPEGAALD | |
| | | VVPPAGTVP | |
| | P1861-C | PSNRWHNGGPSFIGNSSEPKPWPR | 133 |
| TDD2b | toxic domain | PTPTTPQGTSSDTIAEMLNSASQPGRTAGVLDIDGELTP | 134 |
| | | LTSGRPSLPNYIASGHVEGQAAMIMRQQQVQSATVYH | |
| | | DNPNGTCGYCYSQLPTLLPEGAALDVVPPAGTVPPSNR | |
| | | WHNGGPSFIGNSSEPKPWPR | |
| | G1794-N | PTPTTPQGTSSDTIAEMLNSASQPGRTAGVLDIDGELTP | 135 |
| | | LTSG | |
| | G1794-C | RPSLPNYIASGHVEGQAAMIMRQQQVQSATVYHDNPN | 136 |
| | | GTCGYCYSQLPTLLPEGAALDVVPPAGTVPPSNRWHN | |
| | | GGPSFIGNSSEPKPWPR | |
| | P1861-N | PTPTTPQGTSSDTIAEMLNSASQPGRTAGVLDIDGELTP | 137 |
| | | LTSGRPSLPNYIASGHVEGQAAMIMRQQQVQSATVYH | |
| | | DNPNGTCGYCYSQLPTLLPEGAALDVVPPAGTVP | |
| | P1861-C | PSNRWHNGGPSFIGNSSEPKPWPR | 138 |
| TDD3 | toxic domain | MSLPEYDGTTTHGVLVLDDGTQIGFTSGNGDPRYTNYR | 98 |
| | | NNGHVEQKSALYMRENNISNATVYHNNTNGTCGYCN | |
| | | TMTATFLPEGATLTVVPPENAVANNSRAIDYVKTYTGT | |
| | | SNDPKISPRYKGN | |
| | G30-N | MSLPEYDGTTTHGVLVLDDGTQIGFTSGNG | 99 |
| | G30-C | DPRYTNYRNNGHVEQKSALYMRENNISNATVYHNNTN | 100 |
| | | GTCGYCNTMTATFLPEGATLTVVPPENAVANNSRAIDY | |
| | | VKTYTGTSNDPKISPRYKGN | |
| | N94-N | DPRYTNYRNNGHVEQKSALYMRENNISNATVYHNNTN | 139 |
| | | GTCGYCNTMTATFLPEGATLTVVPPEN | |
| | N94-C | AVANNSRAIDYVKTYTGTSNDPKISPRYKGN | 140 |
| TDD4 | toxic domain | HTYHVGKCRLLVHNANCNQEKPVLPKYDGKTTEGVM | 101 |
| | | VTPDGKQISFKSGNSSTPSYPQYKAQSASHVEGKAALY | |
| | | MRENGINEATVFHNNPNGTCGFCDRQVPALLPKGAKL | |
| | | TVVPPSNSVANNVRAIPVPKTYIGNSTVPKIK | |
| | T161-N | HTYHVGKCRLLVHNANCNQEKPVLPKYDGKTTEGVM | 102 |
| | | VTPDGKQISFKSGNSST | |
| | T161-C | PSYPQYKAQSASHVEGKAALYMRENGINEATVFHNNP | 103 |
| | | NGTCGFCDRQVPALLPKGAKLTVVPPSNSVANNVRAIP | |
| | | VPKTYIGNSTVPKIK | |
| | A229-N | HTYHVGKCRLLVHNANCNQEKPVLPKYDGKTTEGVM | 141 |
| | | VTPDGKQISFKSGNSSTPSYPQYKAQSASHVEGKAALY | |
| | | MRENGINEATVFHNNPNGTCGFCDRQVPALLPKGAKL | |
| | | TVVPPSNSVA | |
| | A229-C | NNVRAIPVPKTYIGNSTVPKIK | 142 |
| TDD4b | toxic domain | ANCNQEKPVLPKYDGKTTEGVMVTPDGKQISFKSGNSS | 143 |
| | | TPSYPQYKAQSASHVEGKAALYMRENGINEATVFHNN | |
| | | PNGTCGFCDRQVPALLPKGAKLTVVPPSNSVANNVRAI | |
| | | PVPKTYIGNSTVPKIK | |
| | T161-N | ANCNQEKPVLPKYDGKTTEGVMVTPDGKQISFKSGNSS | 144 |
| | | T | |
| | T161-C | PSYPQYKAQSASHVEGKAALYMRENGINEATVFHNNP | 145 |
| | | NGTCGFCDRQVPALLPKGAKLTVVPPSNSVANNVRAIP | |
| | | VPKTYIGNSTVPKIK | |
| | A229-N | ANCNQEKPVLPKYDGKTTEGVMVTPDGKQISFKSGNSS | 146 |
| | | TPSYPQYKAQSASHVEGKAALYMRENGINEATVFHNN | |
| | | PNGTCGFCDRQVPALLPKGAKLTVVPPSNSVA | |
| | A229-C | NNVRAIPVPKTYIGNSTVPKIK | 147 |
| TDD5 | toxic domain | VQITAIKRWTETATVHNLTVADLHTYYVLAGKTPVLV | 104 |
| | | HNENCGPNLKDLPKDYDRRTVGILDVGTDQLPMISGPG | |
| | | GQSGLLKNLPGRTKANTDHVEAHTAAFLRMNPGIRKA | |
| | | VLYIDYPTGTCGTCGSTLPDMLPEGVQLWVISPRKTEK | |
| | | FAGLPD | |
| | G2299-N | VQITAIKRWTETATVHNLTVADLHTYYVLAGKTPVLV | 105 |
| | | HNENCGPNLKDLPKDYDRRTVGILDVGTDQLPMISGPG | |
| | | G | |
| | G2299-C | QSGLLKNLPGRTKANTDHVEAHTAAFLRMNPGIRKAV | 106 |
| | | LYIDYPTGTCGTCGSTLPDMLPEGVQLWVISPRKTEKF | |
| | | AGLPD | |
| | S2367-N | VQITAIKRWTETATVHNLTVADLHTYYVLAGKTPVLV | 148 |
| | | HNENCGPNLKDLPKDYDRRTVGILDVGTDQLPMISGPG | |
| | | GQSGLLKNLPGRTKANTDHVEAHTAAFLRMNPGIRKA | |
| | | VLYIDYPTGTCGTCGSTLPDMLPEGVQLWVIS | |
| | S2367-C | PRKTEKFAGLPD | 149 |
| TDD6 | toxic domain | SAGAGESGRKTISLPEYDGTTTHGVLVLDDGTQIGFTSG | 107 |
| | | NGDPRYTNYRNNGHVEQKSALYMRENNISNATVYHN | |
| | | NTNGTCGYCNTMTATFLPEGATLTVVPPENAVANNSR | |
| | | AIDYVKTYTGTSNDPKISPRYKGN | |
| | N2313-N | SAGAGESGRKTISLPEYDGTTTHGVLVLDDGTQIGFTSG | 108 |
| | | N | |
| | N2313-C | GDPRYTNYRNNGHVEQKSALYMRENNISNATVYHNNT | 109 |
| | | NGTCGYCNTMTATFLPEGATLTVVPPENAVANNSRAID | |
| | | YVKTYTGTSNDPKISPRYKGN | |
| | R2385-N | SAGAGESGRKTISLPEYDGTTTHGVLVLDDGTQIGFTSG | 150 |
| | | NGDPRYTNYRNNGHVEQKSALYMRENNISNATVYHN | |
| | | NTNGTCGYCNTMTATFLPEGATLTVVPPENAVANNSR | |
| | R2385-C | AIDYVKTYTGTSNDPKISPRYKGN | 151 |
| TDD6b | toxic domain | DPSGYDSQYPCKEEMSAGAGESGRKTISLPEYDGTTTH | 152 |
| | | GVLVLDDGTQIGFTSGNGDPRYTNYRNNGHVEQKSAL | |
| | | YMRENNISNATVYHNNTNGTCGYCNTMTATFLPEGAT | |
| | | LTVVPPENAVANNSRAIDYVKTYTGTSNDPKISPRYKG | |
| | | N | |
| | N2313-N | DPSGYDSQYPCKEEMSAGAGESGRKTISLPEYDGTTTH | 153 |
| | | GVLVLDDGTQIGFTSGN | |
| | N2313-C | GDPRYTNYRNNGHVEQKSALYMRENNISNATVYHNNT | 154 |
| | | NGTCGYCNTMTATFLPEGATLTVVPPENAVANNSRAID | |
| | | YVKTYTGTSNDPKISPRYKGN | |
| | R2385-N | DPSGYDSQYPCKEEMSAGAGESGRKTISLPEYDGTTTH | 155 |
| | | GVLVLDDGTQIGFTSGNGDPRYTNYRNNGHVEQKSAL | |
| | | YMRENNISNATVYHNNTNGTCGYCNTMTATFLPEGAT | |
| | | LTVVPPENAVANNSR | |
| | R2385-C | AIDYVKTYTGTSNDPKISPRYKGN | 156 |
| TDD7 | toxic domain | MGDRLPAFVDGGDTLGIFSRGGIERDLASGVAGPASSL | 157 |
| | | PKGTPGFNGLVKSHVEGHAAALMRQNGIPNAELYINR | |
| | | VPCGSGNGCAAMLPHMLPEGATLRVYGPNGYDRTFTG | |
| | | LPD | |
| | G33-N | MGDRLPAFVDGGDTLGIFSRGGIERDLASGVAG | 158 |
| | G33-C | PASSLPKGTPGFNGLVKSHVEGHAAALMRQNGIPNAEL | 159 |
| | | YINRVPCGSGNGCAAMLPHMLPEGATLRVYGPNGYDR | |
| | | TFTGLPD | |
| | G102-N | MGDRLPAFVDGGDTLGIFSRGGIERDLASGVAGPASSL | 160 |
| | | PKGTPGFNGLVKSHVEGHAAALMRQNGIPNAELYINR | |
| | | VPCGSGNGCAAMLPHMLPEGATLRVYG | |
| | G102-C | PNGYDRTFTGLPD | 161 |
| TDD8 | toxic domain | GGSAVVGAGVVATGAKAVTTGKSLSESQATLSVAQRL | 162 |
| | | LATIGEEGKTAGVLELDGELIPLVSGKSSLPNYAASGHV | |
| | | EGQAALIMRDRGATSGRLLIDNPSGICGYCKSQVATLLP | |
| | | ENATLQVGTPLGTVTPSSRWSASRTFTGNDRDPKPWPR | |
| | G2108-N | GGSAVVGAGVVATGAKAVTTGKSLSESQATLSVAQRL | 163 |
| | | LATIGEEGKTAGVLELDGELIPLVSG | |
| | G2108-C | KSSLPNYAASGHVEGQAALIMRDRGATSGRLLIDNPSGI | 164 |
| | | CGYCKSQVATLLPENATLQVGTPLGTVTPSSRWSASRT | |
| | | FTGNDRDPKPWPR | |
| | T2175-N | GGSAVVGAGVVATGAKAVTTGKSLSESQATLSVAQRL | 165 |
| | | LATIGEEGKTAGVLELDGELIPLVSGKSSLPNYAASGHV | |
| | | EGQAALIMRDRGATSGRLLIDNPSGICGYCKSQVATLLP | |
| | | ENATLQVGTPLGTVT | |
| | T2175-C | PSSRWSASRTFTGNDRDPKPWPR | 166 |
| TDD9 | toxic domain | DIILATLPIGKVGKLRFAPKVESAESMLRSLSQEGKTAG | 167 |
| | | VLDINGELIPLVSGTSSLKNYAASGHVEGQAALIMRER | |
| | | GVASARLIIDNPSGICGYCRSQVPTLLPAGATLEVTTPR | |
| | | GTVPPTARWSNGKTFVGNENDPKPWPR | |
| | G2112-N | DIILATLPIGKVGKLRFAPKVESAESMLRSLSQEGKTAG | 168 |
| | | VLDINGELIPLVSG | |
| | G2112-C | TSSLKNYAASGHVEGQAALIMRERGVASARLIIDNPSGI | 169 |
| | | CGYCRSQVPTLLPAGATLEVTTPRGTVPPTARWSNGKT | |
| | | FVGNENDPKPWPR | |
| | P2179-N | DIILATLPIGKVGKLRFAPKVESAESMLRSLSQEGKTAG | 170 |
| | | VLDINGELIPLVSGTSSLKNYAASGHVEGQAALIMRER | |
| | | GVASARLIIDNPSGICGYCRSQVPTLLPAGATLEVTTPR | |
| | | GTVP | |
| | P2179-C | PTARWSNGKTFVGNENDPKPWPR | 171 |
| TDD10 | toxic domain | PPVASGGLATEVPAYAGSRTAGTLVTPDGAEFPLISGW | 172 |
| | | HPPAASMPQGTPGMNIVTKSHVEAHAAAIMRNQGLSE | |
| | | ATLWINRAPCGGKPGCAAMLPRMVPSGSTLTINVVPNG | |
| | | SAGSIADTLIIRGIG | |
| | G1667-N | PPVASGGLATEVPAYAGSRTAGTLVTPDGAEFPLISG | 173 |
| | G1667-C | WHPPAASMPQGTPGMNIVTKSHVEAHAAAIMRNQGLS | 174 |
| | | EATLWINRAPCGGKPGCAAMLPRMVPSGSTLTINVVPN | |
| | | GSAGSIADTLIIRGIG | |
| | G1746-N | PPVASGGLATEVPAYAGSRTAGTLVTPDGAEFPLISGW | 175 |
| | | HPPAASMPQGTPGMNIVTKSHVEAHAAAIMRNQGLSE | |
| | | ATLWINRAPCGGKPGCAAMLPRMVPSGSTLTINVVPNG | |
| | | SAG | |
| | G1746-C | SIADTLIIRGIG | 176 |
| TDD11 | toxic domain | EIRAKYPTPEEAQLPPYDGDTTYALMYYTDEHGKSHV | 177 |
| | | VELSSGGADDEHSNYAAAGHTEGQAAVIMRQRKITSA | |
| | | VVVHNNTDGTCPFCVAHLPTLLPSGAELRVVPPRSAKA | |
| | | KKPGWIDVSKTFEGNARKPLDNKNKKST | |
| | G1430-N | EIRAKYPTPEEAQLPPYDGDTTYALMYYTDEHGKSHV | 178 |
| | | VELSSGG | |
| | G1430-C | ADDEHSNYAAAGHTEGQAAVIMRQRKITSAVVVHNNT | 179 |
| | | DGTCPFCVAHLPTLLPSGAELRVVPPRSAKAKKPGWID | |
| | | VSKTFEGNARKPLDNKNKKST | |
| | A1498-N | EIRAKYPTPEEAQLPPYDGDTTYALMYYTDEHGKSHV | 180 |
| | | VELSSGGADDEHSNYAAAGHTEGQAAVIMRQRKITSA | |
| | | VVVHNNTDGTCPFCVAHLPTLLPSGAELRVVPPRSAKA | |
| | A1498-C | KKPGWIDVSKTFEGNARKPLDNKNKKST | 181 |
| | G1502-N | EIRAKYPTPEEAQLPPYDGDTTYALMYYTDEHGKSHV | 182 |
| | | VELSSGGADDEHSNYAAAGHTEGQAAVIMRQRKITSA | |
| | | VVVHNNTDGTCPFCVAHLPTLLPSGAELRVVPPRSAKA | |
| | | KKPG | |
| | G1502-C | WIDVSKTFEGNARKPLDNKNKKST | 183 |
| TDD12 | toxic domain | AALLREAYPSMEGATLPPFDGKTTIGLMFYTDASGQYQ | 184 |
| | | VKKLFSGEKVLSNYDATGHVEGKAALIMRNEKITEAV | |
| | | VMHNHPSGTCNYCDKQVETLLPKNATLRVIPPENAKAP | |
| | | TSYWNDQPTTYRGDGKDPKAPSKK | |
| | G1421-N | AALLREAYPSMEGATLPPFDGKTTIGLMFYTDASGQYQ | 185 |
| | | VKKLFSG | |
| | G1421-C | EKVLSNYDATGHVEGKAALIMRNEKITEAVVMHNHPS | 186 |
| | | GTCNYCDKQVETLLPKNATLRVIPPENAKAPTSYWND | |
| | | QPTTYRGDGKDPKAPSKK | |
| | A1488-N | AALLREAYPSMEGATLPPFDGKTTIGLMFYTDASGQYQ | 187 |
| | | VKKLFSGEKVLSNYDATGHVEGKAALIMRNEKITEAV | |
| | | VMHNHPSGTCNYCDKQVETLLPKNATLRVIPPENAKA | |
| | A1488-C | PTSYWNDQPTTYRGDGKDPKAPSKK | 188 |
| TDD13 | toxic domain | ALLREQFPSMDAVTLPPFDGKTTIGYMFYTDANGQYH | 189 |
| | | VRKLYSGGKVLSNYDSSGHVEGMAALIMRKGRITEAV | |
| | | VMHNHPSGTCHYCNGQVETLLPKNAKLKVIPPANAKA | |
| | | PTKYWYDQPVDYLGNSNDPKPPS | |
| | G1411-N | ALLREQFPSMDAVTLPPFDGKTTIGYMFYTDANGQYH | 190 |
| | | VRKLYSGG | |
| | G1411-C | KVLSNYDSSGHVEGMAALIMRKGRITEAVVMHNHPSG | 191 |
| | | TCHYCNGQVETLLPKNAKLKVIPPANAKAPTKYWYDQ | |
| | | PVDYLGNSNDPKPPS | |
| | A1477-N | ALLREQFPSMDAVTLPPFDGKTTIGYMFYTDANGQYH | 192 |
| | | VRKLYSGGKVLSNYDSSGHVEGMAALIMRKGRITEAV | |
| | | VMHNHPSGTCHYCNGQVETLLPKNAKLKVIPPANAKA | |
| | A1477-C | PTKYWYDQPVDYLGNSNDPKPPS | 193 |
| TDD14 | toxic domain | GSSGKNVRMPRDYASELPEYDGKTTHGVLVTNEGKVI | 194 |
| | | QLRSGGKEEPYTGYKAVSASHVEGKAAIWIRENGSSGG | |
| | | TVYHNNTTGTCGYCNSQVKALLPEGVELKIVPPTNAVA | |
| | | KNAQARAVPTINVGNGTQPGRKQK | |
| | G43-N | GSSGKNVRMPRDYASELPEYDGKTTHGVLVTNEGKVI | 195 |
| | | QLRSGG | |
| | G43-C | KEEPYTGYKAVSASHVEGKAAIWIRENGSSGGTVYHN | 196 |
| | | NTTGTCGYCNSQVKALLPEGVELKIVPPTNAVAKNAQ | |
| | | ARAVPTINVGNGTQPGRKQK | |
| | A118-N | GSSGKNVRMPRDYASELPEYDGKTTHGVLVTNEGKVI | 197 |
| | | QLRSGGKEEPYTGYKAVSASHVEGKAAIWIRENGSSGG | |
| | | TVYHNNTTGTCGYCNSQVKALLPEGVELKIVPPTNAVA | |
| | | KNAQA | |
| | A118-C | RAVPTINVGNGTQPGRKQK | 198 |
| TDD15 | toxic domain | GSSGKNVRLPRDYASELPEYDGKTTYGVLVTNEGKVIQ | 199 |
| | | LRSGGKEVPYSGYKAVSASHVEGKAAIWIRENASSGGT | |
| | | VYHNNTTGTCGYCNSQVKALLPEGVELKIVPPANAVA | |
| | | RNSQAKAIPTINVGNATQPGRKP | |
| | G315-N | GSSGKNVRLPRDYASELPEYDGKTTYGVLVTNEGKVIQ | 200 |
| | | LRSGG | |
| | G315-C | KEVPYSGYKAVSASHVEGKAAIWIRENASSGGTVYHN | 201 |
| | | NTTGTCGYCNSQVKALLPEGVELKIVPPANAVARNSQA | |
| | | KAIPTINVGNATQPGRKP | |
| | A390-N | GSSGKNVRLPRDYASELPEYDGKTTYGVLVTNEGKVIQ | 202 |
| | | LRSGGKEVPYSGYKAVSASHVEGKAAIWIRENASSGGT | |
| | | VYHNNTTGTCGYCNSQVKALLPEGVELKIVPPANAVA | |
| | | RNSQA | |
| | A390-C | KAIPTINVGNATQPGRKP | 203 |
| TDD16 | toxic domain | PDPPPPPTPMGNTLPGWDGGKTQGWFVYPDGTERHLIS | 204 |
| | | GYDGPSKFTQGIPGMNGNIKSHVEAHAAALMRQYELS | |
| | | KATLYINRVPCPGVRGCDALLARMLPEGVQLEIIGPNGF | |
| | | KKTYTGLPDPKLKPKGCS | |
| | G1264-N | PDPPPPPTPMGNTLPGWDGGKTQGWFVYPDGTERHLIS | 205 |
| | | GYDG | |
| | G1264-C | PSKFTQGIPGMNGNIKSHVEAHAAALMRQYELSKATLY | 206 |
| | | INRVPCPGVRGCDALLARMLPEGVQLEIIGPNGFKKTYT | |
| | | GLPDPKLKPKGCS | |
| | G1342-C | PDPPPPPTPMGNTLPGWDGGKTQGWFVYPDGTERHLIS | 207 |
| | | GYDGPSKFTQGIPGMNGNIKSHVEAHAAALMRQYELS | |
| | | KATLYINRVPCPGVRGCDALLARMLPEGVQLEIIGPNGF | |
| | | KKTYTG | |
| | G1342-N | LPDPKLKPKGCS | 208 |
| TDD17 | toxic domain | GAATVFGAGRGLGALEEATTAAGIARGAPSLPVYTGG | 209 |
| | | KTTGVLRTATGDMPLVSGYKGPSASMPRGTPGMNGRI | |
| | | KSHVEAHAAAVMRERGIKDATLHINQVPCSSATGCGA | |
| | | MLPRMLPEGAQLRVLGPDGYDQVFIGLPD | |
| | G2087-N | GAATVFGAGRGLGALEEATTAAGIARGAPSLPVYTGG | 210 |
| | | KTTGVLRTATGDMPLVSGYKG | |
| | G2087-C | PSASMPRGTPGMNGRIKSHVEAHAAAVMRERGIKDAT | 211 |
| | | LHINQVPCSSATGCGAMLPRMLPEGAQLRVLGPDGYD | |
| | | QVFIGLPD | |
| | G2156-N | GAATVFGAGRGLGALEEATTAAGIARGAPSLPVYTGG | 212 |
| | | KTTGVLRTATGDMPLVSGYKGPSASMPRGTPGMNGRI | |
| | | KSHVEAHAAAVMRERGIKDATLHINQVPCSSATGCGA | |
| | | MLPRMLPEGAQLRVLG | |
| | G2156-C | PDGYDQVFIGLPD | 213 |
| TDD18 | toxic domain | TNIIDNRPKLPDYDGKTTHGILVTPNSEHIPFSSGNPNPN | 214 |
| | | YKNYIPASHVEGKSAIYMRENGITSGTIYYNNTDGTCPY | |
| | | CDKMLSTLLEEGSVLEVIPPINAKAPKPSWVDKPKTYIG | |
| | | NNKVPKPNK | |
| | G181-N | TNIIDNRPKLPDYDGKTTHGILVTPNSEHIPFSSG | 215 |
| | G181-C | NPNPNYKNYIPASHVEGKSAIYMRENGITSGTIYYNNTD | 216 |
| | | GTCPYCDKMLSTLLEEGSVLEVIPPINAKAPKPSWVDKP | |
| | | KTYIGNNKVPKPNK | |
| | A250-N | TNIIDNRPKLPDYDGKTTHGILVTPNSEHIPFSSGNPNPN | 217 |
| | | YKNYIPASHVEGKSAIYMRENGITSGTIYYNNTDGTCPY | |
| | | CDKMLSTLLEEGSVLEVIPPINAKA | |
| | A250-C | PKPSWVDKPKTYIGNNKVPKPNK | 218 |
| TDD19 | toxic domain | AGCPGDALPPYGTKGSKTTGILDTGNESILLESGENGPG | 219 |
| | | MMVPRDTPGMSGAMPNRAHVEGHTAAIMRNENIRLA | |
| | | DLYINRMPCSGAYGCMVNLPHMLPEGSILRIHVRAKLS | |
| | | DPWTTLPPFVGISDTLWPPSGLNPKIVLP | |
| | G234-N | AGCPGDALPPYGTKGSKTTGILDTGNESILLESGENG | 220 |
| | G234-C | PGMMVPRDTPGMSGAMPNRAHVEGHTAAIMRNENIRL | 221 |
| | | ADLYINRMPCSGAYGCMVNLPHMLPEGSILRIHVRAKL | |
| | | SDPWTTLPPFVGISDTLWPPSGLNPKIVLP | |
| | G321-N | ISDTLWPPSGLNPKIVLP | 222 |
| | G321-C | AGCPGDALPPYGTKGSKTTGILDTGNESILLESGENGPG | 223 |
| | | MMVPRDTPGMSGAMPNRAHVEGHTAAIMRNENIRLA | |
| | | DLYINRMPCSGAYGCMVNLPHMLPEGSILRIHVRAKLS | |
| | | DPWTTLPPFVG | |

SEQ: SEQ ID NO:

### TDD Base Editing Activity at the CCR5 Locus

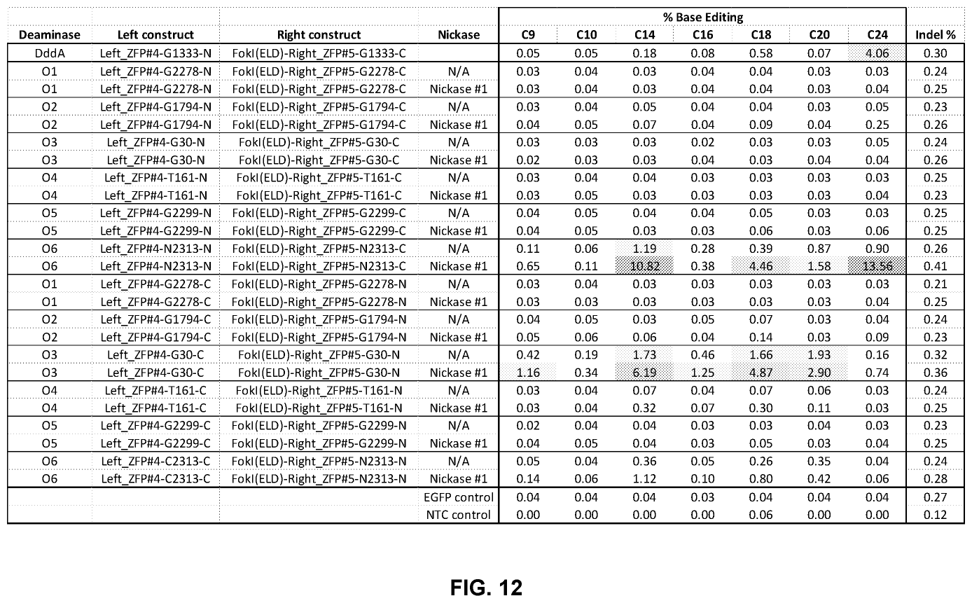

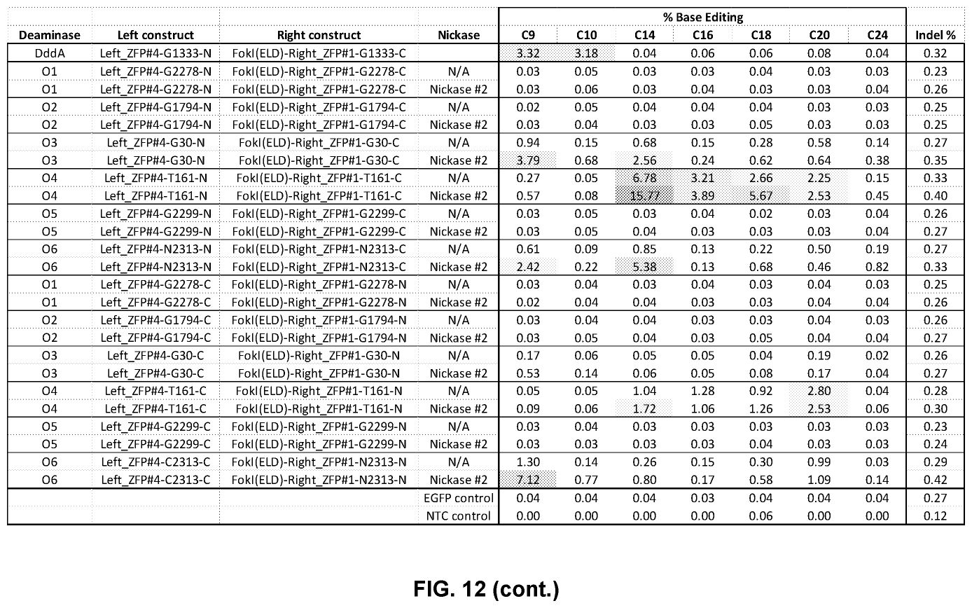

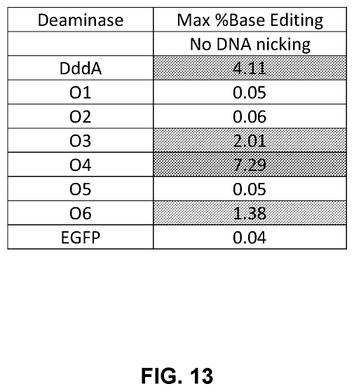

FIG. 12 shows the base editing frequency of TDD1-TDD6 (select splits) at C9, C10, C14, C16, C18, C20, and C24 of target sequence CCR5, with two different pairs of ZFP DNA binding domains (see FIG. 10). Two orientations of each split enzyme were tested (i.e., with the N- and C-terminal halves linked to different members of the ZFP pair for each orientation). In experiments where the base editing system included a nickase, a ZFP-FokI nickase or a CRISPR/Cas9 nickase was used.

FIG. 13 shows a comparison of the highest frequency of editing for each deaminase for any C in the base editing window (based on data shown in FIG. 12 as well as additional replicates). At least three of the TDDs (TDD3, TDD4, and TDD6) demonstrated detectable base editing activity (>0.25% base editing), with TDD4 showing higher maximum activity than DddA.

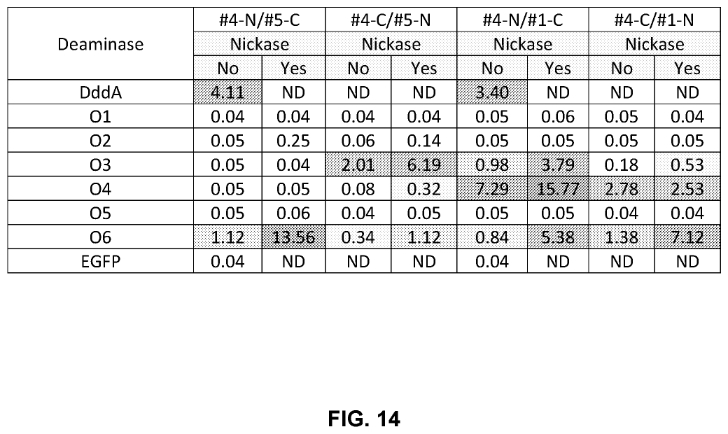

FIG. 14 provides a more detailed analysis of the TDD base editing activity (based on data shown in FIG. 12 as well as additional replicates), showing the highest frequency of editing for any C in the base editing window for the two binding orientations of each TDD to the two different ZFP pairs, with or without nickase activity. Base editing for certain TDDs appeared to be sensitive to the ZFP pair (e.g., TDD4) or the binding orientation (e.g., TDD3). TDD6 seemed to have detectable activity (>0.25% base editing) for every condition under which it was tested, albeit with a binding orientation dependency at least in the context of ZFP#4 and ZFP#5. For each TDD, in some cases, nicking appeared to improve base editing activity (see also FIG. 12).

### TDD Base Editing Activity at the CIITA Locus

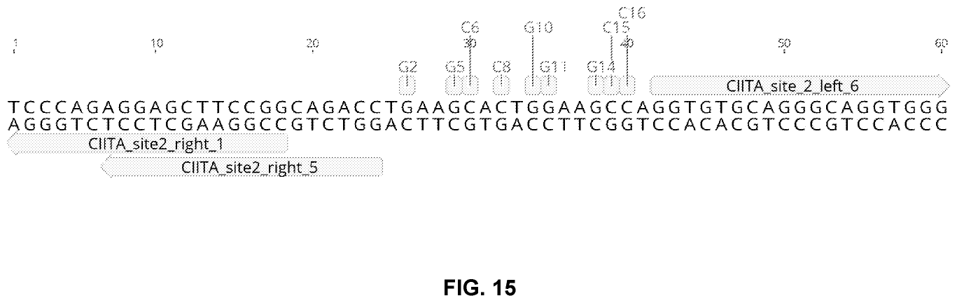

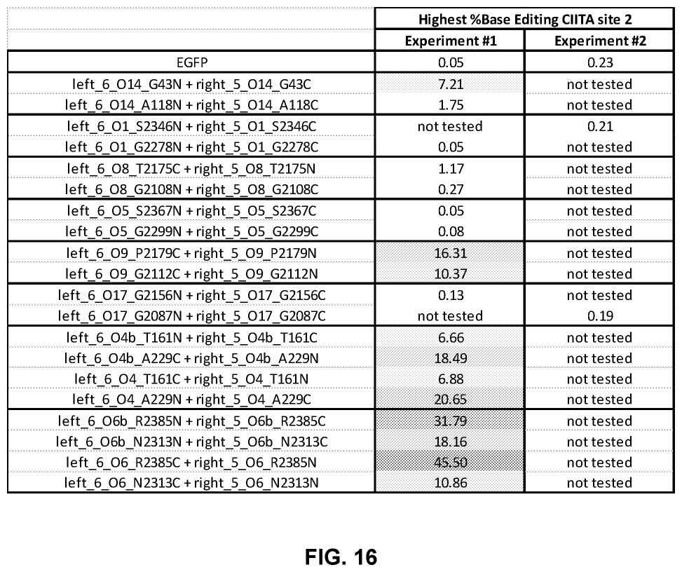

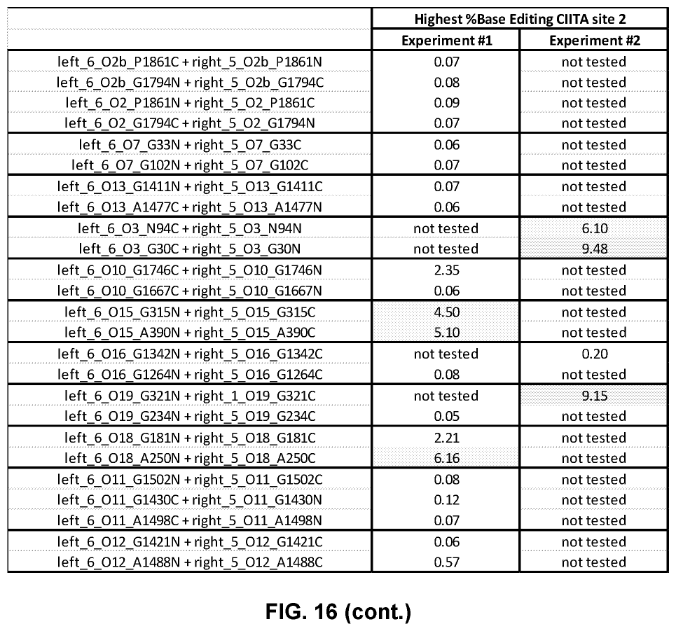

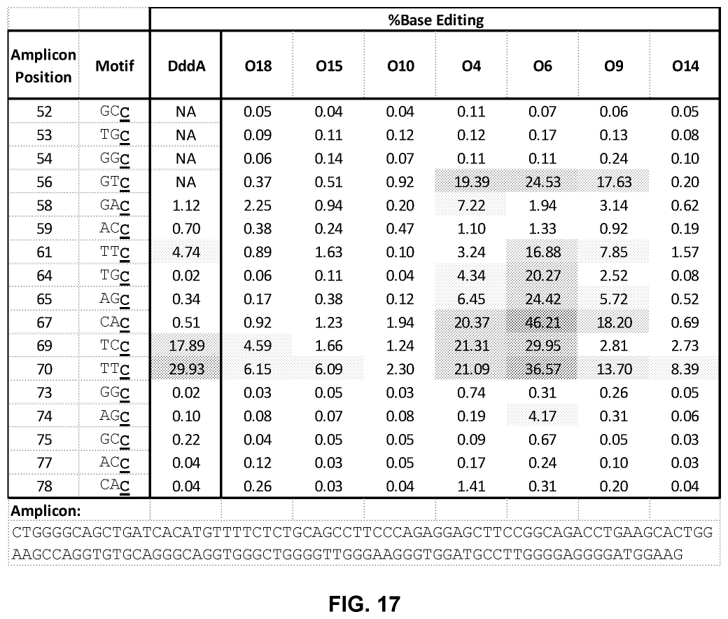

Select TDD split enzymes were tested for base editing at the nucleotides labeled G2, G5, C6, C8, G10, G11, G14, C15 and C16 in target sequence CIITA with the ZFP binding domains shown (“CIITA_site_2_right_1,” “CIITA_site_2_right_5,” and “CIITA_site_2_left_6”) (FIG. 15). FIG. 16 shows a comparison of the highest frequency of editing for each fusion protein pair for any C in the base editing window. TDD3, TDD4, and TDD6, which were active at the CCR5 locus, also demonstrated detectable base editing activity (>0.25% base editing) at the CIITA locus. Eight additional TDDs (TDD8, TDD9, TDD10, TDD12, TDD14, TDD15, TDD18, and TDD19) demonstrated detectable editing as well. Base editing activity appeared to be sensitive to the TDD split position, and in some cases to the variant of the toxic domain used (e.g., TDD4). TDD4 appeared to have significant activity in every condition under which it was tested. Some TDDs also provide an increased targeting density (FIG. 17) with stronger activity at TC and AC sites (compared to DddA; see, e,g., TDD6) as well as activity at GC and CC sites (e.g., TDD6).

### Effect of Different Linkers on TDD Base Editing Activity at the CIITA Locus

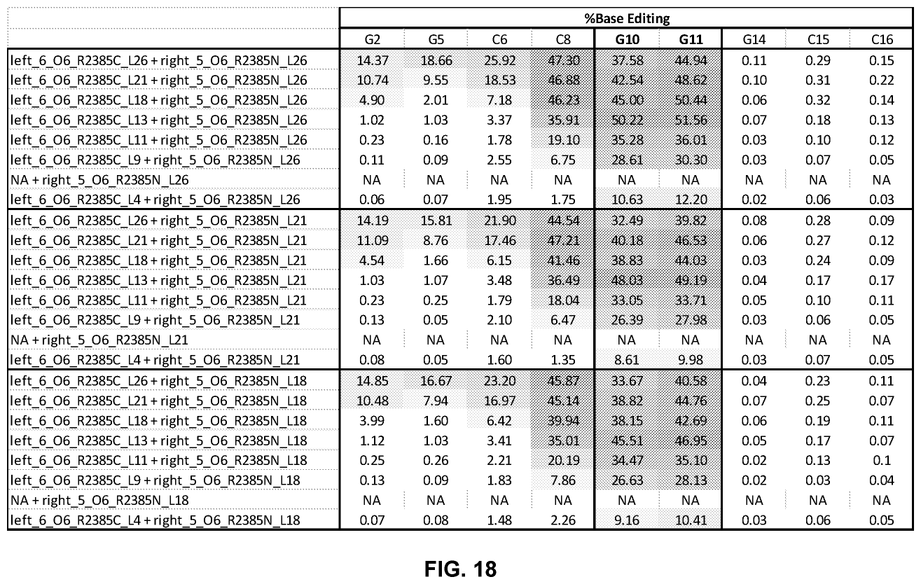

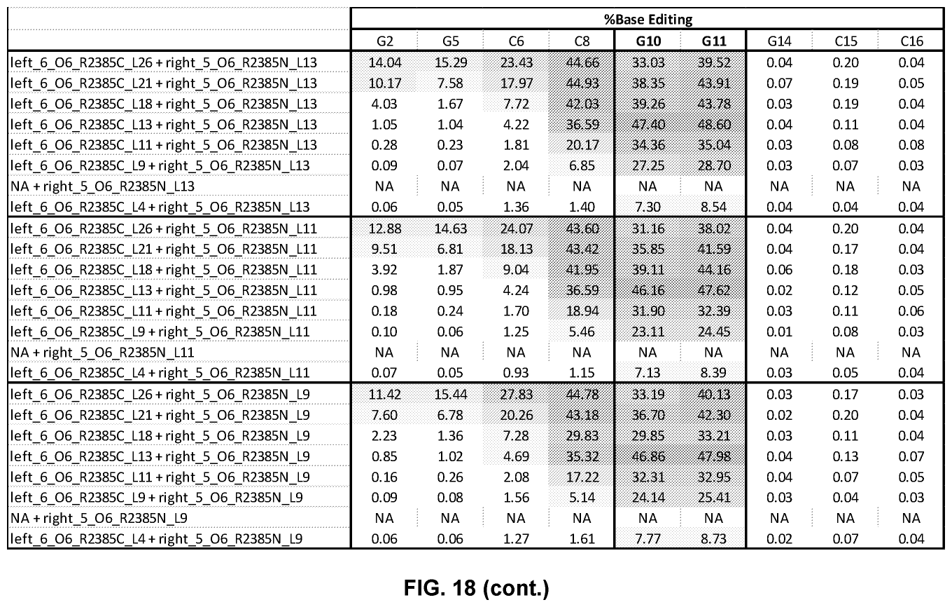

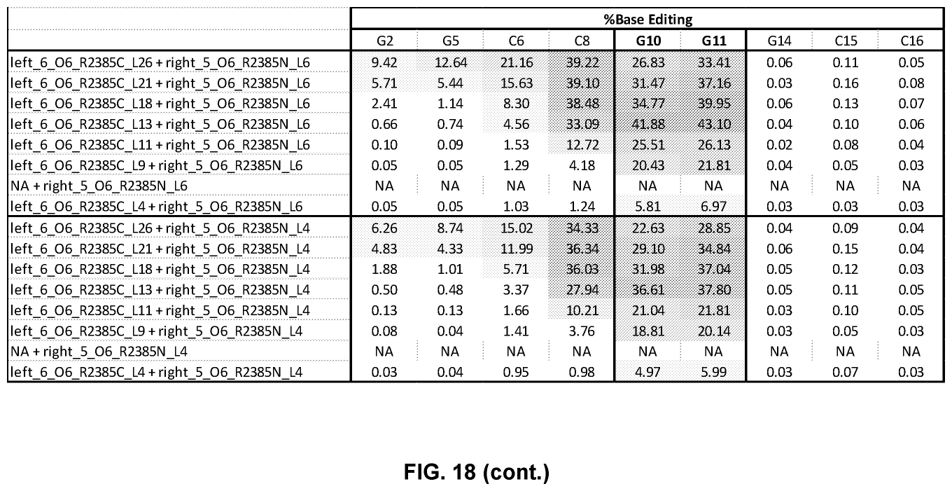

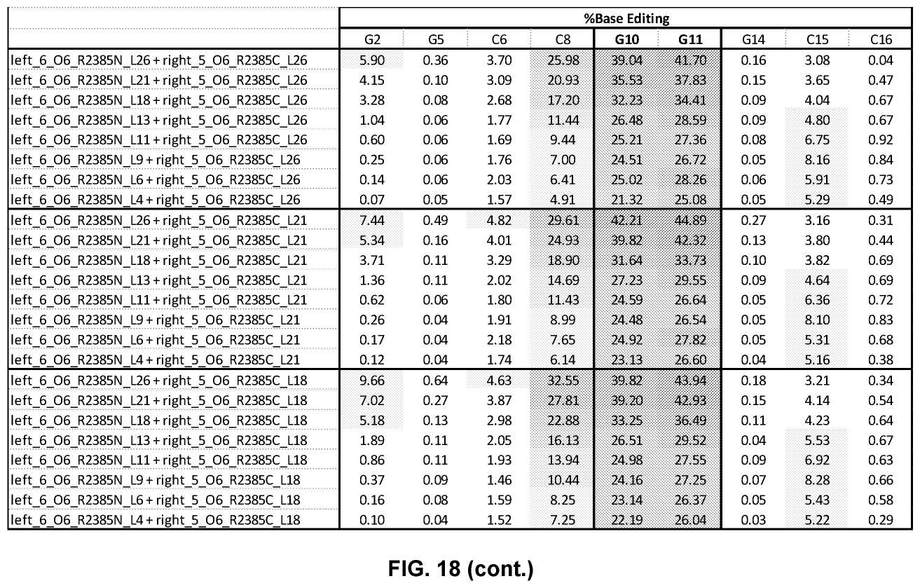

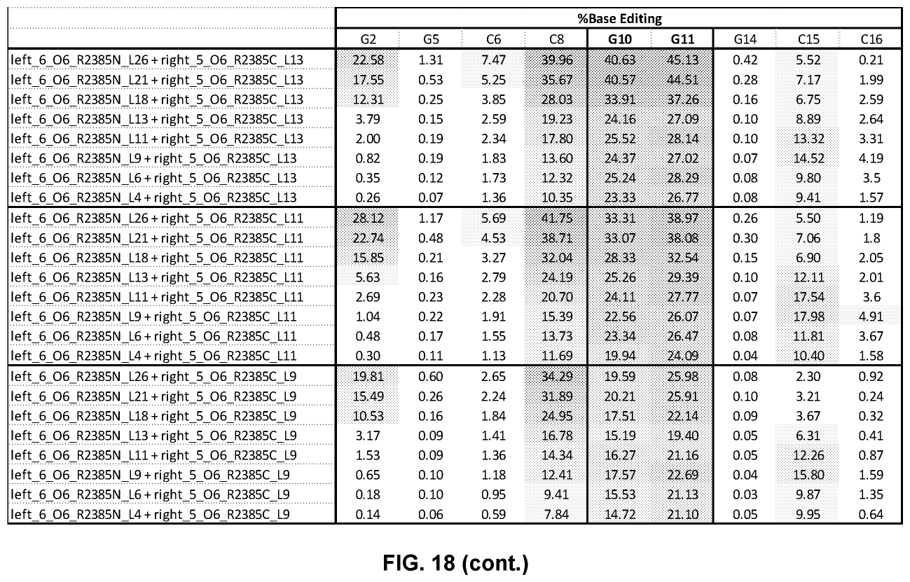

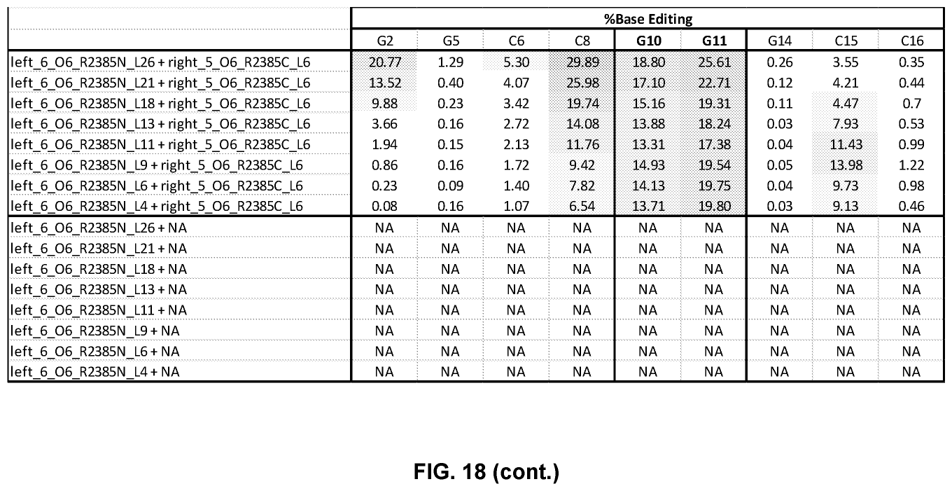

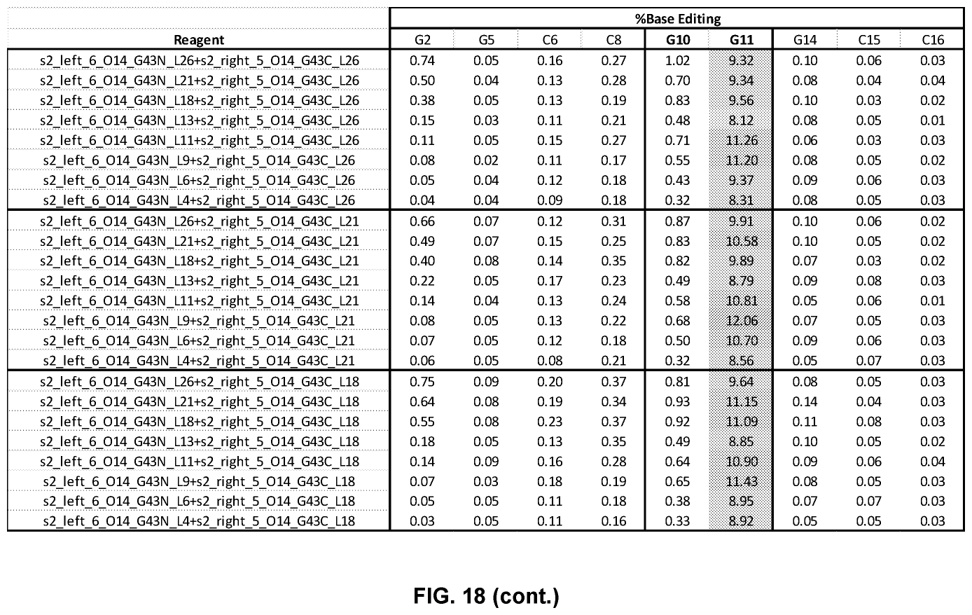

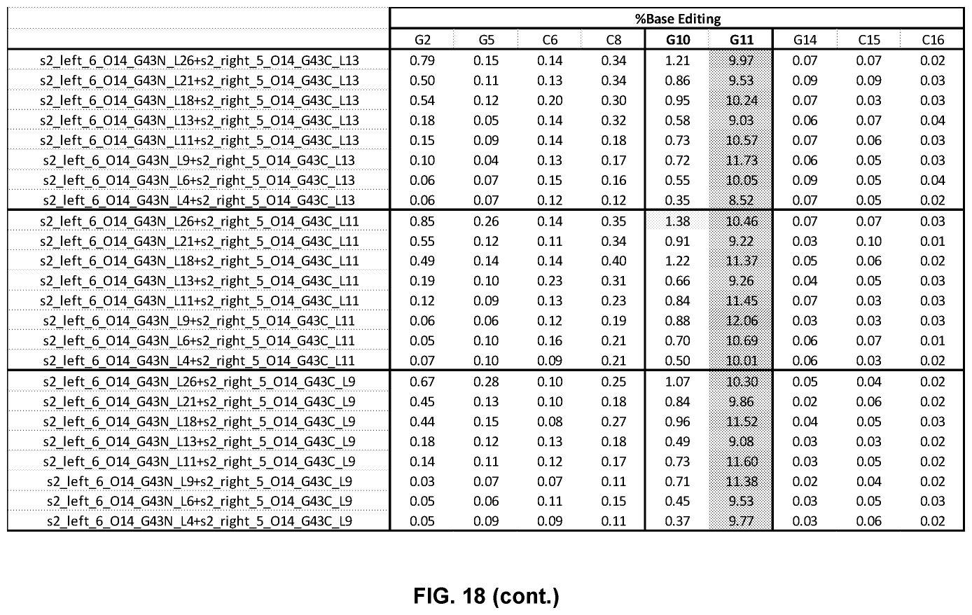

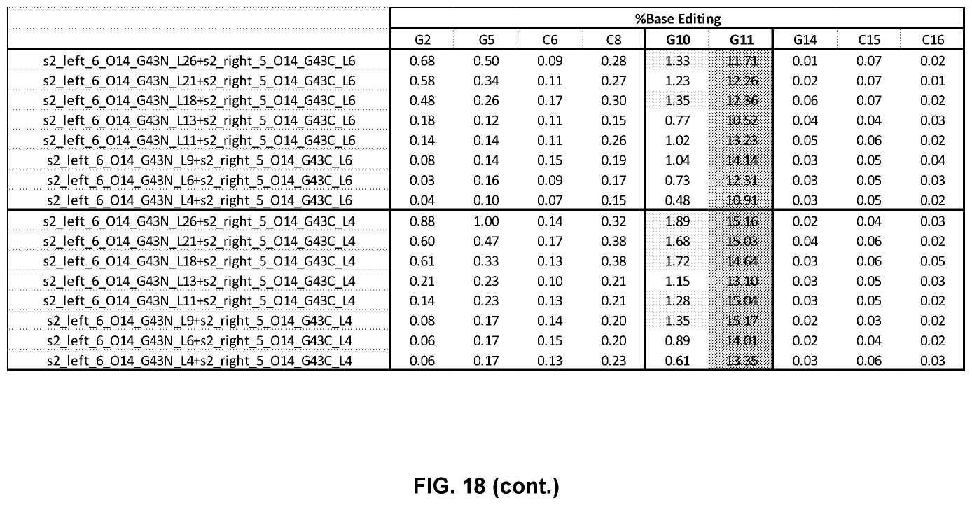

To assess whether base editing activity is affected by different linkers between the deaminase and ZFP domains, the editing frequency of TDD6 at the CIITA locus was assessed with linkers L26, L21, L18, L13, L11, L9, L6, and L4. As shown in FIG. 18, different linker lengths were able to alter the base editing profile within the base editing window. For example, shortening the linker connecting the left ZFP to either the N- or C-terminal TDD split appeared to narrow the activity window. Such alterations may increase base editor precision and specificity. In some cases, the effects of linker length appeared sensitive to the binding orientation of the TDD splits to the ZFP pair or to the TDD (e.g., L4 performance with TDD14).

### Example 7: Targeting Inhibitor TDDI to TDD

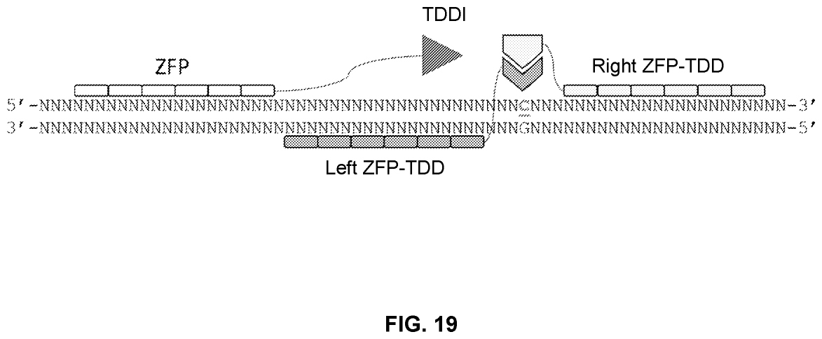

TDD enzymes may be inactivated by TDDIs. For example, the natural DddA enzyme can be inactivated by the Dddl inhibitor. A ZFP or TALE linked TDDI can be targeted to a potential TDD-derived cytosine base editor site, preventing that site from being edited (FIG. 19). The TDDI inhibitor may be linked to the ZFP using a dimerization domain potentiated by a small molecule, thus putting the editing activity under the control of the small molecule.

By designing the targeted TDDI construct to be allele specific, editing can selectively be targeted to certain alleles, e.g., to knock out a detrimental mutant by editing in a stop codon only if the mutation is present. For example, JAK2 V617F can be knocked out by editing in a stop codon only if the V617F mutation is present.

This TDDI approach may also be used to reduce editing at off-target sites, particularly where it cannot be eliminated by other means.

It is also contemplated that other cytidine deaminases and their inhibitors can be used in place of a TDD and TDDI.

1-54. (canceled)

55. A system for changing a cytosine to a thymine in the genome of a cell, comprising a first fusion protein and a second fusion protein, or first and second expression constructs for expressing the first and second fusion proteins, respectively, wherein
a) the first fusion protein comprises:
i) a first zinc finger protein (ZFP) domain that binds to a first sequence in a target genomic region in the cell, and
ii) a first portion of a cytidine deaminase polypeptide, wherein the cytidine deaminase is a toxin-derived deaminase (TDD) comprising SEQ ID NO: 49, 81, 92, 95, 98, 101, 104, 107, 134, 143, 152, 157, 162, 167, 172, 177, 184, 189, 194, 199, 204, 209, 214, or 219, or an amino acid sequence at least 90% identical to;
b) the second fusion protein comprises:
i) a second ZFP domain that binds to a second sequence in the target genomic region, and
ii) a second portion of the cytidine deaminase polypeptide;
c) the first and second portions lack cytidine deaminase activity on their own; and
d) binding of the first fusion protein and the second fusion protein to the target genomic region results in dimerization of the first and second portions, wherein the dimerized portions form an active cytidine deaminase capable of changing a cytosine to a thymine in the target genomic region.

56. The system of claim 55, comprising more than one pair of the first and second fusion proteins, wherein each pair of the fusion proteins binds to a different target genomic region.

57. The system of claim 55, further comprising a nickase that creates a single-stranded DNA break on the unedited or edited strand, wherein the DNA break is no more than about 500 bps from the cytosine to be edited, wherein the nickase is a ZFP-based nickase, a TALE-based nickase, or a CRISPR-based nickase.

58. The system of claim 55, further comprising a third fusion protein or a third expression construct for expressing the third fusion protein in the cell, wherein
I) e) the third fusion protein comprises
i) a ZFP domain that binds to a third sequence in the target genomic region, and
ii) an inhibitory domain for the cytidine deaminase; and
f) binding of the third fusion protein to the target genomic region results in the inhibitory domain binding to, and thereby inhibition of the cytidine deaminase activity of, the dimerized cytidine deaminase portions;
II) the system further comprises a fourth fusion protein or a fourth expression construct for expressing the fourth fusion protein in the cell, wherein
e) the third fusion protein comprises
i) a ZFP domain that binds to a third sequence in the target genomic region, and
ii) a first dimerization domain; and
f) the fourth fusion protein comprises
i) an inhibitory domain for the cytidine deaminase, and
ii) a second dimerization domain capable of partnering with the first dimerization domain in the presence of a dimerization-inducing agent; and
g) binding of the third fusion protein to the target genomic region, and dimerization of the first and second dimerization domains, result in the inhibitory domain binding to, and thereby inhibition of the cytidine deaminase activity of, the dimerized cytidine deaminase portions
III) the system further comprises a fourth fusion protein or a fourth expression construct for expressing the fourth fusion protein in the cell, wherein
e) the third fusion protein comprises
i) a ZFP domain that binds to a third sequence in the target genomic region, and
ii) a first dimerization domain; and
f) the fourth fusion protein comprises
i) an inhibitory domain for the cytidine deaminase, and
ii) a second dimerization domain capable of partnering with the first dimerization domain in the absence of a dimerization-inhibiting agent; and
g) binding of the third fusion protein to the target genomic region, and dimerization of the first and second dimerization domains, result in the inhibitory domain binding to, and thereby inhibition of the cytidine deaminase activity of, the dimerized cytidine deaminase portions.

59. The system of claim 55, wherein the expression constructs are on the same or separate viral vectors, wherein the viral vectors are adeno-associated viral (AAV) vectors, adenoviral vectors, or lentiviral vectors.

60. The system of claim 55, wherein the cytidine deaminase is a TDD that comprises the amino acid sequence of any one of SEQ ID NOs: 72, 86-91, and 117-129 or the toxic domain of a TDD comprising said amino acid sequence.

61. The system of claim 60, wherein the cytidine deaminase is a TDD that comprises the amino acid sequence of SEQ ID NO: 72 or the toxic domain of a TDD comprising said amino acid sequence, wherein the TDD has a mutation at one or more residues selected from Y1307, T1311, S1331, V1346, H1366, N1367, N1368, P1369, E1370, G1371, T1372, F1375, V1392, P1394, P1395, 11399, P1400, V1401, K1402, A1405, and T1406, wherein the residues are numbered with respect to SEQ ID NO: 72.

62. The system of claim 55, wherein the first and second cytidine deaminase portions comprise:
amino acids 1264-1333 and 1334-1427 of SEQ ID NO: 72, respectively;
amino acids 1264-1397 and 1398-1427 of SEQ ID NO: 72, respectively;
amino acids 1264-1404 and 1405-1427 of SEQ ID NO: 72, respectively;
amino acids 1264-1407 and 1408-1427 of SEQ ID NO: 72, respectively;
amino acids 1290-1333 and 1334-1427 of SEQ ID NO: 72, respectively;
amino acids 1290-1397 and 1398-1427 of SEQ ID NO: 72, respectively;
amino acids 1290-1404 and 1405-1427 of SEQ ID NO: 72, respectively;
amino acids 1290-1407 and 1408-1427 of SEQ ID NO: 72, respectively;
SEQ ID NOs: 82 and 83, respectively;
SEQ ID NOs: 84 and 85, respectively;
SEQ ID NOs: 18 and 19, respectively;
SEQ ID NOs: 51 and 52, respectively; or
SEQ ID NOs: 53 and 54, respectively;
or vice-versa.

63. The system of claim 55, wherein
the first and second cytidine deaminase portions respectively comprise SEQ ID NOs: 93 and 94, SEQ ID NOs: 96 and 97, SEQ ID NOs: 99 and 100, SEQ ID NOs: 102 and 103, SEQ ID NOs: 105 and 106, SEQ ID NOs: 108 and 109, SEQ ID NOs: 130 and 131, SEQ ID NOs: 132 and 133, SEQ ID NOs: 135 and 136, SEQ ID NOs: 137 and 138, SEQ ID NOs: 139 and 140, SEQ ID NOs: 141 and 142, SEQ ID NOs: 144 and 145, SEQ ID NOs: 146 and 147, SEQ ID NOs: 148 and 149, SEQ ID NOs: 150 and 151, SEQ ID NOs: 153 and 154, SEQ ID NOs: 155 and 156, SEQ ID NOs: 158 and 159, SEQ ID NOs: 160 and 161, SEQ ID NOs: 163 and 164, SEQ ID NOs: 165 and 166, SEQ ID NOs: 168 and 169, SEQ ID NOs: 170 and 171, SEQ ID NOs: 173 and 174, SEQ ID NOs: 175 and 176, SEQ ID NOs: 178 and 179, SEQ ID NOs: 180 and 181, SEQ ID NOs: 182 and 183, SEQ ID NOs: 185 and 186, SEQ ID NOs: 187 and 188, SEQ ID NOs: 190 and 191, SEQ ID NOs: 192 and 193, SEQ ID NOs: 195 and 196, SEQ ID NOs: 197 and 198, SEQ ID NOs: 200 and 201, SEQ ID NOs: 202 and 203, SEQ ID NOs: 205 and 206, SEQ ID NOs: 207 and 208, SEQ ID NOs: 210 and 211, SEQ ID NOs: 212 and 213, SEQ ID NOs: 215 and 216, SEQ ID NOs: 217 and 218, SEQ ID NOs: 220 and 221, or SEQ ID NOs: 222 and 223;
or vice-versa.

64. A fusion protein comprising
I) i) a zinc finger protein (ZFP) domain that binds to a gene, and ii) a fragment of a cytidine deaminase polypeptide, wherein the cytidine deaminase is a toxin-derived deaminase (TDD) comprising SEQ ID NO: 49, 81, 92, 95, 98, 101, 104, 107, 134, 143, 152, 157, 162, 167, 172, 177, 184, 189, 194, 199, 204, 209, 214, or 219, or an amino acid sequence at least 90% identical to, wherein the ZFP domain and the cytidine deaminase fragment are linked by a peptide linker; or
II) i) a zinc finger protein (ZFP) domain that binds to a gene, and ii) a cytidine deaminase inhibitory domain, wherein the cytidine deaminase is a toxin-derived deaminase (TDD) comprising SEQ ID NO: 49, 81, 92, 95, 98, 101, 104, 107, 134, 143, 152, 157, 162, 167, 172, 177, 184, 189, 194, 199, 204, 209, 214, or 219, or an amino acid sequence at least 90% identical to, wherein the ZFP domain and the inhibitory domain are linked by a peptide linker.

65. The fusion protein of claim 64, wherein the linker comprises any one of SEQ ID NOs: 15-17 and 110-116.

66. A pair of fusion proteins comprising
I) a) a first fusion protein that comprises i) a zinc finger protein (ZFP) domain that binds to a gene, and ii) a first dimerization domain, and
b) a second fusion protein that comprises i) a cytidine deaminase inhibitory domain, wherein the cytidine deaminase is a toxin-derived deaminase (TDD) comprising SEQ ID NO: 49, 81, 92, 95, 98, 101, 104, 107, 134, 143, 152, 157, 162, 167, 172, 177, 184, 189, 194, 199, 204, 209, 214, or 219, or an amino acid sequence at least 90% identical to, and ii) a second dimerization domain,
wherein the first and second dimerization domains can dimerize in the presence of a dimerization-inducing agent, or
II) a) a first fusion protein that comprises i) a zinc finger protein (ZFP) domain that binds to a gene, and ii) a first dimerization domain, and
b) a second fusion protein that comprises i) a cytidine deaminase inhibitory domain, wherein the cytidine deaminase is a toxin-derived deaminase (TDD) comprising SEQ ID NO: 49, 81, 92, 95, 98, 101, 104, 107, 134, 143, 152, 157, 162, 167, 172, 177, 184, 189, 194, 199, 204, 209, 214, or 219, or an amino acid sequence at least 90% identical to, and ii) a second dimerization domain,
wherein the first and second dimerization domains can dimerize in the absence of a dimerization-inhibiting agent.

67. An isolated nucleic acid molecule encoding the fusion protein of claim 64.

68. An expression construct comprising the nucleic acid molecule of claim 67.

69. A viral vector comprising the expression construct of claim 68, wherein the viral vector is an adeno-associated viral vector, an adenoviral vector, or a lentiviral vector.

70. A cell comprising the system of claim 55.

71. A method of changing a cytosine to a thymine in a target genomic region in a cell, comprising delivering the system of claim 55 to the cell.

72. A genetically engineered cell obtained by the method of claim 71.

73. A method of treating a human patient in need thereof, comprising delivering the genetically engineered cell of claim 72 to the patient, wherein the cell is a human cell.

74. The method of claim 73, wherein the patient has cancer, an autoimmune disorder, an autosomal dominant disease, a mitochondrial disorder, sickle cell disease, hemophilia, cystic fibrosis, phenylketonuria, Tay-Sachs, prion disease, color blindness, a lysosomal storage disease, Friedreich's ataxia, or prostate cancer.


SEQUENCE LISTING (245 sequences)

SEQ ID NO: 1 - protein, 42 aa, Artificial Sequence
  source: /note=“Description of Artificial Sequence Synthetic polypeptide”
MDYKDHDGDYKDHDIDYKDDDDKMAPKKKRKVGIHGVPAAMA

SEQ ID NO: 2 - protein, 172 aa, Artificial Sequence
  source: /note=“Description of Artificial Sequence Synthetic polypeptide”
ERPFQCRICMRNFSRSDSLSVHIRTHTGEKPFACDICGRKFAQSGSLTRHTKIHTGSQKPFQCRICMRNFSTSGHLSRHIRTHTGEKPFACDICGRKFAQSGDLTRHTKIHTHPRAPIPKPFQCRICMRNFSMVCCRTLHIRTHTGEKPFACDICGRKFARSANLTRHTKIH

SEQ ID NO: 3 - protein, 139 aa, Artificial Sequence
  source: /note=“Description of Artificial Sequence Synthetic polypeptide”
ERPFQCRICMRNFSRPYTLRLHIRTHTGEKPFACDICGRKFARKYYLAKHTKIHTGSQKPFQCRICMRNFSDDWNLSQHIRTHTGEKPFACDICGRKFARSANLTRHTKIHTGEKPFQCRICMRKFAQSAHRITHTKIH

SEQ ID NO: 4 - protein, 168 aa, Artificial Sequence
  source: /note=“Description of Artificial Sequence Synthetic polypeptide”
ERPFQCRICMRNFSQSGALARHIRTHTGEKPFACDICGRKFALKQHLTRHTKIHTGSQKPFQCRICMRNFSQSGDLTRHIRTHTGEKPFACDICGRKFAQSSDLRRHTKIHTGSQKPFQCRICMRNFSQSAHRKNHIRTHTGEKPFACDICGRKFARSAVRKNHTKIH

SEQ ID NO: 5 - protein, 172 aa, Artificial Sequence
  source: /note=“Description of Artificial Sequence Synthetic polypeptide”
ERPFQCRICMRNFSQSGALARHIRTHTGEKPFACDICGRKFALKQHLTRHTKIHTGSQKPFQCRICMRNFSQSGDLTRHIRTHTGEKPFACDICGRKFAQSSDLRRHTKIHTHPRAPIPKPFQCRICMRNFSRSANLARHIRTHTGEKPFACDICGRKFATNQNRITHTKIH

SEQ ID NO: 6 - protein, 168 aa, Artificial Sequence
  source: /note=“Description of Artificial Sequence Synthetic polypeptide”
ERPFQCRICMRNFSRSDHLSAHIRTHTGEKPFACDICGRKFACRRNLRNHTKIHTGSQKPFQCRICMRNFSMVCCRTLHIRTHTGEKPFACDICGRKFARSANLTRHTKIHTGSQKPFQCRICMRNFSTSSNRKTHIRTHTGEKPFACDICGRKFAQSGHLSRHTKIH

SEQ ID NO: 7 - protein, 139 aa, Artificial Sequence
  source: /note=“Description of Artificial Sequence Synthetic polypeptide”
ERPFQCRICMRNFSDDWNLSQHIRTHTGEKPFACDICGRKFARSANLTRHTKIHTGSQKPFQCRICMRKFAQSAHRITHTKIHTGEKPFQCRICMRNFSQSANRTTHIRTHTGEKPFACDICGRKFAQNAHRKTHTKIH

SEQ ID NO: 8 - protein, 168 aa, Artificial Sequence
  source: /note=“Description of Artificial Sequence Synthetic polypeptide”
ERPFQCRICMRNFSQSGDLTRHIRTHTGEKPFACDICGRKFAQSSDLRRHTKIHTGSQKPFQCRICMRNFSQSAHRKNHIRTHTGEKPFACDICGRKFARSAVRKNHTKIHTGSQKPFQCRICMRNFSQSANRTTHIRTHTGEKPFACDICGRKFARKYYLAKHTKIH

SEQ ID NO: 9 - protein, 172 aa, Artificial Sequence
  source: /note=“Description of Artificial Sequence Synthetic polypeptide”
ERPFQCRICMRNFSQSGDLTRHIRTHTGEKPFACDICGRKFAQSSDLRRHTKIHTHPRAPIPKPFQCRICMRNFSRSANLARHIRTHTGEKPFACDICGRKFATNQNRITHTKIHTGSQKPFQCRICMRNFSQSGDLTRHIRTHTGEKPFACDICGRKFARKDPLKEHTKIH

SEQ ID NO: 10 - protein, 139 aa, Artificial Sequence
  source: /note=“Description of Artificial Sequence Synthetic polypeptide”
ERPFQCRICMRKFAQSGNRTTHTKIHTGEKPFQCRICMRNFSTSSNRKTHIRTHTGEKPFACDICGRKFAAQWTRACHTKIHTGSQKPFQCRICMRNFSLRHHLTRHIRTHTGEKPFACDICGRKFADRTGLRSHTKIH

SEQ ID NO: 11 - protein, 180 aa, Artificial Sequence
  source: /note=“Description of Artificial Sequence Synthetic polypeptide”
ERPFQCRICMRNFSQSGHLARHIRTHTGEKPFACDICGRKFANRHDRAKHTKIHTPNPHRRTDPSHKPFQCRICMRNFSQSADRTKHIRTHTGEKPFACDICGRKFAQSGSLTRHTKIHTHPRAPIPKPFQCRICMRNFSDRSTRITHIRTHTGEKPFACDICGRKFAQNATRINHTKIH

SEQ ID NO: 12 - protein, 143 aa, Artificial Sequence
  source: /note=“Description of Artificial Sequence Synthetic polypeptide”
ERPFQCRICMRNFSQSGHLARHIRTHTGEKPFACDICGRKFANRHDRAKHTKIHTHPRAPIPKPFQCRICMRKFAQSGNRTTHTKIHTGEKPFQCRICMRNFSTSSNRKTHIRTHTGEKPFACDICGRKFAAQWTRACHTKIH

SEQ ID NO: 13 - protein, 180 aa, Artificial Sequence
  source: /note=“Description of Artificial Sequence Synthetic polypeptide”
ERPFQCRICMRNFSDIGYRAAHIRTHTGEKPFACDICGRKFAQSGNLARHTKIHTHPRAPIPKPFQCRICMRNFSQSGHLARHIRTHTGEKPFACDICGRKFANRHDRAKHTKIHTPNPHRRTDPSHKPFQCRICMRNFSQSADRTKHIRTHTGEKPFACDICGRKFAQSGSLTRHTKIH

SEQ ID NO: 14 - protein, 172 aa, Artificial Sequence
  source: /note=“Description of Artificial Sequence Synthetic polypeptide”
ERPFQCRICMRNFSDRSNLSRHIRTHTGEKPFACDICGRKFAQSGDLTRHTKIHTGSQKPFQCRICMRNFSDIGYRAAHIRTHTGEKPFACDICGRKFAQSGNLARHTKIHTHPRAPIPKPFQCRICMRNFSQSGHLARHIRTHTGEKPFACDICGRKFANRHDRAKHTKIH

SEQ ID NO: 15 - protein, 9 aa, Artificial Sequence
  source: /note=“Description of Artificial Sequence Synthetic peptide”
LRGSQLVKS

SEQ ID NO: 16 - protein, 16 aa, Artificial Sequence
  source: /note=“Description of Artificial Sequence Synthetic peptide”
LRGSQLVKSKSEAAAR

SEQ ID NO: 17 - protein, 26 aa, Artificial Sequence
  source: /note=“Description of Artificial Sequence Synthetic peptide”
LRGSQLVKSKSEAAARGGGGSGGGGS

SEQ ID NO: 18 - protein, 44 aa, Artificial Sequence
  source: /note=“Description of Artificial Sequence Synthetic polypeptide”
GSYALGPYQISAPQLPAYNGQTVGTFYYVNDAGGLESKVFSSGG

SEQ ID NO: 19 - protein, 94 aa, Artificial Sequence
  source: /note=“Description of Artificial Sequence Synthetic polypeptide”
PTPYPNYANAGHVEGQSALFMRDNGISEGLVFHNNPEGTCGFCVNMTETLLPENAKMTVVPPEGAIPVKRGATGETKVFTGNSNSPKSPTKGGC

SEQ ID NO: 20 - protein, 83 aa, Artificial Sequence
  source: /note=“Description of Artificial Sequence Synthetic polypeptide”
TNLSDIIEKETGKQLVIQESILMLPEEVEEVIGNKPESDILVHTAYDESTDENVMLLTSDAPEYKPWALVIQDSNGENKIKML

SEQ ID NO: 21 - protein, 131 aa, Artificial Sequence
  source: /note=“Description of Artificial Sequence Synthetic polypeptide”
GSYALGPYQISAPQLPAYNGQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 22 - protein, 181 aa, Artificial Sequence
  source: /note=“Description of Artificial Sequence Synthetic polypeptide”
PTPYPNYANAGHVEGQSALFMRDNGISEGLVFHNNPEGTCGFCVNMTETLLPENAKMTVVPPEGAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 23 - protein, 354 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGALARHIRTHTGEKPFACDICGRKFALKQHLTRHTKIHTGSQKPFQCRICMRNFSQSGDLTRHIRTHTGEKPFACDICGRKFAQSSDLRRHTKIHTHPRAPIPKPFQCRICMRNFSRSANLARHIRTHTGEKPFACDICGRKFATNQNRITHTKIHLRGSQLVKSGSYALGPYQISAPQLPAYNGQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 24 - protein, 361 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSRSDSLSVHIRTHTGEKPFACDICGRKFAQSGSLTRHTKIHTGSQKPFQCRICMRNFSTSGHLSRHIRTHTGEKPFACDICGRKFAQSGDLTRHTKIHTHPRAPIPKPFQCRICMRNFSMVCCRTLHIRTHTGEKPFACDICGRKFARSANLTRHTKIHLRGSQLVKSKSEAAARGSYALGPYQISAPQLPAYNGQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 25 - protein, 357 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSRSDHLSAHIRTHTGEKPFACDICGRKFACRRNLRNHTKIHTGSQKPFQCRICMRNFSMVCCRTLHIRTHTGEKPFACDICGRKFARSANLTRHTKIHTGSQKPFQCRICMRNFSTSSNRKTHIRTHTGEKPFACDICGRKFAQSGHLSRHTKIHLRGSQLVKSKSEAAARGSYALGPYQISAPQLPAYNGQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 26 - protein, 361 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGDLTRHIRTHTGEKPFACDICGRKFAQSSDLRRHTKIHTHPRAPIPKPFQCRICMRNFSRSANLARHIRTHTGEKPFACDICGRKFATNQNRITHTKIHTGSQKPFQCRICMRNFSQSGDLTRHIRTHTGEKPFACDICGRKFARKDPLKEHTKIHLRGSQLVKSKSEAAARGSYALGPYQISAPQLPAYNGQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 27 - protein, 407 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGALARHIRTHTGEKPFACDICGRKFALKQHLTRHTKIHTGSQKPFQCRICMRNFSQSGDLTRHIRTHTGEKPFACDICGRKFAQSSDLRRHTKIHTGSQKPFQCRICMRNFSQSAHRKNHIRTHTGEKPFACDICGRKFARSAVRKNHTKIHLRGSQLVKSKSEAAARPTPYPNYANAGHVEGQSALFMRDNGISEGLVFHNNPEGTCGFCVNMTETLLPENAKMTVVPPEGAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 28 - protein, 371 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSRSDSLSVHIRTHTGEKPFACDICGRKFAQSGSLTRHTKIHTGSQKPFQCRICMRNFSTSGHLSRHIRTHTGEKPFACDICGRKFAQSGDLTRHTKIHTHPRAPIPKPFQCRICMRNFSMVCCRTLHIRTHTGEKPFACDICGRKFARSANLTRHTKIHLRGSQLVKSKSEAAARGGGGSGGGGSGSYALGPYQISAPQLPAYNGQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 29 - protein, 367 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGALARHIRTHTGEKPFACDICGRKFALKQHLTRHTKIHTGSQKPFQCRICMRNFSQSGDLTRHIRTHTGEKPFACDICGRKFAQSSDLRRHTKIHTGSQKPFQCRICMRNFSQSAHRKNHIRTHTGEKPFACDICGRKFARSAVRKNHTKIHLRGSQLVKSKSEAAARGGGGSGGGGSGSYALGPYQISAPQLPAYNGQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 30 - protein, 371 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGALARHIRTHTGEKPFACDICGRKFALKQHLTRHTKIHTGSQKPFQCRICMRNFSQSGDLTRHIRTHTGEKPFACDICGRKFAQSSDLRRHTKIHTHPRAPIPKPFQCRICMRNFSRSANLARHIRTHTGEKPFACDICGRKFATNQNRITHTKIHLRGSQLVKSKSEAAARGGGGSGGGGSGSYALGPYQISAPQLPAYNGQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 31 - protein, 367 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSRSDHLSAHIRTHTGEKPFACDICGRKFACRRNLRNHTKIHTGSQKPFQCRICMRNFSMVCCRTLHIRTHTGEKPFACDICGRKFARSANLTRHTKIHTGSQKPFQCRICMRNFSTSSNRKTHIRTHTGEKPFACDICGRKFAQSGHLSRHTKIHLRGSQLVKSKSEAAARGGGGSGGGGSGSYALGPYQISAPQLPAYNGQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 32 - protein, 371 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGDLTRHIRTHTGEKPFACDICGRKFAQSSDLRRHTKIHTHPRAPIPKPFQCRICMRNFSRSANLARHIRTHTGEKPFACDICGRKFATNQNRITHTKIHTGSQKPFQCRICMRNFSQSGDLTRHIRTHTGEKPFACDICGRKFARKDPLKEHTKIHLRGSQLVKSKSEAAARGGGGSGGGGSGSYALGPYQISAPQLPAYNGQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 33 - protein, 417 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGALARHIRTHTGEKPFACDICGRKFALKQHLTRHTKIHTGSQKPFQCRICMRNFSQSGDLTRHIRTHTGEKPFACDICGRKFAQSSDLRRHTKIHTGSQKPFQCRICMRNFSQSAHRKNHIRTHTGEKPFACDICGRKFARSAVRKNHTKIHLRGSQLVKSKSEAAARGGGGSGGGGSPTPYPNYANAGHVEGQSALFMRDNGISEGLVFHNNPEGTCGFCVNMTETLLPENAKMTVVPPEGAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 34 - protein, 421 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGALARHIRTHTGEKPFACDICGRKFALKQHLTRHTKIHTGSQKPFQCRICMRNFSQSGDLTRHIRTHTGEKPFACDICGRKFAQSSDLRRHTKIHTHPRAPIPKPFQCRICMRNFSRSANLARHIRTHTGEKPFACDICGRKFATNQNRITHTKIHLRGSQLVKSKSEAAARGGGGSGGGGSPTPYPNYANAGHVEGQSALFMRDNGISEGLVFHNNPEGTCGFCVNMTETLLPENAKMTVVPPEGAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 35 - protein, 421 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGDLTRHIRTHTGEKPFACDICGRKFAQSSDLRRHTKIHTHPRAPIPKPFQCRICMRNFSRSANLARHIRTHTGEKPFACDICGRKFATNQNRITHTKIHTGSQKPFQCRICMRNFSQSGDLTRHIRTHTGEKPFACDICGRKFARKDPLKEHTKIHLRGSQLVKSKSEAAARGGGGSGGGGSPTPYPNYANAGHVEGQSALFMRDNGISEGLVFHNNPEGTCGFCVNMTETLLPENAKMTVVPPEGAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 36 - protein, 412 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSDIGYRAAHIRTHTGEKPFACDICGRKFAQSGNLARHTKIHTHPRAPIPKPFQCRICMRNFSQSGHLARHIRTHTGEKPFACDICGRKFANRHDRAKHTKIHTPNPHRRTDPSHKPFQCRICMRNFSQSADRTKHIRTHTGEKPFACDICGRKFAQSGSLTRHTKIHLRGSQLVKSPTPYPNYANAGHVEGQSALFMRDNGISEGLVFHNNPEGTCGFCVNMTETLLPENAKMTVVPPEGAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 37 - protein, 404 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSDRSNLSRHIRTHTGEKPFACDICGRKFAQSGDLTRHTKIHTGSQKPFQCRICMRNFSDIGYRAAHIRTHTGEKPFACDICGRKFAQSGNLARHTKIHTHPRAPIPKPFQCRICMRNFSQSGHLARHIRTHTGEKPFACDICGRKFANRHDRAKHTKIHLRGSQLVKSPTPYPNYANAGHVEGQSALFMRDNGISEGLVFHNNPEGTCGFCVNMTETLLPENAKMTVVPPEGAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 38 - protein, 378 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRKFAQSGNRTTHTKIHTGEKPFQCRICMRNFSTSSNRKTHIRTHTGEKPFACDICGRKFAAQWTRACHTKIHTGSQKPFQCRICMRNFSLRHHLTRHIRTHTGEKPFACDICGRKFADRTGLRSHTKIHLRGSQLVKSKSEAAARPTPYPNYANAGHVEGQSALFMRDNGISEGLVFHNNPEGTCGFCVNMTETLLPENAKMTVVPPEGAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 39 - protein, 411 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSDRSNLSRHIRTHTGEKPFACDICGRKFAQSGDLTRHTKIHTGSQKPFQCRICMRNFSDIGYRAAHIRTHTGEKPFACDICGRKFAQSGNLARHTKIHTHPRAPIPKPFQCRICMRNFSQSGHLARHIRTHTGEKPFACDICGRKFANRHDRAKHTKIHLRGSQLVKSKSEAAARPTPYPNYANAGHVEGQSALFMRDNGISEGLVFHNNPEGTCGFCVNMTETLLPENAKMTVVPPEGAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 40 - protein, 419 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSDIGYRAAHIRTHTGEKPFACDICGRKFAQSGNLARHTKIHTHPRAPIPKPFQCRICMRNFSQSGHLARHIRTHTGEKPFACDICGRKFANRHDRAKHTKIHTPNPHRRTDPSHKPFQCRICMRNFSQSADRTKHIRTHTGEKPFACDICGRKFAQSGSLTRHTKIHLRGSQLVKSKSEAAARPTPYPNYANAGHVEGQSALFMRDNGISEGLVFHNNPEGTCGFCVNMTETLLPENAKMTVVPPEGAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 41 - protein, 328 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRKFAQSGNRTTHTKIHTGEKPFQCRICMRNFSTSSNRKTHIRTHTGEKPFACDICGRKFAAQWTRACHTKIHTGSQKPFQCRICMRNFSLRHHLTRHIRTHTGEKPFACDICGRKFADRTGLRSHTKIHLRGSQLVKSKSEAAARGSYALGPYQISAPQLPAYNGQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 42 - protein, 388 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRKFAQSGNRTTHTKIHTGEKPFQCRICMRNFSTSSNRKTHIRTHTGEKPFACDICGRKFAAQWTRACHTKIHTGSQKPFQCRICMRNFSLRHHLTRHIRTHTGEKPFACDICGRKFADRTGLRSHTKIHLRGSQLVKSKSEAAARGGGGSGGGGSPTPYPNYANAGHVEGQSALFMRDNGISEGLVFHNNPEGTCGFCVNMTETLLPENAKMTVVPPEGAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 43 - protein, 421 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSDRSNLSRHIRTHTGEKPFACDICGRKFAQSGDLTRHTKIHTGSQKPFQCRICMRNFSDIGYRAAHIRTHTGEKPFACDICGRKFAQSGNLARHTKIHTHPRAPIPKPFQCRICMRNFSQSGHLARHIRTHTGEKPFACDICGRKFANRHDRAKHTKIHLRGSQLVKSKSEAAARGGGGSGGGGSPTPYPNYANAGHVEGQSALFMRDNGISEGLVFHNNPEGTCGFCVNMTETLLPENAKMTVVPPEGAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 44 - protein, 429 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSDIGYRAAHIRTHTGEKPFACDICGRKFAQSGNLARHTKIHTHPRAPIPKPFQCRICMRNFSQSGHLARHIRTHTGEKPFACDICGRKFANRHDRAKHTKIHTPNPHRRTDPSHKPFQCRICMRNFSQSADRTKHIRTHTGEKPFACDICGRKFAQSGSLTRHTKIHLRGSQLVKSKSEAAARGGGGSGGGGSPTPYPNYANAGHVEGQSALFMRDNGISEGLVFHNNPEGTCGFCVNMTETLLPENAKMTVVPPEGAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 45 - protein, 338 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRKFAQSGNRTTHTKIHTGEKPFQCRICMRNFSTSSNRKTHIRTHTGEKPFACDICGRKFAAQWTRACHTKIHTGSQKPFQCRICMRNFSLRHHLTRHIRTHTGEKPFACDICGRKFADRTGLRSHTKIHLRGSQLVKSKSEAAARGGGGSGGGGSGSYALGPYQISAPQLPAYNGQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 46 - protein, 371 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSDRSNLSRHIRTHTGEKPFACDICGRKFAQSGDLTRHTKIHTGSQKPFQCRICMRNFSDIGYRAAHIRTHTGEKPFACDICGRKFAQSGNLARHTKIHTHPRAPIPKPFQCRICMRNFSQSGHLARHIRTHTGEKPFACDICGRKFANRHDRAKHTKIHLRGSQLVKSKSEAAARGGGGSGGGGSGSYALGPYQISAPQLPAYNGQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 47 - DNA, 20 bp, Artificial Sequence
  source: /note=“Description of Artificial Sequence Synthetic primer”
acacgacgctcttccgatct

SEQ ID NO: 48 - DNA, 18 bp, Artificial Sequence
  source: /note=“Description of Artificial Sequence Synthetic primer”
gacgtgtgctcttccgat

SEQ ID NO: 49 - protein, 138 aa, Burkholderia cenocepacia
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETKVFTGNSNSPKSPTKGGC

SEQ ID NO: 50 - protein, 144 aa, Artificial Sequence
  source: /note=“Description of Artificial Sequence Synthetic polypeptide”
AIPVKRGATGETKVFTGNSNSPKSPTKGGCPTPYPNYANAGHVEGQSALFMRDNGISEGLVFHNNPEGTCGFCVNMTETLLPENAKMTVVPPEGGGSGGSGSYALGPYQISAPQLPAYNGQTVGTFYYVNDAGGLESKVFSSGG

SEQ ID NO: 51 - protein, 120 aa, Artificial Sequence
  source: /note=“Description of Artificial Sequence Synthetic polypeptide”
AIPVKRGATGETKVFTGNSNSPKSPTKGGCPTPYPNYANAGHVEGQSALFMRDNGISEGLVFHNNPEGTCGFCVNMTETLLPENAKMTVVPPEGGGSGGSGSYALGPYQISAPQLPAYNG

SEQ ID NO: 52 - protein, 24 aa, Artificial Sequence
  source: /note=“Description of Artificial Sequence Synthetic peptide”
QTVGTFYYVNDAGGLESKVFSSGG

SEQ ID NO: 53 - protein, 54 aa, Artificial Sequence
  source: /note=“Description of Artificial Sequence Synthetic polypeptide”
AIPVKRGATGETKVFTGNSNSPKSPTKGGCPTPYPNYANAGHVEGQSALFMRDN

SEQ ID NO: 54 - protein, 90 aa, Artificial Sequence
  source: /note=“Description of Artificial Sequence Synthetic polypeptide”
GISEGLVFHNNPEGTCGFCVNMTETLLPENAKMTVVPPEGGGSGGSGSYALGPYQISAPQLPAYNGQTVGTFYYVNDAGGLESKVFSSGG

SEQ ID NO: 55 - protein, 447 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGDLTRHIRTHTGEKPFACDICGRKFAQSSDLRRHTKIHTHPRAPIPKPFQCRICMRNFSRSANLARHIRTHTGEKPFACDICGRKFATNQNRITHTKIHTGSQKPFQCRICMRNFSQSGDLTRHIRTHTGEKPFACDICGRKFARKDPLKEHTKIHLRGSQLVKSKSEAAARGGGGSGGGGSAIPVKRGATGETKVFTGNSNSPKSPTKGGCPTPYPNYANAGHVEGQSALFMRDNGISEGLVFHNNPEGTCGFCVNMTETLLPENAKMTVVPPEGGGSGGSGSYALGPYQISAPQLPAYNGSGGSTNLSDIIEKETGKQLVIQESILMLPEEVEEVIGNKPESDILVHTAYDESTDENVMLLTSDAPEYKPWALVIQDSNGENKIKML

SEQ ID NO: 56 - protein, 447 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGALARHIRTHTGEKPFACDICGRKFALKQHLTRHTKIHTGSQKPFQCRICMRNFSQSGDLTRHIRTHTGEKPFACDICGRKFAQSSDLRRHTKIHTHPRAPIPKPFQCRICMRNFSRSANLARHIRTHTGEKPFACDICGRKFATNQNRITHTKIHLRGSQLVKSKSEAAARGGGGSGGGGSAIPVKRGATGETKVFTGNSNSPKSPTKGGCPTPYPNYANAGHVEGQSALFMRDNGISEGLVFHNNPEGTCGFCVNMTETLLPENAKMTVVPPEGGGSGGSGSYALGPYQISAPQLPAYNGSGGSTNLSDIIEKETGKQLVIQESILMLPEEVEEVIGNKPESDILVHTAYDESTDENVMLLTSDAPEYKPWALVIQDSNGENKIKML

SEQ ID NO: 57 - protein, 351 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSDRSNLSRHIRTHTGEKPFACDICGRKFAQSGDLTRHTKIHTGSQKPFQCRICMRNFSDIGYRAAHIRTHTGEKPFACDICGRKFAQSGNLARHTKIHTHPRAPIPKPFQCRICMRNFSQSGHLARHIRTHTGEKPFACDICGRKFANRHDRAKHTKIHLRGSQLVKSKSEAAARGGGGSGGGGSQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 58 - protein, 359 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGHLARHIRTHTGEKPFACDICGRKFANRHDRAKHTKIHTPNPHRRTDPSHKPFQCRICMRNFSQSADRTKHIRTHTGEKPFACDICGRKFAQSGSLTRHTKIHTHPRAPIPKPFQCRICMRNFSDRSTRITHIRTHTGEKPFACDICGRKFAQNATRINHTKIHLRGSQLVKSKSEAAARGGGGSGGGGSQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 59 - protein, 351 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGDLTRHIRTHTGEKPFACDICGRKFAQSSDLRRHTKIHTHPRAPIPKPFQCRICMRNFSRSANLARHIRTHTGEKPFACDICGRKFATNQNRITHTKIHTGSQKPFQCRICMRNFSQSGDLTRHIRTHTGEKPFACDICGRKFARKDPLKEHTKIHLRGSQLVKSKSEAAARGGGGSGGGGSQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 60 - protein, 351 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGALARHIRTHTGEKPFACDICGRKFALKQHLTRHTKIHTGSQKPFQCRICMRNFSQSGDLTRHIRTHTGEKPFACDICGRKFAQSSDLRRHTKIHTHPRAPIPKPFQCRICMRNFSRSANLARHIRTHTGEKPFACDICGRKFATNQNRITHTKIHLRGSQLVKSKSEAAARGGGGSGGGGSQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 61 - protein, 447 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSDRSNLSRHIRTHTGEKPFACDICGRKFAQSGDLTRHTKIHTGSQKPFQCRICMRNFSDIGYRAAHIRTHTGEKPFACDICGRKFAQSGNLARHTKIHTHPRAPIPKPFQCRICMRNFSQSGHLARHIRTHTGEKPFACDICGRKFANRHDRAKHTKIHLRGSQLVKSKSEAAARGGGGSGGGGSAIPVKRGATGETKVFTGNSNSPKSPTKGGCPTPYPNYANAGHVEGQSALFMRDNGISEGLVFHNNPEGTCGFCVNMTETLLPENAKMTVVPPEGGGSGGSGSYALGPYQISAPQLPAYNGSGGSTNLSDIIEKETGKQLVIQESILMLPEEVEEVIGNKPESDILVHTAYDESTDENVMLLTSDAPEYKPWALVIQDSNGENKIKML

SEQ ID NO: 62 - protein, 455 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGHLARHIRTHTGEKPFACDICGRKFANRHDRAKHTKIHTPNPHRRTDPSHKPFQCRICMRNFSQSADRTKHIRTHTGEKPFACDICGRKFAQSGSLTRHTKIHTHPRAPIPKPFQCRICMRNFSDRSTRITHIRTHTGEKPFACDICGRKFAQNATRINHTKIHLRGSQLVKSKSEAAARGGGGSGGGGSAIPVKRGATGETKVFTGNSNSPKSPTKGGCPTPYPNYANAGHVEGQSALFMRDNGISEGLVFHNNPEGTCGFCVNMTETLLPENAKMTVVPPEGGGSGGSGSYALGPYQISAPQLPAYNGSGGSTNLSDIIEKETGKQLVIQESILMLPEEVEEVIGNKPESDILVHTAYDESTDENVMLLTSDAPEYKPWALVIQDSNGENKIKML

SEQ ID NO: 63 - protein, 381 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGDLTRHIRTHTGEKPFACDICGRKFAQSSDLRRHTKIHTHPRAPIPKPFQCRICMRNFSRSANLARHIRTHTGEKPFACDICGRKFATNQNRITHTKIHTGSQKPFQCRICMRNFSQSGDLTRHIRTHTGEKPFACDICGRKFARKDPLKEHTKIHLRGSQLVKSKSEAAARGGGGSGGGGSAIPVKRGATGETKVFTGNSNSPKSPTKGGCPTPYPNYANAGHVEGQSALFMRDNSGGSTNLSDIIEKETGKQLVIQESILMLPEEVEEVIGNKPESDILVHTAYDESTDENVMLLTSDAPEYKPWALVIQDSNGENKIKML

SEQ ID NO: 64 - protein, 381 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGALARHIRTHTGEKPFACDICGRKFALKQHLTRHTKIHTGSQKPFQCRICMRNFSQSGDLTRHIRTHTGEKPFACDICGRKFAQSSDLRRHTKIHTHPRAPIPKPFQCRICMRNFSRSANLARHIRTHTGEKPFACDICGRKFATNQNRITHTKIHLRGSQLVKSKSEAAARGGGGSGGGGSAIPVKRGATGETKVFTGNSNSPKSPTKGGCPTPYPNYANAGHVEGQSALFMRDNSGGSTNLSDIIEKETGKQLVIQESILMLPEEVEEVIGNKPESDILVHTAYDESTDENVMLLTSDAPEYKPWALVIQDSNGENKIKML

SEQ ID NO: 65 - protein, 417 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSDRSNLSRHIRTHTGEKPFACDICGRKFAQSGDLTRHTKIHTGSQKPFQCRICMRNFSDIGYRAAHIRTHTGEKPFACDICGRKFAQSGNLARHTKIHTHPRAPIPKPFQCRICMRNFSQSGHLARHIRTHTGEKPFACDICGRKFANRHDRAKHTKIHLRGSQLVKSKSEAAARGGGGSGGGGSGISEGLVFHNNPEGTCGFCVNMTETLLPENAKMTVVPPEGGGSGGSGSYALGPYQISAPQLPAYNGQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 66 - protein, 425 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGHLARHIRTHTGEKPFACDICGRKFANRHDRAKHTKIHTPNPHRRTDPSHKPFQCRICMRNFSQSADRTKHIRTHTGEKPFACDICGRKFAQSGSLTRHTKIHTHPRAPIPKPFQCRICMRNFSDRSTRITHIRTHTGEKPFACDICGRKFAQNATRINHTKIHLRGSQLVKSKSEAAARGGGGSGGGGSGISEGLVFHNNPEGTCGFCVNMTETLLPENAKMTVVPPEGGGSGGSGSYALGPYQISAPQLPAYNGQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 67 - protein, 417 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGDLTRHIRTHTGEKPFACDICGRKFAQSSDLRRHTKIHTHPRAPIPKPFQCRICMRNFSRSANLARHIRTHTGEKPFACDICGRKFATNQNRITHTKIHTGSQKPFQCRICMRNFSQSGDLTRHIRTHTGEKPFACDICGRKFARKDPLKEHTKIHLRGSQLVKSKSEAAARGGGGSGGGGSGISEGLVFHNNPEGTCGFCVNMTETLLPENAKMTVVPPEGGGSGGSGSYALGPYQISAPQLPAYNGQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 68 - protein, 417 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGALARHIRTHTGEKPFACDICGRKFALKQHLTRHTKIHTGSQKPFQCRICMRNFSQSGDLTRHIRTHTGEKPFACDICGRKFAQSSDLRRHTKIHTHPRAPIPKPFQCRICMRNFSRSANLARHIRTHTGEKPFACDICGRKFATNQNRITHTKIHLRGSQLVKSKSEAAARGGGGSGGGGSGISEGLVFHNNPEGTCGFCVNMTETLLPENAKMTVVPPEGGGSGGSGSYALGPYQISAPQLPAYNGQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 69 - protein, 381 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSDRSNLSRHIRTHTGEKPFACDICGRKFAQSGDLTRHTKIHTGSQKPFQCRICMRNFSDIGYRAAHIRTHTGEKPFACDICGRKFAQSGNLARHTKIHTHPRAPIPKPFQCRICMRNFSQSGHLARHIRTHTGEKPFACDICGRKFANRHDRAKHTKIHLRGSQLVKSKSEAAARGGGGSGGGGSAIPVKRGATGETKVFTGNSNSPKSPTKGGCPTPYPNYANAGHVEGQSALFMRDNSGGSTNLSDIIEKETGKQLVIQESILMLPEEVEEVIGNKPESDILVHTAYDESTDENVMLLTSDAPEYKPWALVIQDSNGENKIKML

SEQ ID NO: 70 - protein, 389 aa, Artificial Sequence
  source: /note=“Description of Artificial Sequence Synthetic polypeptide”
MDYKDHDGDYKDHDIDYKDDDDKMAPKKKRKVGIHGVPAAMAERPFQCRICMRNFSQSGHLARHIRTHTGEKPFACDICGRKFANRHDRAKHTKIHTPNPHRRTDPSHKPFQCRICMRNFSQSADRTKHIRTHTGEKPFACDICGRKFAQSGSLTRHTKIHTHPRAPIPKPFQCRICMRNFSDRSTRITHIRTHTGEKPFACDICGRKFAQNATRINHTKIHLRGSQLVKSKSEAAARGGGGSGGGGSAIPVKRGATGETKVFTGNSNSPKSPTKGGCPTPYPNYANAGHVEGQSALFMRDNSGGSTNLSDIIEKETGKQLVIQESILMLPEEVEEVIGNKPESDILVHTAYDESTDENVMLLTSDAPEYKPWALVIQDSNGENKIKML

SEQ ID NO: 71 - protein, 5 aa, Artificial Sequence
  source: /note=“Description of Artificial Sequence Synthetic peptide”
GGGGS

SEQ ID NO: 72 - protein, 1427 aa, Burkholderia cenocepacia
MYEAARVTDPIDHTSALAGFLVGAVLGIALIAAVAFATFTCGFGVALLAGMMAGIGAQALLSIGESIGKMFSSQSGNIITGSPDVYVNSLSAAYATLSGVACSKHNPIPLVAQGSTNIFINGRPAARKDDKITCGATIGDGSHDTFFHGGTQTYLPVDDEVPPWLRTATDWAFTLAGLVGGLGGLLKASGGLSRAVLPCAAKFIGGYVLGEAFGRYVAGPAINKAIGGLFGNPIDVTTGRKILLAESETDYVIPSPLPVAIKRFYSSGIDYAGTLGRGWVLPWEIRLHARDGRLWYTDAQGRESGFPMLRAGQAAFSEADQRYLTRTPDGRYILHDLGERYYDFGQYDPESGRIAWVRRVEDQAGQWYQFERDSRGRVTEILTCGGLRAVLDYETVFGRLGTVTLVHEDERRLAVTYGYDENGQLASVTDANGAGVRQFAYTNGLMTNHMNALGFTSSYVWSKIEGEPRVVETHTSEGENWTFEYDVAGRQTRVRHADGRTAHWRFDAQSQIVEYTDLDGAFYRIKYDAVGMPVMLMLPGDRTVMFEYDDAGRIIAETDPLGRTTRTRYDGNSLRPVEVVGPDGGAWRVEYDQQGRVVSNQDSLGRENRYEYPKALTALPSAHIDALGGRKTLEWNSLGKLVGYTDCSGKTTRTSFDAFGRICSRENALGQRITYDVRPTGEPRRVTYPDGSSETFEYDAAGTLVRYIGLGGRVQELLRNARGQLIEAVDPAGRRVQYRYDVEGRLRELQQDHARYTFTYSAGGRLLTETRPDGILRRFEYGEAGELLGLDIVGAPDPHATGNRSVRTIRFERDRMGVLKVQRTPTEVTRYQHDKGDRLVKVERVPTPSGIALGIVPDAVEFEYDKGGRLVAEHGSNGSVIYTLDELDNVVSLGLPHDQTLQMLRYGSGHVHQIRFGDQVVADFERDDLHREVSRTQGRLTQRSGYDPLGRKVWQSAGIDPEMLGRGSGQLWRNYGYDAAGDLIETSDSLRGSTRFSYDPAGRLISRANPLDRKFEEFAWDAAGNLLDDAQRKSRGYVEGNRLLMWQDLRFEYDPFGNLATKRRGANQTQRFTYDGQDRLITVHTQDVRGVVETRFAYDPLGRRIAKTDTAFDLRGMKLRAETKRFVWEGLRLVQEVRETGVSSYVYSPDAPYSPVARADTVMAEALAATVIDSAKRAARIFHFHTDPVGALQEVTDEAGEVAWAGQYAAWGKVEATNRGVTAARTDQPLRFAGQYADDSTGLHYNTFRFYDPDVGRFINQDPIGLNGGANVYHYAPNPVGWVDPWGLAGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETKVFTGNSNSPKSPTKGGC

SEQ ID NO: 73 - protein, 123 aa, Burkholderia cenocepacia
MYADDFDGEIEIDEVDSLVEFLSRRPAFDANNFVLTFEESGFPQLNIFAKNDIAVVYYMDIGENFVSKGNSASGGTEKFYENKLGGEVDLSKDCVVSKEQMIEAAKQFFATKQRPEQLTWSEL

SEQ ID NO: 74 - DNA, 58 bp, Artificial Sequence
  source: /note=“Description of Artificial Sequence Synthetic primer”
  modified_base (34)..(37): a, c, t, g, unknown or other
acactctttccctacacgacgctcttccgatctnnnncaagtgtgatcacttgggtgg

SEQ ID NO: 75 - DNA, 49 bp, Artificial Sequence
  source: /note=“Description of Artificial Sequence Synthetic primer”
tggagttcagacgtgtgctcttccgatctggattcccgagtagcagatg

SEQ ID NO: 76 - DNA, 214 bp, Artificial Sequence
  source: /note=“Description of Artificial Sequence Synthetic polynucleotide”
  modified_base (1)..(4): a, c, t, g, unknown or other
nnnncaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcc

SEQ ID NO: 77 - protein, 626 aa, Artificial Sequence
  source: /note=“Description of Artificial Sequence Synthetic polypeptide”
MDYKDHDGDYKDHDIDYKDDDDKMAPKKKRKVGIHGVPAAMGQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINFSGAQGSTLDFRPFQCRICMRNFSDRSNLSRHIRTHTGEKPFACDICGRKFAQSGDLTRHTKIHTGSQKPFQCRICMRNFSDIGYRAAHIRTHTGEKPFACDICGRKFAQSGNLARHTKIHTHPRAPIPKPFQCRICMRNFSQSGHLARHIRTHTGEKPFACDICGRKFANRHDRAKHTKIHLRGSQLVKSKSEAAARGGGGSGGGGSPTPYPNYANAGHVEGQSALFMRDNGISEGLVFHNNPEGTCGFCVNMTETLLPENAKMTVVPPEGAIPVKRGATGETKVFTGNSNSPKSPTKGGCSGGSTNLSDIIEKETGKQLVIQESILMLPEEVEEVIGNKPESDILVHTAYDESTDENVMLLTSDAPEYKPWALVIQDSNGENKIKML

SEQ ID NO: 78 - protein, 391 aa, Artificial Sequence
  source: /note=“Description of Artificial Sequence Synthetic polypeptide”
MDYKDHDGDYKDHDIDYKDDDDKMAPKKKRKVGIHGVPAAMGQLVKSELEEKKSELRHKLKYVPHEYIELIEIARNSTQDRILEMKVMEFFMKVYGYRGKHLGGSRKPNGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINFSGAQGSTLDFRPFQCRICMRNFSCSNNLPTHIRTHTGEKPFACDICGRKFADRSNLTRHTKIHTGSQKPFQCRICMRNFSTSGNLTRHIRTHTGEKPFACDICGRKFAQAENLKSHTKIHTGEKPFQCRICMRKFADRSTLRQHTKIHLRQKD

SEQ ID NO: 79 - protein, 543 aa, Artificial Sequence
  source: /note=“Description of Artificial Sequence Synthetic polypeptide”
MDYKDHDGDYKDHDIDYKDDDDKMAPKKKRKVGIHGVPAAMGQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMERYVEENQTRDKHLNPNEWWKVYPSSVTEFKFLFVSGHFKGNYKAQLTRLNHITNCNGAVLSVEELLIGGEMIKAGTLTLEEVRRKFNNGEINFSGAQGSTLDFRPFQCRICMRKFAQSGNRTTHTKIHTGEKPFQCRICMRNFSTSSNRKTHIRTHTGEKPFACDICGRKFAAQWTRACHTKIHTGSQKPFQCRICMRNFSLRHHLTRHIRTHTGEKPFACDICGRKFADRTGLRSHTKIHLRGSQLVKSKSEAAARGGGGSGGGGSGSYALGPYQISAPQLPAYNGQTVGTFYYVNDAGGLESKVFSSGGSGGSTNLSDIIEKETGKQLVIQESILMLPEEVEEVIGNKPESDILVHTAYDESTDENVMLLTSDAPEYKPWALVIQDSNGENKIKML

SEQ ID NO: 80 - protein, 395 aa, Artificial Sequence
  source: /note=“Description of Artificial Sequence Synthetic polypeptide”
MDYKDHDGDYKDHDIDYKDDDDKMAPKKKRKVGIHGVPAAMGQLVKSELEEKKSELRHKLKYVPHEYIELIEIARNSTQDRILEMKVMEFFMKVYGYRGKHLGGSRKPNGAIYTVGSPIDYGVIVDTKAYSGGYNLPIGQADEMQRYVKENQTRNKHINPNEWWKVYPSSVTEFKFLFVSGHFKGNYKAQLTRLNRKTNCNGAVLSVEELLIGGEMIKAGTLTLEEVRRKFNNGEINFSGAQGSTLDFRPFQCRICMRKFARNADRKKHTKIHTGEKPFQCRICMRNFSTSSNRKTHIRTHTGEKPFACDICGRKFAQSGHLSRHTKIHTHPRAPIPKPFQCRICMRNFSDRSALSRHIRTHTGEKPFACDICGRKFATSSNRKTHTKIHLRQKD

SEQ ID NO: 81 - protein, 164 aa, Burkholderia cenocepacia
IGLNGGANVYHYAPNPVGWVDPWGLAGSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATGETKVFTGNSNSPKSPTKGGC

SEQ ID NO: 82 - protein, 115 aa, Artificial Sequence
  source: /note=“Description of Artificial Sequence Synthetic polypeptide”
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRG

SEQ ID NO: 83 - protein, 23 aa, Artificial Sequence
  source: /note=“Description of Artificial Sequence Synthetic peptide”
ATGETKVFTGNSNSPKSPTKGGC

SEQ ID NO: 84 - protein, 118 aa, Artificial Sequence
  source: /note=“Description of Artificial Sequence Synthetic polypeptide”
GSYALGPYQISAPQLPAYNGQTVGTFYYVNDAGGLESKVFSSGGPTPYPNYANAGHVEGQSALFMRDNGISEGLVFHNNPEGTCGFCVNMTETLLPENAKMTVVPPEGAIPVKRGATG

SEQ ID NO: 85 - protein, 20 aa, Artificial Sequence
  source: /note=“Description of Artificial Sequence Synthetic peptide”
ETKVFTGNSNSPKSPTKGGC

SEQ ID NO: 86 - protein, 2358 aa, Streptomyces rubrolavendulae
MSSSDAGRAFGVPENVLARFTRYPGGARRRAGRTARARRLGIVLSAVLSATLLPAEAWAIAPPAPRTGPTLDALQQEEEVDPDPAAMEELDDWDGGPVEPPADYTPTEVTPPTGGTAPVPLDSAGEELVPAGTLPVRIGQASPTEEDPAPPAPSGTWDVTVEPRATTEAAAVDGAIIKLTPPASGSTPVDVELDYGRFEDLFGTEWSSRLKLTQLPECFLTTPELEECGTPITIPTSNDPATGTVRATVDPADGQPQGLAAQSGGGPAVLAATDSASGAGGTYKATSLSATGSWTAGGSGGGFSWSYPLTIPDTPAGPAPKISLSYSSQSVDGRTSVANGQASWIGDGWDYHPGFVERRYRSCNDDRSGTPNNDNSADKEKSDLCWASDNVVMSLGGSTTELVRDDTTGTWVAQNDTGARIEYKDKDGGALAAQTAGYDGEHWVVTTRDGTRYWFGRNTLPGRGAPTNSALTVPVFGNHTGEPCHAATYAASSCTQAWRWNLDYVEDVHGNAMVVDWKKEQNRYAKNEKFKAAVSYDRDAYPTQILYGLRADDLAGPPAGKVVFHAAPRCLESAATCSEAKFESKNYADKQPWWDTPATLHCKAGDENCYVTSPTFWSRVRLSAIETQGQRTPGSTALSTVDRWTLHQSFPKQRTDTHPPLWLESITRVGFGRPDASGNQSSKALPAVTFLPNKVDMPNRVLKSTTDQTPDFDRLRVEVIRTETGGETHVTYSAPCPVGGTRPTPASNGTRCFPVHWSPDPAAFSDENLDKSGYEPPLEWFNKYVVTKVTEMDLVAEQPSVETVYTYEGDAAWAKNTDEYGKPALRTYDQWRGYASVVTRTGTTANTGAADATEQSQTRTRYFRGMSGDAGRAKVHVTLTDVTGTATTVEDLLPYQGMAAETLTYTKAGGDVAARELAFPYSRKTASRARPGLPALEAYRTGTTRTDSIQHISGDRTRAAQNHTTYDDAYGLPTQTYSLTLSPNDSGTLVAGDERCTVTTYVHNTAAHIIGLPDRVRATTGDCAAAPNATTGQIVSDSRTAYDALGAFGTAPVKGLPVQVDTISGGGTSWITSARTEYDALGRATKVTDAAGNSTTTTYSPATGPAFEVTVTNAAGHATTTTLDPGRGSALTVTDQNGRKTTSTYDELGRATGVWTPSRPVNQDASVRFVYQIEDSKVPAVHTRVLRDAGTYEESIELYDGFLRPRQTQREALGGGRIVTETLYNANGSAKEVRDGYLAEGEPARELFVPLSLDQVPSATRTAYDGLGRPVRTTTLHRGVPRHSATTAYGGDWELSRTGMSPDGTTPLSGSRAVKATTDALGRPARIQHFTTQNVSAESVDTTYTYDPRGPLAQVTDAQQNTWTYTYDARGRKTSSTDPDAGAAYFGYNALDQQVWSKDNQGRLQYTTYDVLGRQTELRDDSASGPLVAKWTFDTLPGAKGHPVASTRYNDGAAFTSEVTGYDTEYRPTGNKVTIPSTPMTTGLAGTYTYASTYTPTGKVQSVDLPATPGGLAAEKVITRYDGEDSPTTMSGLAWYTADTFLGPYGEVLRTASGEAPRRVWTTNVYDEDTRRLTRTTAHRETAPHPVSTTTYGYDTVGNITSIADQQPAGTEEQCFSYDPMGRLVHAWTDGNSAVCPRTSTAPGAGPARADVSAGVDGGGYWHSYAFDAIGNRTKLTVHDRTDAALDDTYTYTYGKTLPGNPQPVQPHTLTQVDAVLNEPGSRVEPRSTYAYDTSGNTTQRVIGGDTQTLAWDRRNKLTSVDTNNDGTPDVKYLYDASGNRLVEDDGTTRTLFLGEAEIVVNTAGQAVDARRYYSSPGAPTTIRTTGGKTTGHKLTVMLSDHHSTATTAVELTDTQPVTRRRFDPYGNPRGTEPTTWPDRRTYLGVGIDDPATGLTHIGAREYDASTGRFISVDPVMDLTDPLQMNGYTYANADPINNSDPTGLLLDARGGGTQKCVGTCVKDVTNRKGIPLPPGEEWKHEGEAQTDFNGDGFITVFPTVNVPAKWKKAKKYTEAFYKAVDTACFYGRESCADPEYPSRAHSINNWKGKACKAVGGKCPERLSWGEGPAFAGGFAIAAEEYAGRGGYRGGGARRGSPCKCFLAGTEVLMADGSTKSIEDIKLGDEVVATDPVTGEAGAHPVSALIATENDKRFNELVIITSEGVERLTATHEHPFWSPSEGEWLEAGELRTGMTLRSDSGETLVVAGNRAFTQRARTYNLTVADLHTYYVLAGQTPVLVHNANCGPHLKDLQKDYPRRTVGILDVGTDQLPMISGPGGQSGLLKNLPGRTKANGEHVETHAAAFLRMNPGVRKAVLYIDYPTGTCGTCRSTLPDMLPEGVQLWVISPRRTEKFTGLPD

SEQ ID NO: 87 - protein, 1885 aa, Propionibacterium acidifaciens
MVDLGAYEEPVAFDDGVADALRSAASALSGTLSGQAASRSSWAATASTDFEGHYADVFDANARAACDDCSNIASALDALAADVQTMKDAAASERDRRRQAKEWADRQKDEWAPKSWIDDHLGLDKPPAGPPETPVVDAQAPTVATWSEPAQGQAGGVSSARPDDLRTYSSNVTGANDTVTTQKGTLDGALSDFADRCSWCSIDTSGITTALAAFGANNTNETRWVDTVAAAFEAAGGSGAISAVSDAALDASLQAAGVTQSRQPVDVTAPTIQGDPQTSGYADDPVNTTTGNFIEPETDLAFSGGCASLGFDRVYNSLSAGVGAFGPGWASTADQRLLVTEDGAVWVQPSGRHVVFPRLGNGWDRAHNDTYWLHTTTDTTGPTPGDAPTTGAAGGAGVFVVSDNAGGRWVFDRAGRPVSVSRGPGTRVDHRWDGDRLVGLTHERGRAVTIEWNDHHTRITALTANDGRRVDYGYDPAGRLTEAASAGGTRTYGWNEAGLIATVTDPDGVVEAANTYNEHGQVTSQRSRFGRLSHYTYLPGGVTQVADEDGGRANTWIHDQTGRLVGMVDADGNRQSIGWDQWGNRVQITGRDGRTTVCRYDARGRLITRQEASGARTDYEWDEADRVVQVTVTDTTSSSHGNTSSAGGSGPSVTSYEYEGAGRNPSTVTDPEGGVTRLTWDQNLLTEATDPAGVRVRLGYDGHGDLVSTTNAAGDTARLVRDGAGRVVAAITPLGHRTEYRYDEAGRLASRQDPDGALWRYEHTTGGRLSAVVDPDGGRTVTEYGPGGVEEATTDPLGRRLEQEWDDLGNLAGVRLPDGREWSYVHDGLSRLTETVDPAGGLWRREYDVNGMVAATVDPTGVRRGLAWAADGSVTVSDASGTARVGVDGLGRPVSVSVSSAPAPGEAVPMGMSLEETVGTGAPAPGGAGPDGPDARVVVRDLCGRPVEALDADGGLTRLMRDAAGRLVEEISPAGRSTRYEWDRCGRLSAVIGPDGARTTMAYDAASRLIAQDGPGGRVRVAYDRCGRLSTVTAPGRGKTTWGYDRAGRVRSVRSPAWGLVRFGYDPAGQLTAVTNALGGVTRYDYDECGRLVQVTDPLGHVTRRTYTAADRVETLVDPLGRTTQAGYDAAGRQLWQTDDTGERLAFGWDEAGRLERVATGGEGLPGQTCCALTRPGRRVLRVTGPGGARDELVFDRLGRLARHARGGRTVGEWSWDPDGACTAFTGPDGQRVRYAYDDAGALVRVEGTAFGPVTVRRDTAGRLTGMDGPGLTQRWDRDETGHVIAYRRTKNGVTTSSRVSRDESGRVTAVDGPDGTVRYGYDPAGQLARIEGPDGRRESFTWDKAGHLTRRSVERPGARPETTLYSYDPAGQLASTDGPDGRTLYTWDAAGRRTGQDGPDGHWSYSWAPSGHLTAVTRRTPHDARTWRISRDGLGLPRRIDDTDLAWDLSGPVPALTRFGTHTVTGLPRALAIDGTLTSTGWRPARPTSADDPWAPPPPVVETDGARLGVGGAVGLGGLEILGARVHDPTTFSFLSPDPLDQPPLAPWATNPYSYAANNPLAFTDPTGLRPLTDTDFEAYKHDHGGLGGWIADHKDYLIGGAMVIAGGVLMATGVGGPLGGMLIGAGADTIIQRATTGQVDYGQVAVSGLLGAAGGGAASALLKGGGRLATELGATGLRTAITTGAASGTASGAGGSGYGYLTGPGPHTVSGFLTSTATGAVEGGLLGGASGAAGHGLSTTGKNVLGHFEPTPTTPQGTSSDTIAEMLNSASQPGRTAGVLDIDGELTPLTSGRPSLPNYIASGHVEGQAAMIMRQQQVQSATVYHDNPNGTCGYCYSQLPTLLPEGAALDVVPPAGTVPPSNRWHNGGPSFIGNSSEPKPWPR

SEQ ID NO: 88 - protein, 125 aa, Unknown
  source: /note=“Description of Unknown Lachnospiraceae bacterium sequence”
MSLPEYDGTTTHGVLVLDDGTQIGFTSGNGDPRYTNYRNNGHVEQKSALYMRENNISNATVYHNNTNGTCGYCNTMTATFLPEGATLTVVPPENAVANNSRAIDYVKTYTGTSNDPKISPRYKGN

SEQ ID NO: 89 - protein, 251 aa, Ruminococcus bicirculans
MLAIEKIKSGDKVISTDPETMETSPKTVLETYIREVTTLVHLTVNGEEIVTTVDHPFYVKNQGFIKAGELIVGDELLDSNCNVLLVENHSVELTDEPVTVYNFQVEDFHTYHVGKCRLLVHNANCNQEKPVLPKYDGKTTEGVMVTPDGKQISFKSGNSSTPSYPQYKAQSASHVEGKAALYMRENGINEATVFHNNPNGTCGFCDRQVPALLPKGAKLTVVPPSNSVANNVRAIPVPKTYIGNSTVPKIK

SEQ ID NO: 90 - protein, 2379 aa, Streptomyces cavourensis
MSSSVSGRAFRVSGVLTRITKSWTPGSARRSSASVRHRGRAVRARSLGVTLSAVLAATLLPAEAWAIAPPAPRIGPSLVDLQQEEPADPDQAKIDELSTWSGAPVEPPADYTPTATTPPAGGTAPVALDGAGDDLVPVGNLPVRLGKASPTDEEPDPPAPGGTWDVAVEPRTSTEASDVDGALITVTPPSGGATPVDIELDYGKFEDLFGTAWSSRLRLTQLPECFLTTPELDECTTVVDVPSVNDPSNDTVRATIDPAASPQQGLSTQSGGGPVVLAATDSASGAGGTYKATPFTATGTWTAGGSGGGFSWSYPLTAPAPPAGPAPTISLSYSSQSVDGRTSVANGQASWIGDGWDYNPGFIERRYRSCNDDRSGTPNNAGGKDKKKSDLCWASDNLVMSLGGSATALVHDGTTGAWVAQSDTGARIEYRTRTGSPKTAQTGAYDGEYWVVTTRDGTRYWFGRNTIPGRTAATESALTVPVFGNHSGEPCHATAYADSSCAQAWRWNLDYVEDVHGNAMIVDWKKETNRYARNEKFKEAVAYHRGGYPAQILYGLRADDLNGAPAGKVVFKTAPRCVEDAGTTCSPTGYESDNYADKQPWWDTPATLHCKSGAKNCFVTSPTFWSSVRLTEIETHGRRTPGSTALSLVDSWTLKQSFPKQRTDTHPPLWLESITRTGHGAPNASGEQTSRALPPVSFLPNVVDMPNRVSKGATDETPDFDRLRVETVRTETGGEIHVDYSAPCAVGTAHPSPETNTTRCFPVHYSPDPEALSDEVLAKKPAPVEWFNKYVVQKVTEKDRVARQPDVVTTYAYEGGGAWGRSTDEFTKPKLRTYDQWRGYASVLVRKGVTGADPAAADATEQSQTRMRYFRGMSGDAGRPTVTVKDSTGAETLGEDLAPYQGMPAETVAYTRAGGDVASRILAWPTSRETASQARPGLPALKAHRVATARTETVETISGGRTRTARTVTTYDDTYGLPLTAETLTLTPDGSGGTTTGDRSCSTNTYVHNTAKHLIGLVQRARTTVGTCAQAATASGSDVVSDTRVSYDALDAFGAAPVRGLPFRTDTVGADGTGWVTSARTEYDPLGRATEVRDAKGHVSKVGFVPPTGPAFTTTSTDAKGHTTTTALDPARGTALSVTDANGRRTTSAYDELGRTTAVWSPSRTQGTDKASVLFDYQIEDNKVPATRTRVLRDNGTYEDSVTVYDGLLRPRQAQTEALGGGRIVTETLYNANGAPAETRNGYLAEGEPQTELFVPLSLTQVPSASKTAYDGLGRAVRTTVLHAGDPQHSATVRHEGDRTLTRTGMSADGTTPMPGSRSTATWTDALGRTSKIEHFTATDLSAAIDTRYTYDARGNLAKVTDARDNIWTYTYDARGRLTFSTDPDAGSSSFGYDVLDRQIWSKDSRQRSQHTVYDELGRRTELRDDSAEGPLVAKWTYDTLPGAKGLPVASTRYHEGAEFTSEVTGYDQEYRPTGSRTTIPSTPLTTGLAGTYTYKNTYTPTGLPQSVELPATPGGLAAEKVITRYDGEGSPRTTSGLAWYTVDTVLSPLGQVLRTASGEAPNRVWATHFYDESTGRLDRRITDRETLDPSRISETSYAHDTVGNITSITDTQSPARVDRQCFAYDPMGRLAHAWTAKSPGCPRSSTAQGAGPNRTDVSPSIDGAGYWHSYEFDTIGNRTGMVVHDPADPALDDTYVYTHGVPSEGPLQPATLQPHTLTKVDATVRGPGSTVTSSSTYAYDPSGNTTQRVIGGDTQALTWDRRNKLMSADTDDDGTADVTYLYDASGNRLLEADATTRTLYLGESEIVVDTAGRPVEARRYYSHPGAPTTLRTTGGRTSGHTLTVQLTDHHNTPTASVALTGGQPVTRRMFDPYGNPRGTEPTTWPDRRTYLGVGIDDETTGLTHIGAREYDSVTGRFISADPIIDIADPLQMNGYAYANNNPVTNWDPTGLKSDECGSLYRCGGNQVITTKTTKYQDVNTVARHFEKTASWATLAQWKAEGLGKSPAFGKAKKLTKWKNEHYEKNWTINLVPGMARSWVSGVDAAASAIMPFPTVQAAPLYDSLVSSLGVNTKGRAYANGEGLMDGLSMVGGVGAIAPGIKSGLKAAAKGCGPGNSFTPGTEVALADGTTKPIEDIKIGDEVLATDPETGETRAEKVTAEIRGDGTKNLVKVTIDTDGDRGTDTAEITATDGHPFWVPELGRWIDATDLAPGQWLRTSAGTHVQITAIKRWTETATVHNLTVADLHTYYVLAGKTPVLVHNENCGPNLKDLPKDYDRRTVGILDVGTDQLPMISGPGGQSGLLKNLPGRTKANTDHVEAHTAAFLRMNPGIRKAVLYIDYPTGTCGTCGSTLPDMLPEGVQLWVISPRKTEKFAGLPD

SEQ ID NO: 91 - protein, 2409 aa, Roseburia intestinalis
MKLTYKELEIELELAGLLAVEELVLTQGLNCHAGLTLKILIEEEQRDELVTMSSDAGVTVRELEKTNGQVVFRGKLETVSARRENGLFYLYLEAWSYTMDWDRVKKSRSFQNGALTYMEVVQRVLSGYGQSGVTDHATGGACIPEFLLQYEESDWVFLRRLASHFGTYLLADATDACGKVYFGVPEISYGTVLDRQGYTMEKDMLHYARVLEKEGVLSQEASCWNVTVRFFLRMWETLTFNGIEAVVTAMRLHTEKGELVYSYVLARRAGIRREKEKNPGIFGMSIPATVMERSGNRIRVHFEIDPEYEASEKTKYFTYAIESSSFYCMPEEGSQVHIYFPDHDEQGAVAVHAIRSGEGASGSCSTPENKRFSDPSGSAMDMTPASLQFAPDAGGATVLHLEGGGFLSLTGMDIKLKTQMGMASDKEKPMQDLMICGEQKLTMQIGESSDDCIVMEAGTEVRSALVVQEADSSPAAVPSGDELLSEQEAADAQAREAENNAVKEDMITKKQESKRKIVDGVISLVTVVGLTALTVATGGLAAPFAIAAGVKATFAVADIAEGLDGYSKMNAMDASRPANFLRDTVFGGNQTAYDITSMITDVAFDVVSGKALVGAFSGADKVSKVQKFAGKAMSFWNGICPKTKVANFLFQMGGTMLFGAVNDYLTTGKVDLKNLGLDAFAGLAKGTLGTAGTEKIKRLLNTDNKWVEKAVGILAGTTFGTTVDLGINKLAGRDVDLLQVIKQNLIESGLGQFFGEPIDVVTGAFLITATDFTLPDIREDLRVQRKYNSTSREAGLLGPGWSFSYECRLYCSGNRLHAKLDSGITAVFAWDGSHAVNVTRGCEWLELTGEDDGWRIYDGRNYKCYHYDGQGLLTAAEDRNGQCVRLYYEGERLTRITTPLGYSLDVEIRDGRLVQIRDHMGRTMQYRYENGFLSDVIHMDEGVTHYEYDSNGYLERAVDQAKVTYLENRYDDAGRVVLQTLANGDTYRADYHPEKNRVTIVSSVHDKAVEHWYNEFGEILETSYQDGTKERYEYGENGHRTSRTDRLGRKTTWTYDEAGRLTEEVQPDGLRTVHRYDAAGNEILRTDSAGRETAFEYDGHHNRTAERRTDGLQVRENRSVYDWMGRLTETADAEGNRTQYQYGEAGGKPSVIRFADGETCSFEYDKAGRMMAQEDACGRTEYGYNARNKRALVRDGEGNETRWMYDGMGRLLALYLPKAWKEQHGEYSYSYDFLDRLIHTKNPDGGHERLMRDGEGNVLKRVHPNAYDSCRDDGEGTTYDYDSDGNNIRIHYPDGGCERIFYDSEGNRIRHVMPESYDPQTDDGEGFTYTYDACSRLTGVTGPDGVRQASYTYDPAGNLTEETDAEGRCTYRSYTAFGELKEQLKPALEKDGVMLYERITWQYDRCGNVLLEQRHGGYWDSNGVLVKEDGAGLALRFTYDSRNRRIRVEDGLGAVISCHYDVQGKLVYEEKAVSGEVRQVIHYGYDRAGRLTERKEELDSGLAPLEGEPRYAVTRYRYDGNGNRTGIVTPEGYRILRSYDACDRLVSERVVDDKNGIDRTTSVTYDYAGNITRIVRSGKGLGEWEQGYGYDLKDRIVHVKDCLGPVFSYEYDKNDRRIAETLPQTGMTENGKSGYPKNQNRYRYDVYGRLLTRTDGSGTVQEENRYLPDGRLALSREADGQEIRYAYGAHGREEETGTARSRKAGRAAQKYRYDSRGRITGVVNGNGNETGYDLDAWGRIQNIRQADGGEEGYTYDFAGNVTGTRDANGGVITYRYNSQGKVCEITDQEGNSETFRYDREGRMVLHVDRNGSEVRTTYNVDGNPVLETGTDRNGENRVTRSFEYDASGNVRKAVAGGFCYTYEYRPDGKLLKKSASGRTILSCSYHADGSLESLTDASGKPVFYEYDWRGNLSGVRDENGDMLAAYAHTPGGKLKEICHGNGLCTRYEYDTDGNMIHLHFQRENGETISDLWYEYDLNGNRTLKTGKCILSGDSLTDLAVSYRYDSMDRLTSESRDGEETAYSYDFCGNRLKKLDKSGTEEYHYNRKNQLICRFSEKEKTAYRYDLQGNLLEAAGAEGTEVFSYNAFQQQTAVTMPDGKHLENRYDAEYLRAGTVENGTVTSFSYHNGELLAESSPEGDTISRYIPGYGVAAGWNREKSGYHYYHLDEQNSTAYITGGSCEIENRYEYDAFGVLKNSMEEFHNRILYTGQQYDQTSGQYYLRARFYNPVIGRFVQEDEYRGDGLNLYAYCKNNPVVYYDPSGYDSQYPCKEEMSAGAGESGRKTISLPEYDGTTTHGVLVLDDGTQIGFTSGNGDPRYTNYRNNGHVEQKSALYMRENNISNATVYHNNTNGTCGYCNTMTATFLPEGATLTVVPPENAVANNSRAIDYVKTYTGTSNDPKISPRYKGN

SEQ ID NO: 92 - protein, 154 aa, Streptomyces rubrolavendulae
VAGNRAFTQRARTYNLTVADLHTYYVLAGQTPVLVHNANCGPHLKDLQKDYPRRTVGILDVGTDQLPMISGPGGQSGLLKNLPGRTKANGEHVETHAAAFLRMNPGVRKAVLYIDYPTGTCGTCRSTLPDMLPEGVQLWVISPRRTEKFTGLPD

SEQ ID NO: 93 - protein, 74 aa, Streptomyces rubrolavendulae
VAGNRAFTQRARTYNLTVADLHTYYVLAGQTPVLVHNANCGPHLKDLQKDYPRRTVGILDVGTDQLPMISGPGG

SEQ ID NO: 94 - protein, 80 aa, Streptomyces rubrolavendulae
QSGLLKNLPGRTKANGEHVETHAAAFLRMNPGVRKAVLYIDYPTGTCGTCRSTLPDMLPEGVQLWVISPRRTEKFTGLPD

SEQ ID NO: 95 - protein, 147 aa, Propionibacterium acidifaciens
LSTTGKNVLGHFEPTPTTPQGTSSDTIAEMLNSASQPGRTAGVLDIDGELTPLTSGRPSLPNYIASGHVEGQAAMIMRQQQVQSATVYHDNPNGTCGYCYSQLPTLLPEGAALDVVPPAGTVPPSNRWHNGGPSFIGNSSEPKPWPR

SEQ ID NO: 96 - protein, 56 aa, Propionibacterium acidifaciens
LSTTGKNVLGHFEPTPTTPQGTSSDTIAEMLNSASQPGRTAGVLDIDGELTPLTSG

SEQ ID NO: 97 - protein, 91 aa, Propionibacterium acidifaciens
RPSLPNYIASGHVEGQAAMIMRQQQVQSATVYHDNPNGTCGYCYSQLPTLLPEGAALDVVPPAGTVPPSNRWHNGGPSFIGNSSEPKPWPR

SEQ ID NO: 98 - protein, 125 aa, Unknown; identical to SEQ ID NO: 88
  source: /note=“Description of Unknown Lachnospiraceae bacterium sequence”

SEQ ID NO: 99 - protein, 30 aa, Unknown
  source: /note=“Description of Unknown Lachnospiraceae bacterium sequence”
MSLPEYDGTTTHGVLVLDDGTQIGFTSGNG

SEQ ID NO: 100 - protein, 95 aa, Unknown
  source: /note=“Description of Unknown Lachnospiraceae bacterium sequence”
DPRYTNYRNNGHVEQKSALYMRENNISNATVYHNNTNGTCGYCNTMTATFLPEGATLTVVPPENAVANNSRAIDYVKTYTGTSNDPKISPRYKGN

SEQ ID NO: 101 - protein, 143 aa, Ruminococcus bicirculans
HTYHVGKCRLLVHNANCNQEKPVLPKYDGKTTEGVMVTPDGKQISFKSGNSSTPSYPQYKAQSASHVEGKAALYMRENGINEATVFHNNPNGTCGFCDRQVPALLPKGAKLTVVPPSNSVANNVRAIPVPKTYIGNSTVPKIK

SEQ ID NO: 102 - protein, 53 aa, Ruminococcus bicirculans
HTYHVGKCRLLVHNANCNQEKPVLPKYDGKTTEGVMVTPDGKQISFKSGNSST

SEQ ID NO: 103 - protein, 90 aa, Ruminococcus bicirculans
PSYPQYKAQSASHVEGKAALYMRENGINEATVFHNNPNGTCGFCDRQVPALLPKGAKLTVVPPSNSVANNVRAIPVPKTYIGNSTVPKIK

SEQ ID NO: 104 - protein, 156 aa, Streptomyces cavourensis
VQITAIKRWTETATVHNLTVADLHTYYVLAGKTPVLVHNENCGPNLKDLPKDYDRRTVGILDVGTDQLPMISGPGGQSGLLKNLPGRTKANTDHVEAHTAAFLRMNPGIRKAVLYIDYPTGTCGTCGSTLPDMLPEGVQLWVISPRKTEKFAGLPD

SEQ ID NO: 105 - protein, 76 aa, Streptomyces cavourensis
VQITAIKRWTETATVHNLTVADLHTYYVLAGKTPVLVHNENCGPNLKDLPKDYDRRTVGILDVGTDQLPMISGPGG

SEQ ID NO: 106 - protein, 80 aa, Streptomyces cavourensis
QSGLLKNLPGRTKANTDHVEAHTAAFLRMNPGIRKAVLYIDYPTGTCGTCGSTLPDMLPEGVQLWVISPRKTEKFAGLPD

SEQ ID NO: 107 - protein, 136 aa, Roseburia intestinalis
SAGAGESGRKTISLPEYDGTTTHGVLVLDDGTQIGFTSGNGDPRYTNYRNNGHVEQKSALYMRENNISNATVYHNNTNGTCGYCNTMTATFLPEGATLTVVPPENAVANNSRAIDYVKTYTGTSNDPKISPRYKGN

SEQ ID NO: 108 - protein, 40 aa, Roseburia intestinalis
SAGAGESGRKTISLPEYDGTTTHGVLVLDDGTQIGFTSGN

SEQ ID NO: 109 - protein, 96 aa, Roseburia intestinalis
GDPRYTNYRNNGHVEQKSALYMRENNISNATVYHNNTNGTCGYCNTMTATFLPEGATLTVVPPENAVANNSRAIDYVKTYTGTSNDPKISPRYKGN

SEQ ID NO: 110 - protein, 21 aa, Artificial Sequence
  source: /note=“Description of Artificial Sequence Synthetic peptide”
LRGSQLVKSKSEAAARGGGGS

SEQ ID NO: 111 - protein, 18 aa, Artificial Sequence
  source: /note=“Description of Artificial Sequence Synthetic peptide”
LRGSQLVKSKSEAAARGS

SEQ ID NO: 112 - protein, 13 aa, Artificial Sequence
  source: /note=“Description of Artificial Sequence Synthetic peptide”
LRGSQLVKSKSGS

SEQ ID NO: 113 - protein, 11 aa, Artificial Sequence
  source: /note=“Description of Artificial Sequence Synthetic peptide”
LRGSQLVKSGS

SEQ ID NO: 114 - protein, 9 aa, Artificial Sequence
  source: /note=“Description of Artificial Sequence Synthetic peptide”
LRGSQLVGS

SEQ ID NO: 115 - protein, 6 aa, Artificial Sequence
  source: /note=“Description of Artificial Sequence Synthetic peptide”
LRGSGS

SEQ ID NO: 116 - protein, 4 aa, Artificial Sequence
  source: /note=“Description of Artificial Sequence Synthetic peptide”
LRGS

SEQ ID NO: 117 - protein, 115 aa, Plantactinospora sp.
MGDRLPAFVDGGDTLGIFSRGGIERDLASGVAGPASSLPKGTPGFNGLVKSHVEGHAAALMRQNGIPNAELYINRVPCGSGNGCAAMLPHMLPEGATLRVYGPNGYDRTFTGLPD

SEQ ID NO: 118 - protein, 2198 aa, Streptomyces massasporeus
MSSRPFRKRLPGAVVRRWLGRGAVVASLSLLPQVVVPSGYDFAAQAQSVAARKKLEDRPEAKINKVGVLRPGTSKAPKDKSAPASRKTRERLQEASWPKSGKATAAVTATSEATVNVGGLGMELTQEPAAPAAKSAKSTTKRKATGPAEKVTLRVHSRATAKKAGVNGVLLTVDPARGESNEKAEDTDKLRISLDYSSFSDVYGGNFGPRLSLVKLPACALTTPEKKSCRTQTPVAGADNEAESQTLTGTVPARNLKAGTPMLLAAAADSSGGGGDFSATPLSPTATWEAGGSTGDFTWDYPLRVPPATAGPSPNLSISYNSASVDGRTAGENNQTSLIGEGFSITESYIERKYASCKDDGQSGKGDLCWKYANATLVLNGKAVELVNACADKSACDTAALSEASGGTWKVKNEDGTRVEHLTGASGNGDNNGEYWKVTDASGIQYYFGKHRMPGWSDKGTTDTADDDPSTYSTWAVPVFGDDSGEPCYKSSGFADSSCNQAWRWNLDYVVDTHDNASTYWYSKETNYYSKNADTTVNGTAYTRGGYLNRIDYGLRSDLIYSKPAAQQVRFTYGQRCIVTNGCSSLTKDTKANWPDVPYDMICAANTKCTTQIGPSFFTRQRLIDITTSVWTGTGTTRRDVDTWHLSHDFPDTGDASSPSLWLKSIQNTGKANTTTAAMPPIVFGGIQMPNHVEGSGQDNLRYIKWRVRTIKSETGSTLTVNYSDPDCIWGSSMPSAVDKNTRRCFPVKWSQSGTTPVTDWFHKYVVTSVLQDDPYGHSDTGETYYDYQGGAGWAYSDDEGLTKPSNRTWSQWRGYGKVVTTSGNSEGPRSKKSTLYMRGLNGEKELDGTARVAKVTDSTGTAIDDSRQYAGFVRETIAYNGSDELSGTINTPWSHKTGSHTYSWGTTEAWIVQAGETESRTKISTGTRTVKQKTTYDTTYGMPITVEDSGDATKFGDESCVRTSYARNTSAWLVNRVSRTETYSVPCATIPAIPADVVSDITTAYDAKAVGAAPTQGDITATYRVASYNAADKTPVYQQVSSSTFDKLGRPLTETNALDRTVKTSYVPDDTGYGPLTSKTTTDPKLYTSTTEVDPAWGAASKTTDANGNVTEWSFDALGRLRSVWKPDRSRTLDDAASIVYAYSVNNDKETWVRTDALKADGKTYNSSYEIFDSLLRPRQKQVPAPNGGRVISEMLYDDRGLAYIANSQVHDNSAPSGTLANTYTGSVPASTETVFDAAGRATDSIFRVYGQEKWRTKTDQQGDRTAVTAAAGGTGTLTIVDARGRVTERREFGGPAPTGTDYTRTLYEYTPGGQIKKMTGPDGAVWTYEYDLRGRKTTSTDPDKGSITTTYNDADQPLTATTTLDNVSRTLINDYDELGRPTGTWDGTKDNAHQLTKFTYDSLAKGQPTASIRYVGGTTGKIYSQSVTGYDALNRPKGTKTVIAATDPLVTAGAPQTFTTSTAYNIDGTVQSTSLPAAAGLPAETVKNTYNSLGMLTGTDGMTDYVQHIGYSPYGEIEETRLGTSTEAKQLQVLNRYEDGTRRLANTHTLDQTNAGYTSDVDYVYDATGNVKSVTDKANGKDTQCFAYDGYRRLTEAWTPSSNDCATARSASALGGPAPYWTSWTYKPGGLRDSQTEHKTSGDTKTVYGYPAVNTSGTGQPHTLTSVTVGSGSAKTYTYDEHGNTTKRYSPTGTAQSLTWNIEGELTRLTEGTKTTDYLYDANGELLIRRSPDKTVLYLGGQELHYDTATEKFTAQRYYPAGDATAVRTETGLSWMVDDHHGTASMTVDATTQAVTRRYTKPFGEARGTAPSVWPDDKGFLGKPADTGTGLTHIGAREYDPTLGRFLSVDPVLAPDDHESLNGYAYANNTPVTLSDPTGLRPDGMCGGSSSSCGGGTETWTLNSKGGWDWSYTKTYTKKFTYRTGNGGTRTGTMTTTVRTEVGHKAVRIVFKKGPEPKPAKKDGQCSSCWAMGTNPGYSPGATDDWIDRPKLETWQKVVLGAISVVAAGVILAPAAIVVGEGCLAAAPVCAAEIAEAATGGASGGSAVVGAGVVATGAKAVTTGKSLSESQATLSVAQRLLATIGEEGKTAGVLELDGELIPLVSGKSSLPNYAASGHVEGQAALIMRDRGATSGRLLIDNPSGICGYCKSQVATLLPENATLQVGTPLGTVTPSSRWSASRTFTGNDRDPKPWPR

SEQ ID NO: 119 - protein, 2202 aa, Streptomyces sp.
MAFGIGTSRRGSGGGRGWGRRLVTPVAALALLAPLGEAQDAVAQDAGAVRSGPVQPDVPKPRVSKVKEVKGLGAKKARDRVAAGKKAGAAQAARARREQTAVWPGPDTASIELADDRRAKAELGGASVSVVPENGRKTAASGTAQVTILDQKAADKAGVTGVLLSATADTAGTAEVSVDYSGFASAFGGDWAQRLHLVQLPACVLTTPEKAVCRRQTPLKTDNNASEQSVAAQVALAKAEPGAPSAQSVASAEGPSATVLAVTAAAAGSGASPKGTGDYAATELSPSSAWEAGGSSGAFTWNYGFTVPPAAAGPTPPLALSYDSGSIDGRTATTNNQGSAVGEGFSLTESYIERSYGSCDKDGHADVWDHCWKYDNASIVLNGKSNRLIKDDTSGKWRLEDDDASTVTRSTGADNGDDNGEYWTVTTGDGTKYVFGENKLDGAADQRTNSTWTVPVFGDDSGEPGYDKGDTFAERAVTQAWRWNLDYVEDTSGNASTYWYAKDSNYYPKNKATTANASYTRGGYLKEIRYGLRKDALFTDDADAKVVFAHAERCTVGSCTTLTKDTAKNWPDVPFDAICSSGDSECNAAGPSFFSRKRLTGISTFSWNAASKAFDPVDTWELTQDYYDAGDIGDTTDHVLVLESIKRTAKAGATAIDVNPVTFTYQLRPNRVDGTDDILPLKRHRIETITSETGSITTVTLSQPECKRSTVLDAPQDSNTRPCYPQFWNINGATKASVDWFHKYRVLAVAVDDPTGHNESIEHAYDYAGAAWHYSDDPFTTKNERTWSEWRGYRDVTTYTGALDTTRSKSVSRYMLGMDGDKNTDGTTKSVSTAPLMDTDVDFAALTDSDPYSGQLLQQVTYSGSQPISTSYTNFTHKNTASQTVPDATDHTARWVRPNSSYASTYLTASKTWRTQVTTSRYDDLGMVTSHDDYGQKGLSGDEICTRTWYARNTEAGINSLVSRTRTVGKECSVDDTALDLPADNKRSGDVLSDTATAYDGATWSDSMKPTKGLVTWTGRAKGYASGTPSWQTLTSAAPADFDVLGRPLKVTNAEGQPTTTAYTPVTAGPVTKIISGNPKGFKTTSFLDPRTGQELRTYDANLKKTERVYDALGRLTQVWLPNRDRGSESATFGPSVKFEYTIDNNDPSWVSTAALKKDGKTYATSYAIYDAMLRPLQSQTETSNGGRLLTDTRYDTRGLPYETYANIFDTTSTPNGTYTRAEYGEAPNQNATVFDGAGRPTKSTLLVFGVEKWSTTTSYTGDSTATTALDGGTASRAITNIRGHTVESREYAGKSPADAQYGDGLGVGFASTRTLYTRGGLQKQITGPDDATWSYTYDLFGRQVEAEDPDKGTSSTEYDVLDRATKSTDSRSKSILTAYDELGRMVGTWAGSKTDTNQRTEYTYDKLLKGQPDKSIRYVGGKAGQAYTDTITEYDSLSRPVAASLELPADDPFAKVGALGSASRTLSFRHAYNLDDTVKTAEEPALGGLPSEIIDYGYNNVGQVTSVGGSTGYLLGATYSPLGQPWEQLLGTANTADHKKVSIRNTFEDGTGRLTRSNVKADSQPYMLQDLNYSFDQVGNVTSITDPTTLGGTSSAETQCFTYDSHRRLTEAWTPSQQKCSDPRSTSSLSGPAPYWTSYTYNTAGQRTTETTRKAAGDTTTTYCYTKTDQPHFLTGTTTKGDCATRERTYTPDTTGNTTKRPGASTTQDLAWSEEGKLTKLTENGKATDYLYDATGELLIRNTTSGERVLYTGTTELHLRTDGTTWAQRYYAAGDQTVAMRSNESGTNKLTYLAGDHHGTSSLAISADSTQTVSKRYMTPFGAERGKPTGTAWPDDKGFLSKTTDKTTGLTHIGAREYDPAIGQFISTDPILDPAQPQSLNGYSYANNTPVTAADPSGLWCDSCNDGKGWTRPDGGTRGDENGGKNPDGSVRGTPGFPSTRPTTVGYGNSPGAGKVITDLGSGTPALPPPDVYQDYQPKLPGVGQMGRNGTYMPELSYELNVELYFRERCSFSWTEECESIRAFYTHGEDSHGLPRYWTDVQDIPTVNTCPICENIGFDIILATLPIGKVGKLRFAPKVESAESMLRSLSQEGKTAGVLDINGELIPLVSGTSSLKNYAASGHVEGQAALIMRERGVASARLIIDNPSGICGYCRSQVPTLLPAGATLEVTTPRGTVPPTARWSNGKTFVGNENDPKPWPR

SEQ ID NO: 120 - protein, 1758 aa, Jiangella alba
MRGWVRAVSIPVIVGVLSTALSMPPSFADQEPVARTEATTDGLPTNADEGQRAEPPALIPSENRIPGVGLKSEIESQPTAASVADGPLPSERSDSFFPALAPTPPTIVGYVPTSLAPGCAEWGALRWTHPDSRPNGLVHLYTFELYRDSDDAMVWDQLFDYTLTGAGVVSDVAGDCESILPDPQATPIVELGESYYAKVYAWDGTGWSAPATSSAYPAVALPGLTDEAARGVCVCDTSTGRLYPLNILRADPVNTATGTLTESATDLTIPGVGPAISASRTYNSTDPTVGPLGKGWSFPYFSELESAASSVTYKAEDGQEVEYALQGGAYRLPPGASTRLRSVSGGYQLETKSHQVIGFDQNGRLEYARDSSGQGVSLAYATNGTLDKITDASGREVDVTMDASGKVTAIALSDGRSVSYGYTGDLLTSVTDVRGGVTEYEYDAAGRLAAITDPLGNEVMRSTYDAQGRVISQVDAGGGTWGFEYVDDGAYQTTRTTDPRGGVSRDVYYNNVLVESETAGGAITTYQYDERLRLAATVDPHGRTTRHTYDANDNLLSTTHPNGDREAFTYSSGGDLLTETSPEGRKTTYTYDANHRVATTTDPNGGVTSYTYNTDGQVLTETSPEGNVTEFEYDAQGNRVATISPEGRRTTATFDAYGRLESQTTARGHVAGADPADFTTTFAYDVASNLTSSTDPLGHVTEYEYDLNNRRTTVIDPLDRRTETEFDAAGRVVKIIEPGGAETVHEYDLAGNQVATTDAEGGRTTRTFDLDAHMITMTAARGNEPGAEPADFTWGYEYDGLGNVVEETDSAGGIVSYGYDERYRQTSVTNQANETTTTAYDGDGNTVSVTDPLDRTVSTTYNGLNLPATVTDPAGKVSTVIYDRDGNRTSTTTPLGHKATFTYDGDGMLVQDQTPNGNGRISTYTYDADGNQIRTVDPQGRFTTATFDNAGRVSSRSLWNVTTTYGYDDAGRLTTVTGGDGAVTEYGYNTAGDLVTVTDPNDHVTTHTYDDAHRRTATTDALNRTRTFGYDADGNQTSTVLARGPASGDLARWTVTQSYDELGRRTGVTTGSTASTASYAYDPVGRLTGVTDAGGTTTTVYDDAGQIASVTRGSQAYGYTYDPRGMVKTITQPGGVTVTNTFDDDGRLATTASTNAGTTAFSYDKNNNLTRIDNLAATGLVNRWQQRNYDRADALVSTTTGTGTTTDPTQTVTYSRDGAGRPFVIRRGAGGTQAPGEAHFFDAAGRLAQVCYDASSMFGQNCATADETLAYTYDGAGNRLTETRTGGTTPGTTTYTYDAANQLTQRGNTTYSYDADGNQISDGATSWTYDELNRLVGIDTPTADSQLTYDGLGNRTSVTTGATTRTFSWDINNPLPLLTSVTQGTSTTRYRYGPDAIPVNANINGTNHALLTEDLNSLTTTYNRTTGAKSWTTTYEPFGTPRNTTSTGLTTAQVGLGYTGEYLDPTTGLLNLRARNYNPTLGQFTSTDPVETPQGTPSISPYAYVDNRPTVLTDPSGACFFIDMPWIPGCSEPSWADEVTPATNGVLAGLISAAEDTFYLTGMALGVDWVGYDGDLAQQLFDEAAVEGNYHGETYQQAQLVGGLVALVGGAASTAASLARICTSLVRKIRPPVASGGLATEVPAYAGSRTAGTLVTPDGAEFPLISGWHPPAASMPQGTPGMNIVTKSHVEAHAAAIMRNQGLSEATLWINRAPCGGKPGCAAMLPRMVPSGSTLTINVVPNGSAGSIADTLIIRGIG

SEQ ID NO: 121 - protein, 1526 aa, Burkholderia diffusa
MSDSENRLTRASDSPASGKTQSESKVNTACDSLLDTAGSTYDSLKQPFSSKGGALHHVSEAVNALASLQGAPSQLLNTGIAQIPLLDKMPGMPASVISAAHLGTPHAHSHPPSDGFPLPSMGATIGSGCLSVLIGGLPAARVQDIGIAPTCGGLTPYFNIETGSSNTFIGGMRAARMGIDMTRHCNPMGHAGKSGEEAEGAAEKGEQAASEAAEVSSRARWMGRAGKAWKVGNAAVGPASGVAGAASDAKHHEALAAAMMAAQTAADAAMMLLSNLMGKDPGIEPSMGMLMDGNPTVLIGGFPMPDSQMMWHGAKHGLGKKVKARRADRQKEAAPCRDGHPVDVVRGTAENEFVDYETRIAPGFKWERYYCSGWSEQDGELGFGFRHCFQHELRLLRTRAIYVDALNREYPILRNAAGRYEGVFAGYELEQRDGRRFVLRHGRLGDMTFERASEADRTARLVNHVRDGVESTLEYARNGALMRIDQEKGPGRRRQLIDFRYDDCGHIVELYLTDPQGETKRIVHYRYDTAGCLAASTNPLGAVMSHGYDGRRRMVRETDANGYSFSYRYDSQDRCIESMGQDGLWHVSLDYQPGRTVVTRADGGKWTFLYDEARTVTRIVDPYGGTTERVSGDNGRILREIDSGGRVMRWLYDERGGNTGRMDRWGNRWPTKDEAPVLPNPLAHTVPNTPLALQWGDARHEDLADTLLLPPEIAKIAASFFPPQPFSASTEQCDETGRVIARTDGYGQAERLRLDATGNLLQLCDRDGRDYCYSIASWNLRESETDPLGNTVRYRYSPKQEITAIVDANGNESTYTYDYKSRLTSVTRHGTVRETYAYDVGDRLIEKRDGTGNALLRFEVGEDGLQKTRILASGETHTYKYDHRGNFTRASTDKLDVTLTYDAYGRRTGDKRDGRGIDHSFVGGRLESTTYFGRFVVRYEAGQAGDVMIHTPGGGIHRLRRAADGTVLLRLGNRTNVLYGFDADGRCTGRLSWPEGRTAEIHCVQYRYSAVGELRCVIDSTGGTIEYQYDAAHRLVGESRDGWAVRRFEYDQGGNLLSTPTCQWMRYTEGNRLSSASCGAFRYNSRNHLAEQIEENNRRTTYHYNSMDLLVQVKWSDRQESWRSEYDGLCRRIAKAMGQARTQYFWDGDRLAAEAAPDGRLRIYVYVNEASYLPFMFIDYPSCDAEPESGSAYYVFCNQVGLPERIESAMGLDAWRAEEIEPYGSIRVATGNAIDYDLRWPGHWFDVETGLHYNRFRYFQPTLGRYLQSDPAGQSGGVNLYAYSANPLVFVDVLGLECPHNDKSTTECARCEAKEEVDQREKRDKELAREIYHIEDKYSDSHAGIGLDPDEKKRALEDKIDYDDLVRKREKAREDLLEAEKRLREEEIRAKYPTPEEAQLPPYDGDTTYALMYYTDEHGKSHVVELSSGGADDEHSNYAAAGHTEGQAAVIMRQRKITSAVVVHNNTDGTCPFCVAHLPTLLPSGAELRVVPPRSAKAKKPGWIDVSKTFEGNARKPLDNKNKKST

SEQ ID NO: 122 - protein, 1513 aa, Burkholderia ubonensis
MSEPANRLTRASEPSERHAAQSESKADTACESLLGTVKSTFDPFKQTFSSDGSALHHVSEAVNALASLQSAPSQLLNTGIAQIPLLDKMPGMPAATIGVPHLGTPHAHSHPPSSGFPLPSIGATIGSGCLSVLIGGIPAARVLDIGIAPTCGGLTPYFDIQTGSSNTFFGGMRAARMGIDMTRHCNPMGHVGKSGGKAAGAAEKTEEAASEAAQVTSRAKWMGRAGKAWKVGNAAVGPASGAAGAAADAAHGEELAAAMMAAQTAADAAMMLLGNLMGKDPGIEPSMGTLLAGNPTVLVGGFPLPDSQMMWHGVKHGIGKKVRARIANRRKEVSPCTDGHPVDVVRGTAENEFVDYETKIAPAFKWERYYCSGWSEQDGALGFGFRHCFQHELRLLRTRAIYVDALNREYPILRNAAGRYEGVFAGYELEQRDGRRFLLRHGRHGDMTFERENEADRTARFVSHVRDDVECTLEYARNGALARIAQEDARGLRRQLIDFRYDDRGHIVELCLTDPRGQTRRLAHYRYDAAGCLTVVTDPLGAVTSHGYDDRRRMVRETDANGYSFSYRYDSQGRCIETVGQDGLLHVVLDYQPGRTVVTRADGGKWTFLYDNARTVTRIVDPYGGMTERVIGGDGRILREIDSGGRVMRWLYDERGRNTGRMDRWGNCWPTRDEAPVLPNPLAHTVPVTPLDLQWGEVSPAELTDSVLLSPEIQKVAESLFQQPAFSPSEQHDARGQVVARTDEHGGVERFRRDAAGNIIQVCDKDGRAHHYGIASWSLRESETDSLGNTVRYRYSNKQEITSIVDANGNESAYTYDYKGRITSVMRHGVVRETYTYDAGDRLIEKRDGAGNLLLRFEVGENGLHSKRILASGETHTYEYDRRGNFTKASTDKFDVTRTYDAHGRRTGDKRDGRGIEHVYGDGRLCSTTYFERFTVRYEAEADGEVLIHAPVGGTHRLQRSSDGQILLRLGNGANVLCRFDAHGRCVGRLVWPEGRPKECHRVAYQYSAMGELRRVIANTTGTTEYLYDDAHRLIGESHDGWPVRRFEYDCGGNLLSAPTCQWMRYTEGNRLATASRGAFYYNDRNHLAEQIGENNHRTSYHYNSMDLLVKVTWSDWPEVWTAEYDGLCRRIAKAMGPARTEYYWDGDRLAAEIAPNGQLRIYVYVNETSYLPFMFIDYDGCDAAPESGRGYYVFSNQVGLPEWIEDIAGACVWRAMEIDPYGAIRVAPGNELGYNLRWPGHWLDPETGLHYNRFRSYHSALGRYLQSDPAGQSGGINLYAYTANPLVFVDVLGRECPHLNESSSECSQCENREEAERIRKEMLQSISRRMDIEGDVTGHPGILLTQAELTGKYSHYAEEYKQLLKDIDTKREAEEAALLREAYPSMEGATLPPFDGKTTIGLMFYTDASGQYQVKKLFSGEKVLSNYDATGHVEGKAALIMRNEKITEAVVMHNHPSGTCNYCDKQVETLLPKNATLRVIPPENAKAPTSYWNDQPTTYRGDGKDPKAPSKK

SEQ ID NO: 123 - protein, 1500 aa, Paraburkholderia guartelaensis
MSTPPGNPASPANEPPPPPAPLISPTGNTSVDALASAVNAGAQPFQQLGNPKANTLDRVTNVVSGAVGSLGALDQLLNTGMAMIPGANLVPGMPAAFIGVPHLGVPHAHAHPPSDGVPMPSCGVTIGSGCLSVLYGGMPAARVLDIGLAPTCGGLAPIFEICTGSSNTFIGGARAARMALDLTRHCNPLGMSGAGHAEQDAEKASALKRAMHIAGMAAPVASGGLTAADQAVDGAGAAAVEMTAAQTAADAIAMAMSNLMGKDPGVEPGVGTLIDGDASVLIGGFPMPDALAMLMLGWGLRKKAHAPEGAGEPKRTEQGECKGGHPVDVVRGTAENQFTDYATLDAPEFKWERYYRSDWSERDGALGFGFRHSFQHELRLLRTRAIYVDGHGRAYAFGRSASGRYEDVFAGYELEQQGENRFVLLQATRGEFTFERASAAQASARLVRHVHEGVESALRYAGDGTLRHIEQTAQREQRHRMIDLLYDARGHVVEMRVTDPRGAVLCAARYRYDATGCLVASTDALGASMTYGYDAWRRMIRETDANGYAFSYRYDSDGRCVESAGQDGLWRVLLDYQPGRTVVTQADGGRWTYLYDAARTVTRIVDPYGGATERVIGDDGRIVEEVDSGGRVMRWLYDERGENTGRQDRWGNRWPTRDEAPVLPNPLAHVVPARPLELLWGDARPEDFTDRLLLPPEIEAVAAAAFAPSAAVPKPAEQRDGAGRVIRRTDESGHAECLHRDAAGNVVQLRDKDGRYYGYAIASWNLRESETDPLGNTVRYRYSSKQNITAVVDANGNESRYTYDYKSRLTRVARHDTIRESYVYDTGDRLIEKRDGAGNTLLRFEVGENGLHSKRILASGETHTYEYDRRGNFTRASTDKFEVTLTYDAFGRRTGDKRDGRGVEHSFVGQRLESTTWFGRFVVRYETGPSGDVMIHTPGGDVHRLQRAADGTVLLRLSNSTNVLYKFDENGRCAGRLTWPDGHTSANRCVQYRYSAVGELRQVIDSKGGTTEYQYDDAHRLVGESREGWAFRRFEYDRGGNLLSTPTCQWMRYTEGNRLSGAACGAFCYNSRNHLAEQIGENNRRTTWHYNSMDLLVRVQWSDRQENWSAEYDGLCRRIAKAMGQARTQYFWDGDRLAAEVAPNGQLRIYAYVNETSYLPFMFIDYDGCDAAPESGRTYYVFCNQVGLPEWIEDISRGCVWGVNEIDPYGAICVAPDNELEYNLRWPGHWFDPETDLHYNRFRSYSPVLGRYLQSDPAGQAGGINLYAHTANPLVFIDVLGRECPHGNESSSECSQCADREEAERINAKILQLISKKMSIEDAVTGHPGELIPLPHFEIDKEYSHYAKEYKQLLADIDALAEAREDALLREQFPSMDAVTLPPFDGKTTIGYMFYTDANGQYHVRKLYSGGKVLSNYDSSGHVEGMAALIMRKGRITEAVVMHNHPSGTCHYCNGQVETLLPKNAKLKVIPPANAKAPTKYWYDQPVDYLGNSNDPKPPS

SEQ ID NO: 124 - protein, 137 aa, Pseudoduganella violaceinigra
GSSGKNVRMPRDYASELPEYDGKTTHGVLVTNEGKVIQLRSGGKEEPYTGYKAVSASHVEGKAAIWIRENGSSGGTVYHNNTTGTCGYCNSQVKALLPEGVELKIVPPTNAVAKNAQARAVPTINVGNGTQPGRKQK

SEQ ID NO: 125 - protein, 408 aa, Duganella sp.
MDAETGLVYFQARYYDPQLGRFITQDPYEGDWKTPLSLHHYLYAYANPTTYVDLNGYYARDANEVQRYIIAESNCAKTGSCDAVTALREPSEARQRSAANCKSLDRCREIADDAARSEGDISARIKALQKDLRNGIEANPTTGIKTIWELDKQLEARNISAGAVREAGRHVRWRAFVENRELTDHEKVAPAAEMYGVLSGGRIVIARAVARSSVTRASITQESKTIGVTAEVAPNESLRNTSGDLRASANSARNQPYGNGQSASASPSTNSAGSSGKNVRLPRDYASELPEYDGKTTYGVLVTNEGKVIQLRSGGKEVPYSGYKAVSASHVEGKAAIWIRENASSGGTVYHNNTTGTCGYCNSQVKALLPEGVELKIVPPANAVARNSQAKAIPTINVGNATQPGRKP

SEQ ID NO: 126 - protein, 1354 aa, Chondromyces apiculatus
MLASTWLDLVIGVDLHFELVPPVMAPVPFPHPFVGLVFDPWGLLGGLVISNVMSVATGGSLQGPVLINLMPATTTGTDAKNWMLLPHFIIPPGVMWAPMVRVPKPSIIPGKPIGLELPIPPPGDAVVITGSKTVHAMGANLCRLGDIALSCSDPIRLPTAAILTIPKGMPVLVGGPPALDLMAAAFALIKCKWVANRLHKLVNRIKNARLRNLLNRVVCFFTGHPVDVATGRVMTQATDFELPGPLPLQFERVYASSWADRASPVGRGWSHSLDQAVWLEPGKVVYRAEDGREIELDTFELPGRMLQPGQESFEPLNRLLFRCLDGHRWEVESAEGLVHEFAPVAGDADPAMARLTRKRSRQGHAITLHYDGKGCLTWVQDSGGRIVRFEHDEAGHLTQVSLPHPTQPGWLPHTRYIYSPEGDLVEVVDPLGHRTRYEYVGHLLVRETDRTGLSFYFGYDGTGPGAYCIRTWGDGGIYDHEIDYDKVNRVTFVTDSLGATTTYEMNVANAVVKVIDPRGGETRYEYNDVLWKTEEVEPAGGATRYEYDARGNCTKSTGPDGATVQVEYDARNVPIRAVNPCGEEWQWVYDAQGQLVERIDPLGETTRYEYDKGMVVTITEASGVTTAEYDDSRNLRRVQGPSEAETSYVYDALGRMVVKRSPARVAERLHYDACGRLVTVEQPDGNVWRLAYDGEGNLTEIQDHHQRVRMRYGGYHQMVSRQEAEDTTLFRYDSEGRLVAIENEAGEIYQYELDSCGRAGLERGFDGGCWKYERDAAGRVIKLRKPSGAEARLIYDAMGRLVEVRRSDSAVERFRYRKDGALIEAENSTIQVKFERDALGRVVREMQGGHWVESSYERGARTWVASSLGVHSAIMRDERRSVVAMTAGRGVDEWRVELSRDAFGLETERKLSSGIVSTWARDALGRPRHRGVAHSNNVLFGVEYQWAPGSRLVALIDTERGTTAFHFDERSRLVGAKLPGGRIDRREPDRIGNIYRAQDQRDRTYSDGGILRGAGETRYTHDLDGNLTQKVLPDGATWSYSYNAAGCLKEVERPDGTRVTFAYDALGRRVSKRWGENEVWWLWDRHVPLHEISTRAEPITWLFEPESFAPIAKIEGDRHYDILCDHLGAPTVVLDEAGVVTWRARLDIHAAVQPEIAETECPWRWPGQYEDQETGLYYNRFRYYDPEADRYISQDPLGPVGGLNLYSYAADPLTWSDPLGLQPDPPPPPTPMGNTLPGWDGGKTQGWFVYPDGTERHLISGYDGPSKFTQGIPGMNGNIKSHVEAHAAALMRQYELSKATLYINRVPCPGVRGCDALLARMLPEGVQLEIIGPNGFKKTYTGLPDPKLKPKGCS

SEQ ID NO: 127 - protein, 2169 aa, Sorangium cellulosum
MTACSDSPRLPPSLLELPDTPCPEPDEAASPFPAELPHSATVEAGAIAGSFGVTSTGEATYTIPLVVPPGRAGMQPELAVQYDSASGEGVLGMGFSVTGLSAVTRCPRNLAQDGEIRAVRYDEGDALCLDGKRLVEVGGGGEVVEYRTVPDTFARVVASYEGGWDRARGPKRLRVFTRAGRVLEYGGEPSGQVLAKGGVIRAWWATRVSDRSGNTIDFHYQNETSASEGYTVEHAPRRIEYTGHPRAAATRAIEFVYAPRRPGTGRVLYSRGMALRSSQQLDRIRMLGPGGALVREYRFSYTSGPATGRRLLNAVRECAADGRCKPATRFRWHHGTGPGFAEVGTRLRVPESERGSLMTMDATGDGRDDLVTTDLDLPVDDDNPITNFFVAPNRMAEGGSSSFGALALAHQEMHHAPPSPVQPELGTPIDYNDDGRMDIFLHDVHGRYPDWHVLLATPEGTFRRKSTGIRRKFGIDAPPPLDLNSRNASAHLADVDGDGIADLLQCEDTGSVFTDWTLHLWRPAASGFEPEPSRIPALRGHPCNAETHLADVDSDGKVDLLVYEATITGNGTLFGTTFEALSFVRPGEWTKRATGLPVLKAGSGGRVIVLDVNGDGLPDAVETGFDDGQLRTFINTGDGFAAGVSSLPSFVFDADAFAKLAAPIDHNSDGRQDLLMPIREPGGPVLWKILQATGSTGDGTFAVIDARLPVSEVLVDREITLAHPWAPRVTDVDGDGNQDVVLAVGKELRVFRSRLREEDLLWTVSDGMSAYDPEEAGHVPKVQIEYSHLSAAEPGVRGEQRTYLPRYDTGEPGDGACDYPVRCALGPRRVVSRYAVNNGADRLRTFQVAYRNGKYHRLGRGFLGFGVRIVRDAASGAGSAEFFDNVTFDPSDRSFPLAGHVVREWRWTPEPQQKGVSRVELSYTERLIHAILTNRGKSYFTLPVYQKQRREQGEHRRDSGKTLEEYVRDTWYAPTQVVSRTERLVSAWDAFGNIREESTSTAGVDLTLKVKRTFRNDEDAWLIGLLETQQECSRALSIEQCRTSSRAYDRHGRVRTESAGSDDDDPETVVRVRYTRDAFGNVIHTRAEDAFGGRRKACVSYDAEGVFPYAQRNPEGHVTYTRYDAGHGALEAVVDPNGLATQWAHDGLGRITEERRPDGTTTRATLSRTRDGGPRGDAWRVLRRTATDGGADETVELDGFGRPIRGWAYKARTDDGPAERVVQEIAFDQSGERVARRSLPAAEGTPRERMQVETYGHDATGRIAWHRAAWGAETRYRYLGRTVEVEGPGGRVTTIENDALGRPVRIVDPEGGVTSYAYGPFGGLWTVTDPGDAKTTTERDAYGRVRRHIDPDRGTAVAHYDGFGQQTSTVDALGREVSWKHDRLGRAVERSDEDGTTTWTWDEAEHGVGKLAEVASPEGHRTTYRYDALGRLREEELAIEGERFATTVDYDGHSRPFRLWYPQAEGERRFGVRRIFDAHGHLVGLRNERSREMFWRLEDTDEAGRIRIEEFGNGVTTERSYHETKGRLRRVATMKDHVVLQDLWYGYDDRLNLSSRRDDRLERTEHFRYDKLDRLTCAARHERFCLFETTYAPNGNIREKPDVGEYTYDPEHPHAVRTAGADVFAYDAVGNQVRRPGVEEIRYTAFDLPASITLAGGTGTVDLDYDGDQRRIRKTTPMEQTVYAGDLYERVTDLATGVVEHRYTVRSSERAVAVVTKRAGGEARTLYIHVDHLGSVDLLTEGRGEDAGREVERRSYDAFGARRDPVTWRRAPKAEAPPALLARGFTGHGSDDELGLVHMKGRLYDPKIGRFTTPDPVVSRPLFGQSWNAYSYVLNNPLAYVDPSGFQEAVPEDRGGSSRAAGAEFTSDELGLPPIEELVVARFPEHEARSDADANAMGAEVGGAVPPVDVGVYGTSAGFVPQPGPSSPEHASAASVVGEGLLGAGEGTGELALRVARSLVLSALTFGGYGTYELGRAMWDGYKENGVVGALNAVNPLYQIGRGAADTALAIDRDDYRAAGAAGVKTVIIGAATVFGAGRGLGALEEATTAAGIARGAPSLPVYTGGKTTGVLRTATGDMPLVSGYKGPSASMPRGTPGMNGRIKSHVEAHAAAVMRERGIKDATLHINQVPCSSATGCGAMLPRMLPEGAQLRVLGPDGYDQVFIGLPD

SEQ ID NO: 128 - protein, 273 aa, Clostridium sp.
MVIIGRIDTNESTVSLYQWSLLPATDTNCYKEITVEQYKNNQLVRKVSFSKAFVVNYTESYSNHVGVGTFTLYVRQFCGKDIEVTSQELNSVSNLTPNLPNSVEKDVEVVEIAEKQAVVKSDTSNLKQSNMSITDRLAKQKEKQDNTNIIDNRPKLPDYDGKTTHGILVTPNSEHIPFSSGNPNPNYKNYIPASHVEGKSAIYMRENGITSGTIYYNNTDGTCPYCDKMLSTLLEEGSVLEVIPPINAKAPKPSWVDKPKTYIGNNKVPKPNK

SEQ ID NO: 129 - protein, 339 aa, Unknown
  source: /note=“Description of Unknown Chloroflexia bacterium sequence”
MLYAYGPESVVAERTIVGTTVADAGKAAFRVLDDTLAEGVEHSANKADEAGELIEAVVEQCLRNSFSADTLVTTASGLRPISTIAVGELVLAWDATTRSTGYYPVTAVMLHTDAAQVHLSVGGEHVETTPEHPFYTLERGWVAAGDLWDGAHVRRADGSYALTLVLWLDAEPQVMYNLTVATAHTFFVGVERALVHNAGCPGDALPPYGTKGSKTTGILDTGNESILLESGENGPGMMVPRDTPGMSGAMPNRAHVEGHTAAIMRNENIRLADLYINRMPCSGAYGCMVNLPHMLPEGSILRIHVRAKLSDPWTTLPPFVGISDTLWPPSGLNPKIVLP

SEQ ID NO: 130 - protein, 142 aa, Streptomyces rubrolavendulae
VAGNRAFTQRARTYNLTVADLHTYYVLAGQTPVLVHNANCGPHLKDLQKDYPRRTVGILDVGTDQLPMISGPGGQSGLLKNLPGRTKANGEHVETHAAAFLRMNPGVRKAVLYIDYPTGTCGTCRSTLPDMLPEGVQLWVIS

SEQ ID NO: 131 - protein, 12 aa, Streptomyces rubrolavendulae
PRRTEKFTGLPD

SEQ ID NO: 132 - protein, 123 aa, Propionibacterium acidifaciens
LSTTGKNVLGHFEPTPTTPQGTSSDTIAEMLNSASQPGRTAGVLDIDGELTPLTSGRPSLPNYIASGHVEGQAAMIMRQQQVQSATVYHDNPNGTCGYCYSQLPTLLPEGAALDVVPPAGTVP

SEQ ID NO: 133 - protein, 24 aa, Propionibacterium acidifaciens
PSNRWHNGGPSFIGNSSEPKPWPR

SEQ ID NO: 134 - protein, 134 aa, Artificial Sequence
  source: /note=“Description of Artificial Sequence Synthetic polypeptide”
PTPTTPQGTSSDTIAEMLNSASQPGRTAGVLDIDGELTPLTSGRPSLPNYIASGHVEGQAAMIMRQQQVQSATVYHDNPNGTCGYCYSQLPTLLPEGAALDVVPPAGTVPPSNRWHNGGPSFIGNSSEPKPWPR

SEQ ID NO: 135 - protein, 43 aa, Artificial Sequence
  source: /note=“Description of Artificial Sequence Synthetic polypeptide”
PTPTTPQGTSSDTIAEMLNSASQPGRTAGVLDIDGELTPLTSG

SEQ ID NO: 136 - protein, 91 aa, Artificial Sequence; identical to SEQ ID NO: 97
  source: /note=“Description of Artificial Sequence Synthetic polypeptide”

SEQ ID NO: 137 - protein, 110 aa, Artificial Sequence
  source: /note=“Description of Artificial Sequence Synthetic polypeptide”
PTPTTPQGTSSDTIAEMLNSASQPGRTAGVLDIDGELTPLTSGRPSLPNYIASGHVEGQAAMIMRQQQVQSATVYHDNPNGTCGYCYSQLPTLLPEGAALDVVPPAGTVP

SEQ ID NO: 138 - protein, 24 aa, Artificial Sequence
  source: /note=“Description of Artificial Sequence Synthetic peptide”
PSNRWHNGGPSFIGNSSEPKPWPR

SEQ ID NO: 139 - protein, 64 aa, Unknown
  source: /note=“Description of Unknown Lachnospiraceae bacterium sequence”
DPRYTNYRNNGHVEQKSALYMRENNISNATVYHNNTNGTCGYCNTMTATFLPEGATLTVVPPEN

SEQ ID NO: 140 - protein, 31 aa, Unknown
  source: /note=“Description of Unknown Lachnospiraceae bacterium sequence”
AVANNSRAIDYVKTYTGTSNDPKISPRYKGN

SEQ ID NO: 141 - protein, 121 aa, Ruminococcus bicirculans
HTYHVGKCRLLVHNANCNQEKPVLPKYDGKTTEGVMVTPDGKQISFKSGNSSTPSYPQYKAQSASHVEGKAALYMRENGINEATVFHNNPNGTCGFCDRQVPALLPKGAKLTVVPPSNSVA

SEQ ID NO: 142 - protein, 22 aa, Ruminococcus bicirculans
NNVRAIPVPKTYIGNSTVPKIK

SEQ ID NO: 143 - protein, 129 aa, Artificial Sequence
  source: /note=“Description of Artificial Sequence Synthetic polypeptide”
ANCNQEKPVLPKYDGKTTEGVMVTPDGKQISFKSGNSSTPSYPQYKAQSASHVEGKAALYMRENGINEATVFHNNPNGTCGFCDRQVPALLPKGAKLTVVPPSNSVANNVRAIPVPKTYIGNSTVPKIK

SEQ ID NO: 144 - protein, 39 aa, Artificial Sequence
  source: /note=“Description of Artificial Sequence Synthetic polypeptide”
ANCNQEKPVLPKYDGKTTEGVMVTPDGKQISFKSGNSST

SEQ ID NO: 145 - protein, 90 aa, Artificial Sequence; identical to SEQ ID NO: 103
  source: /note=“Description of Artificial Sequence Synthetic polypeptide”

SEQ ID NO: 146 - protein, 107 aa, Artificial Sequence
  source: /note=“Description of Artificial Sequence Synthetic polypeptide”
ANCNQEKPVLPKYDGKTTEGVMVTPDGKQISFKSGNSSTPSYPQYKAQSASHVEGKAALYMRENGINEATVFHNNPNGTCGFCDRQVPALLPKGAKLTVVPPSNSVA

SEQ ID NO: 147 - protein, 22 aa, Artificial Sequence
  source: /note=“Description of Artificial Sequence Synthetic peptide”
NNVRAIPVPKTYIGNSTVPKIK

SEQ ID NO: 148 - protein, 144 aa, Streptomyces cavourensis
VQITAIKRWTETATVHNLTVADLHTYYVLAGKTPVLVHNENCGPNLKDLPKDYDRRTVGILDVGTDQLPMISGPGGQSGLLKNLPGRTKANTDHVEAHTAAFLRMNPGIRKAVLYIDYPTGTCGTCGSTLPDMLPEGVQLWVIS

SEQ ID NO: 149 - protein, 12 aa, Streptomyces cavourensis
PRKTEKFAGLPD

SEQ ID NO: 150 - protein, 112 aa, Roseburia intestinalis
SAGAGESGRKTISLPEYDGTTTHGVLVLDDGTQIGFTSGNGDPRYTNYRNNGHVEQKSALYMRENNISNATVYHNNTNGTCGYCNTMTATFLPEGATLTVVPPENAVANNSR

SEQ ID NO: 151 - protein, 24 aa, Roseburia intestinalis
AIDYVKTYTGTSNDPKISPRYKGN

SEQ ID NO: 152 - protein, 151 aa, Artificial Sequence
  source: /note=“Description of Artificial Sequence Synthetic polypeptide”
DPSGYDSQYPCKEEMSAGAGESGRKTISLPEYDGTTTHGVLVLDDGTQIGFTSGNGDPRYTNYRNNGHVEQKSALYMRENNISNATVYHNNTNGTCGYCNTMTATFLPEGATLTVVPPENAVANNSRAIDYVKTYTGTSNDPKISPRYKGN

SEQ ID NO: 153 - protein, 55 aa, Artificial Sequence
  source: /note=“Description of Artificial Sequence Synthetic polypeptide”
DPSGYDSQYPCKEEMSAGAGESGRKTISLPEYDGTTTHGVLVLDDGTQIGFTSGN

SEQ ID NO: 154 - protein, 96 aa, Artificial Sequence; identical to SEQ ID NO: 109
  source: /note=“Description of Artificial Sequence Synthetic polypeptide”

SEQ ID NO: 155 - protein, 127 aa, Artificial Sequence
  source: /note=“Description of Artificial Sequence Synthetic polypeptide”
DPSGYDSQYPCKEEMSAGAGESGRKTISLPEYDGTTTHGVLVLDDGTQIGFTSGNGDPRYTNYRNNGHVEQKSALYMRENNISNATVYHNNTNGTCGYCNTMTATFLPEGATLTVVPPENAVANNSR

SEQ ID NO: 156 - protein, 24 aa, Artificial Sequence
  source: /note=“Description of Artificial Sequence Synthetic peptide”
AIDYVKTYTGTSNDPKISPRYKGN

SEQ ID NO: 157 - protein, 115 aa, Plantactinospora sp.; identical to SEQ ID NO: 117

SEQ ID NO: 158 - protein, 33 aa, Plantactinospora sp.
MGDRLPAFVDGGDTLGIFSRGGIERDLASGVAG

SEQ ID NO: 159 - protein, 82 aa, Plantactinospora sp.
PASSLPKGTPGFNGLVKSHVEGHAAALMRQNGIPNAELYINRVPCGSGNGCAAMLPHMLPEGATLRVYGPNGYDRTFTGLPD

SEQ ID NO: 160 - protein, 102 aa, Plantactinospora sp.
MGDRLPAFVDGGDTLGIFSRGGIERDLASGVAGPASSLPKGTPGFNGLVKSHVEGHAAALMRQNGIPNAELYINRVPCGSGNGCAAMLPHMLPEGATLRVYG

SEQ ID NO: 161 - protein, 13 aa, Plantactinospora sp.
PNGYDRTFTGLPD

SEQ ID NO: 162 - protein, 153 aa, Streptomyces massasporeus
GGSAVVGAGVVATGAKAVTTGKSLSESQATLSVAQRLLATIGEEGKTAGVLELDGELIPLVSGKSSLPNYAASGHVEGQAALIMRDRGATSGRLLIDNPSGICGYCKSQVATLLPENATLQVGTPLGTVTPSSRWSASRTFTGNDRDPKPWPR

SEQ ID NO: 163 - protein, 63 aa, Streptomyces massasporeus
GGSAVVGAGVVATGAKAVTTGKSLSESQATLSVAQRLLATIGEEGKTAGVLELDGELIPLVSG

SEQ ID NO: 164 - protein, 90 aa, Streptomyces massasporeus
KSSLPNYAASGHVEGQAALIMRDRGATSGRLLIDNPSGICGYCKSQVATLLPENATLQVGTPLGTVTPSSRWSASRTFTGNDRDPKPWPR

SEQ ID NO: 165 - protein, 130 aa, Streptomyces massasporeus
GGSAVVGAGVVATGAKAVTTGKSLSESQATLSVAQRLLATIGEEGKTAGVLELDGELIPLVSGKSSLPNYAASGHVEGQAALIMRDRGATSGRLLIDNPSGICGYCKSQVATLLPENATLQVGTPLGTVT

SEQ ID NO: 166 - protein, 23 aa, Streptomyces massasporeus
PSSRWSASRTFTGNDRDPKPWPR

SEQ ID NO: 167 - protein, 143 aa, Streptomyces sp.
DIILATLPIGKVGKLRFAPKVESAESMLRSLSQEGKTAGVLDINGELIPLVSGTSSLKNYAASGHVEGQAALIMRERGVASARLIIDNPSGICGYCRSQVPTLLPAGATLEVTTPRGTVPPTARWSNGKTFVGNENDPKPWPR

SEQ ID NO: 168 - protein, 53 aa, Streptomyces sp.
DIILATLPIGKVGKLRFAPKVESAESMLRSLSQEGKTAGVLDINGELIPLVSG

SEQ ID NO: 169 - protein, 90 aa, Streptomyces sp.
TSSLKNYAASGHVEGQAALIMRERGVASARLIIDNPSGICGYCRSQVPTLLPAGATLEVTTPRGTVPPTARWSNGKTFVGNENDPKPWPR

SEQ ID NO: 170 - protein, 120 aa, Streptomyces sp.
DIILATLPIGKVGKLRFAPKVESAESMLRSLSQEGKTAGVLDINGELIPLVSGTSSLKNYAASGHVEGQAALIMRERGVASARLIIDNPSGICGYCRSQVPTLLPAGATLEVTTPRGTVP

SEQ ID NO: 171 - protein, 23 aa, Streptomyces sp.
PTARWSNGKTFVGNENDPKPWPR

SEQ ID NO: 172 - protein, 128 aa, Jiangella alba
PPVASGGLATEVPAYAGSRTAGTLVTPDGAEFPLISGWHPPAASMPQGTPGMNIVTKSHVEAHAAAIMRNQGLSEATLWINRAPCGGKPGCAAMLPRMVPSGSTLTINVVPNGSAGSIADTLIIRGIG

SEQ ID NO: 173 - protein, 37 aa, Jiangella alba
PPVASGGLATEVPAYAGSRTAGTLVTPDGAEFPLISG

SEQ ID NO: 174 - protein, 91 aa, Jiangella alba
WHPPAASMPQGTPGMNIVTKSHVEAHAAAIMRNQGLSEATLWINRAPCGGKPGCAAMLPRMVPSGSTLTINVVPNGSAGSIADTLIIRGIG

SEQ ID NO: 175 - protein, 116 aa, Jiangella alba
PPVASGGLATEVPAYAGSRTAGTLVTPDGAEFPLISGWHPPAASMPQGTPGMNIVTKSHVEAHAAAIMRNQGLSEATLWINRAPCGGKPGCAAMLPRMVPSGSTLTINVVPNGSAG

SEQ ID NO: 176 - protein, 12 aa, Jiangella alba
SIADTLIIRGIG

SEQ ID NO: 177 - protein, 140 aa, Burkholderia diffusa
EIRAKYPTPEEAQLPPYDGDTTYALMYYTDEHGKSHVVELSSGGADDEHSNYAAAGHTEGQAAVIMRQRKITSAVVVHNNTDGTCPFCVAHLPTLLPSGAELRVVPPRSAKAKKPGWIDVSKTFEGNARKPLDNKNKKST

SEQ ID NO: 178 - protein, 44 aa, Burkholderia diffusa
EIRAKYPTPEEAQLPPYDGDTTYALMYYTDEHGKSHVVELSSGG

SEQ ID NO: 179 - protein, 96 aa, Burkholderia diffusa
ADDEHSNYAAAGHTEGQAAVIMRQRKITSAVVVHNNTDGTCPFCVAHLPTLLPSGAELRVVPPRSAKAKKPGWIDVSKTFEGNARKPLDNKNKKST

SEQ ID NO: 180 - protein, 112 aa, Burkholderia diffusa
EIRAKYPTPEEAQLPPYDGDTTYALMYYTDEHGKSHVVELSSGGADDEHSNYAAAGHTEGQAAVIMRQRKITSAVVVHNNTDGTCPFCVAHLPTLLPSGAELRVVPPRSAKA

SEQ ID NO: 181 - protein, 28 aa, Burkholderia diffusa
KKPGWIDVSKTFEGNARKPLDNKNKKST

SEQ ID NO: 182 - protein, 116 aa, Burkholderia diffusa
EIRAKYPTPEEAQLPPYDGDTTYALMYYTDEHGKSHVVELSSGGADDEHSNYAAAGHTEGQAAVIMRQRKITSAVVVHNNTDGTCPFCVAHLPTLLPSGAELRVVPPRSAKAKKPG

SEQ ID NO: 183 - protein, 24 aa, Burkholderia diffusa
WIDVSKTFEGNARKPLDNKNKKST

SEQ ID NO: 184 - protein, 137 aa, Burkholderia ubonensis
AALLREAYPSMEGATLPPFDGKTTIGLMFYTDASGQYQVKKLFSGEKVLSNYDATGHVEGKAALIMRNEKITEAVVMHNHPSGTCNYCDKQVETLLPKNATLRVIPPENAKAPTSYWNDQPTTYRGDGKDPKAPSKK

SEQ ID NO: 185 - protein, 45 aa, Burkholderia ubonensis
AALLREAYPSMEGATLPPFDGKTTIGLMFYTDASGQYQVKKLFSG

SEQ ID NO: 186 - protein, 92 aa, Burkholderia ubonensis
EKVLSNYDATGHVEGKAALIMRNEKITEAVVMHNHPSGTCNYCDKQVETLLPKNATLRVIPPENAKAPTSYWNDQPTTYRGDGKDPKAPSKK

SEQ ID NO: 187 - protein, 112 aa, Burkholderia ubonensis
AALLREAYPSMEGATLPPFDGKTTIGLMFYTDASGQYQVKKLFSGEKVLSNYDATGHVEGKAALIMRNEKITEAVVMHNHPSGTCNYCDKQVETLLPKNATLRVIPPENAKA

SEQ ID NO: 188 - protein, 25 aa, Burkholderia ubonensis
PTSYWNDQPTTYRGDGKDPKAPSKK

SEQ ID NO: 189 - protein, 134 aa, Paraburkholderia guartelaensis
ALLREQFPSMDAVTLPPFDGKTTIGYMFYTDANGQYHVRKLYSGGKVLSNYDSSGHVEGMAALIMRKGRITEAVVMHNHPSGTCHYCNGQVETLLPKNAKLKVIPPANAKAPTKYWYDQPVDYLGNSNDPKPPS

SEQ ID NO: 190 - protein, 45 aa, Paraburkholderia guartelaensis
ALLREQFPSMDAVTLPPFDGKTTIGYMFYTDANGQYHVRKLYSGG

SEQ ID NO: 191 - protein, 89 aa, Paraburkholderia guartelaensis
KVLSNYDSSGHVEGMAALIMRKGRITEAVVMHNHPSGTCHYCNGQVETLLPKNAKLKVIPPANAKAPTKYWYDQPVDYLGNSNDPKPPS

SEQ ID NO: 192 - protein, 111 aa, Paraburkholderia guartelaensis
ALLREQFPSMDAVTLPPFDGKTTIGYMFYTDANGQYHVRKLYSGGKVLSNYDSSGHVEGMAALIMRKGRITEAVVMHNHPSGTCHYCNGQVETLLPKNAKLKVIPPANAKA

SEQ ID NO: 193 - protein, 23 aa, Paraburkholderia guartelaensis
PTKYWYDQPVDYLGNSNDPKPPS

SEQ ID NO: 194 - protein, 137 aa, Pseudoduganella violaceinigra; identical to SEQ ID NO: 124

SEQ ID NO: 195 - protein, 43 aa, Pseudoduganella violaceinigra
GSSGKNVRMPRDYASELPEYDGKTTHGVLVTNEGKVIQLRSGG

SEQ ID NO: 196 - protein, 94 aa, Pseudoduganella violaceinigra
KEEPYTGYKAVSASHVEGKAAIWIRENGSSGGTVYHNNTTGTCGYCNSQVKALLPEGVELKIVPPTNAVAKNAQARAVPTINVGNGTQPGRKQK

SEQ ID NO: 197 - protein, 118 aa, Pseudoduganella violaceinigra
GSSGKNVRMPRDYASELPEYDGKTTHGVLVTNEGKVIQLRSGGKEEPYTGYKAVSASHVEGKAAIWIRENGSSGGTVYHNNTTGTCGYCNSQVKALLPEGVELKIVPPTNAVAKNAQA

SEQ ID NO: 198 - protein, 19 aa, Pseudoduganella violaceinigra
RAVPTINVGNGTQPGRKQK

SEQ ID NO: 199 - protein, 136 aa, Duganella sp.
GSSGKNVRLPRDYASELPEYDGKTTYGVLVTNEGKVIQLRSGGKEVPYSGYKAVSASHVEGKAAIWIRENASSGGTVYHNNTTGTCGYCNSQVKALLPEGVELKIVPPANAVARNSQAKAIPTINVGNATQPGRKP

SEQ ID NO: 200 - protein, 43 aa, Duganella sp.
GSSGKNVRLPRDYASELPEYDGKTTYGVLVTNEGKVIQLRSGG

SEQ ID NO: 201 - protein, 93 aa, Duganella sp.
KEVPYSGYKAVSASHVEGKAAIWIRENASSGGTVYHNNTTGTCGYCNSQVKALLPEGVELKIVPPANAVARNSQAKAIPTINVGNATQPGRKP

SEQ ID NO: 202 - protein, 118 aa, Duganella sp.
GSSGKNVRLPRDYASELPEYDGKTTYGVLVTNEGKVIQLRSGGKEVPYSGYKAVSASHVEGKAAIWIRENASSGGTVYHNNTTGTCGYCNSQVKALLPEGVELKIVPPANAVARNSQA

SEQ ID NO: 203 - protein, 18 aa, Duganella sp.
KAIPTINVGNATQPGRKP

SEQ ID NO: 204 - protein, 132 aa, Chondromyces apiculatus
PDPPPPPTPMGNTLPGWDGGKTQGWFVYPDGTERHLISGYDGPSKFTQGIPGMNGNIKSHVEAHAAALMRQYELSKATLYINRVPCPGVRGCDALLARMLPEGVQLEIIGPNGFKKTYTGLPDPKLKPKGCS

SEQ ID NO: 205 - protein, 42 aa, Chondromyces apiculatus
PDPPPPPTPMGNTLPGWDGGKTQGWFVYPDGTERHLISGYDG

SEQ ID NO: 206 - protein, 90 aa, Chondromyces apiculatus
PSKFTQGIPGMNGNIKSHVEAHAAALMRQYELSKATLYINRVPCPGVRGCDALLARMLPEGVQLEIIGPNGFKKTYTGLPDPKLKPKGCS

SEQ ID NO: 207 - protein, 120 aa, Chondromyces apiculatus
PDPPPPPTPMGNTLPGWDGGKTQGWFVYPDGTERHLISGYDGPSKFTQGIPGMNGNIKSHVEAHAAALMRQYELSKATLYINRVPCPGVRGCDALLARMLPEGVQLEIIGPNGFKKTYTG

SEQ ID NO: 208 - protein, 12 aa, Chondromyces apiculatus
LPDPKLKPKGCS

SEQ ID NO: 209 - protein, 140 aa, Sorangium cellulosum
GAATVFGAGRGLGALEEATTAAGIARGAPSLPVYTGGKTTGVLRTATGDMPLVSGYKGPSASMPRGTPGMNGRIKSHVEAHAAAVMRERGIKDATLHINQVPCSSATGCGAMLPRMLPEGAQLRVLGPDGYDQVFIGLPD

SEQ ID NO: 210 - protein, 58 aa, Sorangium cellulosum
GAATVFGAGRGLGALEEATTAAGIARGAPSLPVYTGGKTTGVLRTATGDMPLVSGYKG

SEQ ID NO: 211 - protein, 82 aa, Sorangium cellulosum
PSASMPRGTPGMNGRIKSHVEAHAAAVMRERGIKDATLHINQVPCSSATGCGAMLPRMLPEGAQLRVLGPDGYDQVFIGLPD

SEQ ID NO: 212 - protein, 127 aa, Sorangium cellulosum
GAATVFGAGRGLGALEEATTAAGIARGAPSLPVYTGGKTTGVLRTATGDMPLVSGYKGPSASMPRGTPGMNGRIKSHVEAHAAAVMRERGIKDATLHINQVPCSSATGCGAMLPRMLPEGAQLRVLG

SEQ ID NO: 213 - protein, 13 aa, Sorangium cellulosum
PDGYDQVFIGLPD

SEQ ID NO: 214 - protein, 127 aa, Clostridium sp.
TNIIDNRPKLPDYDGKTTHGILVTPNSEHIPFSSGNPNPNYKNYIPASHVEGKSAIYMRENGITSGTIYYNNTDGTCPYCDKMLSTLLEEGSVLEVIPPINAKAPKPSWVDKPKTYIGNNKVPKPNK

SEQ ID NO: 215 - protein, 35 aa, Clostridium sp.
TNIIDNRPKLPDYDGKTTHGILVTPNSEHIPFSSG

SEQ ID NO: 216 - protein, 92 aa, Clostridium sp.
NPNPNYKNYIPASHVEGKSAIYMRENGITSGTIYYNNTDGTCPYCDKMLSTLLEEGSVLEVIPPINAKAPKPSWVDKPKTYIGNNKVPKPNK

SEQ ID NO: 217 - protein, 104 aa, Clostridium sp.
TNIIDNRPKLPDYDGKTTHGILVTPNSEHIPFSSGNPNPNYKNYIPASHVEGKSAIYMRENGITSGTIYYNNTDGTCPYCDKMLSTLLEEGSVLEVIPPINAKA

SEQ ID NO: 218 - protein, 23 aa, Clostridium sp.
PKPSWVDKPKTYIGNNKVPKPNK

SEQ ID NO: 219 - protein, 142 aa, Unknown
  source: /note=“Description of Unknown Chloroflexia bacterium sequence”
AGCPGDALPPYGTKGSKTTGILDTGNESILLESGENGPGMMVPRDTPGMSGAMPNRAHVEGHTAAIMRNENIRLADLYINRMPCSGAYGCMVNLPHMLPEGSILRIHVRAKLSDPWTTLPPFVGISDTLWPPSGLNPKIVLP

SEQ ID NO: 220 - protein, 37 aa, Unknown
  source: /note=“Description of Unknown Chloroflexia bacterium sequence”
AGCPGDALPPYGTKGSKTTGILDTGNESILLESGENG

SEQ ID NO: 221 - protein, 105 aa, Unknown
  source: /note=“Description of Unknown Chloroflexia bacterium sequence”
PGMMVPRDTPGMSGAMPNRAHVEGHTAAIMRNENIRLADLYINRMPCSGAYGCMVNLPHMLPEGSILRIHVRAKLSDPWTTLPPFVGISDTLWPPSGLNPKIVLP

SEQ ID NO: 222 - protein, 18 aa, Unknown
  source: /note=“Description of Unknown Chloroflexia bacterium sequence”
ISDTLWPPSGLNPKIVLP

SEQ ID NO: 223 - protein, 124 aa, Unknown
  source: /note=“Description of Unknown Chloroflexia bacterium sequence”
AGCPGDALPPYGTKGSKTTGILDTGNESILLESGENGPGMMVPRDTPGMSGAMPNRAHVEGHTAAIMRNENIRLADLYINRMPCSGAYGCMVNLPHMLPEGSILRIHVRAKLSDPWTTLPPFVG

SEQ ID NO: 224 - DNA, 45 bp, Artificial Sequence
  source: /note=“Description of Artificial Sequence Synthetic primer”
  modified_base (21)..(24): a, c, t, g, unknown or other
acacgacgctcttccgatctnnnnctggggcagctgatcacatgt

SEQ ID NO: 225 - DNA, 39 bp, Artificial Sequence
  source: /note=“Description of Artificial Sequence Synthetic primer”
gacgtgtgctcttccgatctcttccatcccctccccaag

SEQ ID NO: 226 - DNA, 141 bp, Artificial Sequence
  source: /note=“Description of Artificial Sequence Synthetic polynucleotide”
  modified_base (1)..(4): a, c, t, g, unknown or other
nnnnctggggcagctgatcacatgttttctctgcagccttcccagaggagcttccggcagacctgaagcactggaagccaggtgtgcagggcaggtgggctggggttgggaagggtggatgccttggggaggggatggaag

SEQ ID NO: 227 - DNA, 68 bp, Homo sapiens
tcttcattacacctgcagctctcattttccatacagtcagtatcaattctggaagaatttccagacat

SEQ ID NO: 228 - DNA, 68 bp, Homo sapiens
atgtctggaaattcttccagaattgatactgactgtatggaaaatgagagctgcaggtgtaatgaaga

SEQ ID NO: 229 - DNA, 59 bp, Homo sapiens
tacacctgcagctctcattttccatacagtcagtatcaattctggaagaatttccagac

SEQ ID NO: 230 - DNA, 59 bp, Homo sapiens
gtctggaaattcttccagaattgatactgactgtatggaaaatgagagctgcaggtgta

SEQ ID NO: 231 - DNA, 53 bp, Homo sapiens
tgcagctctcattttccatacagtcagtatcaattctggaagaatttccagac

SEQ ID NO: 232 - DNA, 53 bp, Homo sapiens
gtctggaaattcttccagaattgatactgactgtatggaaaatgagagctgca

SEQ ID NO: 233 - DNA, 39 bp, Homo sapiens
tacacctgcagctctcattttccatacagtcagtatcaa

SEQ ID NO: 234 - DNA, 39 bp, Homo sapiens
ttgatactgactgtatggaaaatgagagctgcaggtgta

SEQ ID NO: 235 - DNA, 111 bp, Homo sapiens
ctttaccagatctcaaaaagaaggtcttcattacacctgcagctctcattttccatacagtcagtatcaattctggaagaatttccagacattaaagatagtcatcttggg

SEQ ID NO: 236 - DNA, 111 bp, Homo sapiens
cccaagatgactatctttaatgtctggaaattcttccagaattgatactgactgtatggaaaatgagagctgcaggtgtaatgaagaccttctttttgagatctggtaaag

SEQ ID NO: 237 - DNA, 81 bp, Homo sapiens
tacacctgcagctctcattttccatacagtcagtatcaattctggaagaatttccagacattaaagatagtcatcttgggg

SEQ ID NO: 238 - DNA, 81 bp, Homo sapiens
ccccaagatgactatctttaatgtctggaaattcttccagaattgatactgactgtatggaaaatgagagctgcaggtgta

SEQ ID NO: 239 - DNA, 60 bp, Homo sapiens
tcccagaggagcttccggcagacctgaagcactggaagccaggtgtgcagggcaggtggg

SEQ ID NO: 240 - DNA, 60 bp, Homo sapiens
cccacctgccctgcacacctggcttccagtgcttcaggtctgccggaagctcctctggga

SEQ ID NO: 241 - protein, 172 aa, Artificial Sequence
  source: /note=“Description of Artificial Sequence Synthetic polypeptide”
ERPFQCRICMRNFSRSAHLSRHIRTHTGEKPFACDICGRKFATSGHLSRHTKIHTHPRAPIPKPFQCRICMRNFSDSSHRTRHIRTHTGEKPFACDICGRKFAAKWNLDAHTKIHTGSQKPFQCRICMRNFSRPYTLRLHIRTHTGEKPFACDICGRKFALRHHLTRHTKIH

SEQ ID NO: 242 - protein, 168 aa, Artificial Sequence
  source: /note=“Description of Artificial Sequence Synthetic polypeptide”
ERPFQCRICMRNFSQSGHLARHIRTHTGEKPFACDICGRKFARKWTLQGHTKIHTGSQKPFQCRICMRNFSIRSTLRDHIRTHTGEKPFACDICGRKFAHRSSLRRHTKIHTGSQKPFQCRICMRNFSQSGNLARHIRTHTGEKPFACDICGRKFARNVDLIHHTKIH

SEQ ID NO: 243 - protein, 168 aa, Artificial Sequence
  source: /note=“Description of Artificial Sequence Synthetic polypeptide”
ERPFQCRICMRNFSIRSTLRDHIRTHTGEKPFACDICGRKFAHRSSLRRHTKIHTGSQKPFQCRICMRNFSQSGNLARHIRTHTGEKPFACDICGRKFARNVDLIHHTKIHTGSQKPFQCRICMRNFSRSDVLSEHIRTHTGEKPFACDICGRKFATSGHLSRHTKIH

SEQ ID NO: 244 - DNA, 137 bp, Homo sapiens
ctggggcagctgatcacatgttttctctgcagccttcccagaggagcttccggcagacctgaagcactggaagccaggtgtgcagggcaggtgggctggggttgggaagggtggatgccttggggaggggatggaag

SEQ ID NO: 245 - protein, 4 aa, Artificial Sequence
  source: /note=“Description of Artificial Sequence Synthetic peptide”
SGGS